(19) United States
(12) Patent Application Publication
(10) Pub. No.: US 2024/0417781 A1
(43) Pub. Date: Dec. 19, 2024

(54) HIGH RESOLUTION MULTIPLEX METHOD FOR DETECTING AT LEAST TWO TARGETS WITH A DISTANCE OF BEYOND THE DIFFRACTION LIMIT IN A SAMPLE

(71) Applicant: Resolve BioSciences GmbH, Monheim am Rhein (DE)

(72) Inventors: Cheng Frank Zhong, Monheim am Rhein (DE); Christian Korfhage, Monheim am Rhein (DE); Frank Reinecke, Monheim am Rhein (DE); Stephan Tirier, Monheim am Rhein (DE)

(73) Assignee: Resolve BioSciences GmbH, Monheim am Rhein (DE)

(21) Appl. No.: 18/743,591

(22) Filed: Jun. 14, 2024

Related U.S. Application Data
(60) Provisional application No. 63/508,682, filed on Jun. 16, 2023.

(51) Int. Cl. C12Q 1/6823; C12Q 1/6804; C12Q 1/6876; G01N 21/64

(52) U.S. Cl. CPC C12Q 1/6823; C12Q 1/6804; C12Q 1/6876; G01N 21/6486; C12Q 2600/156

(57) ABSTRACT

The technology provided herein relates to high resolution multiplex methods and kits for detecting different analytes in a sample by sequential signal-encoding of said analytes, wherein the method allows a differentiation of targets which distance is below the diffraction limit of optical microscopes, i.e. targets with spatial optical overlap. The disclosed methods also include in vitro methods for screening, identifying and/or testing a substance and/or drug and in vitro methods for diagnosis of a disease, and an optical multiplexing system.

### CROSS REFERENCE TO RELATED APPLICATIONS

The present application claims the benefit of priority to U.S. Prov Ser. No. 63/508,682, filed Jun. 16, 2023, the disclosure of which is hereby incorporated by reference in its entirety.

### SEQUENCE LISTING

This application contains a Sequence Listing in computer readable form entitled RES-PA09-PCT_Seqlisting.xml generated Jun. 16, 2023, having a size of about 275 kb. The computer readable form is incorporated herein by reference in its entirety.

In the accompanying sequence listing SEQ ID Nos. 1-1247 refer to nucleotide sequences of exemplary target-specific oligonucleotides. The oligonucleotides listed consist of a target specific binding site (5′-end) a spacer/linker sequence (gtaac or tagac) and the unique identifier sequence, which is the same for all oligonucleotides of one probe set.

In the accompanying sequence listing SEQ ID Nos. 1248-1397 refer to nucleotide sequences of exemplary decoding oligonucleotides.

In the accompanying sequence listing SEQ ID Nos. 1398-1400 refer to the nucleotide sequences of exemplary signal oligonucleotides. For each signal oligonucleotide the corresponding fluorophore is present twice. One fluorophore is covalently linked to the 5′-end and one fluorophore is covalently linked to the 3′-end. SEQ ID No. 1398 comprises at its 5′ terminus “5Alex488N”, and at its 3′ terminus “3AlexF488N”. SEQ ID No. 1399 comprises at its 5′ terminus “5Alex546”, and at its 3′ terminus 3Alex546N. SEQ ID No. 1400 comprises at its 5′ terminus and at its 3′ terminus “Atto594”.

### FIELD OF THE DISCLOSURE

The technology provided herein relates to high resolution multiplex methods and kits for detecting different analytes in a sample by sequential signal-encoding of said analytes, wherein the method allows a differentiation of targets which distance is below the diffraction limit of optical microscopes, i.e. targets with spatial optical overlap. The disclosed methods also include in vitro methods for screening, identifying and/or testing a substance and/or drug and in vitro methods for diagnosis of a disease, and an optical multiplexing system. The technology provided herein further relates to methods and kits for detecting a polymorphic analyte in a sample by specific signal-encoding of said polymorphic analyte. The method allows to detect targets which are polymorphic, modified, or allelic variants of a known or intended target. Some such allelic variants or other variants are nucleic acids, such as those that show a certain variability such as local variability in their nucleic acid sequence of between 85% to 99% sequence identity relative to a known or intended target. Often this variation is localized, such as may result from a differentially retained or excised exon in one splice variant transcript relative to an expected target, or a differentially modified base or amino acid residue. Therefore the technology allows the detection of for example polymorphic genetic loci or transcripts in samples originating from different sources (e.g. different subjects, different organs, different cell types, different species, benign and malign forms, etc.) with the same set of probes, rather than requiring a cell type, transcript type, allele type, post-transcriptional or post-translational modification-type or other specific probe set. As such the disclosed methods are more versatile and robust against disruptive factors as compared to conventional methods. The disclosed methods also include in vitro methods for screening, identifying and/or testing a substance and/or drug and in vitro methods for diagnosis of a disease, and optical systems consistent with use of these durable probe sets.

### BACKGROUND

The analysis and detection of small quantities of analytes in biological and non-biological samples has become a routine practice in the clinical and analytical environment. Numerous analytical methods have been established for this purpose. Some of them use encoding techniques assigning a particular readable code to a specific first analyte which differs from a code assigned to a specific second analyte.

One of the prior art techniques in this field is the so-called ‘single molecule fluorescence in situ hybridization’ (smFISH) essentially developed to detect mRNA molecules in a sample. In Lubeck et al. (2014), Single cell in situ RNA profiling by sequential hybridization, Nat. Methods11 (4), p. 360-361, the mRNAs of interest are detected via specific directly labeled probe sets. After one round of hybridization and detection, the set of mRNA specific probes is eluted from the mRNAs and the same set of probes with other (or the same) fluorescent labels is used in the next round of hybridization and imaging to generate gene specific color-code schemes over several rounds. The technology needs several differently tagged probe sets per transcript and needs to denature these probe sets after every detection round. Thus, the approach is both costly and harsh to the sample target.

A further development of this technology does not use directly labeled probe sets. Instead, the oligonucleotides of the probe sets provide nucleic acid sequences that serve as initiator for hybridization chain reactions (HCR), a technology that enables signal amplification; see Shah et al. (2016), In situ transcription profiling of single cells reveals spatial organization of cells in the mouse hippocampus, Neuron 92 (2), p. 342-357.

Another technique referred to as ‘multiplexed error robust fluorescence in situ hybridization’ (merFISH) is described by Chen et al. (2015), RNA imaging. Spatially resolved, highly multiplexed RNA profiling in single cells, Science 348 (6233): aaa6090. There, the mRNAs of interest are detected via specific probe sets that provide additional sequence elements for the subsequent specific hybridization of fluorescently labeled oligonucleotides. Each probe set provides four different sequence elements out of a total of 16 sequence elements. After hybridization of the specific probe sets to the mRNAs of interest, the so-called readout hybridizations are performed. In each readout hybridization one out of the 16 fluorescently labeled oligonucleotides complementary to one of the sequence elements is hybridized. All readout oligonucleotides use the same fluorescent color. After imaging, the fluorescent signals are destroyed via illumination and the next round of readout hybridization takes place without a denaturing step. As a result, a binary code is generated for each mRNA species. A unique signal signature of 4 signals in 16 rounds is created using only a single hybridization round for binding of specific probe sets to the mRNAs of interest, followed by 16 rounds of hybridization of readout oligonucleotides labeled by a single fluorescence color. A limit to this system is that the readout pattern associated with a target is limited by the number of sites to which the fluorescently labeled oligonucleotides may bind. As the labeled oligonucleotides are not removed, the labeled oligo binding portion of the target probe is gradually more occupied throughout the unique signal generation process. Accordingly, the number of unique signals that can be generated from a set of concurrently used target probes is limited, and accordingly the number of unique targets that may be assayed in a single reaction is limited as well.

A further development of this technology improves the throughput by using two different fluorescent colors, eliminating the signals via disulfide cleavage between the readout-oligonucleotides and the fluorescent label and an alternative hybridization buffer; see Moffitt et al. (2016), High-throughput single-cell gene-expression profiling with multiplexed error-robust fluorescence in situ hybridization, Proc. Natl. Acad. Sci. USA. 113 (39), p. 11046-11051. However, even using two colors for unique pattern generation, there is still an upper limit to the number of targets that may be assayed in a single reaction.

Xia et al. (2019) (https://doi.org/10.1073/pnas.1912459116) further increased the gene throughput of MERFISH and achieved 10,000 plex using 23 rounds of hybridization and 3 color channels. To reduce the impact of crowding and diffraction limited spots, this version of MERFISH uses expansion microscopy to increase the voxel space in which individual transcripts can be detected.

A technology referred to as ‘intron seqFISH’ is described in Shah et al. (2018), Dynamics and spatial genomics of the nascent transcriptome by intron seqFISH, Cell 117 (2), p. 363-376. There, the mRNAs of interest are detected via specific probe sets that provide additional sequence elements for the subsequent specific hybridization of fluorescently labeled oligonucleotides. Each probe set provides one out of 12 possible sequence elements (representing the 12 ‘pseudocolors’ used) per color-coding round. Each color-coding round consists of four serial hybridizations. In each of these serial hybridizations, three readout probes, each labeled with a different fluorophore, are hybridized to the corresponding elements of the mRNA-specific probe sets. After imaging, the readout probes are stripped off by a 55% formamide buffer and the next hybridization follows. After 5 color-coding rounds with 4 serial hybridizations each, the color-codes are completed.

A further development of this technology, termed ‘seqFISH+’ (https://doi.org/10.1038/s41586-019-1049-y), used the same principle of pseudocolors, but encodes individual transcripts in one of three color channels separately to eliminate chromatic aberrations. To reduce the impact of crowding and diffraction limited spots, seqFISH+dilutes signals into 4 color coding rounds with 20 serial hybridizations each in combination with subpixel localization of spots. Thereby, seqFISH+achieves 10,000plex smRNA-FISH, but with very high false positive rates (FPR=0.22).

Another technology, CosMX™ SMI, (He et al., 2022) (https://doi.org/10.1038/s41587-022-01483-z) utilizes a limited number (˜5) of up to 130 nt long target probes that contain a target binding domain complementary the transcript of interest and four readout domains that bind fluorescent reporters. Reporters consist of branched DNA structures that contain up to 60 fluorophores, thereby complementing for the low number of target probes. Each target probe contains a unique set of four different sequence elements out of a total of 64 sequence elements (16 sequence elements*4 colors).

Individual transcripts are binary encoded using a 64-bit barcode and 4 signals per transcript (one per color) over 16 rounds of hybridization. Like the seqFISH technology, CosMX™ uses subpixel localization of spots enabled by the dilution of signals to reduce the impact of crowding and diffraction limited spots.

EP 0 611 828 discloses the use of a bridging element to recruit a signal generating element to probes that specifically bind to an analyte. A more specific statement describes the detection of nucleic acids via specific probes that recruit a bridging nucleic acid molecule. This bridging nucleic acids eventually recruit signal generating nucleic acids. This document also describes the use of a bridging element with more than one binding site for the signal generating element for signal amplification like branched DNA.

Player et al. (2001), Single-copy gene detection using branched DNA (bDNA) in situ hybridization, J. Histochem. Cytochem. 49 (5), p. 603-611, describe a method where the nucleic acids of interest are detected via specific probe sets providing an additional sequence element. In a second step, a preamplifier oligonucleotide is hybridized to this sequence element. This preamplifier oligonucleotide comprises multiple binding sites for amplifier oligonucleotides that are hybridized in a subsequent step. These amplifier oligonucleotides provide multiple sequence elements for the labeled oligonucleotides. This way a branched oligonucleotide tree is built up that leads to an amplification of the signal.

A further development of this method referred to as is described by Wang et al. (2012), RNAscope: a novel in situ RNA analysis platform for formalin-fixed, paraffin-embedded tissues, J. Mol. Diagn. 14 (1), p.22-29, which uses another design of the mRNA-specific probes. Here two of the mRNA-specific oligonucleotides have to hybridize in close proximity to provide a sequence that can recruit the preamplifier oligonucleotide. This way the specificity of the method is increased by reducing the number of false positive signals.

Choi et al. (2010), Programmable in situ amplification for multiplexed imaging of mRNA expression, Nat. Biotechnol. 28 (11), p. 1208-1212, disclose a method known as ‘HCR-hybridization chain reaction’. The mRNAs of interest are detected via specific probe sets that provide an additional sequence element. The additional sequence element is an initiator sequence to start the hybridization chain reaction. Basically, the hybridization chain reaction is based on metastable oligonucleotide hairpins that self-assemble into polymers after a first hairpin is opened via the initiator sequence.

A further development of the technology uses so called split initiator probes that have to hybridize in close proximity to form the initiator sequence for HCR, similarly to the RNAscope technology, this reduces the number of false positive signals; see Choi et al. (2018), Third-generation in situ hybridization chain reaction: multiplexed, quantitative, sensitive, versatile, robust. Development 145 (12).

Mateo et al. (2019), Visualizing DNA folding and RNA in embryos at single-cell resolution, Nature Vol, 568, p. 49ff., disclose a method called ‘optical reconstruction of chromatin structure (ORCA). This method is intended to make the chromosome line visible.

EP 2 992 115 B1 describes a method of sequential single molecule hybridization and provides technologies for detecting and/or quantifying nucleic acids in cells, tissues, organs or organisms through sequential barcoding.

The methods known in the art, however, have numerous disadvantages. In particular, they are inflexible, expensive, complex, time consuming and quite often provide non-accurate results. In particular, the encoding capacities of the existing methods are low and do not meet the requirements of modern molecular biology and medicine.

Furthermore, the methods known so far do not provide a reliable signal in cases in which the target, for example spatially overlapping transcripts, cannot be differentiated by optical methods due to the diffraction limit, i.e. the minimal distance a which two signal spots can be differentiated in a microscope. However, if the detection of spots is limited by the resolution of the microscope, it is not possible to identify transcripts that are very close to each other. This is e.g. relevant for detection of fusion genes (cancer) or for detection of co-localization of different transcript types (e.g. transcriptional hubs in the nucleus). In addition, transcripts with higher expression levels are also problematic because the high number of signals affects the detection of other (especially lowly expressed) genes.

Many of the methods known in the art, however, have numerous disadvantages. In particular, they are often inflexible, expensive, complex, time-consuming and quite often provide non-accurate results.

In particular, the encoding capacities of the existing methods are low, such that the number of targets to be assayed by a single probe set is low relative to the total transcript, proteome or locus diversity of a sample. Thus, they struggle to meet the demands of modern molecular biology and medicine with regard to capturing the total amount of information available from a sample.

Furthermore, many of the methods in use or known in the art often rely upon a single probe or binding site per target. These approaches achieve specificity of target binding, but they do not provide a reliable signal in cases in which the target is polymorphic, e.g., differs in a few nucleotides, for example because of genetic variations, SNPs, variable intron-sequences, different gene loci, variable transcripts, differential expression, differences between cells, organs, subjects, or species, for example. As a result, many of the methods in use may under-perform when used on a sample which may differ from what is expected in target nucleic acid or amino acid sequence. This type of difference is often the case among individuals from groups whose genomic information is under-represented, or which differ from an expected genome in ways which have not been assayed. Using a method that cannot account or adjust for these differences, individuals whose genomic diversity is underrepresented in databases from which probes are generated are likely to be underserved by these single-target binder assays, and the clinical advice that arises from these assays is more likely to fail these individuals. Similarly, when probing for a highly variant target such as a retroviral transcript or genomic target, the sheer amount of variation within a population of viral or other hypervariable target genomes is likely to impede a true measurement of their presence in the sample.

This is particularly relevant for detecting a large number of different samples from different origins in a reliable manner. For example, a screen for breast cancer in blood-samples from different female patients can only be analyzed in a high-throughput manner, if the probes used facilitate the same robust signal independent of individual variations. Other prominent examples include mass-screenings for contagious diseases in a population during a pandemic or stratification of patient-groups for the dosage and use of certain medicaments based on a certain genetic profile as is often used in companion diagnostics.

Against this background, it is an object underlying the present disclosure to provide a method by means of which the disadvantages of the prior art methods can be reduced or even overcome.

### SUMMARY OF THE DISCLOSURE

The present disclosure pertains to novel high resolution multiplex methods and kits for detecting different analytes in a sample beyond the diffraction limit by sequential signal-encoding of said analytes.

In a first aspect two analytic sets are combined in one method to be applied on the same tissue section. The method is generally characterized by the following steps
i. Hybridization of target probes for both analytic sets simultaneously before the first run.
ii. Tails of the target probes are unique for both sets, e.g., 300 tails (for 300 transcripts) are used for the first analytic set and 25 tails (for 25 transcripts) for the second analytic set. This requires also different decoder sets for both sets.
iii. Both runs generate independent datasets that can be combined in silico afterwards.

Thereby, it was surprisingly found that the multiplexing capability could be enhanced without increasing optical crowding and spatial overlapping transcripts could be detected which otherwise would be invisible due to the diffraction limit of the microscope.

In a further aspect, embodiments of the disclosure pertain in particular to a multiplex method for detecting different analytes in a sample beyond the diffraction limit by sequential signal-encoding of said analytes, comprising the steps of:
(A1) contacting the sample with a first set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(A2) contacting the sample with a second set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
wherein (optionally) the number of probes and/or targets of first set of analyte-specific probes according to step A1 (i.e. the transcript plexity of A1) is at least 10 times higher than the number of probes and/or targets of the second set of analyte-specific probes according to step A2 (i.e. the transcript plexity of A2); and
(B1) contacting the sample with at least a first set of decoding oligonucleotides per analyte, wherein in each set of decoding oligonucleotides for an individual analyte each decoding oligonucleotide of the for the first set of analyte-specific probes according to step A1 comprises:
(aa) an identifier connector element (t) comprising a nucleotide sequence which is essentially complementary to at least a section of the unique identifier sequence of the identifier element (T) of the corresponding analyte-specific probe set A1, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t); and
(B2) contacting the sample with at least a second set of decoding oligonucleotides per analyte, wherein in each set of decoding oligonucleotides for an individual analyte of each decoding oligonucleotide for the second set of analyte-specific probes according to step A2 comprises:
(aa) an identifier connector element (t) comprising a nucleotide sequence which is essentially complementary to at least a section of the unique identifier sequence of the identifier element (T) of the corresponding analyte-specific probe set A2, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the first connect element (t);
(C) contacting the sample with at least a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.
(D) Detecting the signal caused by the signal element;
(E) selectively removing the decoding oligonucleotides and signal oligonucleotides from the sample, thereby essentially maintaining the specific binding of the analyte-specific probes to the analytes to be encoded;
(F) Performing at least three (3) further cycles comprising steps B) to E) to generate an encoding scheme with a code word per analyte, wherein in particular the last cycle may stop with step (D).

In yet a further aspect, embodiments of this disclosure relate to kits for multiplex analyte encoding beyond the diffraction limit, comprising
(A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
(A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising
(aa) a binding element(S) that specifically interacts with one of the different analytes to be encoded, and
(bb) an identifier element (T) comprising a nucleotide sequence which is unique to the analyte to be encoded (unique identifier sequence),
wherein the analyte-specific probes of a particular set of analyte-specific probes differ from the analyte-specific probes of another set of analyte-specific probes in the nucleotide sequence of the identifier element (T),
wherein the analyte-specific probes in each set of analyte-specific probes binds to the same analyte and comprises the same nucleotide sequence of the identifier element (T) which is unique to said analyte; and
wherein the number of probes and/or targets of first set of analyte-specific probes according to step A1 (i.e. the transcript plexity of A1) is at least 10 times higher than the number of probes and/or targets of the second set of analyte-specific probes according to step A2 (i.e. the transcript plexity of A2); and
(B) at least one set of decoding oligonucleotides per analyte set A1 and A2, wherein in each set of decoding oligonucleotides for an individual analyte each decoding oligonucleotide comprises:
(aa) an identifier connector element (t) comprising a nucleotide sequence which is essentially complementary to at least a section of the unique identifier sequence of the identifier element (T) of the corresponding analyte-specific probe set, and
(bb) a translator element (c) comprising a nucleotide sequence allowing a specific hybridization of a signal oligonucleotide;
wherein the decoding oligonucleotides of a set for an individual analyte differ from the decoding oligonucleotides of another set for a different analyte in the identifier connect element (t); and
(C) a set of signal oligonucleotides, each signal oligonucleotide comprising:
(aa) a translator connector element (C) comprising a nucleotide sequence which is essentially complementary to at least a section of the nucleotide sequence of a translator element (c) comprised in a decoding oligonucleotide, and
(bb) a signal element.

In a third aspect, embodiments of this disclosure relate to in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure.

In a fourth aspect, embodiments of this disclosure provide in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the multiplex method according to the present disclosure.

In a fifth aspect, some embodiments of this disclosure relate to optical multiplexing systems suitable for the method according to the present disclosure, comprising: at least one reaction vessel for containing the kits or part of the kits according to any one of the claim; a detection unit comprising a microscope, in particular a fluorescence microscope; a camera; a liquid handling device.

In a sixth aspect, some embodiments provide in vitro methods for screening, identifying and/or testing a substance and/or drug comprising:
(a) contacting a test sample comprising a sample with a substance and/or drug
(b) detecting different analytes in a sample by sequential signal-encoding of said analytes with a method according to the present disclosure.

In a seventh aspect, the methods according to the present disclosure are used for the co-localization of different analytes, in particular of analytes of different molecular groups such as RNA and protein, or RNA and DNA, or DNA and protein, or two different RNA molecules, or two different DNA sequences, or two different proteins.

Furthermore, in an eight aspect the methods according to the present disclosure are used for the co-localization of at least two different features of a single molecule, in particular of two parts of a nucleic acid molecule (differential splicing, fusion transcripts, fusion genes; and/or two parts of a protein or a dimerized protein.

In a further aspect, the methods according to the present disclosure are used for the Co-localization of abundant and rare transcripts (to minimize loss in sensitivity induced by the abundant transcript).

According to the present disclosure, unique tags (identifier) are used per target (e.g. mRNA of one single gene) or for a target group. Groups can be formed to be indicative for a certain identity, process, biological function, or disease (examples: cell type, inflammation, signal processing, cancer).

Importantly, the methods and kits according to the present disclosure leads to a reduction of complexity. Many different probes with different binding sequences share the same (one per target) unique tag. These tags have reduced the sequence complexity (to one per target) and also have predetermined constant properties (e.g., thermodynamic stability).

Advantages of the methods and kits according to the present disclosure are:
a) Full flexibility of the process to determine the identity of the tag, e.g. use more or less signals and/or rounds, varying numbers of fluorophores, number of total signals per tag→lower numbers of targets (e.g. 20) can be identified with high confidence in less rounds (e.g. 4) than a large number of targets (e.g. 100, these need 8 rounds for the same level of confidence), even if in both cases the exact same unique tags are used.
b) All unique tags are used (recycled) in many consecutive rounds of hybridization and all primary probes contribute (provide information about their identity) in every round of identification.
c) As all tags share the same predefined properties (e.g. thermodynamic stability which allows for selective denaturing).

In some advantageous embodiments, the unique tags are design as follow:
No cross-hybridization between all oligonucleotides of the process (probes, decoders, readout), so that all tag sequences are usable together (compatible)
No cross-hybridization between connector elements (bridges) of different unique tags
Stability of hybridization of the unique tags should be in a narrow range: as stable as possible (fast hybridization, i.e., short cycle times) but significantly different (in this case less stable) than the primary probe (for differential denaturation, without removing primary probes)

Therefore, the present description pertains in particular to the usage of a set of labeled and unlabeled nucleic acid sequences for specific quantitative and/or spatial detection of different analytes via specific hybridization. The technology allows the discrimination of more different analytes than different detection signals are available. The discrimination is realized via sequential signal-coding of the analytes achieved by several cycles of specific hybridization, detection of signals and selective elution of the hybridized nucleic acid sequences. In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds. The utilization of decoding-oligonucleotides leads to a sequential signal-coding technology that is e.g., more flexible, cheaper, simpler, faster and/or more accurate than other methods.

Furthermore, the present disclosure pertains to the use of improved decoding-oligonucleotides to increase the efficiency of the encoding scheme. The so called “multi-decoders” allows the recruiting of more than just one signal oligonucleotide and therefore can generate new signal types by utilizing the combination of two or more different signal-oligonucleotides without decreasing the brightness of the signals.

Furthermore, due to the use of a first set of analyte-specific probes according to step A1 (i.e. the transcript plexity of A1) which can be at least 10 times higher in numbers than the number of probes and/or targets of the second set of analyte-specific probes according to step A2 (i.e. the transcript plexity of A2) as well as the use of at least two different sets of decoding oligonucleotides for set A1 and set A2, spatially overlapping targets inside a tissue and or cell culture sample which distance is beyond the diffraction limit can be detected. Further advantages pertain to an improvement of the overall signal to noise ratio, the signal spread (such as signals of transcripts with higher expression levels can be detected together with lowly expressed genes) and the multiplexing capability without increasing optical crowding.

The present disclosure pertains to novel methods and kits for the resilient detection of an unknown variant of a polymorphic analyte in a sample by specific signal-encoding of said analyte.

Pursuant to this resilient detection, disclosed herein are compositions, kits, methods and systems related to the redundant labeling and detection of an analyte such as a protein or a nucleic acid, for example a genomic locus or a transcript. Through practice of the methods or use of the compositions, kits or systems herein, an analyte is multiply, independently tagged with a common identifier element that is independently delivered by a plurality of probes that independently target the analyte to independently and redundantly deliver the common identifier element to the analyte. Consequently, upon contacting a sample comprising a target analyte to a population of probes that are independently binding but tagged with a common identifier element, one generates a target analyte that is redundantly, multiply tagged by the identifier element. Often, the identifier element is a barcode or analyte specific or analyte identifying oligonucleotide, so as to facilitate downstream localization of multiple copies of a decoder oligonucleotide to the target analyte. Decoder oligonucleotides are often at least bi-partite, comprising a region that anneals to an identifier element, and a second region that anneals to a signal element. Upon binding of a population of decoding oligonucleotides to the identifier element, one coats the analyte with a plurality of signal element binding sites, to which a plurality of signal elements may bind. Upon further addition of a plurality of signal element probes, one generates target analytes that are multiply labeled by the signal element probes, facilitating detection of the analyte in a sample.

The methods, compositions and systems herein exhibit a number of features that facilitate analyte detection. Firstly, the plurality of probes that bind the analyte directly share a common identifier element but do not use common analyte binding elements. Consequently, probes of a plurality of probes that target an analyte act independently to deliver the identifier element to the analyte. The identifier element identifies the analyte target, but does not identify any individual probe, as the probes of an analyte binding set are commonly labeled using the same identifier element. Thus, the delivery of identifier elements to a target analyte is resilient to modest or local changes of target analyte nucleic acid sequence, splice pattern, amino acid sequence, phosphorylation status or folding. In some cases, the plurality of probes that bind the analyte directly are designed to comprise a third oligomeric region that differs among the plurality of probes that bind the analyte directly, and that is accessible when the probes are bound to analytes, such that independent probe binding to an analyte can be assayed, for example using fluorescent probes that are specific to or that differentially target the third oligomeric region of the analyte binding probes. Thus, distinct from assaying for the analyte, one may assay for binding by particular probes of the analyte-binding probes to the analyte.

Once an analyte is coated with multiple copies of an identifier element, the analytes may be contacted to populations of decoder probes. The decoder probes serve a number of purposes. Firstly, the decoder probes replace the relatively diverse identifier element moieties with decoding moieties, which may in some systems or methods be binary or exhibit other lower order diversity. Secondly, the decoder probes often exhibit melting temperatures below that of the probes that bind the analyte directly. As a consequence, the decoder probes may be iteratively removed and replaced, so as to swap out one decoder moiety with another. This replacement may be done in a temporally defined sequence, so as to establish, over a series of iterative annealing and removal steps, a temporal pattern of decoding moieties bond to the analyte. This pattern may be a binary or higher order sequence of decoding moieties, depending upon the diversity of the decoding moieties used.

The value of such a temporal pattern of labeling of the analyte by the decoding probes is understood in the context of addition of signal element probes prior to removal of the decoding probes in any particular round of decoder probe addition. Signal elements probes comprise a specificity region that may anneal to or otherwise bind to the decoding moieties of the decoder probes. Signal element probes further comprise a fluorophore or other detection moiety that corresponds to the specificity region for the particular signal element probes. Thus, upon iterative rounds of decoding probe binding, signal probe binding, and detection moiety detection, one can observe a temporal order of signals that correspond to the temporal pattern of administration of decoding probes to the identifier-element labeled analyte.

Although the analyte-binding probes are specific to the expected sequence of their target, all of the other reagents of the systems, compositions and methods herein can be generated efficiently in bulk. Decoder probes, for example, have an identifier element binding moiety and a signal element binding moiety (often a distinct oligo for each purpose). The identifier moieties require some degree of diversity such that analyte binding probe populations may be distinguished from one another. That is, the diversity of identifier moieties is often greater than the diversity of analytes to be detected. However, the decoder probes do not need to be specific to analytes of any particular experiment and may be efficiently synthesized in bulk. The decoder probe signal element biding moieties require even less diversity, and in some systems are often bipartite (although higher order complexity, such as having three, four or more than four distinct signal element binding moieties are also consistent with the disclosure herein). Signal probes require substantially less diversity and may comprise only two populations in some cases. For example, signal probes may comprise a first population having a first signal element and a first detection moiety, and a second population having a second signal element and a second detection moiety. Signal complexity sufficient to identify a particular analyte is accomplished not through an individual signal element signal but through a pattern of signal element signals, determined by the pattern of delivery of decoder elements to the identifier element-labeled analyte. As a consequence, exquisite analyte specificity may be achieved without relying on a large diversity of detection moieties such as fluorophores, and without covalent binding of detection moieties to analyte specific probes.

Particularly relevant to the disclosure herein, although the analyte-binding probes are specific to their target, target detection is not exclusively reliant on any particular target sequence. Because the analyte binding probes bind to different target sites in the analyte, the overall analyte binding process is not reliant on any one of the target binding sites to be known and bound with certainty. As a result, the analyte binding probes, as a diverse population, are resilient to allelic or other variation that may arise in one or another of the target sites withing the analyte, so as to accurately detect an analyte even in individuals whose genome may differ from a predetermined genomic standard, as is often the case among minorities or other individuals who may be ‘diverse’ relative to the genome sequence available. Similarly, the analyte binding probes are resilient to variation that may arise in, for example, a rapidly evolving pathogen population, such as has been seen in the genomic progression of Covid-19 variants.

Accordingly, disclosed herein are compositions comprising a target analyte bound to a subset of an identically tagged analyte binding probes, wherein each analyte binding probe is designed to bind to an expected target analyte but wherein the target analyte of a sample differs from the expected target analyte in sequence such that some analyte binding probe subsets will bind the sample analyte while others, directed to a region that differs from the expected analyte sequence, will not bind the target analyte. Accordingly, in the composition at least some of the identically labeled target analyte probes are directed to a region or regions of the sample target analyte that are as expected and will bind to the target analyte, while others among the identically labeled target analyte binding probes are directed to a region or regions of the target analyte that differ from that expected for the target analyte, and will not bind the target analyte.

A few exemplary features of the systems, methods and compositions disclosed herein are as follows. The analyte identifier label is not specific to any individual probe and does not identify any particular probe. Rather, the identifier corresponds to the analyte to be detected, but does not specify any individual probe.

Consequently, at least two probes on an analyte targeting population will be tagged with a common identifier label, such that failure of one of the probes to bind to the analyte will not preclude detection of the analyte. This feature of the technology herein provides a level of resilience not seen in many approaches in the prior art. Variations in analyte identity, such as the existence of alternative splicing or genomic allelic variation in nucleic acid analytes, as is often seen among various populations, or of variations in protein amino acid resides at a given position or of post-translational modification such as phosphorylation, will not preclude analyte detection. In cases where analyte detection probes additionally are labeled with probe-specific oligomers, then separate FISH-type probe hybridization may be used to detect the annealing success of individual analyte-binding probes. However, unlike these approaches, analyte detection is not dependent on individual probe binding, and identifier labels are not specific to any particular individual probe.

An additional feature of the approaches herein is that the temporal order of signal signatures is independent of and not determined by the identifier label sequence. The order of signal signatures is determined by the user's selection of the order of decoder probe administration. It is not determined by or limited by the identifier label sequence. Consequently, there is considerable flexibility in both the identifier label, which does not need to be so long as to accommodate the full extent of signal sequence diversity, and there is also considerable flexibility in the temporal order of the detection signals, as they are not constrained by the identifier label sequence. Instead, the detection signals are determined by the temporal order of decoder probe addition and removal. The identifiers are bound by decoding probes successively at a single nucleic acid label binding site. Order of binding by the linking probes at the single nucleic acid label binding site identifies the analyte target. Notably, the decoder probes are not covalently fluorophore labeled, which allows efficient bulk production of a small number of fluorophore-labeled signal probe populations. This conveys a considerable cost savings and allows the specification of a temporal order of signals to identify a particular analyte.

The systems, compositions and methods herein comprise three categories of ‘stains’ or probes: analyte binding stains, decoding probes or stains, and fluorophore labeled signal probes or stains. Of these, the decoding probes and the signal probes do not comprise sequence that is specific to any particular analyte target. Consequently, the decoding probes and the signal probes may be synthesized in bulk, independent of any particular experiment, and need not be tailored to any particular target. None of the decoding probes are specific to any target, and none of the signal probes are specific to any target.

That is, the decoding probes and signal probes do not (or are not designed intentionally to) comprise target-specific sequence. Accordingly, the impact of variation from expected target specific sequence in an analyte is reduced.

The analyte binding stains comprise regions that bind to different portions of a target analyte, such that although the target analyte may be detected with specificity, the detection is not reliant upon any single particular target analyte region being present exactly as predicted. Two analyte binding stains may bind to different targets and, effectively, show no co-specificity as to their target region binding; however, because the target regions are selected to be part of a common target analyte, the analyte binding stain population as a whole is able to specifically detect the target analyte even though the individual stain molecules share no specificity as to their specific target sequences. Accordingly, variation in a single analyte target sequence, arising from, for example, a lineage specific SNP, a splice variant, or an innovation in a genome such as a hypervariable pathogen genome, will not disrupt performance of the analyte binding stain set as a whole in its detection of the target analyte. Rather, it will merely result in a composition for which some members of a subset of target analyte binding probes directed to a region of the target analyte which is as expected will bind to the target analyte, while all members of the target analyte binding probes directed to a region that varies, often substantially, from an expected sequence will not bind but will remain in solution.

In some embodiments, additional probe specific oligomers are added to analyte-detection probe populations. This is not required for the main function of the systems, methods and compositions herein, but may in some cases confer additional benefits. In a first aspect a method is disclosed for detecting a polymorphic analyte in a sample by specific signal-encoding of said polymorphic analyte is disclosed. In one embodiment of that aspect at least two polymorphic analyte-specific probes form a set of polymorphic analyte-specific probes. In another embodiment of that aspect the signal element is not specific to a single polymorphic analyte-specific probe. In another embodiment of that aspect the signal element is specific to at least a set of the polymorphic analyte-specific probes. In yet another embodiment of that aspect the signal element does not identify specific to a single polymorphic analyte-specific probe. In yet another embodiment of that aspect the signal element does identify at least a set of the polymorphic analyte-specific probes. In yet another embodiment of that aspect the at least two probes of the set, preferably a plurality of polymorphic analyte-specific probes of polymorphic analyte-specific probes share a common label. In yet another embodiment of that aspect the set of polymorphic analyte-specific probes share a common target. In yet another embodiment of that aspect the common target is a genetic locus. In yet another embodiment of that aspect the common target is a transcript. In yet another embodiment of that aspect the failure of a sub-set of polymorphic analyte-specific probes to bind to the common target does not preclude the detection of the common target. In yet another embodiment of that aspect the allelic variation in the common target does not preclude the detection of the common target. In yet another embodiment of that aspect the allelic variation comprises at least one insertion, deletion, or single nucleotide variation relative to an expected common target. In yet another embodiment of that aspect the signal element is specific to a common target. In yet another embodiment of that aspect the signal element identifies a common target.

In a second aspect, a method for detecting a polymorphic analyte in a sample by specific signal-encoding of said polymorphic analyte is disclosed, comprising the steps of: (A) contacting the sample with a set of polymorphic analyte-specific probes for encoding different variations of the same polymorphic analyte, each analyte-specific probe interacting with a different variation and/or sub-structure of the polymorphic analyte, wherein the polymorphic analyte is present in the sample in at least as two or more variant forms of a specific nucleic acid sequence and wherein the sequences of each polymorphic analyte are related to each other with a sequence identity of between 85% to 99%, each polymorphic analyte-specific probe comprising (aa) a binding element(S) that specifically interacts with one of the at least two or more variants and/or sub-structure of the polymorphic analyte to be encoded, and (bb) an identifier element (T) comprising a nucleotide sequence which is unique to the polymorphic analyte to be encoded (unique set identifier sequence), wherein each set of polymorphic analyte-specific probes differ from another set of polymorphic analyte-specific probes in the nucleotide sequence of the identifier element (T); and (B) contacting the sample with at least a first set of decoding oligonucleotides per polymorphic analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique set identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element facilitating a signal which is specific for the polymorphic analyte; and (D) Detecting the signal caused by the signal element; (E) Optionally selectively removing the decoding oligonucleotides and signal oligonucleotides from the sample, thereby essentially maintaining the specific binding of the analyte-specific probes to the analytes to be encoded; (F) Performing further cycles comprising steps A) to E) for each further polymorphic analyte to generate an encoding scheme with a specific signal per set of polymorphic analytes, wherein in particular the last cycle may stop with step (D).

Depending on the design of the probe set, the detected signal may label a gene family, a single gene, a specific genetic locus, a benign or malign genetic variation, genetic specificity or biomarker in a cluster of patients, a virus-genome, a certain subset of nucleic acid variation (e.g. a group of SNPs, a mutation, a virus-variant, a gene-variant, a transcript, isoforms, splice-variants, etc.).

As such, the technology allows the high-throughput detection of nucleic acids with variable sequences, for example of at least 60%, at least 65%, at least 70%, at least 75%, at least 80%, at least 85%, at least 90%, and up to 92%, up to 95%, up to 98%, up to 99%, up to 99.9% sequence identity or greater. In some embodiments between 65% to 99%, between 70% to 99%, between 75% to 99%, between 80% to 99%, between 85% to 99%, between 85% to 98%, between 85% to 95%, between 90% to 95% sequence identity in a sample. Similarly, the technology allows the high throughput detection of nucleic acids with variable sequences, for example, varying in at least 1, 2, 3, 4, 5, 6, 7, 8, 9, 10 or more than 10 bases, or varying by the presence of an insertion/deletion of at least 1, 2, 3, 4, 5, 6 7, 8, 9, 10, 15, 20, 30, 40, 50, 100, or more than 100 bases, or harboring at least 1, 2, 3, 4, 5, 6, 7, 8, 9, 10 or more than 10 SNPs relative to one another, or harboring at least 1, 2, 3, 4, 5, 6, 7, 8, 9, 10 or more than 10 modifications such as methylation sites relative to one another.

The technology is also more robust than conventional methods, since the signal is still detectable even if just a subset of probes bind the target. As such the paradigm “the label identifies the probe” is overcome and the rule “the label identifies the target” applies instead.

Accordingly, disclosed herein are methods comprising designing target analyte probe sets to an expected target analyte, and using these probe sets to assay for an analyte that differs from the expected analyte at at least one but not all of the positions targeted by probes of the probe sets. Similarly, disclosed herein are compositions resulting from practice of said method, such that a sample target analyte that differs from an expected analyte is bound at at least one but not all of the positions targeted by probes of the probe sets designed to an expected target analyte.

In a similar aspect, embodiments of the disclosure pertain in particular to a kit for polymorphic analyte encoding, comprising reagents for practicing the steps of: (A) contacting the sample with a set of polymorphic analyte-specific probes for encoding different variations of the same polymorphic analyte, wherein the analyte-specific probes are directed to an expected target analyte, each analyte-specific probe interacting with a different variation and/or sub-structure of the polymorphic analyte, wherein the polymorphic analyte is present in the sample in at least as two or more variant forms of a specific nucleic acid sequence and in some cases wherein the sequences of each polymorphic analyte are related to each other with a local or global sequence identity of between 85% 60 to 99%, such as 85%-99%, each polymorphic analyte-specific probe comprising (aa) a binding element(S) that specifically interacts with one of the at least two or more variants and/or sub-structure of the polymorphic analyte to be encoded, and (bb) an identifier element (T) comprising a nucleotide sequence which is unique to the polymorphic analyte to be encoded (unique set identifier sequence), wherein each set of polymorphic analyte-specific probes differ from another set of polymorphic analyte-specific probes in the nucleotide sequence of the identifier element (T); and (B) contacting the sample with at least a first set of decoding oligonucleotides per polymorphic analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique set identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element facilitating a signal which is specific for the polymorphic analyte; and (G) Detecting the signal caused by the signal element; (H) Optionally selectively removing the decoding oligonucleotides and signal oligonucleotides from the sample, thereby essentially maintaining the specific binding of the analyte-specific probes to the analytes to be encoded; (I) Performing further cycles comprising steps A) to E) for each further polymorphic analyte to generate an encoding scheme with a specific signal per set of polymorphic analytes, wherein in particular the last cycle may stop with step (D). In various embodiments, the global lor local sequence identity is, for example, 85% to 99%, or for example at least 60%, 65%, 70%, 75%, 80%, 85%, 90%, 95%, 96%, 97%, 98%, or 99% or more.

Some embodiments of this disclosure relate to in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure.

In another aspect, embodiments of this disclosure provide in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the multiplex method according to the present disclosure. In various embodiments, the disease is assayed for in plants, in animals or in fungi or other targets. In various embodiments, the multiplex method targets native host target analytes, pathogen target analytes, or both native host target analytes and pathogen target analytes. Similarly, in some cases the method is used to assay for non-disease interactions such as mutual, commensal or other host interactions such as plant nodulation or human microbiome assays.

In yet another aspect, some embodiments of this disclosure relate to optical multiplexing systems suitable for the method according to the present disclosure, comprising: at least one reaction vessel for containing the kits or part of the kits according to any one of the claim; a detection unit comprising a microscope, in particular a fluorescence microscope; a camera; and a liquid handling device.

In yet another aspect, some embodiments provide in vitro methods for screening, identifying and/or testing a substance and/or drug comprising: (a) contacting a test sample comprising a sample with a substance and/or drug; (b) detecting different analytes in a sample by sequential signal-encoding of said analytes with a method according to the present disclosure.

According to the present disclosure, unique tags (identifier) are used per target (e.g. mRNA of one single gene) or for a target group. Groups can be formed to be indicative for a certain identity, process, biological function or disease (examples: cell type, inflammation, signal processing, cancer).

Surprisingly, the methods and kits according to the present disclosure lead to the reduction of complexity. Many different probes with different binding sequences share the same (one per target) unique or probe set specific tag. These tags have reduced the sequence complexity (to one per target) and also have predetermined constant properties (e.g. thermodynamic stability).

Advantages of the methods and kits according to the present disclosure in various embodiments comprise one or more of: a) Full flexibility of the process to determine polymorphic analytes, e.g. targets (such as nucleic acids) encoding different variations of the same polymorphic analyte, wherein each analyte-specific probe interacts with a different variation and/or sub-structure of the polymorphic analyte, for example different exon regions, differen intron regions, different orthologs, paralogs and/or xenologs, and/or other genetic variations (SNPs, Indels, CNVs, etc.); b) use more or less signals and/or rounds, varying numbers of fluorophores, number of total signals per tag such that lower numbers of targets (e.g. 20) can be identified with high confidence in less rounds (e.g. 4) than a large number of targets (e.g. 100, these need 8 rounds for the same level of confidence), even if in both cases the exact same unique tags are used. c) All unique tags are used (recycled) in many consecutive rounds of hybridization and all primary probes contribute (provide information about their identity) in every round of identification.

d) As all tags share the same predefined properties (e.g. thermodynamic stability which allows for selective denaturing).

In some advantageous embodiments, the unique tags are design as follow: No cross-hybridization occurs between all oligonucleotides of the process (probes, decoders, readout), so that all tag sequences are usable together (compatible); and No cross-hybridization occurs between connector elements (bridges) of different unique tags; Stability of hybridization of the unique tags should be in a narrow range: as stable as possible (fast hybridization, i.e. short cycle times) but significantly different (in this case less stable) than the primary probe (for differential denaturation, without removing primary probes).

Therefore, the present description pertains in particular to the usage of a set of labeled and unlabeled nucleic acid sequences or stains for specific quantitative and/or spatial detection of different analytes in parallel via specific hybridization. The technology allows the discrimination of more different analytes than different detection signals are available. The discrimination is realized via sequential signal-coding of the analytes achieved by several cycles of specific hybridization, detection of signals and selective elution of the hybridized nucleic acid sequences. In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds. The utilization of decoding-oligonucleotides leads to a sequential signal-coding technology that is e.g. more flexible, cheaper, simpler, faster and/or more accurate than other methods.

Furthermore, the present disclosure pertains to the use of improved decoding-oligonucleotides to increase the efficiency of the encoding scheme. The so called “multi-decoders” allows the recruiting of more than just one signal oligonucleotide and therefore can generate new signal types by utilizing the combination of two or more different signal-oligonucleotides without decreasing the brightness of the signals.

Further advantages pertain to an improvement of the overall signal to noise ratio, the signal spread (that is, signals of transcripts with higher expression levels can be detected together with lowly expressed genes) and the multiplexing capability without increasing optical crowding.

Further advantages pertain to the resilience of target analyte detection, in that variation at a particular target site in an analyte does not preclude detection of that analyte. This resilience allows the detection of rapidly evolving target analytes as well as target analytes in individuals whose genomes may vary from an expected sequence or a sequence from which the probe set was generated, without loss in detection performance.

The present disclosure allows the detection of polymorphic analytes by detecting related genes selected from the group comprising gene copies, diploid genes, homeologs such as orthologs, paralogs, xenologs, gametologs, similar sequences, such as in some cases sequences exhibiting with at least 85% sequence identity for example, gene copies with mutations, deletions, insertions, inversions, and/or SNPs, thereby clustering related genes to a single signal. In some embodiments by detecting coding (e.g., exon) and noncoding regions (e.g., promoter, intron) in related genes, thereby clustering related genes to a single signal.

Before the disclosure is described in detail, it is to be understood that this disclosure is not limited to the particular component parts of the steps of the methods described. It is also to be understood that the terminology used herein is for purposes of describing particular embodiments only and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1: Embodiment where the analyte is a nucleic acid and the probe set comprises oligonucleotides specifically binding to the analyte at different locations. The probes comprise a unique identifier sequence allowing hybridization of decoding oligonucleotides.

FIG. 2: Embodiment where the analyte is a protein and the probe set comprises proteins (here: antibodies) specifically binding to the analyte at different locations. The probes comprise a unique identifier sequence allowing hybridization of decoding oligonucleotides.

FIG. 3: Flowchart of the method according to the disclosure.

FIG. 4: Alternative options for the application of decoding and signal oligonucleotides.

FIG. 5: Example for signal encoding of three different nucleic acid sequences by two different signal types and three detection rounds; in this example, the encoding scheme includes error detection.

FIG. 6: Number of generated code words (logarithmic scale) against number of detection cycles.

FIG. 7: Calculated total efficiency of a 5-round encoding scheme based on single step efficiencies.

FIG. 8: Comparison of relative transcript abundances between different experiments.

FIG. 9: Correlation of relative transcript abundances between different experiments.

FIG. 10: Comparison of intercellular distribution of signals.

FIG. 11: Comparison of intracellular distribution of signals.

FIG. 12: Distribution pattern of different cell cycle dependent transcripts.

FIG. 13: Detection of multiple targets using an 8-round code with 2 labels (A and B) and no label (-). The targets 1, 2, 3, 4, 5, 20, and n are represented. The rounds 1, 2, 3, and 8 of the coding scheme are represented. Herein, the blank is part of the code.

FIG. 14: Detection of multiple targets can be performed by an encoding scheme using a detectable marker. The ending scheme may comprise also the “0” as a marker. That means that at a specific position the transcript is not detected. Consequently, the encoding scheme may be represented by the following constructs using only two gene specific probes:
1) With detectable label F: detectable during imaging
2) With detectable label F and quencher Q: not detectable during imaging
3) With quencher Q: not detectable during imaging
4) Without label F: not detectable during imaging
5) Without signaling oligonucleotide: not detectable during imaging
6) With a decoder oligonucleotide that cannot recruit a signaling oligonucleotide
7) Without decoder oligonucleotide: not detectable during imaging

FIG. 15: Possible structures of a multi-decoder. The numbers depict the examples. (A) is the unique identifier sequence, (a) is the corresponding sequence of the decoding oligonucleotide or multi-decoder and (c1) to (c3) are different sequence elements, that specifically bind to different signal oligonucleotides. Examples 2 to 5 show different versions of multi decoders. The order of the different sequence elements as well as the number of signal oligonucleotide binding elements is not fixed. Example 1 shows a normal decoding oligonucleotide since there is only one signal oligonucleotide binding element (c1).

FIG. 16: Example for signal encoding of three different nucleic acid sequences by using multi-decoders and two different signal oligonucleotides creating three different signal types and three detection rounds. In this example, the encoding scheme includes error detection and correction.

FIG. 17: Number of generated codewords (logarithmic scale) against number of detection cycles. The number of codewords for merFISH does not exponentially increase with the number of detection cycles but gets less effective with each added round. In contrast, the number of codewords for intronSeqFISH, the method of the present disclosure without using multi-decoders, the method with multi-decoders increases exponentially. The slope of the curve for the method using multi-decoders is much higher than that of the prior invention, leading to more than 20 000 000 times more code words usable after 20 rounds of detection.

FIG. 18A through FIG. 18E: Comparison of two consecutive MC runs targeting the same tails. FIG. 18A Overview of experimental setup. FIG. 18B Barplot showing transcript counts in different tissue regions of first and second set. FIG. 18C Scatterplot of transcript counts per genes of first and second set. FIG. 18D Barplot showing false positive rates (fractions of detected blank barcodes) in different tissue regions of first and second set. FIG. 18E Boxplot of signal intensities per imaging round. Top: Channel 1; Bottom: Channel 2. Left: first set; Right: second set.

### DETAILED DESCRIPTION OF THE DISCLOSURE

Disclosed herein are novel high-resolution multiplex methods and kits for detecting different analytes in a sample beyond the diffraction limit by sequential signal-encoding of said analytes. Furthermore, disclosed herein are novel methods for detecting a polymorphic analyte in a sample by specific signal-encoding of said polymorphic analyte in a manner that is resilient to the variation in the polymorphic analyte.

The present disclosure describes the usage of at least two sets of labeled and unlabeled nucleic acid sequences for specific quantitative and/or spatial detection of different analytes via specific hybridization. The technology allows the discrimination of more different analytes than different detection signals are available. The discrimination may be realized via sequential signal-coding of the analytes achieved by several cycles of specific hybridization, detection of signals and selective elution of the hybridized nucleic acid sequences.

In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed which in turn increases the coding capacity achieved with a certain number of detection rounds.

Furthermore, in contrast to other state-of-the-art methods using already “decoding-oligonucleotides”, at least two different sets of “decoding-oligonucleotides” are used directed to each of the at least two different analytic sets, in order to allow the detection of different subgroups of targets within one analytical run and that any spatial resolving limitation no longer applies.

The first analytical set is optimized with respect to specificity, sensitivity and affinity by the following steps:

The target sequence is scanned, and every position (each base) is extended until the predicted hybridization of the complementary part (prospect probe candidate) reaches the minimal required binding stability. After this step, candidates containing homopolymers, repeated motifs or low complexity (e.g., only consisting of two different bases) are discarded. The remaining probe candidates are used to find stable binding sites in all other transcripts from the same organism to detect off-target binding. Probe candidates with high affinity to non-targeted sequences are discarded. The final list of candidates is scored and ranked by on-target binding characteristics: Binding on multiple targeted transcript variants increases the score (depending on the transcript annotation class, common and canonical transcript score highest, low evidence transcripts or non-coding isoforms lowest), and a bonus is given if the binding region overlaps the protein coding region of the gene. Finally, highest ranked probe candidates are selected, avoiding large overlaps between probes.

In one aspect of the method of the present invention, the analytical rounds can be arranged consecutively, meaning the detection round(s) of a first analytical set is finished before the detection round(s) of a second analytical set start.

In a second aspect of the method of the present invention, the analytical rounds can be arranged interleaved, meaning the detection rounds of a first analytical set and a second analytical set alternate in a certain pattern, e.g.: Detection round 1=1st round of detection of first analytical set; Detection round 2=1st round of detection of second analytical set; Detection round 3=2nd round of detection of first analytical set; Detection round 4=2nd round of detection of second analytical set; Repeat or extend.

The utilization of decoding-oligonucleotides leads to a sequential signal-coding technology that is more flexible, cheaper, simpler, faster and/or more accurate than other methods and allows a resolution beyond the diffraction limit of optical microscopes.

It is one advantage of the present invention to detect a larger number of different targets (e.g., transcripts or genes) with fewer detection cycles as compares to the prior art.

Furthermore, the present disclosure describes the usage of set of polymorphic analyte-specific probes for encoding different variations of the same polymorphic analyte, each analyte-specific probe interacting with a different variation and/or sub-structure of the polymorphic analyte, wherein the polymorphic analyte is present in the sample in at least as two or more variant forms of a specific nucleic acid sequence and wherein the sequences of each polymorphic analyte are related to each other with a local or global sequence identity of, for example, between 85% to 99%. The technology allows the discrimination of more different analytes than different detection signals are available. The discrimination may be realized via sequential signal-coding of the analytes achieved by several cycles of specific hybridization, detection of signals and selective elution of the hybridized nucleic acid sequences.

In contrast to other state-of-the-art methods, the oligonucleotides providing the detectable signal are not directly interacting with sample-specific nucleic acid sequences but are mediated by so called “decoding-oligonucleotides”. This mechanism decouples the dependency between the analyte-specific oligonucleotides and the signal oligonucleotides. The use of decoding-oligonucleotides allows a much higher flexibility while dramatically decreasing the number of different signal oligonucleotides needed, which in turn increases the coding capacity achieved with a certain number of detection rounds.

The utilization of decoding-oligonucleotides leads to a sequential signal-coding technology that is more flexible, cheaper, simpler, faster and/or more accurate than other methods, and is durable to detection of target analytes that differ from an expected target analyte sequence at some analyte binding probe binding sites.

### A. Definitions

According to the present disclosure an “analytical set” is a set of probes specific for a subgroup of targets or a particular analyte. Preferably the targets within a first analytical set directed to a subgroup of targets which do not usually show a high spatial overlap, whereas a second analytical set directed to a subgroup of targets which may spatially overlap with the first subgroup and/or may add additional information which allows a further differentiation of the signals found with the first analytical set. For example, a first analytical set may be directed to known activating mutations in promotor-structures with a high prevalence for cancer. The second analytical set may be directed to genes associated with cancer development when over-expressed. Thus, a colocalization of signals detected with the first and the second analytical set may indicate activated promotors in genes associated with cancer.

Sets comprise subpopulations or subsets of probes that are directed to different locations of a target analyte. In some cases, “probes” of a probe set refer to at least one member of a first subset and at least one member of a second subset. So, for example, if a target analyte is bound by multiple “probes” of a target analyte probe set, the reference is understood to refer to at least one representative of a first subset and at least one member of a second subset.

In yet another embodiment fusion genes may be detected by this method. Fusion genes, and the fusion proteins that come from them, may occur naturally in the body when part of the DNA from one chromosome moves to another chromosome. Fusion proteins produced by this change may lead to the development of some types of cancer. The disclosed method allows the detection of co-localization other normally distinct gene loci and therefore the detection of certain cancer types, e.g. cancers which are the result of the fusion of the following genes: Adenoid cystic carcinoma as result of MYB-NFIB and/or NFIB-HMGA2 fusion; mucoepidermoid carcinoma as a result of MECT1-MAL2 fusion; follicular thyroid carcinoma as a result of PAX87-PPARG fusion; breast carcinoma as a result of ETV6-NTRK3 and/or FGFR3-AFF3 and/or FGFR2-CASP7 and/or FGFR2-CCDC6 and/or ERLINK2-FGFR1 fusion; Ewing sarcoma as a result of EWSR1-FLI1 fusion; Small round cell tumors of bone BCOR-CCNB3 fusion; Synovial sarcoma as a result of SS18-SSX1 and/or SS18-SSX2 fusion; glioblastoma multiforme as a result of FGFR3-TACC3 and/or FGFR1-TACC1 fusion; pilocytic astrocytoma as a result of KIAA1967-BRAF fusion; lung cancer as a result of EML4-ALK and/or FGFR3-TACC3 and/or FGFR3-KIAA1967 and/or BAG4-FGFR1 fusion; clear cell renal cell carcinoma as a result of SFP!-TFE3 and/or TFG-GPR128 fusion; bladder cancer as a result of FGFR3-TACC3 and/or FGFr3-BAIAP2L1 fusion; prostate cancer as a result of TMPRSS2-ERG/ETV1/ETV4 and/or SLC45A3-FGFR2 fusion; ovarian cancer as a result of ESRRA-C11orf20 fusion; and/or colorectal cancer as a result of PTPRK-RSPO3 and/or EIF3E-RSPO2 fusion. Of course, also other combinations can be envisaged, for example tissue-type-specific probes with cancer markers, proto-oncogenic targets in different combinations, etc.

In one embodiment, a first analytical set may be directed to known transcripts of a certain gene. The second analytical set may be directed to another set of transcripts of the same gene to get information about differential splicing. Thus, a colocalization signals detected with the first and the second analytical set may detect differentially spliced exons of transcripts of the same gene.

According to the present disclosure an “analyte” is the subject to be specifically detected as being present or absent in a sample and, in case of its presence, to encode it. It can be any kind of entity, including a protein, polypeptide, protein or a nucleic acid molecule (e.g. RNA, PNA or DNA) of interest. The analyte provides at least one site for specific binding with analyte-specific probes. Sometimes herein the term “analyte” is replaced or modified by “target”. An “analyte” according to the disclosure includes a complex of subjects, e.g., at least two individual nucleic acid, protein or peptides molecules. In an embodiment of the disclosure an “analyte” excludes a chromosome. In another embodiment of the disclosure an “analyte” excludes DNA. A broad range of entities may serve as analytes, including a protein, polypeptide, protein or a nucleic acid molecule (e.g. RNA, PNA or DNA) of interest. The analyte is often selected to provide at least two distinct binding sites, such that representatives of at least two distinct subsets of probes of a target analyte probe set are able to independently and concurrently bind to the target analyte.

In some embodiments, an analyte may be or may comprise a “coding sequence”, “encoding sequence”, “structural nucleotide sequence” “transcribed sequence,′ “spliced sequence,” reverse transcribed sequence,” “noncoding RNA” or “structural nucleic acid molecule” which refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, RNA, EST and recombinant nucleotide sequences. A coding sequence is often bound by noncoding sequences, such as 5′ and 3′ Untranslated regions, or UTRs. Alternately, an analyte may be a noncoding sequence, such as rRNA, tRNA, miRNA, snRNA, Xist, or other transcribed but untranslated RNA molecule. An analyte is in some cases the product of a reverse-transcription reaction.

A “sample” as referred to herein is a composition in liquid or solid form suspected of comprising the analyte or analytes to be encoded for example through the annealing or labeling process disclosed herein. In particular, the sample is often a biological sample, preferably comprising biological tissue, further preferably comprising biological entities such as cells, viruses, and/or extracts and/or part of cells or morphological structures. For example, the cell is a prokaryotic cell or a eukaryotic cell, in particular a mammalian cell, in particular a human cell. In some embodiments, the biological tissue, biological cells, extracts and/or part of cells are fixed. In particular, the analytes are fixed in a permeabilized sample, such as a cell-containing sample. The sample may also be fixed to a surface. Often, a sample preserves structural or positional information, such that detection of an analyte conveys not only information as to the presence of the analyte in the sample, but also conveys positional information as to the analyte relative to known or identifiable structures in the sample, such tissues, cells, or subcellular structures such as the nucleus, nucleolus, mitochondria or other subcellular structures, as well as relative to other analytes concurrently or subsequently detected. Accordingly, analyte detection often conveys positional information as to that analyte in the sample. In an embodiment of the disclosure the sample is a biological sample, preferably comprising biological tissue, further preferably comprising biological cells. A biological sample may be derived from an organ, organoids, cell cultures, stem cells, cell suspensions, primary cells, samples infected by viruses, bacteria or fungi, eukaryotic or prokaryotic samples, smears, disease samples, or a tissue section. Often, a sample preserves the positional information of the source from which it was taken, such that a target analyte position can be determined within the sample.

Samples consistent with the disclosure herein comprise a target analyte or a homologue or orthologue of a target analyte having a sequence of nucleic acid bases or amino acid residues that is unknown or that is known to differ or believed to differ from a known target analyte sequence from which the target analyte probe set is designed. Accordingly, in cases where the difference at a probe subset binding site differs sufficiently from that of an expected target analyte sequence, when a sample is contacted to a target analyte probe set consistent with the disclosure herein, in some embodiments a composition is formed wherein a target analyte is bound by members of a first subset of target analyte probes but is not bound by any members of at least a second subset of the target analyte probes. Nonetheless, the sample target analyte is identified in the sample, demonstrating the durability of the methods and compositions herein.

As used in the present disclosure, “cell”, “cell line”, and “cell culture” can be used interchangeably and all such designations include progeny. Thus, the words “transformants” or “transformed cells” include the primary subject cell and cultures derived therefrom without regard for the number of transfers. It is also understood that all progenies may not be precisely identical in DNA content, due to deliberate or inadvertent mutations. Mutant progeny that has the same functionality as screened for in the originally transformed cell are included.

An “encoding scheme” may describe a set of “code words” that are associated with the analytes to be detected. Each “code word” refers to one of the analytes and can be distinguished from all other “code words”. A code word hereby is a sequence of signs provided by the detection cycles of the method. A sign within a “code word” is a detectable signal or the absence of a signal. A “code word” does not need to comprise of all different signals used in the method. The number of signs in a “code word” is defined by the number of detection cycles.

Importantly, the number or identity of signals in an encoding scheme is not inherent in the analyte binding probe set, the decoding set or the signal probe set. Rather, the encoding scheme is determined by the user, according to the sequence of decoding oligos added to and removed from the sample. Accordingly, there is substantial flexibility in selection of an encoding scheme, and the length of a code word in an encoding scheme may be tailored to attain the degree of specificity per target analyte in a multi-analyte probing reaction.

An “oligonucleotide” as used herein, refers to a short nucleic acid molecule, such as DNA, PNA, LNA or RNA. The length of the oligonucleotides is within the range 4-200 nucleotides (nt), preferably 6-80 nt, more preferably 8-60 nt, more preferably 10-50 nt, more preferably 12 to 35 depending on the number of consecutive sequence elements. The nucleic acid molecule can be fully or partially single-stranded. The oligonucleotides may be linear or may comprise hairpin or loop structures. The oligonucleotides may comprise modifications such as biotin, labeling moieties, blocking moieties, or other modifications.

The “analyte-specific probe” consists of at least two elements, namely the so-called binding element(S) which specifically interacts with one of the analytes, and a so-called identifier element (T) comprising the ‘unique identifier sequence’. The binding element(S) may be a nucleic acid such as a hybridization sequence or an aptamer, or a peptidic structure such as an antibody.

Also, a “probe” comprises or consists of at least two elements, namely the so-called binding element(S) which specifically interacts with one of the analytes, and a so-called identifier element (T) comprising the ‘unique identifier sequence’. The binding element(S) may be a nucleic acid such as a hybridization sequence or an aptamer, or a peptidic structure such as an antibody.

In particular, in some embodiments the binding element(S) comprises moieties which are affinity moieties from affinity substances or affinity substances in their entirety selected from the group consisting of antibodies, antibody fragments, receptor ligands, enzyme substrates, lectins, cytokines, lymphokines, interleukins, angiogenic or virulence factors, allergens, peptidic allergens, recombinant allergens, allergen-idiotypic antibodies, autoimmune-provoking structures, tissue-rejection-inducing structures, immunoglobulin constant regions and their derivatives, mutants or combinations thereof. In further advantageous embodiments, the antibody fragment is a Fab, an scFv; a single domain, or a fragment thereof, a bis scFv, Fab2, Fab3, minibody, maxibody, diabody, triabody, tetrabody or tandab, in particular a single-chain variable fragment (scFv).

The “unique identifier sequence” as comprised by the analyte-specific probe is unique in its sequence compared to at least one other unique identifier. “Unique” in this context means that it specifically identifies only one target analyte in a given sample assay, for example a transcript encoding, such as Cyclin A, Cyclin D, Cyclin E etc., or, alternatively, it specifically identifies only a group of analytes, independently whether the group of analytes comprises a gene family or not. Therefore, the analyte or a group of analytes to be encoded by this unique identifier can be distinguished from all other analytes or groups of analytes that are to be encoded based on the unique identifier sequence of the identifier element (T). Or, in other words, in exemplary embodiments there is only one ‘unique identifier sequence’ for a particular analyte or a group of analytes, but not more than one, i.e. not even two. Due to the uniqueness of the unique identifier sequence the identifier element (T) hybridizes to exactly one type of decoding oligonucleotides. The length of the unique identifier sequence is within the range 8-60 nt, preferably 12-40 nt, more preferably 14-20 nt, depending on the number of analytes encoded and the stability of interaction needed.

In one embodiment modified oligonucleotides may be used to crosslink the hybridized elements permanently (i.e. covalently), e.g. to establish a permanent connection between analyte specific probe and decoding oligonucleotide(s); between decoding oligonucleotide(s) and signal oligonucleotide(s); and/or analyte specific probe and decoding oligonucleotide(s) and signal oligonucleotide(s).

A unique identifier may be a sequence element of the analyte-specific probe, attached directly or by a linker, a covalent bond or high affinity binding modes, e.g. antibody-antigen interaction, streptavidin-biotin interaction etc. It is understood that the term “analyte specific probe” includes a plurality of probes which may differ in their binding elements(S) in a way that each probe binds to the same analyte but possibly to different parts thereof, for instance to different (e.g. neighboring) or overlapping sections of the nucleotide sequence comprised by the nucleic acid molecule to be encoded. However, each of the plurality of the probes comprises the same identifier element (T). Accordingly, the unique identifier sequence identifies the target analyte but not the specific target analyte probe relative to other subsets of target analyte probes directed to the same target analyte. Thus, the target analyte probes are resilient to variation in the target analyte which may interfere with binding of one subset of probes but not another to the target analyte.

A “bipartite labeling probe” comprises a binding sequence capable of hybridizing the analyte and a binding probe sequence capable of binding a detectable signal molecule like a fluorophore or a nucleic acid sequence comprising a fluorophore.

A “decoding oligonucleotide” or an “adapter” or a/adapter segment” consists of at least two sequence elements. One sequence element that can specifically bind to a unique identifier sequence, referred to as an “identifier connector element “(t) or “first connector element” (t), and a second sequence element specifically binding to a signal oligonucleotide, referred to as “translator element” (c). The length of the sequence elements is within the range 8-60 nt, preferably 12-40 nt, more preferably 14-20 nt, de-pending on the number of analytes to be encoded, the stability of interaction needed, and the number of different signal oligonucleotides used. The length of the two sequence elements may or may not be the same.

In this respect it is important to note that the decoding-oligonucleotide may be adapted to the specific needs for the respective test or diagnosis. For example, a patient sample may be first screened for general tumor markers, and then in consecutive rounds for markers of specific tumor subtypes (e.g. breast cancer) or even molecular subtypes of a specific cancer (e.g. HER2-negative vs. HER2-positive). In those cases, not only the analytical set, but also the decoding-oligonucleotides may be designed to provide a specific pattern, which allows to maximize the information gained from a sample.

In some advantageous embodiments, the decoding oligonucleotide in the kits and/or methods of the present disclosure may be a “multi-decoder”. A “multi-decoder” is a decoding oligonucleotide that consists of at least three sequence elements. One sequence element (the identifier connector element (t)) can specifically bind to a unique identifier sequence (identifier element (T)) and at least two other sequence elements (translator elements (c)) specifically bind different signal oligonucleotides (each of these sequence elements specifically binds a signal oligonucleotide that differs to all other signal oligonucleotides recruited by other elements of the multi-decoder). The length of the sequence elements is within the range 8-60 nt, preferably 12-40 nt, more preferably 14-20 nt, depending on the number of analytes detected, the stability needed, and the number of different signal oligonucleotides used. The length of the sequence elements may or may not be the same.

Therefore, in some advantageous embodiments, the decoding oligonucleotide is a multi-decoder comprising an identifier connector element (t) comprising a nucleotide sequence which is essentially complementary to at least a section of the unique identifier sequence of the identifier element (T) of the corresponding analyte-specific probe set, and at least two translator elements (c) comprising each a nucleotide sequence allowing a specific hybridization of a different signal oligonucleotide.

Therefore, the first translator element binds a different signal oligonucleotide as the second translator element. In particular, the signal oligonucleotides differ in the signal element comprised in the signal oligonucleotide, e.g., in the kind of the fluorophore.

A “signal oligonucleotide” or a “reporter” as used herein comprises at least two elements, a so-called “translator connector element” (C) or “second connector element” (C) having a nucleotide sequence specifically hybridizable to at least a section of the nucleotide sequence of the translator element (c) of the decoding oligonucleotide, and a “signal element” which provides a detectable signal. This element can either actively generate a detectable signal or provide such a signal via manipulation, e.g. fluorescent excitation. Typical signal elements are, for example, enzymes that catalyze a detectable reaction, fluorophores, radioactive elements or dyes.

A “set” refers to a plurality of moieties or subjects, e.g. analyte-specific probes or decoding oligonucleotides, whether the individual members of said plurality are identical or different from each other. In an analyte specific probe set, the analyte specific probes are identical in the identifier element (T) but may comprise a different binding element(S) for specifically interacting with the same analyte but for specifically interacting with different sub-structures of the same analyte to be encoded.

“Selective denaturation” may be the process of eliminating bound decoding oligonucleotides and signal oligonucleotides with highest efficiency while at the same time the target specific probes have to stay hybridized with the highest efficiency.

The total efficiency of these two combined events may to be at least 0.22 for two detection cycles, 0.37 for three detection cycles, 0.47 for four detection cycles, 0.55 for five detection cycles, 0.61 for six detection cycles, 0.65 for seven detection cycles, 0.69 for eight detection cycles, 0.72 for nine detection cycles and 0.74 for 10 detection cycles, 0.76 for 11 detection cycles and 0.78 for 12 detection cycles.

In an embodiment of the disclosure a single set refers to a plurality of oligonucleotides:

An “analyte specific probe set” refers to a plurality of moieties or subjects, e.g. analyte-specific probes that are different from each other and bind to independent regions of the analyte. A single analyte specific probe set is further characterized by the same unique identifier.

A “decoding oligonucleotide set” refers to a plurality of decoding oligonucleotides specific for a certain unique identifier needed to realize the encoding independent of the length of the code word. Each and all of the decoding oligonucleotides included in a “decoding oligonucleotide set” bind to the same unique identifier element (T) of the analyte-specific probe.

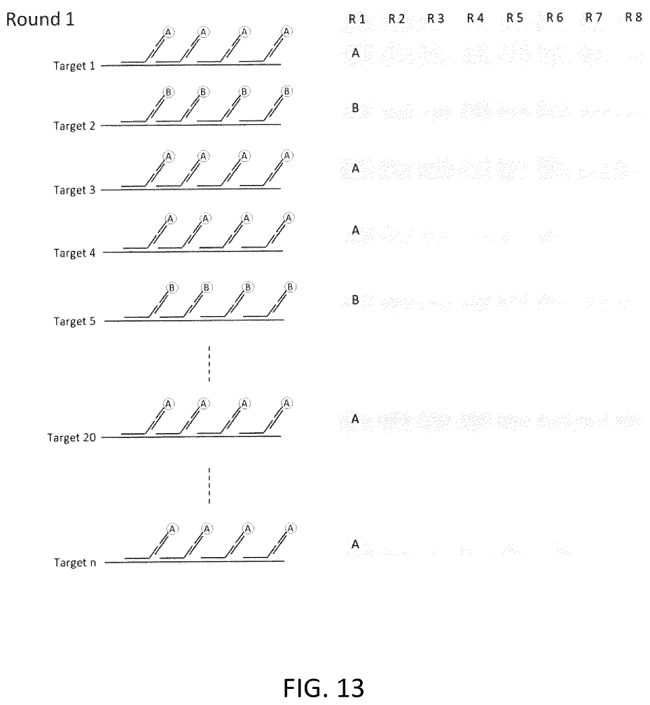

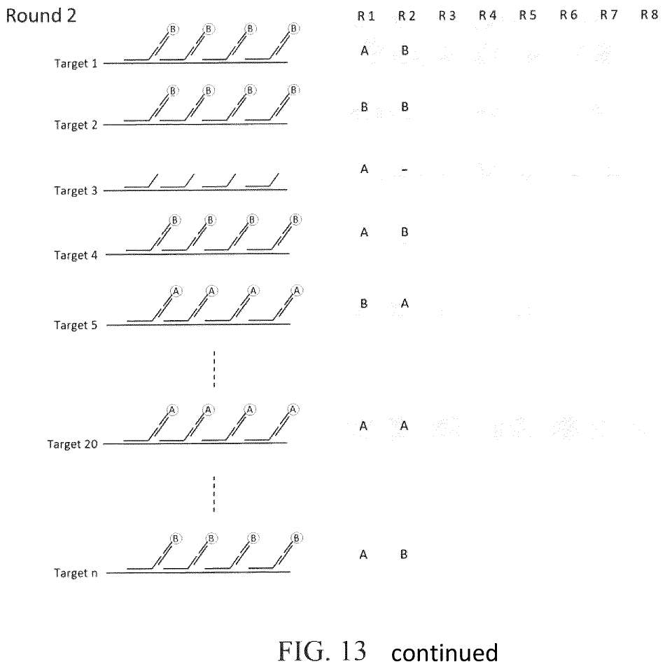

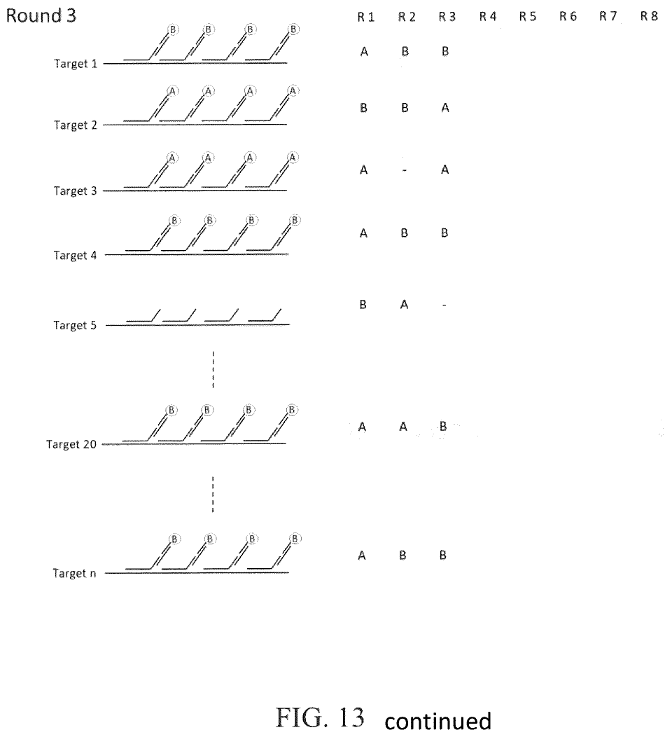

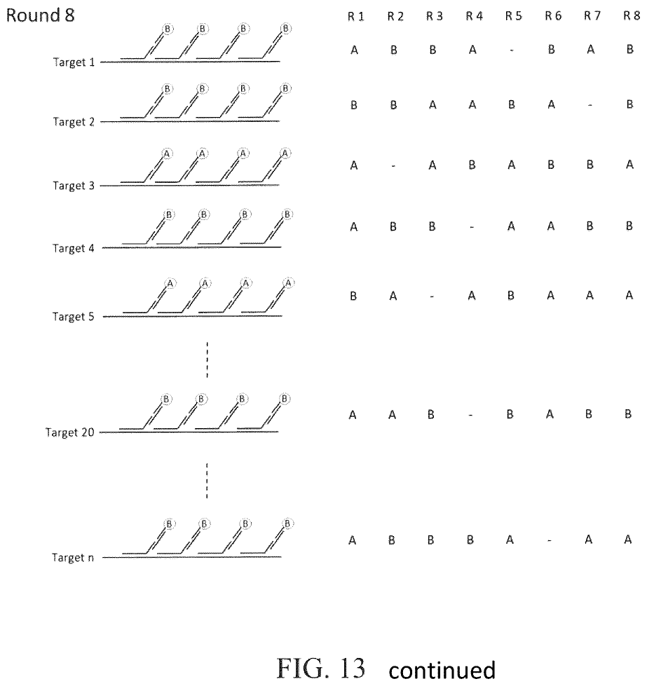

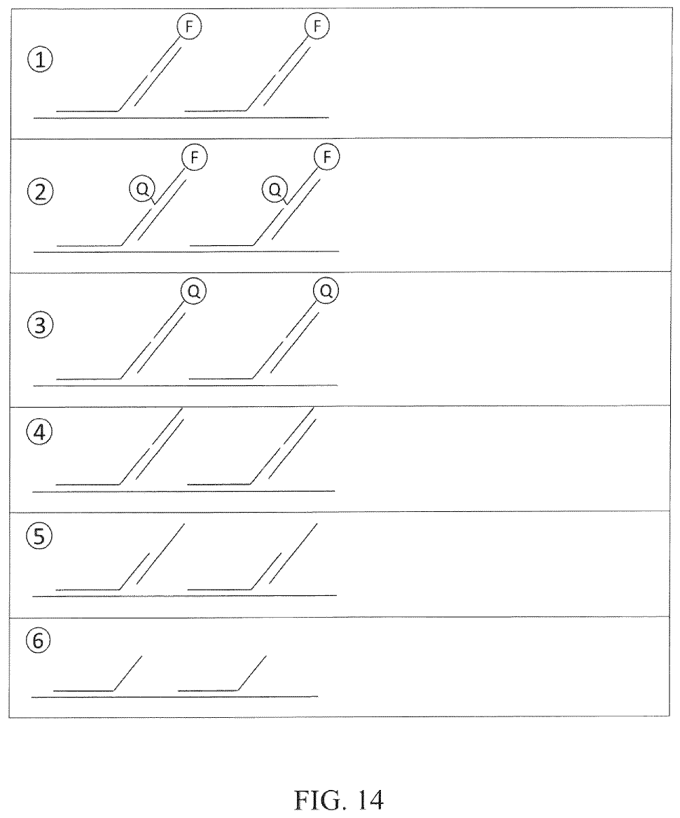

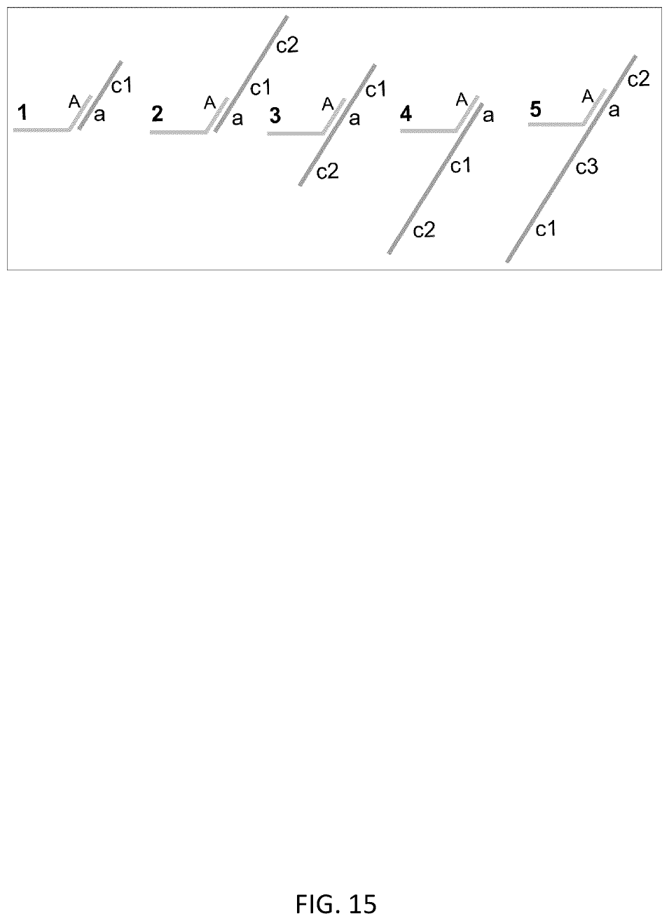

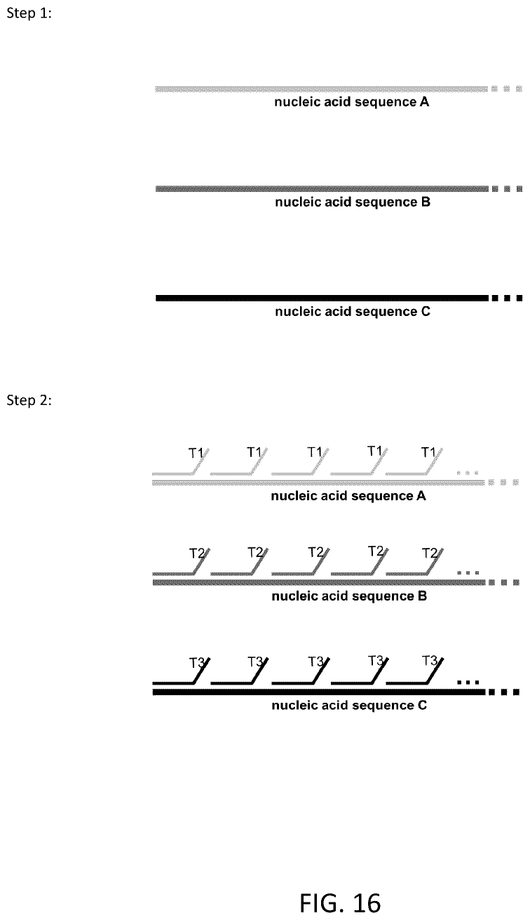

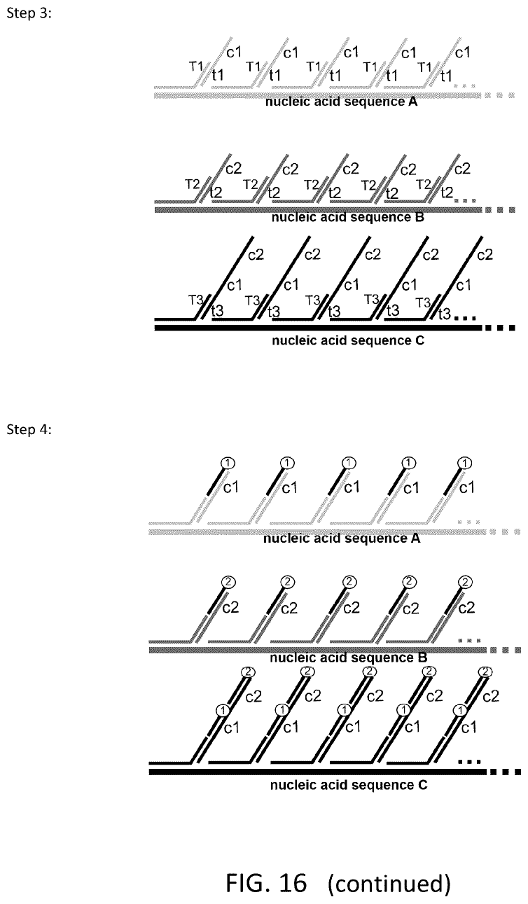

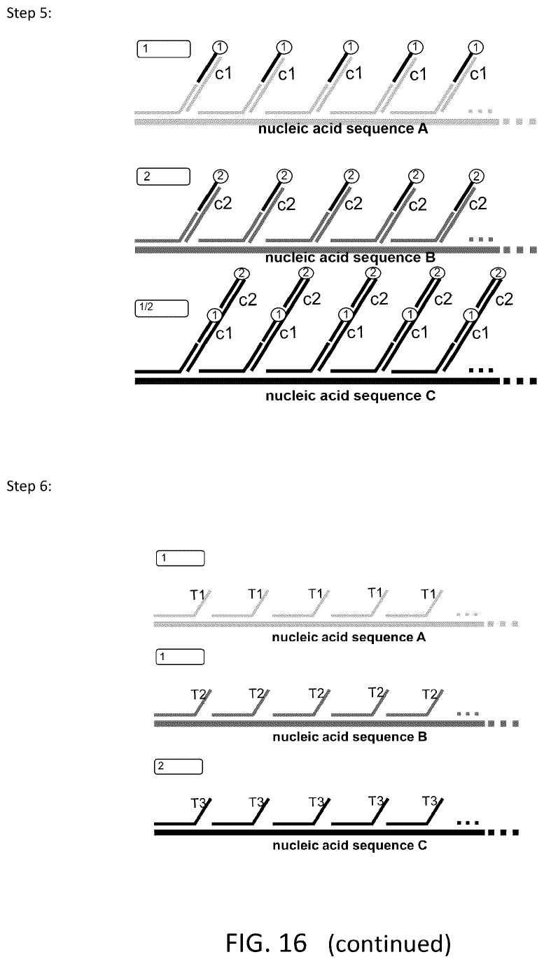

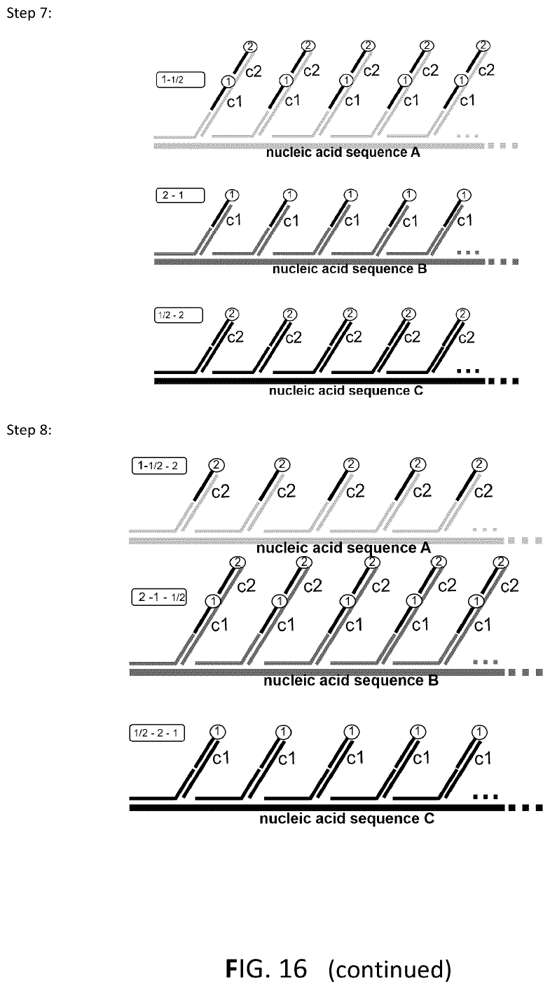

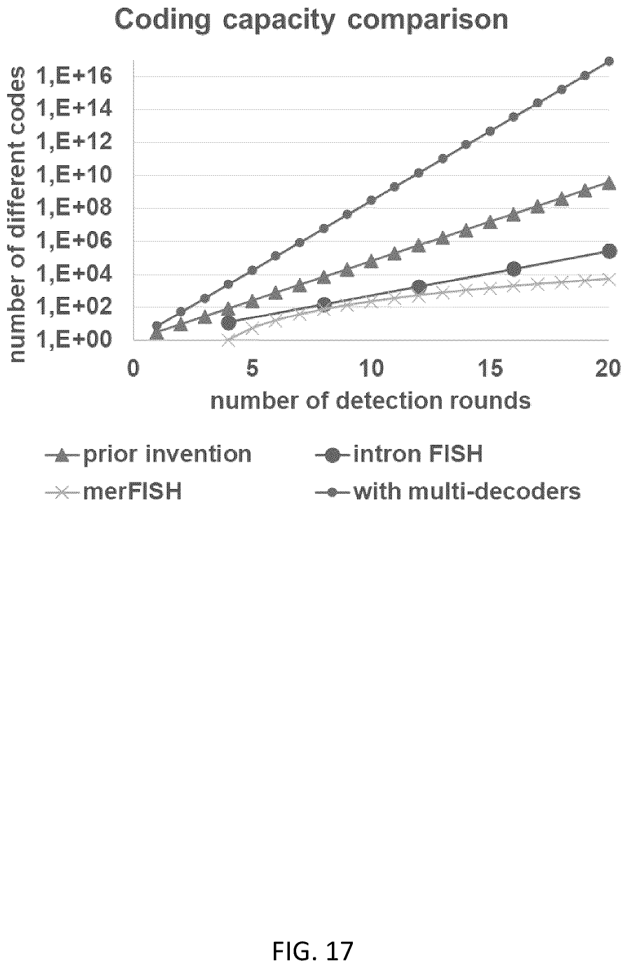

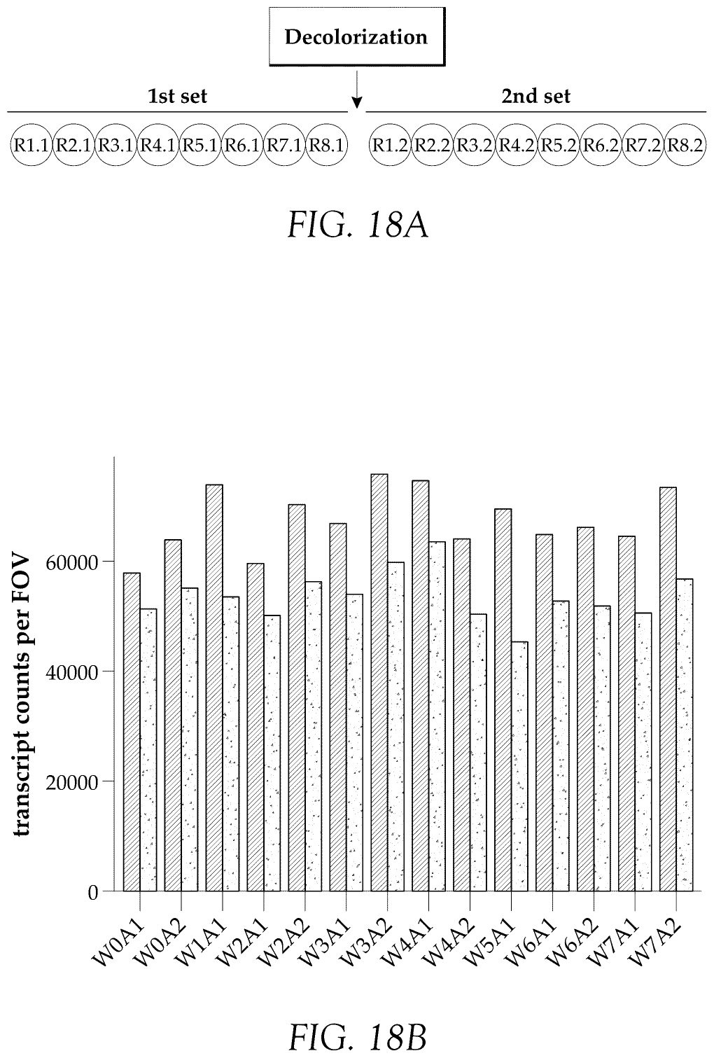

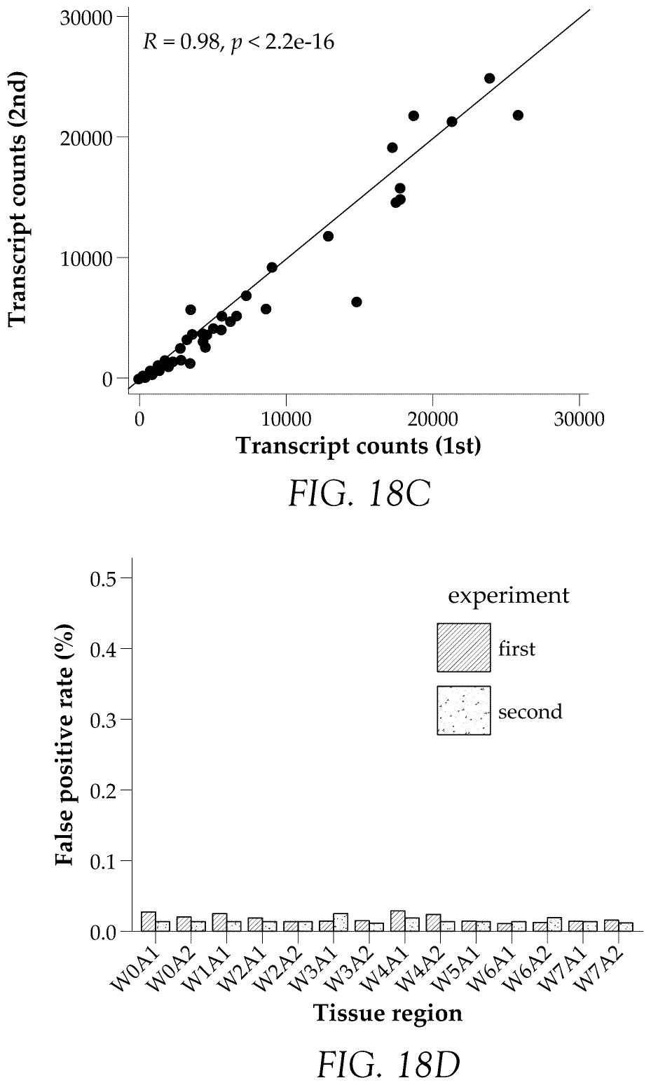

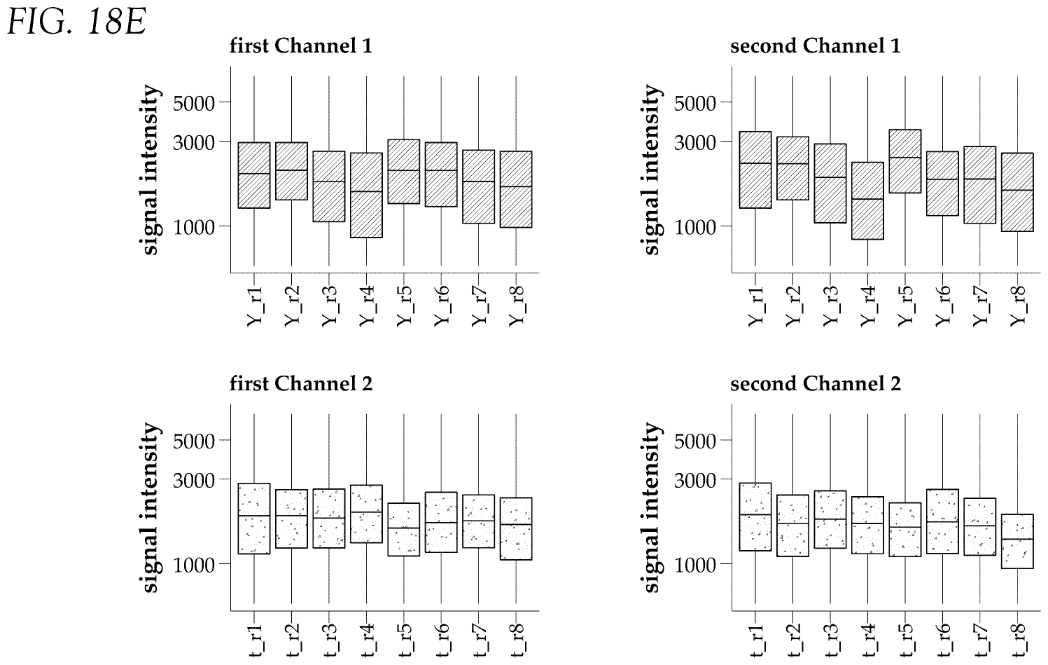

In certain embodiments, this pattern of binding or hybridization of the decoding oligonucleotides may be converted into a “codeword.” For example, the codewords could be also “101” and “110” for an analyte, where a value of 1 represents binding and a value of 0 represents no binding. The codewords may also have longer lengths in other embodiments (see FIG. 13). A codeword can be directly related to a specific unique identifier sequence of a analyte-specific probe. Accordingly, different analyte-specific probe may match certain codewords, which can then be used to identify the different analytes of the analyte-specific probe based on the binding patterns of the decoding oligonucleotide. However, if no binding is evident, then the codeword would be “000” in this example.

The values in each codeword can also be assigned in different fashions in some embodiments. For example, a value of 0 could represent binding while a value of 1 represents no binding. Similarly, a value of 1 could represent binding of a secondary nucleic acid probe with one type of signaling entity while a value of 0 could represent binding of a secondary nucleic acid probe with another or a second type of distinguishable signaling entity. These signaling entities could be distinguished, for example, via different colors of fluorescence. In some cases, values in codewords need not be confined to 0 and 1. The values could also be drawn from larger alphabets, such as ternary (e.g., 0, 1, and 2) or quaternary (e.g., 0, 1, 2, and 3) systems. Each different value could, for example, be represented by a different distinguishable signaling entity, including (in some cases) one value that may be represented by the absence of signal.

The codewords for each analyte may be assigned sequentially or may be assigned at random. For instance, a first analyte may be assigned to 101, while a second nucleic acid target may be assigned to 110. In addition, in some embodiments, the codewords may be assigned using an error-detection system or an error-correcting system, such as a Hamming system, a Golay code, or an extended Hamming system (or a SECDED system, i.e., single error correction, double error detection). Generally speaking, such systems can be used to identify where errors have occurred, and in some cases, such systems can also be used to correct the errors and determine what the correct codeword should have been. For example, a codeword such as 001 may be detected as invalid and corrected using such a system to 101, e.g., if 001 is not previously assigned to a different target sequence. A variety of different error-correcting codes can be used, many of which have previously been developed for use within the computer industry; however, such error-correcting systems have not typically been used within biological systems. For example, one or more digits of the codeword could be reserved for an internal control (e.g., checksum). A similar approach is for example used in the case of credit card numbers, but not known for biological systems. Additional examples of such error-correcting codes are discussed in more detail below.

Thus, in one embodiment an error-correction system is integrated in the binding element(S) and/or identifier element (T) and/or identifier connector element (t) and/or translator element (c) and/or translator connector element (C) and/or signal element.

“Essentially complementary” means, when referring to two nucleotide sequences, that both sequences can specifically hybridize to each other under stringent conditions, thereby forming a hybrid nucleic acid molecule with a sense and an antisense strand connected to each other via hydrogen bonds (Watson-and-Crick base pairs). “Essentially complementary” includes not only perfect base-pairing along the entire strands, i.e. perfect complementary sequences but also imperfect complementary sequences which, however, still have the capability to hybridize to each other under stringent conditions. Among experts it is often well accepted that an “essentially complementary” sequence has at least 88% sequence identity to a fully or perfectly complementary colinear sequence.

“Percent sequence identity” or “percent identity” in turn means that a sequence is compared to a claimed or described sequence after alignment of the sequence to be compared (the “Compared Sequence”) with the described or claimed sequence (the “Reference Sequence”). The percent identity is then determined according to the following formula: percent identity=100 [1-(C/R)] wherein C is the number of differences between the Reference Sequence and the Compared Sequence over the length of alignment between the Reference Sequence and the Compared Sequence, wherein
(i) each base or amino acid in the Reference Sequence that does not have a corresponding aligned base or amino acid in the Compared Sequence and
(ii) each gap in the Reference Sequence and
(iii) each aligned base or amino acid in the Reference Sequence that is different from an aligned base or amino acid in the Compared Sequence, constitutes a difference and (iv) the alignment has to start at position 1 of the aligned sequences;
and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity, then the Compared Sequence has the specified minimum percent identity to the Reference Sequence even though alignments may exist in which the herein above calculated percent identity is less than the specified percent identity.

In the “incubation” steps as understood herein the respective moieties or subjects such as probes or oligonucleotide, are brought into contact with each other under conditions well known to the skilled person allowing a specific binding or hybridization reaction, e.g. pH, temperature, salt conditions etc.

Such steps may therefore be preferably carried out in a liquid environment such as a buffer system which is well known in the art.

The “removing” steps according to the disclosure may include the washing away of the moieties or subjects to be removed such as the probes or oligonucleotides by certain conditions, e.g. pH, temperature, salt conditions etc., as known in the art.

It is understood that in an embodiment of the method according to the present disclosure a plurality of analytes can be encoded. This requires the use of different sets of analyte-specific probes in step (1). The analyte-specific probes of a particular set differ from the analyte-specific probes of another set. This means that the analyte-specific probes of set 1 bind to analyte 1, the analyte-specific probes of set 2 bind to analyte 2, the analyte-specific probes of set 3 bind to analyte 3, etc. In this embodiment also the use of different sets of decoding oligonucleotides is required in the methods according to the present disclosure.

The decoding oligonucleotides of a particular set differ from the decoding oligonucleotides of another set. This means, the decoding oligonucleotides of set 1 bind to the analyte-specific probes of above set 1 of analyte-specific probes, the decoding oligonucleotides of set 2 bind to the analyte-specific probes of above set 2 of analyte-specific probes, the decoding oligonucleotides of set 3 bind to the analyte-specific probes of above set 3 of analyte-specific probes, etc.

In this embodiment where a plurality of analytes is to be encoded the different sets of analyte-specific probes may be provided as a premixture of different sets of analyte-specific probes and/or the different sets of decoding oligonucleotides may be provided as a premixture of different sets of decoding oligonucleotides. Each mixture may be contained in a single vial. Alternatively, the different sets of analyte-specific probes and/or the different sets of decoding oligonucleotides may be provided in consecutive steps (as outlined before).

A “kit” is a combination of individual elements useful for carrying out the use and/or method of the disclosure, wherein the elements are optimized for use together in the methods. The kits may also contain additional reagents, chemicals, buffers, reaction vials etc. which may be useful for carrying out the method according to the disclosure. Such kits unify all essential elements required to work the method according to the disclosure, thus minimizing the risk of errors. Therefore, such kits also allow semi-skilled laboratory staff to perform the method according to the present disclosure.

In yet another aspect the kit may be designed for the use of technical laymen, for examples in environments which necessitate quick analysis of samples for certain markers, such as airports, border control, customs control, personalized medicine, self-diagnosis at home, etc. Thus, the invention also encompasses such uses which do not necessitate the handling of a skilled personal, but only fundamentally trained personal following step-by-step instructions and “ready-to-use”-kits.

The term “quencher” or “quencher dye” or “quencher molecule” refers to a dye or an equivalent molecule, such as nucleoside guanosine (G) or 2′-deoxyguanosine (dG), which is capable of reducing the fluorescence of a fluorescent reporter dye or donor dye. A quencher dye may be a fluorescent dye or non-fluorescent dye. When the quencher is a fluorescent dye, its fluorescence wavelength is typically substantially different from that of the reporter dye and the quencher fluorescence is usually not monitored during an assay. Some embodiments of the present disclosure disclose signal oligonucleotides comprising a quencher and/or a quencher in combination with a signal element (see FIG. 14), and therefore the signal oligonucleotides is not detectable during imaging. Quenching and other optical effects can also be used in order to identify co-localization of certain signals. For example, the co-localization of a green and red signal may result in a yellow signal, thereby emphasizing a certain maximal distance between both signals. Thus, another level of information can be added by the choice of the fluorescent reporter dye(s).

In an embodiment of the disclosure the sample is a biological sample, preferably comprising biological tissue, further preferably comprising biological cells. A biological sample may be derived from an organ, organoids, cell cultures, stem cells, cell suspensions, primary cells, samples infected by viruses, bacteria or fungi, eukaryotic or prokaryotic samples, smears, disease samples, a tissue section.

Consistent with the above, disclosed herein are compositions, methods, kits and compositions related to the durable identification of target analytes in samples for which the sequence of the target analyte is either unknown or known to differ from an expected or known target analyte sequence obtained from another source or the same source at a different time or cell or tissue. The approaches herein relate to the application of cutting-edge molecular techniques to a substantially broader range of sample sources, thereby allowing individuals with uncharacterized target analyte sequences to nonetheless benefit from the technical advances related to target analyte detection and localization. Similarly, the disclosure herein relates to enabling the detection of some embodiments of rapidly evolving target analytes such as oncogenes or viral genomes despite their sequences being unknown.

Due to the use of multiple probe subsets in a given target probe set that target nonoverlapping regions of a target analyte, a probe or set of probes may be designed to bind the analyte at a plurality of distinct positions, even though the analyte's actual sequence may differ from that of the expected sequence such that at least some of the set of probes do not bind to the analyte. Nonetheless, the remainder of the set of probes remains able to bind the analyte such that it may be detected despite varying from the expected sequence. Variations of this type often occur in diverse populations, such as various human populations, or among rapidly evolving populations, such as viral or other pathogen populations.

Accordingly, some use of the kits, methods and systems herein result in a composition comprising a target analyte that is bound by probes that are targeted to some of the plurality of target positions on the target analyte, while the subset of probes that target a target position on the target analyte for which there is a variation between the target analyte and the expected sequence exhibit no target binding. Thus, for the target analyte, some sets of probes directed to the target analyte exhibit binding to the target analyte, while other sets of probes directed to the target analyte (particularly to regions of the target analyte for which there is variation between the actual and the expected sequence) exhibit little or in some cases absolutely no binding to the target analyte. A composition resulting from such a use of the methods, kits or systems herein comprises a Probe set comprising a plurality of nonoverlapping probe subsets directed to nonoverlapping target regions on a target analyte, wherein at least one probe subset comprises members bound to the target analyte, while at least a second probe subset does not comprise members bound to the target analyte. Often, the target analyte differs in its sequence from the expected target analyte sequence at the binding site of the second probe subset, for example by at least one SNP, an insertion or deletion (in/del) a translocation, a duplication junction, a post-transcriptional modification, an epigenomic modification or other variation impacting binding of the second probe subset to the analyte. The composition further comprises decoding oligos bound to the first probe subset bound to the target analyte at the first target analyte binding site, and signal probes bound to the decoding oligos, which are in turn bound to the first probe subset bound to the target analyte at the first target analyte binding site. In some cases, the signal probes are bound to the decoding oligos at a lower melting temperature than the bridging oligos are bound to the first set of target oligos or than the first set of target oligos are bound to the target analyte, such that the signal probes may be replaced through melting without disrupting the binding of the first target probe subset to the target analyte. Alternately, in some cases all of the probe categories are melted off and the entire annealing process is repeated so as to enable attribution of a multi-signal profile to the target analyte.

### Durability of the Systems, Methods, Compositions and Kits

The systems, methods, kits and compositions herein facilitate the resilient, durable detection of a target analyte despite or independent of there being some variation in the target analyte relative to an expected sequence or chemical composition.

Furthermore, this variation tolerance is exhibited across the full range of analytes detected in a particular implementation of the system, without the synthesizing of degenerate probes having, variant-redundant′ or, variant-accommodating′ or, variant-specific probes' such as are made when one synthesizes probes having bases with unspecified or only partially specified identities at a certain position-that is, probe populations for which a particular position is referred to as “N” because, within the probe population, probes having each of the four bases A, T, G, C, at the position specified as “N” are all present. Such probes have a defect in that, as the number of degenerate “N” positions increases in a probe, the number of probes necessary to have one probe exhibiting the specific base at each of the positions increases by 4^{N}. That is, in a degenerate probe set, a probe that matches a target at all of, say, 4 positions will represent only (¼)⁴, or 1/256 of the total probe population. Thus, though degenerate probes are useful for accommodating single SNPs, as the number or variations increases, the number of probes that need to be generated becomes prohibitive, particularly when target analytes are to be detected on the scale contemplated here. Additionally, the system herein does not rely upon knowledge of where specific variations may occur, or the type of variations. Degenerate probes, in contrast, require that the site of a potential variation be known, and that the type of variation at that site (SNP, in/del of known size, for example) also be known. The systems, methods, kits and compositions herein are durable in that they accommodate a broad range of variations and do not require a priori knowledge of either the identity or the location of potential variations beterrn and expected target analyte and a target analyte in a sample.

Furthermore, no additional fluorophores and no additional fluorophore-labeled oligos are required to facilitate this detection, despite it being able to scale onto levels necessary to accommodate the entire transcriptome or proteome of a particular cell, tissue, organ or multiple organs.

A broad range of target analyte variability is tolerated by the approaches herein. So long as probes of one subset of the set of probes targeting the target analyte are able to bind to the target analyte, then the target analyte will be detected. That is, changes outside of the binding site of at least one subset of the binding probes, including sequence variation up to the point that the target is unrecognizable outside of that biding site, or deletion or splicing event resulting in a substantial portion of the target analyte sequence being absent in a sample variant, does not preclude detection of the analyte. Furthermore, no knowledge of the variation landscape of the target analyte is required.

Within a particular probe binding site, the percent difference among colinear sequences is determined by the hybridization conditions for the probe and template. Generally, a probe may bind to a target analyte site if there is a sequence identity of at least 60%, at least 65%, at least 70%, at least 75%, at least 80%, at least 85%, at least 90%, or at least 95%. In some cases, the probe will tolerate a sequence identity of 85%-99%. For the purpose of assessing sequence identity, sequences are assumed to be colinear and of equal length, and post-transcriptional, post-translational or epigenetic modifications (pseudo-uridylation, phosphorylation and cytosine methylation, respectively are offered as examples, though many more are known in the art) that impact base pairing or epitope identification are considered to be nonidentical.

So long as a single probe site is preserved in a target analyte, there is substantial tolerance of variation throughout the remainder of the target analyte. That is, a target analyte that exhibits, for example a uniformly distributed 50% identity to an expected target analyte sequence may not be detected, but a target analyte exhibiting less than that level of identity, o even substantial deletions, replacements, insertions, or other substantial variation may nonetheless be detected so long as at least one probe site is preserved having sequence identity sufficient for probe binding.

Accordingly, practice of the methods, systems and kits herein may result in a composition comprising a molecule comprising a target analyte to which a first probe is bound at a first probe site of the target analyte, and to which in some cases a decoding oligo and a signal oligo are indirectly attached, and may further comprise a second probe that is directed to a second site of the expected target analyte and that comprises a decoding oligo-binding site common with the first probe, but which is not attached to the sample target analyte.

The methods herein are particularly qualified to encode, identify, detect, count or quantify analytes or single analytes molecules in a biological sample, i.e. such as a sample which contains nucleic acids or proteins as said analytes. It is understood that the biological sample may be in a form as it is in its natural environment (i.e. liquid, semi-liquid, solid, for example.), or processed, e.g. as a dried film on the surface of a device which may be re-liquefied before the method is carried out.

In another embodiment of the disclosure prior to step (2) the biological tissue and/or biological cells are fixed. For example, in some embodiments, the cell and/or the tissue is fixed prior to introducing the probes, e.g., to preserve the positions of the analytes like nucleic acids within the cell. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like. In one embodiment, a cell may be fixed using Hepes-glutamic acid buffer-mediated organic solvent (HOPE).

This measure has the advantage that the analytes to be encoded, e.g. the nuclei acids or proteins, are immobilized and cannot escape. In doing so, the analytes then prepared for a better detection or encoding by the method according to the disclosure.

In yet a further embodiment within the set of analyte-specific probes the individual analyte-specific probes comprise binding elements (S1, S2, S3, S4, S5) which specifically interact with different sub-structures of one of the analytes to be encoded.

By this measure the method becomes even more robust and reliable because the signal intensity obtained at the end of the method or a cycle, respectively, is increased. It is understood that the individual probes of a set while binding to the same analyte differ in their binding position or binding site at or on the analyte. The binding elements S1, S2, S3, S4, S5 etc. of the first, second, third fourth, fifth etc. analyte-specific probes therefore bind to or at a different position which, however, may or may not overlap.

Beneficially, probes of target analyte probe subsets S1, S2, S3, S4, and S5, for example do not need to all bind to the target analyte for the detection to succeed. Because the probe subsets are all labeled using a common unique identifier, the system is tolerant to sequence variation severe enough to disrupt binding at up to all but one of the target analyte probe subset binding sites.

In an advantageous embodiment, the present disclosure pertains to kit for multiplex analyte encoding, comprising: (A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), and (A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), and wherein the analyte-specific probes in each set of analyte-specific probes binds to the same analyte and comprises the same nucleotide sequence of the identifier element (T) which is unique to said analyte; and wherein (optionally) the number of probes and/or targets of first set of analyte-specific probes according to step A1 (i.e., the transcript plexity of A1) is at least 10 times higher than the number of probes and/or targets of the second set of analyte-specific probes according to step A2 (i.e., the transcript plexity of A2); and (B1) at least a first set of decoding oligonucleotides per analyte, wherein in each set of decoding oligonucleotides for an individual analyte each decoding oligonucleotide comprises: (aa) an identifier connector element (t) comprising a nucleotide sequence which is essentially complementary to at least a section of the unique identifier sequence of the identifier element (T) of the corresponding analyte-specific probe set, and (bb) a translator element (c) comprising a nucleotide sequence allowing a specific hybridization of a signal oligonucleotide; and (B2) at least a second set of decoding oligonucleotides per analyte, wherein in each set of decoding oligonucleotides for an individual analyte each decoding oligonucleotide comprises: (aa) an identifier connector element (t) comprising a nucleotide sequence which is essentially complementary to at least a section of the unique identifier sequence of the identifier element (T) of the corresponding analyte-specific probe set, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the identifier connect element (t); and (C) a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element.

Similarly, in an advantageous embodiment, the present disclosure pertains to a kit for multiplex analyte encoding, comprising: (A) contacting the sample with a set of polymorphic analyte-specific probes for encoding different variations of the same polymorphic analyte, each analyte-specific probe interacting with a different variation and/or sub-structure of the polymorphic analyte, wherein the polymorphic analyte is present in the sample in at least as two or more variant forms of a specific nucleic acid sequence and wherein the sequences of each polymorphic analyte are related to each other, in some cases with a sequence identity of between 85% to 99%, each polymorphic analyte-specific probe comprising (aa) a binding element(S) that specifically interacts with one of the at least two or more variants and/or sub-structure of the polymorphic analyte to be encoded, and (bb) an identifier element (T) comprising a nucleotide sequence which is unique to the polymorphic analyte to be encoded (unique set identifier sequence), wherein each set of polymorphic analyte-specific probes differ from another set of polymorphic analyte-specific probes in the nucleotide sequence of the identifier element (T); and (B) contacting the sample with at least a first set of decoding oligonucleotides per polymorphic analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique set identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element facilitating a signal which is specific for the polymorphic analyte; and detecting the signal caused by the signal element; optionally selectively removing the decoding oligonucleotides and signal oligonucleotides from the sample, thereby essentially maintaining the specific binding of the analyte-specific probes to the analytes to be encoded; erforming further cycles comprising steps A) to E) for each further polymorphic analyte to generate an encoding scheme with a specific signal set of polymorphic analytes, wherein in particular the last cycle may stop with step (D).

A multiplex method or assay allow the simultaneously measurement of multiple analytes according to the present disclosure it may be used to determine the presence or absence of a plurality of predetermined (known) analytes like nucleic acid target sequences in a sample. A analyte may be “predetermined” in that its sequence is known to design a probe that binds to the that target.

Alternately, an analyte may be predetermined in that it has an expected sequence from which a probe or set of probes may be designed to bind the analyte at a plurality of distinct positions, even though the analyte's actual sequence may differ from that of the expected sequence such that at least some of the set of probes do not bind to the analyte. Nonetheless, the remainder of the set of probes remains able to bind the analyte such that it may be detected despite varying from the expected sequence. Variations fo this type often occur in diverse populations, such as various human populations, or among rapidly evolving populations, such as viral or other pathogen populations.

In some advantageous embodiments according to the present disclosure at least 2, at least 5, at least 10, at least 15, at least 20, in particular at least 25, in particular at least 30 different analytes are detected and/or quantified in a sample. For example, there may be at least 5, at least 10, at least 20, at least 50, at least 75, at least 100, at least 300, at least 1,000, at least 3,000, at least 10,000, or at least 30,000 distinguishable analyte-specific probes that are applied to a sample, e.g., simultaneously or sequentially.

In the multiplexing methods of the present disclosure, in particular at least 2 different subgroups of analytes (e.g., mRNA molecules), tags with spatially overlap (i.e. a distance beyond the diffraction limit of the respective microscope) are targeted. Thus, the present invention allows an increased resolution and higher “information density” as compared to conventional methods.

With higher “information density” the number of diagnostic conclusions is meant, which can be drawn from a single sample, thereby reducing the number of samples needed and the time needed for a specific result. It also decreases the workload of the personnel performing the analysis and/or allow a higher throughput of samples as compared to conventional methods.

In some advantageous embodiments, at least 4 rounds to collect information for identification of the analyte are carried out, wherein multiple readout increases the accuracy of identification and avoids false positives. The unique tag can be identified by various techniques, including hybridization, e.g., with labeled probes, directly or indirectly or by sequencing (by synthesis, ligation). In particular, the identity of the tag can be encoded with one single signal (binary code), two or more signals, wherein the signal can be a fluorescent label (e.g., attached to an oligonucleotide). In some advantageous embodiments according to the present disclosure, the kit does not comprise sets of analyte-specific probes as defined under item A1) and A2).

Preferably, if the analyte in the kits or methods according to the present disclosure is a nucleic acid, each set of analyte-specific probes comprises at least five (5) analyte-specific probes, in particular at least ten (10) or at least fifteen (15) analyte-specific probes, in particular at least twenty (20) analyte-specific probes which specifically interact with different sub-structures of the same analyte. Nucleic acid analyte includes specific DNA molecules, e.g. genomic DNA, nuclear DNA, mitochondrial DNA, viral DNA, bacterial DNA, extra- or intracellular DNA etc., and specific mRNA molecules, e.g. hnRNA, miRNA, viral RNA, bacterial RNA, extra- or intracellular RNA, for example.

Preferably, if the analyte in the kits or methods according to the present disclosure is a peptide, a polypeptide or a protein, each set of analyte-specific probes comprises at least two (2) analyte-specific probes, in particular at least three (3) analyte-specific probes, in particular at least four (4) analyte-specific probes which specifically interact with different sub-structures of the same analyte.

In some advantageous embodiments according to the present disclosure the kit comprises at least two different sets of signal oligonucleotides, wherein the signal oligonucleotides in each set comprise a different signal element and comprise a different connector element (C).

In particular, the kit may comprise at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide.

In some embodiments the kit comprises at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets for at least one analyte differ in the translator element (c) comprising a nucleotide sequence allowing a specific hybridization of a signal oligonucleotide.

In some advantageous embodiments, the number of different sets of decoding oligonucleotides per analyte comprising different translator elements (c) corresponds to the number of different sets of signal oligonucleotides comprising different connector elements (C). However, the decoding oligonucleotides in a particular set of decoding oligonucleotides may interact with identical identifier elements (T) which are unique to a particular analyte. In particular, all sets of decoding oligonucleotides for the different analytes may comprise the same type(s) of translator element(s) (c).

In another aspect, the present disclosure is generally directed to a methods including acts of exposing a sample to a plurality of analyte-specific probes; for each of the analyte-specific probes, determining binding of the analyte-specific probes within the sample; creating codewords based on the binding of the analyte-specific probes, the decoding oligonucleotides and the signal oligonucleotides; and for at least some of the codewords, matching the codeword to a valid codeword. In certain embodiments, this pattern of binding or hybridization of the analyte-specific probes, the decoding oligonucleotides and the signal oligonucleotides may be converted into a “codeword.” For example, for instance, the codewords may be “101” and “110” for a first analyte and a second analyte, respectively, where a value of 1 represents binding and a value of 0 represents no binding of decoding oligonucleotides and/or the binding of signal oligonucleotides without and/or quenched signal element. The analyte in the detection round/cycle is therefore not detectable during imaging.

To create such a zero (0) in a codeword for an individual analyte the kit may comprise: (D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

To create such a zero (0) in a codeword for an individual analyte the kit may comprise: (D) at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and comprise a translator element that does not interact/bind to a signal oligonucleotide due to an unstable binding sequence and/or due to the translator element is to short (c) comprising a nucleotide sequence allowing a specific hybridization of a signal oligonucleotide.

In some advantageous embodiments, the kit comprises: (D) at least two (2) different sets of non-signal decoding oligonucleotides for binding to at least two different identifier elements (T) of analyte-specific probes, each set of non-signal decoding oligonucleotides interacting with a different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments, the different sets of non-signal decoding oligonucleotides may be comprised in a pre-mixture of different sets of non-signal decoding oligonucleotides or exist separately.

Furthermore, in some advantageous embodiments the kit may comprise: (E) a set of non-signal oligonucleotides, each non-signal oligonucleotide comprising: (aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

In some advantageous embodiments, the kit comprises: (E) at least two sets of non-signal oligonucleotides, each non-signal oligonucleotide comprising: (aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

In some advantageous embodiments, the different sets of non-signal oligonucleotides may be comprised in a pre-mixture of different sets of non-signal oligonucleotides or exist separately.

Further, in some embodiments the decoding oligonucleotides in a particular set of decoding oligonucleotides interacts with identical identifier elements (T) which are unique to a particular analyte, or which distinguish the target analyte from at least one other target analyte in the sample.

In some advantageous embodiments, the different sets of decoding oligonucleotides may be comprised in a pre-mixture of different sets of decoding oligonucleotides or exist separately. In some advantageous embodiments, the different sets of analyte-specific probes may be comprised in a pre-mixture of different sets of analyte-specific probes or exist separately. In some advantageous embodiments, the different sets of signal oligonucleotides may be comprised in a pre-mixture of different sets of signal oligonucleotides or exist separately.

In some advantageous embodiments, a mixture of decoding oligonucleotides and/or multi-decoders is provided that specifically hybridize to the unique identifier sequences of the probe sets. In some embodiments, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide. In some embodiments multi-decoders comprising at least three sequence elements are used, a first element that is complementary to the unique identifier sequences of the corresponding probe set and at least to additional sequence elements (translator elements) that provide sequences for the specific hybridization of at least two different signal oligonucleotides. The translator elements define the type of signals that are recruited to the multi-decoder. Different possible structures of a multi-decoder can be seen in FIG. 15. Since a multi-decoder does recruit a full signal oligonucleotide per translator element, the brightness of the signals in each channel is not lower than the brightness of signals with decoding oligonucleotides.

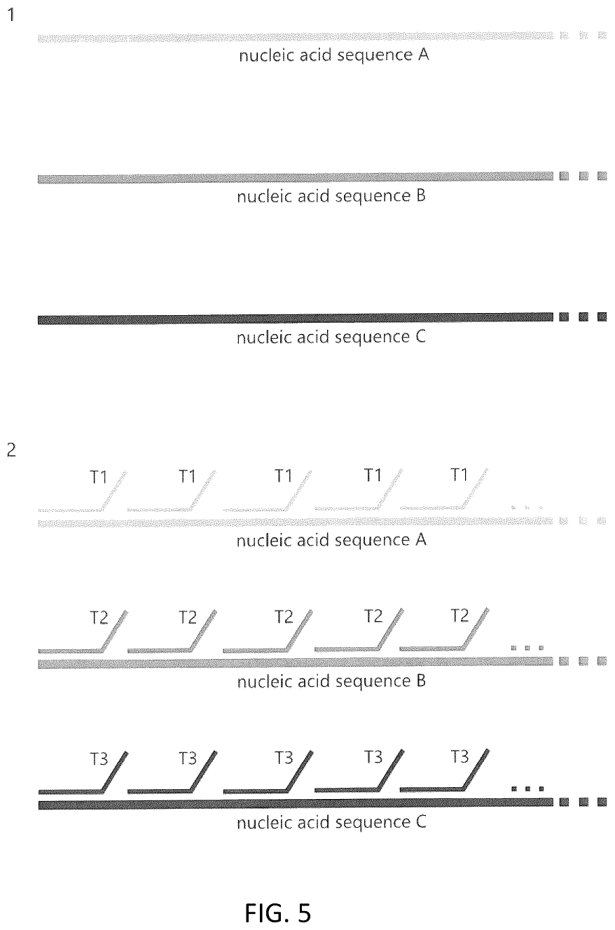

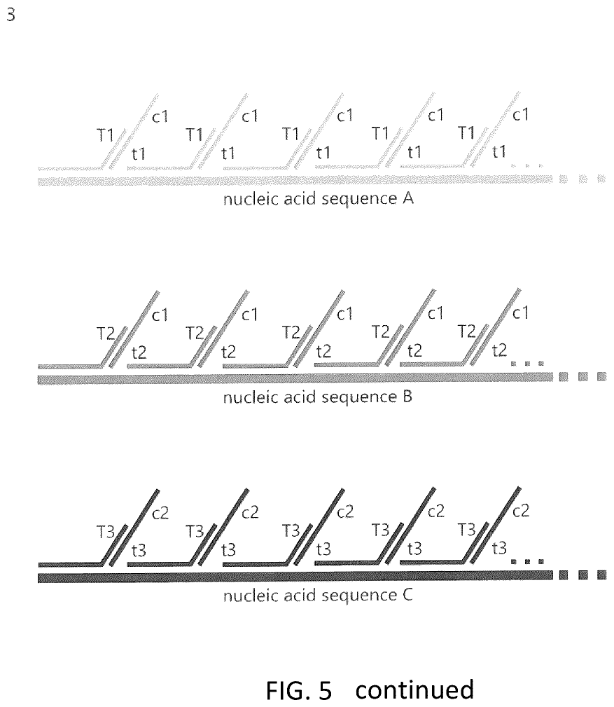

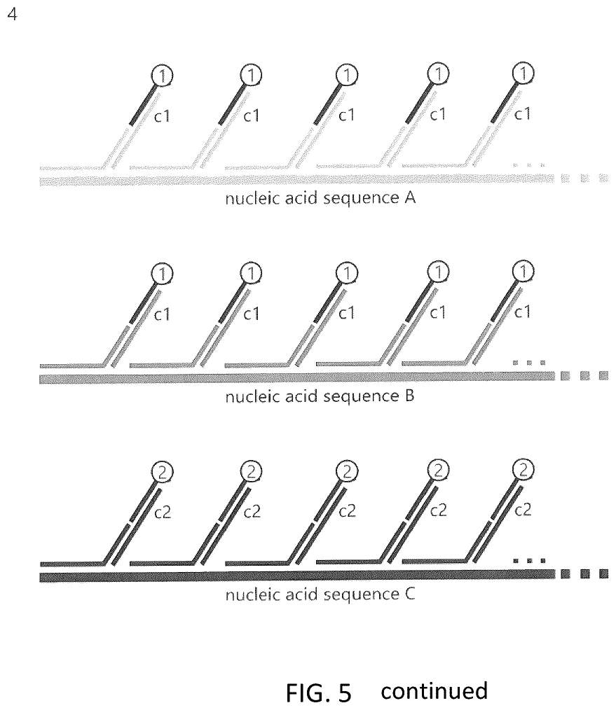

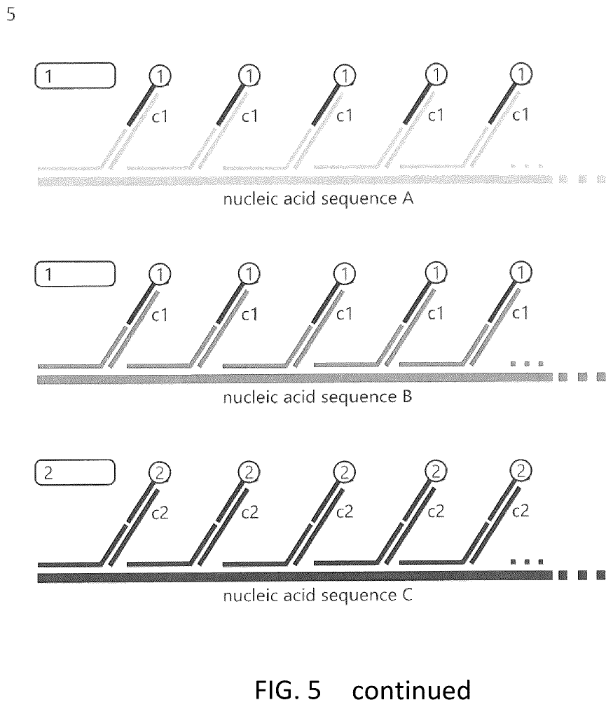

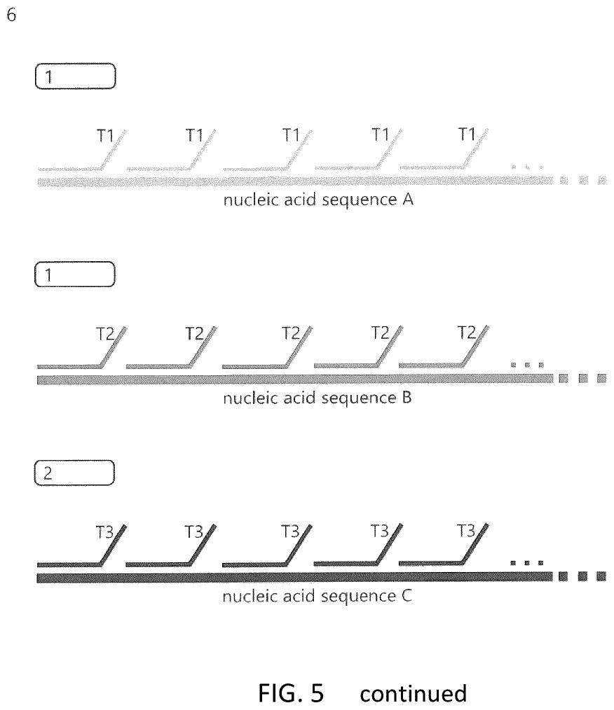

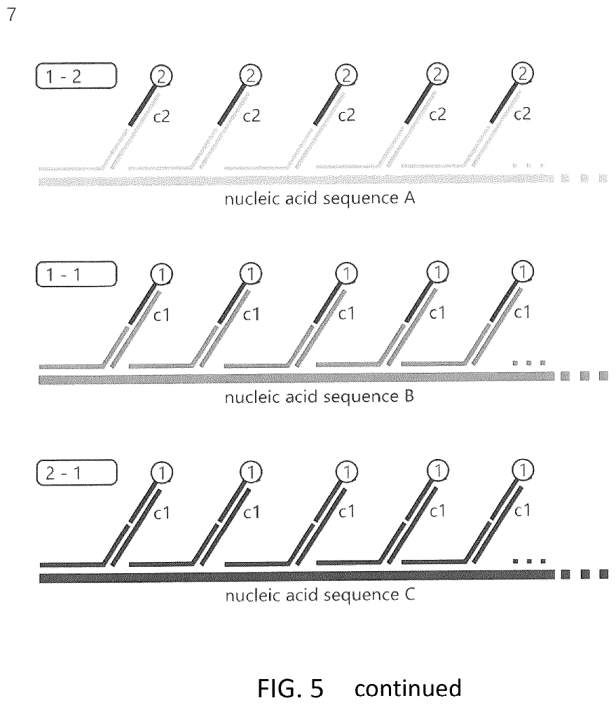

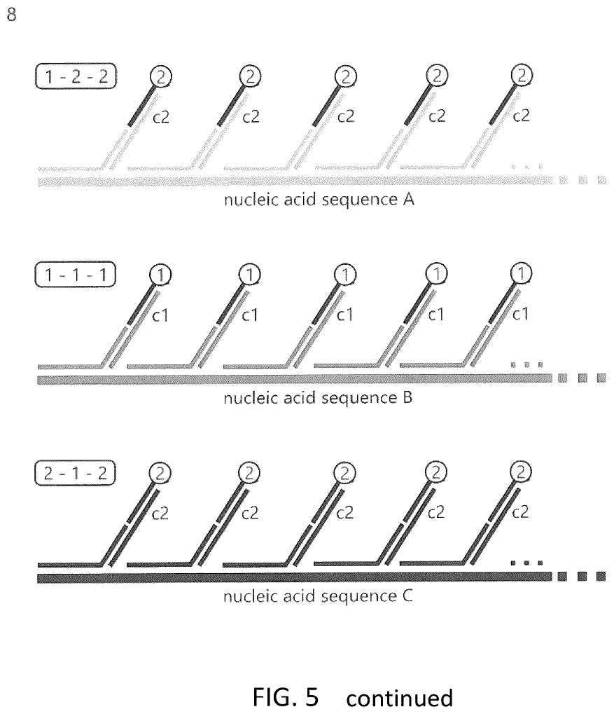

The usage of multi-decoders increases further the efficiency of the encoding scheme. FIG. 16 shows a possible encoding scheme using multi-decoders based upon the same conditions used for the examples with the decoding oligonucleotide with two sequence elements. One can clearly see that the multi-decoder-based encoding scheme can create a higher hamming distance, with the same number of rounds and the same number of different signal oligonucleotides used in the example of FIG. 5.

As mentioned above the analyte to be encoded may be a nucleic acid, preferably DNA, PNA or RNA, in particular mRNA, a peptide, polypeptide, a protein, and/or mixtures thereof.

In some advantageous embodiments, the binding element(S) comprises an amino acid sequence allowing a specific binding to the analyte to be encoded. The binding element(S) may comprise moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof.

In some advantageous embodiments, the binding element(S) may comprise or is an antibody or an antibody fragment selected from the group consisting of Fab, scFv; single domain, or a fragment thereof, bis scFv, F (ab) 2, F (ab) 3, minibody, diabody, triabody, tetrabody and tandab.

The present disclosure pertains in particular to a multiplex method for detecting different analytes in a sample by sequential signal-encoding of said analytes, comprising the steps of: (A1) contacting the sample with at least a first set of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and (A2) contacting the sample with at least a second set of analyte-specific probes for encoding of at least 20 different analytes, each set of analyte-specific probes interacting with a different analyte, wherein if the analyte is a nucleic acid each set of analyte-specific probes comprises at least five (5) analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and wherein (optionally) the number of probes and/or targets of first set of analyte-specific probes according to step A1 (i.e., the transcript plexity of A1) is at least 10 times higher than the number of probes and/or targets of the second set of analyte-specific probes according to step A2 (i.e. the transcript plexity of A2); and (B1) contacting the sample with at least a first set of decoding oligonucleotides per analyte set according to A1, wherein in each set of decoding oligonucleotides for an individual analyte each decoding oligonucleotide comprises: (aa) an identifier connector element (t) comprising a nucleotide sequence which is essentially complementary to at least a section of the unique identifier sequence of the identifier element (T) of the corresponding analyte-specific probe set, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (B2) contacting the sample with at least a second set of decoding oligonucleotides per analyte set according to A2, wherein in each set of decoding oligonucleotides for an individual analyte each decoding oligonucleotide comprises: (aa) an identifier connector element (t) comprising a nucleotide sequence which is essentially complementary to at least a section of the unique identifier sequence of the identifier element (T) of the corresponding analyte-specific probe set, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element; (D) Detecting the signal caused by the signal element; (E) selectively removing the decoding oligonucleotides and signal oligonucleotides from the sample, thereby essentially maintaining the specific binding of the analyte-specific probes to the analytes to be encoded; (F) Performing at least three (3) further cycles comprising steps B) to E) to generate an encoding scheme with a code word per analyte, wherein in particular the last cycle may stop with step (D).

Similarly, the present disclosure pertains in particular to a method for detecting a polymorphic analyte in a sample by specific signal-encoding of said polymorphic analyte. Methods variously comprise one or more of the steps of (A) contacting the sample with a set of polymorphic analyte-specific probes for encoding different variations of the same polymorphic analyte, each analyte-specific probe interacting with a different variation and/or sub-structure of the polymorphic analyte, wherein the polymorphic analyte is present in the sample in at least as two or more variant forms of a specific nucleic acid sequence and wherein the sequences of each polymorphic analyte are related to each other with a sequence identity of, for example, between 85% to 99%, each polymorphic analyte-specific probe comprising (aa) a binding element(S) that specifically interacts with one of the at least two or more variants and/or sub-structure of the polymorphic analyte to be encoded, and (bb) an identifier element (T) comprising a nucleotide sequence which is unique to the polymorphic analyte to be encoded (unique set identifier sequence), wherein each set of polymorphic analyte-specific probes differ from another set of polymorphic analyte-specific probes in the nucleotide sequence of the identifier element (T)., In some cases, the probes of a polymorphic analyte-specific probe set are non-overlapping, such that binding of probes of a first subset of probes to the target analyte is independent of binding of probes of a second subset of probes to the same target analyte; (B) contacting the sample with at least a first set of decoding oligonucleotides per polymorphic analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique set identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element facilitating a signal which is specific for the polymorphic analyte; and detecting the signal caused by the signal element; (E) optionally selectively removing the decoding oligonucleotides and signal oligonucleotides from the sample, thereby essentially maintaining the specific binding of the analyte-specific probes to the analytes to be encoded; performing further cycles comprising steps A) to E) for each further polymorphic analyte to generate an encoding scheme with a specific signal per set of polymorphic analytes, wherein in particular the last cycle may stop with step (D).

As mentioned above, the method according to the present disclosure comprises selectively removing the decoding oligonucleotides and signal oligonucleotides from the sample, thereby essentially maintaining the specific binding of the analyte-specific probes to the analyte to be encoded. In particular all steps are performed sequentially. However, some steps may be performed simultaneously, in particular the contacting steps A) to C), in particular B) and C).

By this measure the requirements for another round/cycle of binding further decoding oligonucleotides to the same analyte-specific probes are established, thus finally resulting in a code or encoding scheme comprising more than one signal. This step is realized by applying conditions and factors well known to the skilled person, e.g., pH, temperature, salt conditions, oligonucleotide concentration, polymers, etc.

In another embodiment of the present disclosure, the method may comprise repeating steps (B)-(E) at least three times to generate an encoding scheme. With this measure a code of four signals in case of four cycles/rounds which are carried out by the user, where ‘n’ is an integer representing the number of rounds. The encoding capacity of the method according to the disclosure is herewith increased depending on the nature of the analyte and the needs of the operator. In an embodiment of the disclosure, the said encoding scheme is predetermined and allocated to the analyte to be encoded.

However, this measure enables a precise experimental set-up by providing the appropriate sequential order of the employed decoding and signal oligonucleotides and, therefore, allows the correct allocation of a specific analyte to a respective encoding scheme. The decoding oligonucleotides which are used in repeated steps (B)-(D2) may comprise a translator element (c2) which is identical with the translator element (c1) of the decoding oligonucleotides used in previous steps (B)-(E). In another embodiment of the disclosure decoding oligonucleotides are used in repeated steps (B)-(E) comprising a translator element (c2) which differs from the translator element (c1) of the decoding oligonucleotides used in previous steps (B)-(E). It is understood that the decoding elements may or may not be changed from round to round, i.e., in the second round (B)-(E) comprising the translator element c2, in the third round (B)-(E) comprising the translator element c3, in the fourth round (B)-(E) comprising the translator element c4 etc., wherein ‘n’ is an integer representing the number of rounds. The signal oligonucleotides which are used in repeated steps (B)-(E) may comprise a signal element which is identical with the signal element of the decoding oligonucleotides used in previous steps (B)-(E). In a further embodiment of the disclosure signal oligonucleotides are used in repeated steps (B)-(E) comprising a signal element which differs from the signal element of the decoding oligonucleotides used in previous steps (B)-(E). In some embodiments no-signal oligonucleotides and/or no-signal decoding oligonucleotides for an individual analyte are used, resulting to the value 0 in the codeword for this cycle/position. In some embodiments in a repeated cycle no decoding oligonucleotides for an individual analyte is contacted with the sample resulting also to the value 0 in the codeword for this cycle/position.

By this measure each round the same or a different signal is provided resulting in an encoding scheme characterized by a signal sequence consisting of numerous different signals. This measure allows the creation of a unique code or code word which differs from all other code words of the encoding scheme. In another embodiment of the disclosure, the binding element(S) of the analyte-specific probe comprises a nucleic acid comprising a nucleotide sequence allowing a specific binding to the analyte to be encoded, preferably a specific hybridization to the analyte to be encoded.

In some advantageous embodiments, all steps are automated, in particular wherein steps B) to F) are automated, in particular by using a robotic system and/or an optical multiplexing system according to the present disclosure. In some examples, the steps may be performed in a fluidic system.

As mentioned above, with the methods according to the present disclosure an encoding scheme with a code word per analyte set is generated. Therefore, each analyte set may be associated with a specific code word, wherein said code word comprise a number of positions, and wherein each position corresponds to one cycle resulting in a plurality of distinguishable encoding schemes with the plurality of code words. In particular, said encoding scheme may be predetermined and allocated to the analyte to be encoded.

In some advantageous embodiments, the code words obtained for the individual analytes in the performed cycles comprise the detected signals and additionally at least one element corresponding to no detected signal like 0.1 or 0,1,2 etc. (see also FIG. 13 and FIG. 14). In particular, no signal is detected for at least one analyte within at least one cycle if using a non-signal probe according to FIG. 14, Nr. 2 to 4, or a non-signal decoding oligonucleotide as shown in FIG. 14, Nr. 5, or if in one cycle no decoding oligonucleotide is contacted with the corresponding identifier sequence comprised on analyte-specific probe interacting with the corresponding analyte in the sample. In this cycle the position has the value zero (0).

In some advantageous embodiments, at least for one individual analyte a position of the code word is zero (0). In particular, the code word zero (0) is generated by using no decoding oligonucleotides having an identifier connector element (t) comprising a nucleotide sequence which is essentially complementary to at least a section of the unique identifier sequence of the identifier element (T) of a corresponding analyte-specific probe for an individual analyte. As mentioned above, in some embodiments, if at least for one individual analyte a position of the code word is zero (0) in this cycle no corresponding decoding oligonucleotides having an identifier connector element (t) comprising a nucleotide sequence which is essentially complementary to at least a section of the unique identifier sequence of the identifier element (T) of a corresponding analyte-specific probe for an individual analyte are used.

Furthermore, in some advantageous embodiments, the sample is contacted with at least two different sets of signal oligonucleotides, wherein the signal oligonucleotides in each set comprise a different signal element and comprise a different connector element (C).

In more particular embodiments, the sample is contacted with at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide.

In more particular embodiments, the sample is contacted with at least two different sets of decoding oligonucleotides per analyte, wherein the decoding oligonucleotides comprised in these different sets comprise the same identifier connector element (t) comprising a nucleotide sequence which is essentially complementary to at least a section of the unique identifier sequence of the identifier element (T) of the corresponding analyte-specific probe set, and wherein the decoding oligonucleotides of the different sets per analyte differ in the translator element (c) comprising a nucleotide sequence allowing a specific hybridization of a signal oligonucleotide; wherein only one set of decoding oligonucleotides per analyte is used per cycle, and/or wherein different sets of decoding oligonucleotides are used in different cycles in combination with the corresponding set of signal oligonucleotides in the same cycle.

In some advantageous embodiments, the number of different sets of decoding oligonucleotides per analyte comprising different translator elements (c) corresponds to the number of different sets of signal oligonucleotides comprising different connector elements (C). All sets of decoding oligonucleotides for the different analytes may comprise the same type(s) of translator element(s) (c).

In another aspect the translator element(s) (c) may differ between sets.

In some advantageous embodiments of the method according to the present disclosure, the sample is contacted with at least a set of non-signal decoding oligonucleotides for binding to a particular identifier element (T) of analyte-specific probes, wherein the decoding oligonucleotides in the same set of non-signal decoding oligonucleotides interacting with the same different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

As mentioned above, the sample may be contacted with at least two (2) different sets of non-signal decoding oligonucleotides for binding to at least two different identifier elements (T) of analyte-specific probes, each set of non-signal decoding oligonucleotides interacting with a different identifier element (T), wherein each non-signal decoding oligonucleotide comprises an identifier connector element (t) comprising a nucleotide sequence which is essentially complementary to at least a section of a unique identifier sequence, and does not comprise a translator element (c) comprising a nucleotide sequence allowing a specific hybridization of a signal oligonucleotide.

In some advantageous embodiments of the method according to the present disclosure, the different sets of non-signal decoding oligonucleotides may be comprised in a pre-mixture of different sets of non-signal decoding oligonucleotides or exist separately.

Furthermore, in some advantageous embodiments of the method according to the present disclosure, the sample is contacted with a set of non-signal oligonucleotides, each non-signal oligonucleotide comprising: (aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

In further embodiments, the sample may be contacted with: at least two sets of non-signal oligonucleotides, each non-signal oligonucleotide comprising: (aa) a translator connector element (C) comprising a nucleotide sequence which is essentially complementary to at least a section of the nucleotide sequence of the translator element (c), and (bb) a quencher (Q), a signal element and a quencher (Q), or does not comprise a signal element.

As mentioned above, the different sets of non-signal oligonucleotides may be comprised in a pre-mixture of different sets of non-signal oligonucleotides or exist separately.

In further embodiments, the decoding oligonucleotides in a particular set of decoding oligonucleotides interacts with identical identifier elements (T) which are unique to a particular analyte.

As mentioned above, the different sets of decoding oligonucleotides may be comprised in a pre-mixture of different sets of decoding oligonucleotides or exist separately as well as the different sets of analyte-specific probes may be comprised in a pre-mixture of different sets of analyte-specific probes or exist separately as well the different sets of signal oligonucleotides may be comprised in a pre-mixture of different sets of signal oligonucleotides or exist separately.

In some advantageous embodiments of the method according to the present disclosure, the binding element(S) comprise a nucleic acid comprising a nucleotide sequence allowing a specific binding to the analyte to be encoded, preferably a specific hybridization to the analyte to be encoded. However, it is not uncommon for the analyte to differ in sequence from that which is expected, such that at least some of the sequence used to generate at least some of the probe set does not correspond to the sequence of the target analyte. Through use of multiple nonoverlapping probes in a probe set targeting multiple distinct predicted regions, often adjacent regions, of the target analyte, a probe set may be generated that is resilient to these variations in the actual target analyte relative to the expected target analyte sequence, such that the target analyte is detected despite differing from the expected target analyte in nucleic acid or amino acid sequence.

In some advantageous embodiments of the method according to the present disclosure, after step A) and before step B) the non-bound analyte-specific probes may be removed, in particular by washing, further after step B) and before step C) the non-bound decoding oligonucleotides may be removed, in particular by washing further, after step C) and before step D) the non-bound signal oligonucleotides may be removed, in particular by washing.

In some advantageous embodiments of the method according to the present disclosure, the analyte specific probes may be incubated with the sample, thereby allowing a specific binding of the analyte specific probes to the analytes to be encoded, further the decoding oligonucleotides may be incubated with the sample, thereby allowing a specific hybridization of the decoding oligonucleotides to identifier elements (T) of the respective analyte-specific probes, further the signal oligonucleotides may be incubated with the sample, thereby allowing a specific hybridization of the signal oligonucleotides to translator elements (T) of the respective decoding oligonucleotides.

As mentioned above, the analyte to be encoded may be a nucleic acid, preferably DNA, PNA, RNA, in particular mRNA, a peptide, polypeptide, a protein or combinations thereof. Therefore, the binding element(S) may comprise an amino acid sequence allowing a specific binding to the analyte to be encoded. Examples for a binding element(S) are moieties which are affinity moieties from affinity substances or affinity substances in their entirety selected from the group consisting of antibodies, antibody fragments, anticalin proteins, receptor ligands, enzyme substrates, lectins, cytokines, lymphokines, interleukins, angiogenic or virulence factors, allergens, peptidic allergens, recombinant allergens, allergen-idiotypical antibodies, autoimmune-provoking structures, tissue-rejection-inducing structures, immunoglobulin constant regions and combinations thereof. In particular, the binding element(S) is an antibody, or an antibody fragment selected from the group consisting of Fab, scFv; single domain, or a fragment thereof, bis scFv, Fab 2, Fab 3, minibody, diabody, triabody, tetrabody and tandab.

By this measure the method is further developed to such an extent that the encoded analytes can be detected by any means which is adapted to visualize the signal element. Examples of detectable physical features include e.g., light, chemical reactions, molecular mass, radioactivity, etc.

In some advantageous embodiments, the signal caused by the signal element, therefore in particular the binding of the signal oligonucleotides to the decoding oligonucleotides, interacting with the corresponding analyte probes, bound to the respective analyte is determined by: Imaging at least a portion of the sample; and/or Using an optical imaging technique; and/or Using a fluorescence imaging technique; and/or Multi-color fluorescence imaging technique; and/or Super-resolution fluorescence imaging technique.

Since the signals are encoded in certain colors, in some aspects color filters may be employed to differentiate the signals of each color vs. the total signal. Thereby facilitating additional information.

The kits and method according to the present disclosure may be used ideally for in vitro methods for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases.

Further, the kits and method according to the present disclosure may be used also ideally for in vitro methods for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment.

Further, the kits and method according to the present disclosure may be used also ideally for in vitro methods for screening, identifying and/or testing a substance and/or drug comprising: contacting a test sample comprising a sample with a substance and/or drug; and detecting different analytes in a sample by sequential signal-encoding of said analytes with a method according to the present disclosure.

An optical multiplexing system suitable for the method according to the present disclosure, comprising: a reaction vessel for containing the kits or part of the kits according to the present disclosure; a detection unit comprising a microscope, in particular a fluorescence microscope; a camera; a liquid handling device.

In some embodiments, optical multiplexing system may comprise further a heat and cooling device and/or a robotic system.

In some embodiments, the method according to the present disclosure encodes a nucleic acid analyte, such as an mRNA, e.g. such an mRNA coding for a particular protein.

In some advantageous embodiments, the method described herein is used for specific detection of many different analytes. The technology allows to distinguish a higher number of analytes than different signals are available. The process includes at least four consecutive rounds of specific binding, signal detection and selective denaturation (if a next round is required), eventually producing a signal code. To decouple the dependency between the analyte specific binding and the oligonucleotides providing the detectable signal, a so called “decoding”-oligonucleotide is introduced. The decoding oligonucleotide transcribes the information of the analyte specific probe set to the signal oligonucleotides.

In some advantageous embodiments, a method described herein is used to detect one or more analytes in a sample despite those one or more analytes differing from an expected sequence in a way that may not be known during the process of probe generation. Such differences may cause a single-probe system to fail to detect or to substantially underrepresent the amount of the target analyte in the sample. Through the methods herein, analytes are redundantly, distinctly detected through the use of probes targeting multiple nonverlapping regions. As a result, the methods, compositions, and systems herein are resilient to differences such as SNPs, splice variations, local in/dels, or other differences that naturally arise among diverse populations of individuals. At the same time, the probe compositions, methods and systems herein do not suffer from the lack of specificity, lower stringency or higher cost that often arises from generating probe populations that comprise one or more variable base positions, and that nonetheless are still not resilient to the full range of variations that arise among diverse populations of individuals, be they human populations, cancer cell populations or rapidly evolving viral or other pathogen populations.

Some methods consistent with the disclosure herein comprise the steps 1. providing one or more analyte specific probe sets, the set of analyte specific probes consist of one or more different probes, each differing in the binding moiety that specifically interacts with the analyte, all probes of a single probe set are tethered to a sequence element (unique identifier), that is unique to a single probe set or sufficient to distinguish that probe set from at least some other probe sets applied to the sample, and allows the specific hybridization of a decoding oligonucleotide, 2. specific binding of the probe sets to their target binding sites of the analyte, 3. eliminating non-bound probes (e.g. by a wash step), 4. providing a mixture of decoding oligonucleotides that specifically hybridize to the unique identifier sequences of the probe sets, the decoding oligonucleotides comprise of at least two sequence elements, a first element that is complementary to the unique identifier sequences of the corresponding probe set and a second sequence element (translator element) that provides a sequence for the specific hybridization of a signal oligonucleotide, the translator element defines the type of signal that is recruited to the decoding oligonucleotide, 5. specific hybridization of the decoding oligonucleotides to the unique identifier sequences provided by the bound probe sets, 6, eliminating non-bound decoding oligonucleotides (e.g. by washing step), 7, providing a mixture of signal oligonucleotides, comprising of a signal that can be detected and a nucleic acid sequence that specifically hybridizes to the translator element of one of the decoding oligonucleotides used in the former hybridization step, 8, specific hybridization of the signal oligonucleotides, 9, eliminating non-bound signal oligonucleotides, 10, detection of the signals, 11, selective release of decoding oligonucleotides and signal oligonucleotides while the binding of specific probe sets to the analyte is almost or completely unaffected, 12, eliminating released decoding oligonucleotide and signal oligonucleotides (e.g. by a washing step) while the binding of specific probes sets to the analytes is almost or completely unaffected, repeating the steps 4 to 12 at least three times until the detection of a sufficient number of signals to generate an encoding scheme for each different analyte of interest.

It is to be understood that the before-mentioned features and those to be mentioned in the following cannot only be used in the combination indicated in the respective case, but also in other combinations or in an isolated manner without departing from the scope of the disclosure.

The disclosure is now further explained by means of embodiments resulting in additional features, characteristics and advantages of the disclosure. The embodiments are of pure illustrative nature and do not limit the scope or range of the disclosure. The features mentioned in the specific embodiments are general features of the disclosure which are not only applicable in the specific embodiment but also in an isolated manner in the context of any embodiment of the disclosure.

Methods disclosed herein are used for specific detection of many different analytes. The technology allows distinguishing a higher number of analytes than different signals are available. The process preferably includes at least two consecutive rounds of specific binding, signal detection and selective denaturation (if a next round is required), eventually producing a signal code. To decouple the dependency between the analyte specific binding and the oligonucleotides providing the detectable signal, a so called “decoding” oligonucleotide is introduced. The decoding oligonucleotide transcribes the information of the respective analyte specific probe set to the signal oligonucleotides.

The present disclosure pertains further to methods of detecting an analyte, comprising:
attaching a plurality of analyte-specific probes to the analyte, wherein the analyte-specific probes independently attach to the analyte and wherein the analyte-specific probes share a common identifier segment (T); annealing a plurality of first decoding oligonucleotides to the analyte-specific probes, wherein the first decoding oligonucleotides share a first common region that is reverse complementary to the common identifier segment and a second common region; annealing a first signal oligonucleotide to at least one of the plurality of first decoding oligonucleotides such that an oligo tethered to the first signal oligonucleotide is reverse complementary to the second common region; detecting the first signal oligonucleotide; removing the plurality of first decoding oligonucleotides; annealing a plurality of second decoding oligonucleotides to the analyte-specific probes, wherein the second decoding oligonucleotides share a first common region that is reverse complementary to the common identifier segment and a second decoding oligonucleotides second common region that differs from the second common region of the first decoding; annealing a second signal oligonucleotide to at least one of the plurality of second decoding oligonucleotides such that an oligo tethered to the signal oligonucleotide is reverse complementary to the second decoding oligonucleotide second common region; and detecting the second signal oligonucleotide.

In particular, in the above-mentioned embodiment a second aliquot of a plurality of first decoding oligonucleotides is annealed to the analyte-specific probes. Furthermore, a first aliquot of a plurality of first decoding oligonucleotides is annealed to the analyte-specific probes.

In some further advantageous embodiments, the present disclosure pertains to a method of assigning an analyte to a position in an image, comprising assigning a fluorescence pattern to the analyte, observing the fluorescence pattern at the position in the image, and assigning the analyte to the position, in particular wherein observing the fluorescence pattern comprises repeating steps of labeling the position using a fluorophore tagged oligo drawn from a re-accessible pool, performing a single excitation at the position in the image, and contacting the analyte to a denaturant, in particular wherein observing the fluorescence pattern comprises repeating steps of labeling the position using a fluorophore tag-recruiting bridging oligo drawn from a re-accessible pool, performing a single excitation at the position in the image, and contacting the analyte to a denaturant. This assignment is resilient to variations in the target analyte that result in some subsets of a target probe failing to bind to the target analyte, but nonetheless leads to detection and localization of the target analyte in the sample.

In some further advantageous embodiments, the present disclosure pertains to a composition comprising a cell having nucleic acids distributed therein, wherein a first nucleic acid is tagged by a first plurality of probes that target adjacent segments of the first nucleic acid and that share a common first tether segment; a second nucleic acid is tagged by a second plurality of probes that target adjacent segments of the second nucleic acid and that share a common second tether segment; and a third nucleic acid is tagged by a third plurality of probes that target adjacent segments of the third nucleic acid and that share a common third tether segment; a first adapter population comprising molecules having a first tether reverse complementary region and a first fluorophore adapter tether; a second adapter population comprising molecules having a second tether reverse complementary region and a second fluorophore adapter tether; a third adapter population comprising molecules having a third tether reverse complementary region and a first fluorophore adapter tether; a population of first fluorophores having a first tether reverse complementary region; and a population of second fluorophores having a second tether reverse complementary region.

In some further advantageous embodiments, the present disclosure pertains to a method of assigning coded fluorescence patterns to a plurality of target analytes in a cell, comprising: subjecting the cell to a plurality of detection rounds, each detection round comprising: contacting the cell to representatives of the same at least two populations of tagged fluorescence moieties, and removing the fluorescent moieties after a single excitation event, in particular wherein the number of patterns detectable increases exponentially with the number of detection rounds, wherein the fluorescence moieties are not tagged with nucleic acid tags that are specific to the target nucleic acids, and wherein separate aliquots of common tagged fluorescence moieties are used across multiple detection rounds.

In particular, with the above notified method a total decoding efficiency of at least 20%, at least 30%, at least 40%, at least 50%, at least 60% may be achieved. In some aspects the total decoding efficiency is 100% or less, 99% or less, 98.5% or less, 90% or less, 80% or less. In some aspects the total decoding efficiency between 20% and 100%, between 30% and 90%, between 40% and 80%.

In some further advantageous embodiments, the present disclosure pertains to a method of assigning coded fluorescence patterns to a plurality of target analytes in a cell, comprising: contacting a target to a bipartite labeling probe, the bipartite labeling probe comprising a target-specific moiety and a fluorophore-specifying moiety; contacting the bipartite labeling probe to a first aliquot of a fluorophore reservoir comprising no more than two populations of fluorophores; replacing the fluorophore specifying moiety in the bipartite probe, and contacting the bipartite labeling probe to a second aliquot of the fluorophore reservoir comprising the same no more than two populations.

In some embodiments of the above notified method, replacing the fluorophore specifying moiety in the bipartite probe comprises denaturing a binding between a target-specific moiety and a fluorophore-specifying moiety after subjecting the bipartite labeling probe bound to a fluorophore of the fluorophore to excitation energy. In particular, replacing the fluorophore specifying moiety in the bipartite probe comprises drawing from one of no more than two fluorophore specifying moiety reservoirs.

In some further advantageous embodiments, the present disclosure pertains to a method of detecting an analyte, comprising: attaching a plurality of probes to the analyte, in particular a nucleic acid, wherein the probes independently attach/anneal to the analyte and wherein the probes share a common identifier segment; annealing a plurality of first adapter segments to the probes, wherein the first adapter segments share a first common region that is reverse complementary to the common identifier segment and a second common region, in particular configured to accommodate a single reporter/selected from no more than two reporter categories; annealing a first reporter to at least one of the plurality of first adapter segments such that an oligo tethered to the first reporter is reverse complementary to the second common region; detecting the first reporter; removing the plurality of first adapter segments, in particular without annealing a second reporter to the at least one of the plurality of first adapter segments; annealing a plurality of second adapter segments to the probes, wherein the second adapter segments share a first common region that is reverse complementary to the common identifier segment and a second adapter second common region that differs from the second common region of the first adapter segments, in particular configured to accommodate a single reporter/selected from no more than two reporter categories; annealing a second reporter to at least one of the plurality of second adapter segments such that an oligo tethered to the second reporter is reverse complementary to the second adapter second common region; and detecting the second reporter, in particular without annealing a third reporter to the at least one of the plurality of first adapter segments.

Embodiments of the present disclosure pertains to the following items:

Item 1: A multiplex method for detecting different analytes in a sample beyond the diffraction limit by sequential signal-encoding of said analytes, comprising the steps of: (A1) contacting the sample with a first set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and (A2) contacting the sample with a second set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and wherein (optionally) the number of probes and/or targets of first set of analyte-specific probes according to step A1 (i.e. the transcript plexity of A1) is at least 10 times higher than the number of probes and/or targets of the second set of analyte-specific probes according to step A2 (i.e. the transcript plexity of A2); and (B1) contacting the sample with at least a first set of decoding oligonucleotides per analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (B2) contacting the sample with at least a second set of decoding oligonucleotides per analyte, wherein in each set of decoding oligonucleotides for an individual analyte of each decoding oligonucleotide for the second set of analyte-specific probes according to step A2 comprises: (aa) an identifier connector element (t) comprising a nucleotide sequence which is essentially complementary to at least a section of the unique identifier sequence of the identifier element (T) of the corresponding analyte-specific probe set A2, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element; and (D) Detecting the signal caused by the signal element; (E) selectively removing the decoding oligonucleotides and signal oligonucleotides from the sample, thereby essentially maintaining the specific binding of the analyte-specific probes to the analytes to be encoded; (F) Performing at least three (3) further cycles comprising steps B) to E) to generate an encoding scheme with a code word per analyte, wherein in particular the last cycle may stop with step (D).

Item 2: The method according to item 1, wherein steps A1 and A2 as well as steps B1 and B2 can be performed in consecutive cycles of the steps in the order (A1, B1, C, D, E and F), and then (A2, B2, C, D, E and F) n; or in interwoven cycles of the steps in the order (A1, A2, B1, B2, C, D, E and F) n, wherein n is the number of cycles and at least 3.

Item 3: A kit for multiplex analyte encoding beyond the diffraction limit, comprising: (A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and (A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and wherein the number of probes and/or targets of first set of analyte-specific probes according to step A1 (the transcript plexity of A1) is at least 10 times higher than the number of probes and/or targets of the second set of analyte-specific probes according to step A2 (i.e. the transcript plexity of A2); and (B) at least one set of decoding oligonucleotides per analyte set A1 and A2, wherein in each set of decoding oligonucleotides for an individual analyte each decoding oligonucleotide comprises: (aa) an identifier connector element (t) comprising a nucleotide sequence which is essentially complementary to at least a section of the unique identifier sequence of the identifier element (T) of the corresponding analyte-specific probe set, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the identifier connect element (t); and (C) a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element.

Item 4: The method according to item 1 for in vitro diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure.

Item 5: The method according to item 1 for the diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the multiplex method according to the present disclosure.

Item 6: An optical multiplexing system suitable for the method according to item 1, comprising: at least one reaction vessel for containing the kits or part of the kits according to item 3; a detection unit comprising a microscope, in particular a fluorescence microscope a camera; a liquid handling device.

Item 7: A method for screening, identifying and/or testing a substance and/or drug comprising: contacting a test sample comprising a sample with a substance and/or drug; and detecting different analytes in a sample by sequential signal-encoding of said analytes with a method according to item 1.

### Methods and Examples

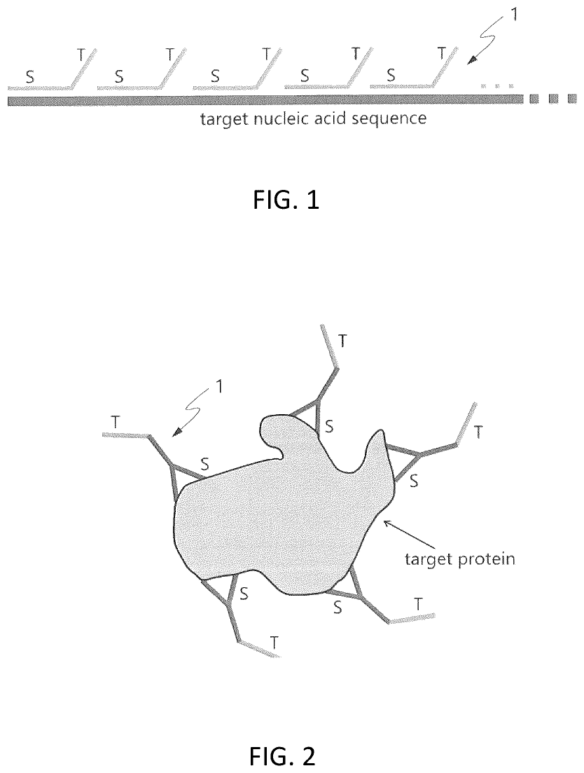

In an application variant, the analyte or target is nucleic acid, e.g., DNA or RNA, and the probe set comprises oligonucleotides that are partially or completely complementary to the whole sequence or a subsequence of the nucleic acid sequence to be detected (FIG. 1). The nucleic acid sequence specific oligonucleotide probe sets comprising analyte-specific probes (1) including a binding element(S) that specifically hybridizes to the target nucleic acid sequence to be detected, and an identifier element (T) comprising a nucleotide sequence which is unique to said set of analyte-specific probes (unique identifier sequence).

In a further application variant, the analyte or target is a protein, and the probe set comprises one or more proteins, e.g. antibodies (FIG. 2). The protein specific probe set comprising analyte-specific probes (1) including a binding element (T) such as the (hyper-) variable region of an antibody, that specifically interacts with the target protein to be detected, and the identifier element (T).

In a further application variant, at least one analyte is a nucleic acid and at least a second analyte is a protein and at least the first probe set binds to the nucleic acid sequence and at least the second probe set binds specifically to the protein analyte. Other combinations are possible as well.

An Embodiments of the general method of the present disclosure may be:

Step 1: Applying the at least 20 analyte- or target-specific probe sets. The target nucleic acid sequence is incubated with a probe set consisting of oligonucleotides with sequences complementary to the target nucleic acid. In this example, a probe set of 5 different probes is shown, each comprising a sequence element complementary to an individual subsequence of the target nucleic acid sequence (S1 to S5). In this example, the regions do not overlap. Each of the oligonucleotides targeting the same nucleic acid sequence comprises the identifier element or unique identifier sequence (T), respectively.

Step 2: Hybridization of the probe set. The probe set is hybridized to the target nucleic acid sequence under conditions allowing a specific hybridization. After the incubation, the probes are hybridized to their corresponding target sequences and provide the identifier element (T) for the next steps.

Step 3: Eliminating non-bound probes. After hybridization, the unbound oligonucleotides are eliminated, e.g., by washing steps.

Step 4: Applying the decoding oligonucleotides. The decoding oligonucleotides consisting of at least two sequence elements (t) and (c) are applied. While sequence element (t) is complementary to the unique identifier sequence (T), the sequence element (c) provides a region for the subsequent hybridization of signal oligonucleotides (translator element).

Step 5: Hybridization of decoding oligonucleotides. The decoding oligonucleotides are hybridized with the unique identifier sequences of the probes (T) via their complementary first sequence elements (t). After incubation, the decoding oligonucleotides provide the translator sequence element (c) for a subsequent hybridization step.

Step 6: Eliminating the excess of decoding oligonucleotides. After hybridization, the unbound decoding oligonucleotides are eliminated, e.g., by washing steps.

Step 7: Applying the signal oligonucleotide. The signal oligonucleotides are applied. The signal oligonucleotides comprise at least one second connector element (C) that is essentially complementary to the translator sequence element (c) and at least one signal element that provides a detectable signal (F).

Step 8: Hybridization of the signal oligonucleotides. The signal oligonucleotides are hybridized via the complementary sequence connector element (C) to the translator element (c) of decoding oligonucleotide. After incubation, the signal oligonucleotides are hybridized to their corresponding decoding oligonucleotides and provide a signal (F) that can be detected.

Step 9: Eliminating the excess of signal oligonucleotides. After hybridization, the unbound signal oligonucleotides are eliminated, e.g., by washing steps.

Step 10: Signal detection. The signals provided by the signal oligonucleotides are detected. The following steps (steps 11 and 12) are unnecessary for the last detection round.

Step 11: Selective denaturation. The hybridization between the unique identifier sequence (T) and the first sequence element (t) of the decoding oligonucleotides is dissolved. The destabilization can be achieved via different mechanisms well known to the trained person like for example: increased temperature, denaturing agents, etc. The target- or analyte-specific probes are not affected by this step.

Step 12: Eliminating the denatured decoding oligonucleotides. The denatured decoding oligonucleotides and signal oligonucleotides are eliminated (e.g., by washing steps) leaving the specific probe sets with free unique identifier sequences, reusable in a next round of hybridization and detection (steps 4 to 10). This detection cycle (steps 4 to 12) is repeated at least four times until the planned encoding scheme is completed.

Another Embodiment of the general method of the present disclosure using multi-decoders may be (FIG. 16):

Step 1: Target nucleic acids: In this example three different target nucleic acids (A), (B) and (C) have to be detected and differentiated by using only two different types of signal oligonucleotides. Before starting the experiment, a certain encoding scheme is set. In this example, the three different nucleic acid sequences are encoded by three rounds of detection with three different signal types (1), (2) and (½) and a resulting hamming distance of 3 to allow for error detection. The planed code words are:
sequence A: (1)-(1)-(2)
sequence B: (2)-(2)-(½)
sequence C: (½)-(½)-(1)

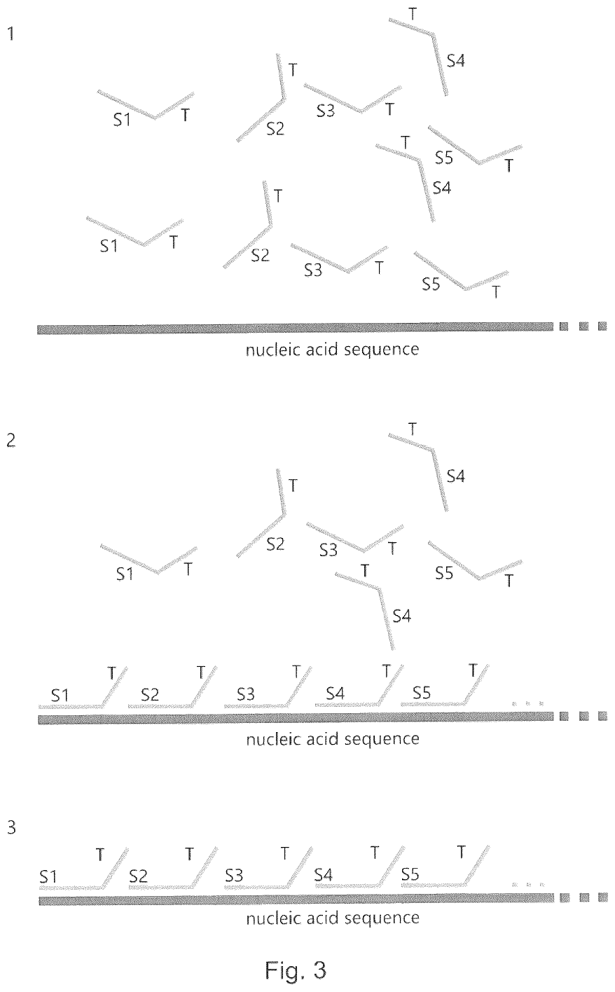

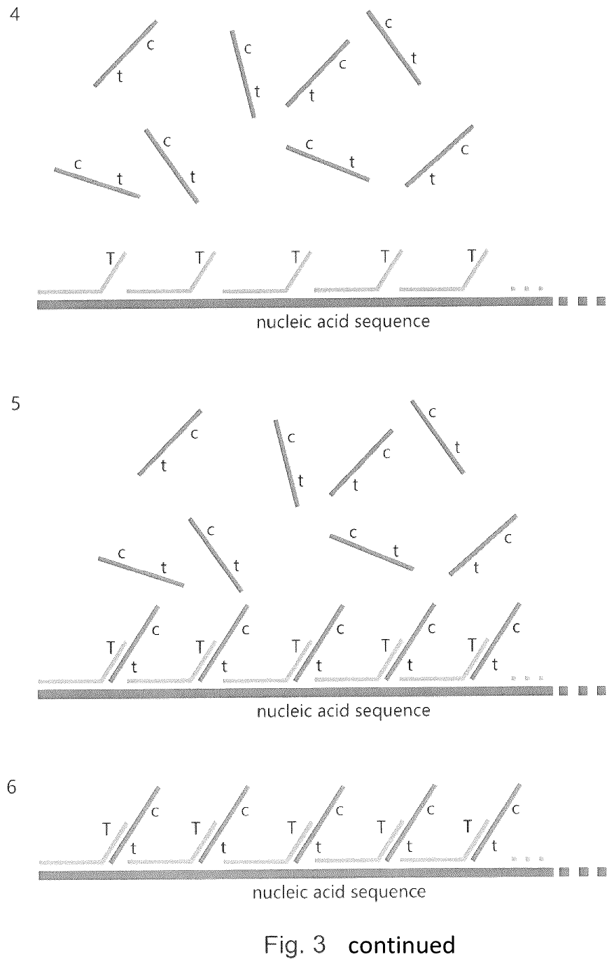

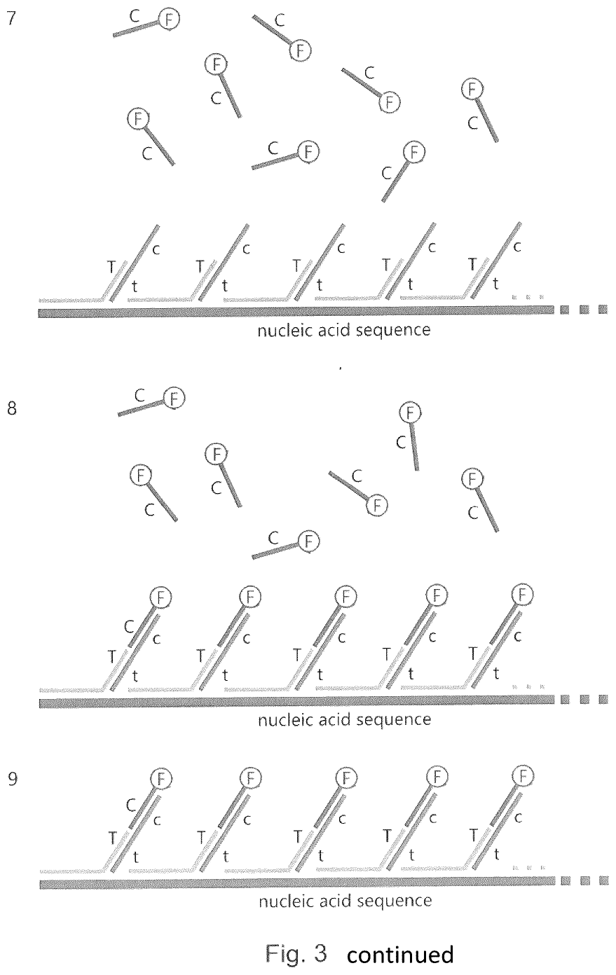

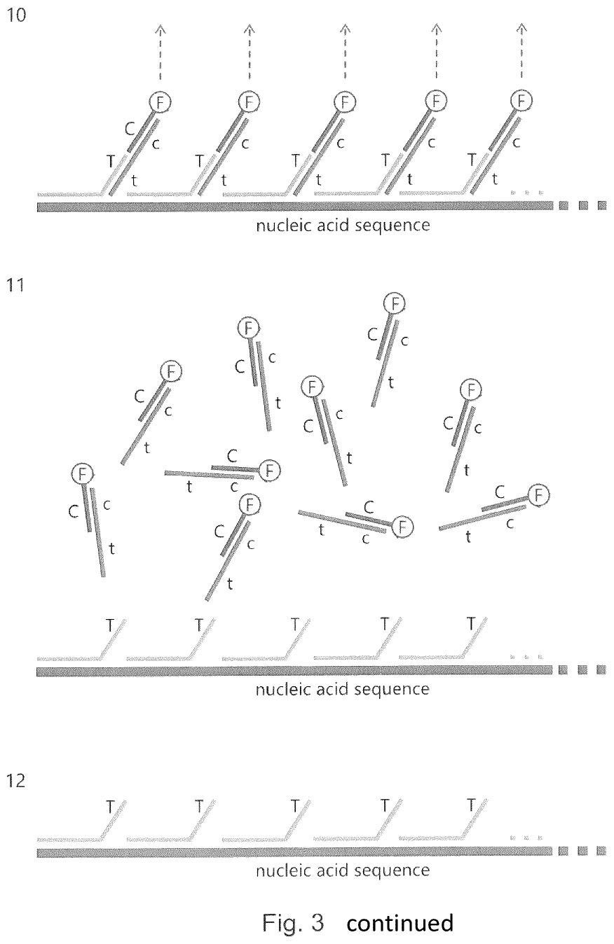

Step 2: Hybridization of the probe sets: For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (A) is labeled with (T1), sequence (B) with (T2) and sequence (C) with (T3). The illustration in FIG. 16 summarizes Steps 1 to 3 of FIG. 3.

Step 3: Hybridization of the decoding oligonucleotides and multi-decoders: For each unique identifier present, a certain decoding oligonucleotide or multi-decoder is applied specifically hybridizing to the corresponding unique identifier sequence by its first sequence element (here (t1) to (T1), (t2) to (T2) and (t3) to (T3)). Each of the decoding oligonucleotides or multi-decoders provides a translator or two translator elements that define the signals that will be generated after hybridization of signal oligonucleotides. Here nucleic acid sequence (A) is labeled with (c1), (B) is labeled with (c2) and (C) is labeled with both translator elements (c1) and (c2) resulting in the signal (½). The illustration in FIG. 16 summarizes steps 4 to 6 of FIG. 3.

Step 4: Hybridization of signal oligonucleotides: For each type of translator element, a signal oligonucleotide with a certain signal, differentiable from signals of other signal oligonucleotides, is applied. This signal oligonucleotide can specifically hybridize to the corresponding translator element. The illustration in FIG. 16 summarizes steps 7 to 9 of FIG. 3

Step 5: Signal detection for the encoding scheme: The different signals are detected. Note that in this example the nucleic acids (A), (B) and (C) can already be distinguished after the first round of detection. This is in contrast to the step 5 of FIG. 5 explained by the additional signal type (½) that can be realized due to multi-decoders. Although nucleic acid sequences can already be distinguished, the additional rounds contribute to the planned hamming distance of 3. The illustration in FIG. 16 corresponds to step 10 of FIG. 3.

Step 6: Selective denaturation: The decoding (and signal) oligonucleotides and/or multi-decoders of all nucleic acid sequences to be detected are selectively denatured and eliminated as described in steps 11 and 12 of FIG. 3. Afterwards the unique identifier sequences of the different probe sets can be used for the next round of hybridization and detection.

Step 7: Second round of detection: A next round of hybridization and detection is done as described in steps 3 to 5. Note that in this new round the mix of different decoding oligonucleotides and multi-decoders is changed. For example, decoding oligonucleotide of nucleic acid sequence (A) used in the first round comprised of sequence elements (t1) and (c1) while the new multi decoder of round 2 comprises of the sequence elements (t1), (c1) and (c2). Note that now a hamming distance of 2 is already given after 2 rounds, which is the final result of the example in FIG. 3 after 3 rounds.

Step 8: Third round of detection: Again, a new combination of decoding oligonucleotides and/or multi-decoders is used leading to new signal combinations. After signal detection, the resulting code words for the three different nucleic acid sequences are not only unique and therefore distinguishable but comprise a hamming distance of 3 to other code words. Due to the hamming distance, an error in the detection of the signals (signal exchange) would not result in a valid code word and therefore could be detected and because of hamming distance 3 also corrected, in contrast to the encoding scheme of FIG. 3. This way three different nucleic acids can be distinguished in three detection rounds with two different signals, allowing an error detection and correction.

Note that in every round of detection, the type of signal provided by a certain unique identifier is controlled by the use of a certain decoding oligonucleotide. As a result, the sequence of decoding oligonucleotides applied in the detection cycles transcribes the binding specificity of the probe set into a unique signal sequence.

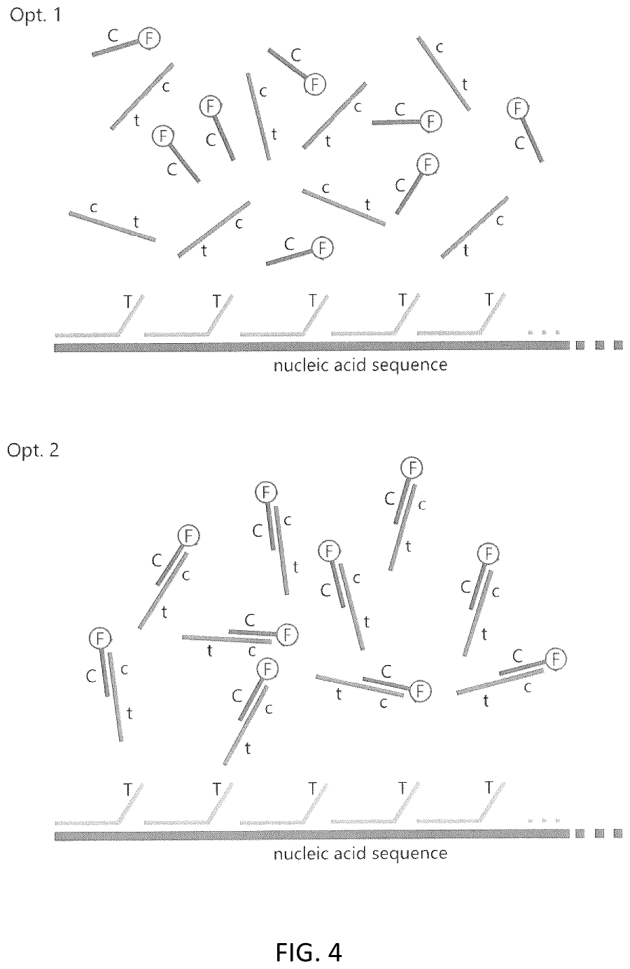

The steps of decoding oligonucleotide hybridization (steps 4 to 6) and signal oligonucleotide hybridization (steps 7 to 9) can also be combined in two alternative ways as shown in FIG. 4.

Opt. 1: Simultaneous hybridization. Instead of the steps 4 to 9 of FIG. 3, specific hybridization of decoding oligonucleotides and signal oligonucleotides can also be done simultaneously leading to the same result as shown in step 9 of FIG. 3, after eliminating the excess decoding- and signal oligonucleotides.

Opt. 2: Preincubation. Additionally, to option 1 of FIG. 3, decoding- and signal oligonucleotides can be preincubated in a separate reaction before being applied to the target nucleic acid with the already bound specific probe set.

1. Example for signal encoding of three different nucleic acid sequences by two different signal types and three detection rounds

FIG. 3 shows the general concept of generation and detection of specific signals mediated by decoding oligonucleotides. It does not show the general concept of encoding that can be achieved by this procedure. To illustrate the use of the process shown in FIG. 3 for the generation of an encoding scheme, FIG. 5 shows a general example for a multiple round encoding experiment with three different nucleic acid sequences. In this example, the encoding scheme includes error detection.

Step 1: Target nucleic acids. In this example three different target nucleic acids (A), (B) and (C) have to be detected and differentiated by using only two different types of signals. Before starting the experiment, a certain encoding scheme is set. In this example, the three different nucleic acid sequences are encoded by three rounds of detection with two different signals (1) and (2) and a resulting hamming distance of 2 to allow for error detection. The planed code words are:
sequence A: (1)-(2)-(2);
sequence B: (1)-(1)-(1);
sequence C: (2)-(1)-(2).

Step 2: Hybridization of the probe sets. For each target nucleic acid, an own probe set is applied, specifically hybridizing to the corresponding nucleic acid sequence of interest. Each probe set provides a unique identifier sequence (T1), (T2) or (T3). This way each different target nucleic acid is uniquely labeled. In this example sequence (T) is labeled with (T1), sequence (B) with (T2) and sequence (C) with (T3). The illustration summarizes steps 1 to 3 of FIG. 3.

Step 3: Hybridization of the decoding oligonucleotides. For each unique identifier present, a certain decoding oligonucleotide is applied specifically hybridizing to the corresponding unique identifier sequence by its first sequence element (here (t1) to (T1), (t2) to (T2) and (t3) to (T3)). Each of the decoding oligonucleotides provides a translator element that defines the signal that will be generated after hybridization of signal oligonucleotides. Here nucleic acid sequences (A) and (B) are labeled with the translator element (c1) and sequence (C) is labeled with (c2). The illustration summarizes steps 4 to 6 of FIG. 3.

Step 4: Hybridization of signal oligonucleotides. For each type of translator element, a signal oligonucleotide with a certain signal (2), differentiable from signals of other signal oligonucleotides, is applied. This signal oligonucleotide can specifically hybridize to the corresponding translator element. The illustration summarizes steps 7 to 9 of FIG. 3.

Step 5: Signal detection for the encoding scheme. The different signals are detected. Note that in this example the nucleic acid sequence (C) can be distinguished from the other sequences by the unique signal (2) it provides, while sequences (A) and (B) provide the same kind of signal (1) and cannot be distinguished after the first cycle of detection. This is due to the fact that the number of different nucleic acid sequences to be detected exceeds the number of different signals available. The illustration corresponds to step 10 of FIG. 3.

Step 6: Selective denaturation. The decoding (and signal) oligonucleotides of all nucleic acid sequences to be detected are selectively denatured and eliminated as described in steps 11 and 12 of FIG. 3. Afterwards the unique identifier sequences of the different probe sets can be used for the next round of hybridization and detection.

Step 7: Second round of detection. A next round of hybridization and detection is done as described in steps 3 to 5. Note that in this new round the mix of different decoding oligonucleotides is changed. For example, decoding oligonucleotide of nucleic acid sequence (A) used in the first round comprised of sequence elements (t1) and (c1) while the new decoding oligonucleotide comprises of the sequence elements (t1) and (c2). Note that now all three sequences can clearly be distinguished due to the unique combination of first and second round signals.

Step 8: Third round of detection. Again, a new combination of decoding oligonucleotides is used leading to new signal combinations. After signal detection, the resulting code words for the three different nucleic acid sequences are not only unique and therefore distinguishable but comprise a hamming distance of 2 to other code words. Due to the hamming distance, an error in the detection of the signals (signal exchange) would not result in a valid code word and therefore could be detected. By this way three different nucleic acids can be distinguished in three detection rounds with two different signals, allowing error detection.

### 2. Advantages Over Prior Art Technologies

### Coding Strategy

Compared to state-of-the-art methods, one particular advantage of the method according to the disclosure is the use of decoding oligonucleotides breaking the dependencies between the target specific probes and the signal oligonucleotides.

Without decoupling target specific probes and signal generation, two different signals can only be generated for a certain target if using two different molecular tags. Each of these molecular tags can only be used once. Multiple readouts of the same molecular tag do not increase the information about the target. In order to create an encoding scheme, a change of the target specific probe set after each round is required (SeqFISH) or multiple molecular tags must be present on the same probe set (like merFISH, intronSeqFISH).

Following the method according to the disclosure, different signals are achieved by using different decoding oligonucleotides reusing the same unique identifier (molecular tag) and a small number of different, mostly cost-intensive signal oligonucleotides. This leads to several advantages in contrast to the other methods.

(1) The encoding scheme is not defined by the target specific probe set as it is the case for all other methods of prior art. Here the encoding scheme is transcribed by the decoding oligonucleotides. This leads to a much higher flexibility concerning the number of rounds and the freedom in signal choice for the codewords. Looking on the methods of prior art (e.g., merFISH or intronSeqFISH), the encoding scheme (number, type and sequence of detectable signals) for all target sequences is predefined by the presence of the different tag sequences on the specific probe sets (4 of 16 different tags per probe set in the case of merFISH and 5 of 60 different tags in the case of intron FISH). In order to produce a sufficient number of different tags per probe set, the methods use rather complex oligonucleotide designs with several tags present on one target specific oligonucleotide. In order to change the encoding scheme for a certain target nucleic acid, the specific probe set has to be replaced. The method according to the disclosure describes the use of a single unique tag sequence (unique identifier) per analyte, because it can be reused in every detection round to produce a new information. The encoding scheme is defined by the order of decoding oligonucleotides that are used in the detection rounds. Therefore, the encoding scheme is not predefined by the specific probes (or the unique tag sequence) but can be adjusted to different needs, even during the experiment. This is achieved by simply changing the decoding oligonucleotides used in the detection rounds or adding additional detection rounds.

(2) The number of different signal oligonucleotides must match the number of different tag sequences with methods of prior art (16 in the case of merFISH and 60 in the case of intronSeqFISH). Using the method according to the disclosure, the number of different signal oligonucleotides matches the number of different signals used. Due to this, the number of signal oligonucleotides stays constant for the method described here and never exceeds the number of different signals but increases with the complexity of the encoding scheme in the methods of prior art (more detection rounds more different signal oligonucleotides needed). As a result, the method described here leads to a much lower complexity (unintended interactions of signal oligonucleotides with environment or with each other) and dramatically reduces the cost of the assay since the major cost factor are the signal oligonucleotides.

(3) In the methods of prior art, the number of different signals generated by a target specific probe set is restricted by the number of different tag sequences the probe set can provide. Since each additional tag sequence increases the total size of the target specific probe, there is a limitation to the number of different tags a single probe can provide. This limitation is given by the size dependent increase of several problems (unintended inter- and intramolecular interactions, costs, diffusion rate, stability, errors during synthesis etc.). Additionally, there is a limitation of the total number of target specific probes that can be applied to a certain analyte. In the case of nucleic acids, this limitation is given by the length of the target sequence and the proportion of suitable binding sites. These factors lead to severe limitations in the number of different signals a probe set can provide (4 signals in the case of merFISH and 5 signals in the case of intronSeqFISH). This limitation substantially affects the number of different code words that can be produced with a certain number of detection rounds. In the approach of the disclosure only one tag is needed and can be freely reused in every detection round. This leads to a low oligonucleotide complexity/length and at the same time to the maximum encoding efficiency possible (number of colors^{number of rounds}). The vast differences of coding capacity of our method compared to the other methods is shown in FIGS. 1 and 5. Due to this approach of the disclosure a much lower number of detection rounds is needed to produce the same amount of information. A lower number of detection rounds is connected to lower cost, lower experimental time, lower complexity, higher stability and success rate, lower amount of data to be collected and analyzed and a higher accuracy of the results.

### Coding Capacity

All three methods compared in the Table 1a below use specific probe sets that are not denatured between different rounds of detection. For intronSeqFISH there are four detection rounds needed to produce the pseudo colors of one coding round, therefore data is only given for rounds 4, 8, 12, 16 and 20. The merFISH-method uses a constant number of 4 signals, therefore the data starts with the smallest number of rounds possible. After 8 detection rounds our method exceeds the maximum coding capacity reached with 20 rounds of merFISH (depicted with one asterisk) and after 12 rounds of detection the maximum coding capacity of intron FISH is exceeded (depicted with two asterisks). For the method according to the disclosure usage of 3 different signals is assumed (as is with intronSeqFISH).

TABLE 1a
Comparison of coding capacity

| | CODING CAPACITY | | |
|---|---|---|---|
| | Method | | |
| NUMBER OF | of the present | intron | |
| ROUNDS: | disclosure | FISH | merFISH |
| 1 | 3 | — | — |
| 2 | 9 | — | — |
| 3 | 27 | — | — |
| 4 | 81 | 12 | 1 |
| 5 | 243 | — | 5 |
| 6 | 729 | — | 15 |
| 7 | 2187 | — | 35 |
| 8* | 6561 | 144 | 70 |
| 9 | 19683 | — | 126 |
| 10 | 59049 | — | 210 |
| 11 | 177147 | — | 330 |
| 12** | 531441 | 1728 | 495 |
| 13 | 1594323 | — | 715 |
| 14 | 4782969 | — | 1001 |
| 15 | 14348907 | — | 1365 |
| 16 | 43046721 | 20736 | 1820 |
| 17 | 129140163 | — | 2380 |
| 18 | 387420489 | — | 3060 |
| 19 | 1162261467 | — | 3876 |
| 20 | 3486784401 | 248832 | 4845 |

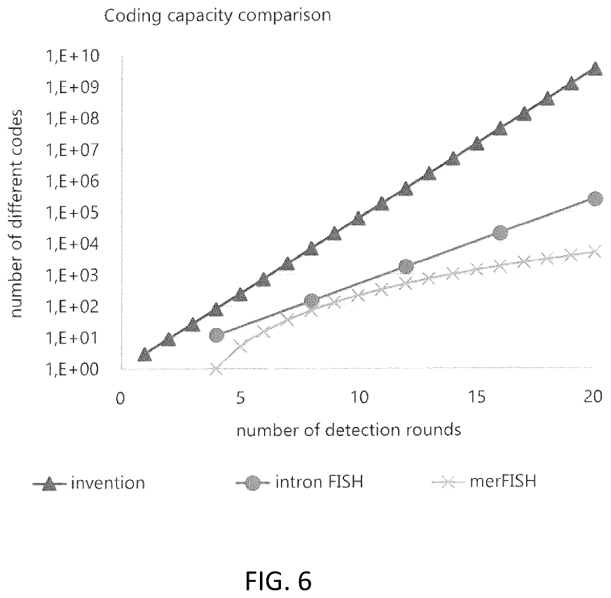

As shown in FIG. 6 the number of codewords for merFISH does not exponentially increase with the number of detection cycles but gets less effective with each added round. In contrast, the number of codewords for intronSeqFISH in the method according to the disclosure increases exponentially. The slope of the curve for the proposed method is much higher than that of intron FISH, leading to more than 10,000 times more code words usable after 20 rounds of detection.

Note that this maximum efficiency of coding capacity is also reached in case of seqFISH, where specific probes are denatured after every detection round and a new probe set is specifically hybridized to the target sequence for each detection round. However, this method has major downsides to technologies using only one specific hybridization for their encoding scheme (all other methods):
(1) For the efficient denaturation of the specific probes, rather crude conditions must be used (high temperatures, high concentrations of denaturing agent, long incubation times) leading to much higher probability for the loss or the damage of the analyte.
(2) For each round of detection an own probe set has to be used for every target nucleic acid sequence. Therefore, the number of specific probes needed for the experiment scales with the number of different signals needed for the encoding scheme. This dramatically increases the complexity and the cost of the assay.
(3) Because the hybridization efficiency of every target nucleic acid molecule is subject to some probabilistic effects, the fluctuations of signal intensity between the different detection rounds is much higher than in methods using only one specific hybridization event, reducing the proportion of complete codes.
(4) The time needed for the specific hybridization is much longer than for the hybridization of signal oligonucleotides or decoding oligonucleotides (as can be seen in the method parts of the intronSeqFISH, merFISH and seqFISH publications), which dramatically increases the time needed to complete an experiment.

Due to these reasons all other methods use a single specific hybridization event and accept the major downside of lower code complexity and therefore the need of more detection rounds and a higher oligonucleotide design complexity.

The method according to the disclosure combines the advantages of seqFISH (mainly complete freedom concerning the encoding scheme) with all advantages of methods using only one specific hybridization event while eliminating the major problems of such methods.

Note that the high numbers of code words produced after 20 rounds can also be used to introduce higher hamming distances (differences) between different codewords, allowing error detection of 1, 2 or even more errors and even error corrections. Therefore, even very high coding capacities are still practically relevant.

As mentioned above, the usage of multi-decoders further increases the coding capacity of the encoding scheme. Instead of being limited to the having exactly the same number of different signal types as different signal oligonucleotides and corresponding translator elements, the use of multi-decoders increases the signal types that can be used to: (N×(N+1))/2 (with N being the number of different signal oligonucleotides used). For the code used in table 1 with 3 different signal oligonucleotides this would mean the following 7 different signal types could be used:

(S1), (S2), (S3), (S1/S2), (S1/S3), (S2/S3), (S1/S2/S3). The effect to the coding efficiency can be seen in Table 1b and FIG. 17.

TABLE 1b

| | CODING CAPACITY | | | |
|---|---|---|---|---|
| | | Method of the | | |
| NUMBER | Method of | present disclosure | | |
| OF | the present | with multi- | intron | |
| ROUNDS: | disclosure | decoders | FISH | merFISH |
| 1 | 3 | 7 | — | — |
| 2 | 9 | 49 | — | — |
| 3 | 27 | 343 | — | — |
| 4 | 81 | 2401 | 12 | 1 |
| 5* | 243 | 16807 | — | 5 |
| 6 | 729 | 117649 | — | 15 |
| 7** | 2187 | 823543 | — | 35 |
| 8 | 6561 | 5764801 | 144 | 70 |
| 9 | 19683 | 40353607 | — | 126 |
| 10 | 59049 | 282475249 | — | 210 |
| 11 | 177147 | 1977326743 | — | 330 |
| 12*** | 531441 | 1.3841 × 10\hat{ }10 | 1728 | 495 |
| 13 | 1594323 | 9.6889 × 10\hat{ }10 | — | 715 |
| 14 | 4782969 | 6.7822 × 10\hat{ }11 | — | 1001 |
| 15 | 14348907 | 4.7476 × 10\hat{ }12 | — | 1365 |
| 16 | 43046721 | 3.3233 × 10\hat{ }13 | 20736 | 1820 |
| 17 | 129140163 | 2.3263 × 10\hat{ }14 | — | 2380 |
| 18 | 387420489 | 1.6284 × 10\hat{ }15 | — | 3060 |
| 19 | 1162261467 | 1.1399 × 10\hat{ }16 | — | 3876 |
| 20 | 3486784401 | 7.9792 × 10\hat{ }16 | 248832 | 4845 |

Table 1b shows the coding capacity of the four methods. All four methods compared in the table use specific probe sets that are not denatured between different rounds of detection. For intronSeqFISH there are four detection rounds needed to produce the pseudo colors of one coding round, therefore data is only given for rounds 4, 8,12,16 and 20. The merFISH-method uses a constant number of 4 signals, therefore the data starts with the smallest number of rounds possible. After 4 detection rounds the method with multi-decoders as described here exceeds the maximum coding capacity reached with 20 rounds of merFISH (depicted with one asterisk), after 7 rounds of detection the maximum coding capacity of intron FISH is exceeded (depicted with two asterisks) and after 12 rounds of detection the maximum coding capacity of the method of the present disclosure is exceeded (depicted with three asterisks). The usage of 3 different signal oligonucleotides is assumed (as is with intronSeqFISH).

3. Selective denaturation, oligonucleotide assembly and reuse of unique identifiers are surprisingly efficient

A key factor of the method according to the disclosure is the consecutive process of decoding oligonucleotide binding, signal oligonucleotide binding, signal detection and selective denaturation. In order to generate an encoding scheme, this process has to be repeated several times (depending on the length of the code word). Because the same unique identifier is reused in every detection cycle, all events from the first to the last detection cycle depend on each other. Additionally, the selective denaturation depends on two different events: While the decoding oligonucleotide has to be dissolved from the unique identifier with highest efficiency, specific probes have to stay hybridized with highest efficiency.

Due to this the efficiency E of the whole encoding process can be described by the following equation:

$$E = {B}_{s p} \times {( {B}_{d e} \times {B}_{s i} \times {E}_{d e} \times {S}_{s p} )}^{n}$$

Based on this equation the efficiency of each single step can be estimated for a given total efficiency of the method. The calculation is hereby based on the assumption that each process has the same efficiency. The total efficiency describes the portion of successfully decodable signals of the total signals present.

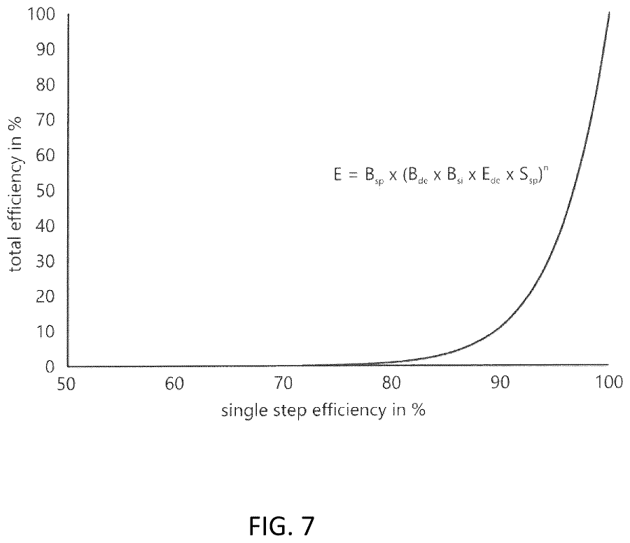

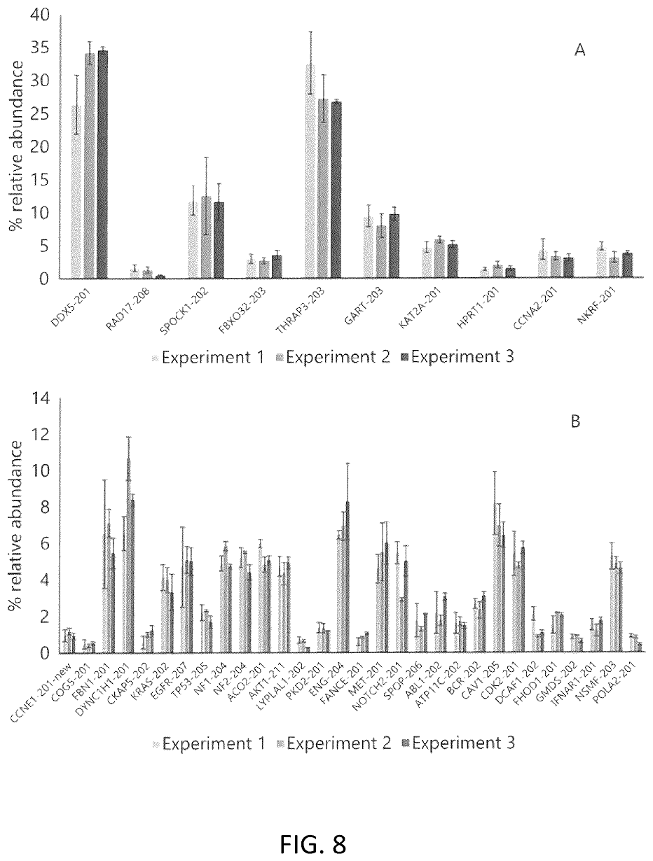

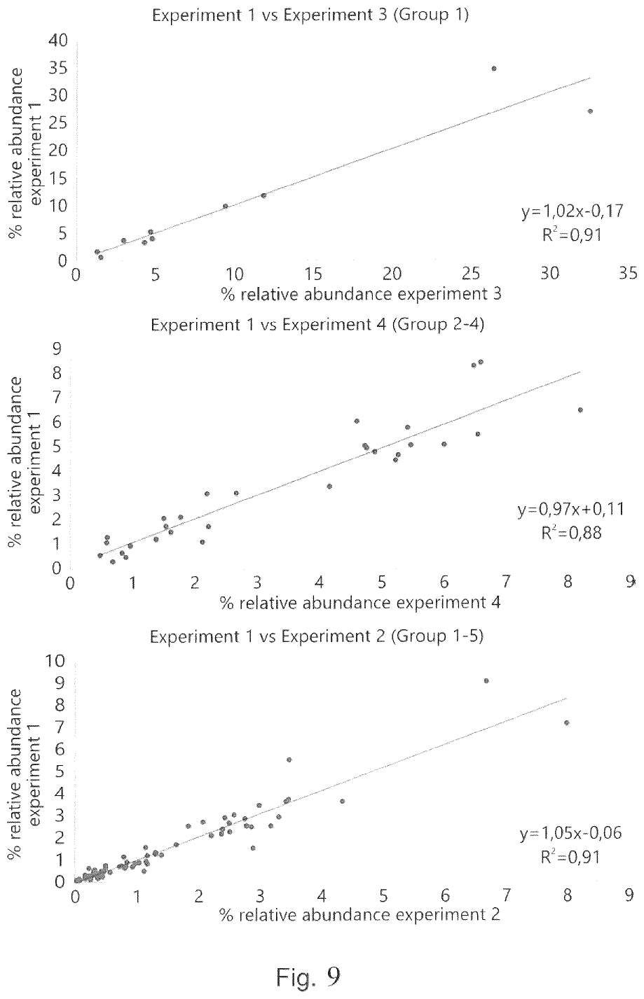

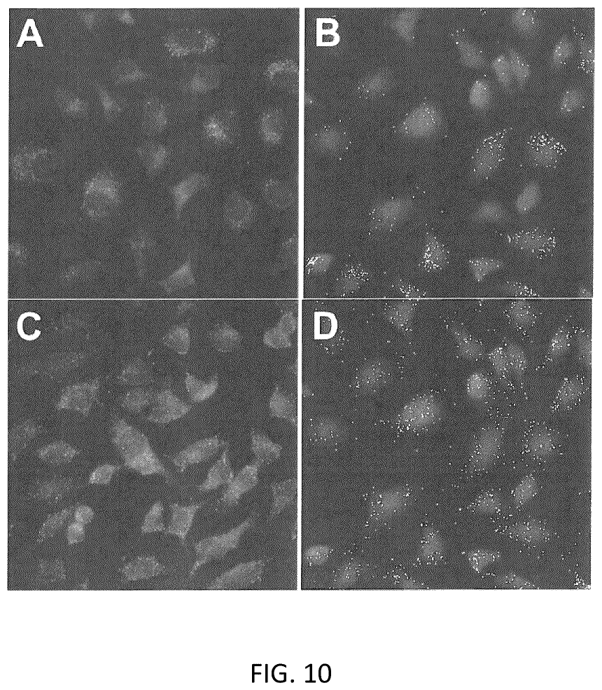

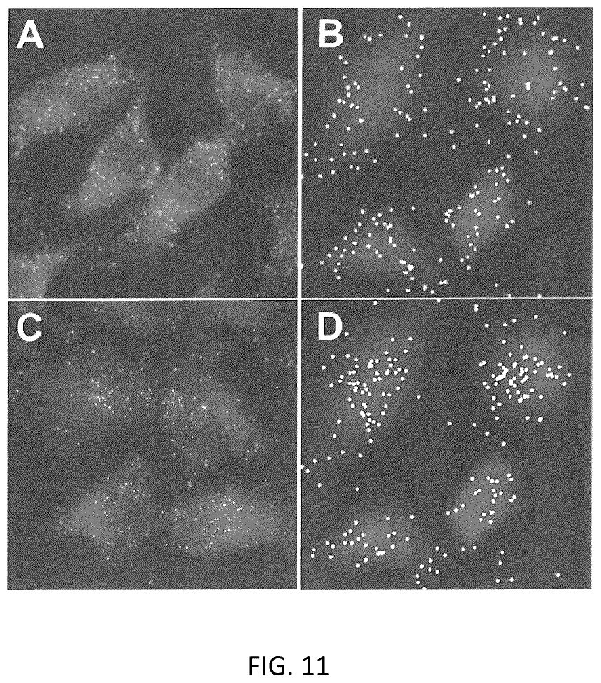

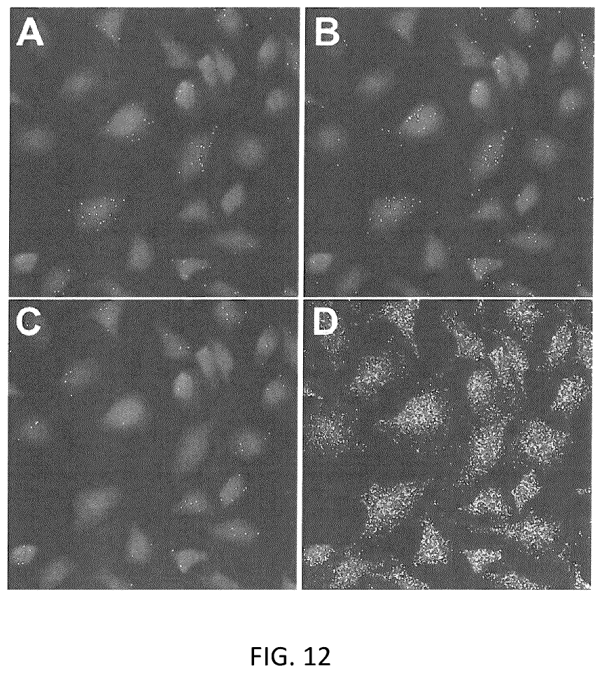

The total efficiency of the method is dependent on the efficiency of each single step of the different factors described by the equation. Under the assumption of an equally distributed efficiency the total efficiency can be plotted against the single step efficiency as shown in FIG. 7. As can be seen, a practically relevant total efficiency for an encoding scheme with 5 detection cycles can only be achieved with single step efficiencies clearly above 90%. For example, to achieve a total efficiency of 50% an average efficiency within each single step of 97.8% is needed. These calculations are even based on the assumption of a 100% signal detection and analysis efficiency. Due to broad DNA melting curves of oligonucleotides of a variety of sequences, the inventors assumed prior to experiments that the selective denaturation would work less efficient for denaturation of decoding oligonucleotides and that sequence specific binding probes are not stable enough. In contrast to this assumption, we found a surprising effectiveness of all steps and a high stability of sequence specific probes during selective denaturation.

Experimentally, the inventors achieved a total decoding efficiency of about 30% to 65% based on 5 detection cycles. A calculation of the efficiency of each single step (Bsp, Bde, Bsi, Ede, Ssp) by the formula given above revealed an average efficiency of about 94.4% to 98%. These high efficiencies are very surprising and cannot easily be anticipated by a well-trained person in this field.

Resilience to variation in Target Analyte

As discussed elsewhere herein, due to the use of multiple probes in a given set that target nonoverlapping regions of a target analyte, a probe or set of probes may be designed to bind the analyte at a plurality of distinct positions, even though the analyte's actual sequence may differ from that of the expected sequence such that at least some of the set of probes do not bind to the analyte. Nonetheless, the remainder of the set of probes remains able to bind the analyte such that it may be detected despite varying from the expected sequence. Variations of this type often occur in diverse populations, such as various human populations, or among rapidly evolving populations, such as viral or other pathogen populations.

Accordingly, some use of the kits, methods and systems herein result in a composition comprising a target analyte that is bound by probes that are targeted to some of the plurality of target positions on the target analyte, while the subset of probes that target a target position on the target analyte for which there is a variation between the target analyte and the expected sequence exhibit no target binding. Thus, for the target analyte, some sets of probes directed to the target analyte exhibit binding to the target analyte, while other sets of probes directed to the target analyte (particularly to regions of the target analyte for which there is variation between the actual and the expected sequence) exhibit little or in some cases absolutely no binding to the target analyte. A composition resulting from such a use of the methods, kits or systems herein comprises a Probe set comprising a plurality of nonoverlapping probe subsets directed to nonoverlapping target regions on a target analyte, wherein at least one probe subset comprises members bound to the target analyte, while at least a second probe subset does not comprise members bound to the target analyte. Often, the target analyte differs in its sequence from the expected target analyte sequence at the binding site of the second probe subset, for example by at least one SNP, an insertion or deletion (in/del) a translocation, a duplication junction, a post-transcriptional modification, an epigenomic modification or other variation impacting binding of the second probe subset to the analyte. The composition further comprises decoding oligos bound to the first probe subset bound to the target analyte at the first target analyte binding site, and signal probes bound to the decoding oligos, which are in turn bound to the first probe subset bound to the target analyte at the first target analyte binding site. In some cases, the signal probes are bound to the decoding oligos at a lower melting temperature than the bridging oligos are bound to the first set of target oligos or than the first set of target oligos are bound to the target analyte, such that the signal probes may be replaced through melting without disrupting the binding of the first target probe subset to the target analyte. Alternately, in some cases all of the probe categories are melted off and the entire annealing process is repeated so as to enable attribution of a multi-signal profile to the target analyte.

The disclosure herein is further understood in light of the following sets of numbered embodiments. 1. A method for detecting a polymorphic analyte in a sample by specific signal-encoding of said polymorphic analyte. 2. The method according to any of the embodiments herein, such as number 1, wherein at least two polymorphic analyte-specific probes form a set of polymorphic analyte-specific probes. 3. The method according to any previous embodiment, such as number 1, wherein the signal element is not specific to a single polymorphic analyte-specific probe. 4. The method according to any of the previous embodiments, wherein the signal element is specific to at least a set of the polymorphic analyte-specific probes. 5. The method according to any of the previous embodiments, wherein the signal element does not identify a single polymorphic analyte-specific probe. 6. The method according to any of the previous embodiments, wherein the signal element does identify at least a set of the polymorphic analyte-specific probes. 7. The method according to any of the previous embodiments, wherein at least two probes of the set, preferably a plurality of polymorphic analyte-specific probes of polymorphic analyte-specific probes share a common label. 8. The method according to any of the previous embodiments, wherein a set of polymorphic analyte-specific probes share a common target. 9. The method according to any of any previous embodiment, such as number 8, wherein the common target is a genetic locus. 10. The method according to any of any previous embodiment, such as numbers 8 to 9, wherein the common target is a transcript. 11. The method according to any of any previous embodiment, such as numbers 8 to 10, wherein the failure of a sub-set of polymorphic analyte-specific probes to bind to the common target, does not preclude the detection of the common target. 12. The method according to any previous embodiment, such as numbers 8 to 11, wherein allelic variation in the common target, does not preclude the detection of the common target. 13. The method according to any previous embodiment, such as number 12, wherein allelic variation comprises at least one insertion, deletion, or single nucleotide variation relative to an expected common target. 14. The method according to any of the previous embodiments, wherein the signal element is specific to a common target. 15. The method according to any of the previous embodiments, wherein the signal element identifies a common target. 16. The method according to any of the previous embodiments, comprising the steps of: (A) contacting the sample with at least a first set of polymorphic analyte-specific probes for encoding different variations of the same polymorphic analyte, each analyte-specific probe interacting with a different variation and/or sub-structure of the polymorphic analyte, wherein the polymorphic analyte is present in the sample in at least as two or more variant forms of a specific nucleic acid sequence and wherein the sequences of each polymorphic analyte are related to each other with a sequence identity of between 85% to 99%, each polymorphic analyte-specific probe comprising (aa) a binding element(S) that specifically interacts with one of the at least two or more variants and/or sub-structure of the polymorphic analyte to be encoded, and (bb) an identifier element (T) comprising a nucleotide sequence which is unique to the polymorphic analyte to be encoded (unique set identifier sequence), wherein each set of polymorphic analyte-specific probes differ from another set of polymorphic analyte-specific probes in the nucleotide sequence of the identifier element (T); and (B) contacting the sample with at least a first set of decoding oligonucleotides per polymorphic analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique set identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element facilitating a signal which is specific for the polymorphic analyte; and Detecting the signal caused by the signal element; Optionally selectively removing the decoding oligonucleotides and signal oligonucleotides from the sample, thereby essentially maintaining the specific binding of the analyte-specific probes to the analytes to be encoded; Performing further cycles comprising steps A) to E) for each further polymorphic analyte to generate an encoding scheme with a specific signal per set of polymorphic analytes, wherein in particular the last cycle may stop with step (D). 17. The method according to any of the previous embodiments, wherein the temporal order of adding one set of polymorphic analyte-specific probes is independent of adding the decoding oligonucleotide, the signal oligonucleotide and/or the signal element. 18. The method according to any of the previous embodiments, wherein the signal oligonucleotide and the signal element are not covalently linked to each other. 19. The method according to any of the previous embodiments, wherein the number of common targets detectable increases exponentially as a function of the number of rounds of detection. 20. The method according to any of the previous embodiments, for in vitro diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the method according to the present disclosure. 21. The method according to any of the previous embodiments, for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the method according to the present disclosure. 22. A kit for polymorphic analyte encoding, comprising: (A) contacting the sample with a set of polymorphic analyte-specific probes for encoding different variations of the same polymorphic analyte, each analyte-specific probe interacting with a different variation and/or sub-structure of the polymorphic analyte, wherein the polymorphic analyte is present in the sample in at least as two or more variant forms of a specific nucleic acid sequence and wherein the sequences of each polymorphic analyte are related to each other with a sequence identity of between 85% to 99%, each polymorphic analyte-specific probe comprising (aa) a binding element(S) that specifically interacts with one of the at least two or more variants and/or sub-structure of the polymorphic analyte to be encoded, and (bb) an identifier element (T) comprising a nucleotide sequence which is unique to the polymorphic analyte to be encoded (unique set identifier sequence), wherein each set of polymorphic analyte-specific probes differ from another set of polymorphic analyte-specific probes in the nucleotide sequence of the identifier element (T); and (B) contacting the sample with at least a first set of decoding oligonucleotides per polymorphic analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique set identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element facilitating a signal which is specific for the polymorphic analyte; and Detecting the signal caused by the signal element; Optionally selectively removing the decoding oligonucleotides and signal oligonucleotides from the sample, thereby essentially maintaining the specific binding of the analyte-specific probes to the analytes to be encoded; Performing further cycles comprising steps A) to E) for each further polymorphic analyte to generate an encoding scheme with a specific signal per set of polymorphic analytes, wherein in particular the last cycle may stop with step (D). 23. An optical multiplexing system suitable for the method according to any of the previous embodiments, comprising at least: at least one reaction vessel for containing the kits or part of the kits according to any previous embodiment, such as number 4; a detection unit comprising a microscope, in particular a fluorescence microscope a camera a liquid handling device. 24. A method for screening, identifying and/or testing a substance and/or drug comprising: (a) contacting a test sample comprising a sample with a substance and/or drug (b) detecting different analytes in a sample by sequential signal-encoding of said analytes with a method according to according to any of the any previous embodiment 1 to 19. 25. A method for detecting a variant analyte in a sample, comprising: contacting the sample to a population of binding probes, wherein members of the population of binding probes comprise i) non-overlapping analyte binding regions and ii) a copy of a common identifier element, such that no individual member of the population of binding probes is identifiable by the identifier element, to form an identifier element bound analyte complex; contacting the identifier element bound analyte complex to a first decoder element population, the first decoder element population comprising probes having a region complementary to the identifier element and a region having a first signal-element binding domain; contacting the identifier element bound analyte complex to a first signal element probe comprising a first fluorophore; detecting a first fluorophore emission spectrum; removing the first decoder element population; contacting the identifier element bound analyte complex to a second decoder element population, the second decoder element population comprising probes having a region complementary to the identifier element and a region having a second signal-element binding domain; contacting the identifier element bound analyte complex to a second signal element probe comprising a second fluorophore; detecting a second fluorophore emission spectrum; and associating a pattern of first fluorophore emission spectra and second fluorophore emission spectra consistent with an order of addition of first decoder element populations and second decoder element populations with a pattern designated for an original analyte for which the variant analyte is a variant, wherein the variant analyte differs from the original analyte such that at least some of the members of the population of binding probes are capable of binding to the original analyte but not to the variant analyte. 26. The method of any previous embodiment, such as number 25, wherein the variant analyte is an allelic variant of the original analyte, and the original analyte is a sequenced genomic locus. 27. The method of any previous embodiment, such as number 25, wherein the variant analyte is an alternatively spliced transcript variant of the original analyte, and the original analyte is a transcript having an expected splice pattern. 28. The method of any previous embodiment, such as number 25, wherein the variant analyte is a protein variant of the original analyte, and the original analyte is a protein having a known nucleic acid sequence. 29. The method of any previous embodiment, such as number 28, wherein the protein variant comprises a post-translational modification. 30. The method of any previous embodiment, such as number 28, wherein the population of binding probes comprises antibodies. 31. The method of any previous embodiment, such as number 28, wherein the population of binding probes comprises aptamers. 32. The method of any previous embodiment, such as number 25, wherein the identifier element is not specific to any individual probe of the population of binding probes. 33. The method of any previous embodiment, such as number 25, wherein the identifier element does not identify any individual probe of the population of binding probes. 34. The method of any previous embodiment, such as number 25, wherein the identifier element is common to at least two probes of the population of binding probes. 35. The method of any previous embodiment, such as number 25, wherein the identifier element is not specific to any individual probe of the population of binding probes. 36. The method of any one of any previous embodiment, such as numbers 25-31 or 32-35, wherein failure of at least some members of the different members of the population of binding probes to bind the variant analyte does not preclude formation of the identifier element bound analyte complex. 37. The method of any one of any previous embodiment, such as numbers 25-31 or 32-35, wherein the order of addition of first decoder element populations and second decoder element populations is not specified by the common identifier. 38. The method of any one of any previous embodiment, such as numbers 25-31 or 32-35, wherein the order of observation of first fluorophore signal and second fluorophore signal is not specified by the common identifier. 39. A method for detecting an analyte in a sample, comprising: contacting the sample to a population of binding probes, wherein members of the population of binding probes comprise i) non-overlapping analyte binding regions and ii) a copy of a common identifier element, such that no individual member of the population of binding probes is identifiable by the identifier element, to form an identifier element bound analyte complex; contacting the identifier element bound analyte complex to a first decoder element population, the first decoder element population comprising probes having a region complementary to the identifier element and a region having a first signal-element binding domain; contacting the identifier element bound analyte complex to a first signal element probe comprising a first fluorophore; detecting a first fluorophore emission spectrum; removing the first decoder element population; contacting the identifier element bound analyte complex to a second decoder element population, the second decoder element population comprising probes having a region complementary to the identifier element and a region having a second signal-element binding domain; contacting the identifier element bound analyte complex to a second signal element probe comprising a second fluorophore; detecting a second fluorophore emission spectrum; and associating a pattern of first fluorophore emission spectra and second fluorophore emission spectra consistent with an order of addition of first decoder element populations and second decoder element populations with a pattern designated for the analyte. 40. The method of any previous embodiment, such as number 39, wherein the identifier element is not specific to any individual probe. 41. The method of any previous embodiment, such as number 39, wherein the identifier element does not identify any individual probe. 42. The method of any previous embodiment, such as number 39, wherein at least two probes share a common identifier element. 43. The method of any previous embodiment, such as number 39, wherein allelic variation in the analyte relative to a reference from which the population of binding probes was generated does not preclude detection of the analyte. 44. The method of any previous embodiment, such as number 39, wherein the pattern of first fluorophore emission spectra and second fluorophore emission spectra consistent with an order of addition of first decoder element populations and second decoder element populations is independent of sequence of the identifier element. 45. The method of any previous embodiment, such as number 39, wherein the pattern of first fluorophore emission spectra and second fluorophore emission spectra consistent with an order of addition of first decoder element populations and second decoder element populations is not specified by sequence of the identifier element. 46. The method of any previous embodiment, such as number 39, wherein the order of addition of first decoder element populations and second decoder element populations is independent of sequence of the identifier element. 47. The method of any previous embodiment, such as number 39, wherein the order of addition of first decoder element populations and second decoder element populations is not specified by sequence of the identifier element. 48. The method of any previous embodiment, such as number 39, wherein the decoder probes bind the identifier element successively at a single binding site. 49. The method of any previous embodiment, such as number 39, wherein the decoder probes are not covalently bound to fluorophores. 50. The method of any previous embodiment, such as number 39, wherein the decoder probes and the signal element probes do not comprise analyte-specific sequence. 51. The method of any previous embodiment, such as number 39, wherein the decoder probes and the signal element probes do not comprise analyte binding moieties. 52. The method of any previous embodiment, such as number 39, wherein the decoder probes and the signal element probes do not comprise analyte binding sequences. 53. The method of any previous embodiment, such as number 39, wherein the decoder probes are not unique to any analyte. 54. The method of any previous embodiment, such as number 39, wherein the signal probes are not unique to any analyte. 55. The method of any previous embodiment, such as number 39, wherein the signal probes are not unique to any decoder probes. 56. The method of any previous embodiment, such as number 39, wherein the number of distinct analytes to be concurrently detected in a single sample, increases exponentially as a function of the number rounds of fluorophore detection.

Similarly, the disclosure is further understood in view of the following numbered embodiments. 1. A multiplex-method for detecting different analytes in a sample beyond the diffraction limit by sequential signal-encoding of said analytes. 2. The method according to any of the previous embodiments, wherein different analytes are contacted with at least one set of analyte-specific probes and wherein at least one set of decoding oligonucleotides per analyte per set of multi-decoding oligonucleotides is used. 3. The method according to any of the previous embodiments, wherein at least one set of signal oligonucleotides per one set of decoding oligonucleotides per analyte is used. 4. The method according to any of the previous embodiments, wherein at least a first set of decoding oligonucleotides and a second set of decoding oligonucleotides is used to identify an analyte in a sample. 5. The method according to any of the previous embodiments, wherein at least the first set of decoding oligonucleotides and the second set of decoding oligonucleotides are added to the analyte in consecutive steps. 6. The method according to any of the previous embodiments, wherein the signals are optically distinct from each other to allow the detection of different analytes in a sample beyond the diffraction limit. 7. The method according to any of the previous embodiments, wherein optical filters and/or computational methods are used in order to differentiate the at least two signals and to allow the detection of different analytes in a sample beyond the diffraction limit. 8. The method according to any of the previous embodiments, wherein the detection limit is either because of low spatial distance between each analyte and/or low abundance of at least one of the analytes. 9. The method according to any of the previous embodiments, wherein the at least two signals enable the detection of analytes within a sample, which are beyond the detection limit of a single signal. 10. The method according to any of the previous embodiments, for detecting different analytes in a sample beyond the diffraction limit by sequential signal-encoding of said analytes, comprising the steps of: (A1) contacting the sample with a first set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and (A2) contacting the sample with a second set of analyte-specific probes for encoding different analytes, each analyte-specific probe interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and wherein (optionally) the number of probes and/or targets of first set of analyte-specific probes according to step A1 (i.e. the transcript plexity of A1) is at least 10 times higher than the number of probes and/or targets of the second set of analyte-specific probes according to step A2 (i.e. the transcript plexity of A2); and (B1) contacting the sample with at least a first set of decoding oligonucleotides per analyte, wherein in each set of decoding oligonucleotides for an individual analyte each decoding oligonucleotide of the for the first set of analyte-specific probes according to step A1 comprises: (aa) an identifier connector element (t) comprising a nucleotide sequence which is essentially complementary to at least a section of the unique identifier sequence of the identifier element (T) of the corresponding analyte-specific probe set A1, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); and (B2) contacting the sample with at least a second set of decoding oligonucleotides per analyte, wherein in each set of decoding oligonucleotides for an individual analyte of each decoding oligonucleotide for the second set of analyte-specific probes according to step A2 comprises: (aa) an identifier connector element (t) comprising a nucleotide sequence which is essentially complementary to at least a section of the unique identifier sequence of the identifier element (T) of the corresponding analyte-specific probe set A2, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the first connect element (t); (C) contacting the sample with at least a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element; and Detecting the signal caused by the signal element; selectively removing the decoding oligonucleotides and signal oligonucleotides from the sample, thereby essentially maintaining the specific binding of the analyte-specific probes to the analytes to be encoded; Performing at least three (3) further cycles comprising steps B) to E) to generate an encoding scheme with a code word per analyte, wherein in particular the last cycle may stop with step (D). 11. The method according to any of the previous embodiments, wherein steps A1 and A2 as well as steps B1 and B2 can be performed in consecutive cycles of the steps in the order (A1, B1, C, D, E and F) n and then (A2, B2, C, D, E and F) n; or in interwoven cycles of the steps in the order (A1, A2, B1, B2, C, D, E and F) n, wherein n is the number of cycles and at least 3. 12. The method according to any of the previous embodiments, for the detection of a cancer selected from adenoid cystic carcinoma, mucoepidermoid carcinoma, follicular thyroid carcinoma, breast carcinoma, Ewing sarcoma, small round cell tumors of bone, synovial sarcoma, glioblastoma multiforme, pilocytic astrocytoma, lung cancer, clear cell renal cell carcinoma, bladder cancer, prostate cancer, ovarian cancer and colorectal cancer and/or any combination thereof. 13. The method according to any of the previous embodiments, for in vitro diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases comprising the use of the multiplex method according to the present disclosure. 14. The method according to any of the previous embodiments, for diagnosis of a disease in plants selected from the group comprising: diseases caused by biotic stress, preferably by infectious and/or parasitic origin, or diseases caused by abiotic stress, preferably caused by nutritional deficiencies and/or unfavorable environment, said method comprising the use of the multiplex method according to the present disclosure. 15. The method according to any of the previous embodiments, wherein an error-correction system is integrated in the binding element(S) and/or identifier element (T) and/or identifier connector element (t) and/or translator element (c) and/or translator connector element (C) and/or signal element. 16. The method according to any of the previous embodiments, wherein at least 200 different genes can be identified with 8 or less rounds of detection. 17. A kit for multiplex analyte encoding beyond the diffraction limit, comprising: (A1) at least a first set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and (A2) at least a second set of analyte-specific probes for encoding different analytes, each set of analyte-specific probes interacting with a different analyte, wherein if the analyte is a nucleic acid each set of analyte-specific probes comprises analyte-specific probes which specifically interact with different sub-structures of the same analyte, each analyte-specific probe comprising (aa) a binding element(S) that specifically interacts with one of the different analytes to be encoded, and (bb) an identifier element (T) comprising a nucleotide sequence which is unique to the analyte to be encoded (unique identifier sequence), wherein the analyte-specific probes of a particular set of analyte-specific probes differ from the analyte-specific probes of another set of analyte-specific probes in the nucleotide sequence of the identifier element (T), wherein the analyte-specific probes in each set of analyte-specific probes binds to the same analyte and comprises the same nucleotide sequence of the identifier element (T) which is unique to said analyte; and wherein the number of probes and/or targets of first set of analyte-specific probes according to step A1 (i.e. the transcript plexity of A1) is at least 10 times higher than the number of probes and/or targets of the second set of analyte-specific probes according to step A2 (i.e. the transcript plexity of A2); and (B) at least one set of decoding oligonucleotides per analyte set A1 and A2, wherein in each set of decoding oligonucleotides for an individual analyte each decoding oligonucleotide comprises: (aa) an identifier connector element (t) comprising a nucleotide sequence which is essentially complementary to at least a section of the unique identifier sequence of the identifier element (T) of the corresponding analyte-specific probe set, and (bb) a translator element (c) comprising a nucleotide sequence allowing a specific hybridization of a signal oligonucleotide; wherein the decoding oligonucleotides of a set for an individual analyte differ from the decoding oligonucleotides of another set for a different analyte in the identifier connect element (t); and (C) a set of signal oligonucleotides, each signal oligonucleotide comprising: (aa) a translator connector element (C) comprising a nucleotide sequence which is essentially complementary to at least a section of the nucleotide sequence of a translator element (c) comprised in a decoding oligonucleotide, and (bb) a signal element. 19. An optical multiplexing system suitable for the method according to embodiments 1-16, comprising at least: at least one reaction vessel for containing the kits or part of the kits according to embodiment 17; a detection unit comprising a microscope, in particular a fluorescence microscope a camera a liquid handling device. 20. A method for screening, identifying and/or testing a substance and/or drug comprising: (a) contacting a test sample comprising a sample with a substance and/or drug (b) detecting different analytes in a sample by sequential signal-encoding of said analytes with a method according to embodiments 1-16. 21. A method according any one of embodiments 1 to 16 for the co-localization of different molecular groups such as RNA and protein, or RNA and DNA, or DNA and protein, or two different RNA molecules, or two different DNA sequences, or two different proteins. 22. A method according any one of embodiments 1 to 16 for the co-localization of at least two different features of a single molecule a. two parts of a nucleic acid molecule (differential splicing, fusion transcripts, fusion genes; b. two parts of a protein or a dimerized protein; 23. A method according any one of embodiments 1 to 16 for the Co-localization of abundant and rare transcripts.

### EXAMPLES

1. Signal encoding of two subgroups of nucleic acid sequences with spatial overlap

The table below shows examples of implementations using two subgroups (analytic sets) in consecutive runs:

| | | Transcript | | | | |
|---|---|---|---|---|---|---|
| | | Plexity | Run 1 | | Run 2 | |
| | Total | (Set 1/ | Fluidics | Plexity | Fluidics | Plexity |
| | rounds | Set 2) | rounds | 1 | rounds | 2 |
| 1.0 | 9 | 100/2 | 8 | 100 (129) | 1 | 2 |
| 2.0 | 16 | 300/25 | 10 | 300 (419) | 6 | 25 (29) |
| 2.1 | 16 | 500/50 | 10 | 500 (685) | 6 | 50 (60) |
| Improved | | | | | | |
| transcript | | | | | | |
| detection | | | | | | |

For “run 1.0” n=8 rounds of hybridization and imaging were used to detect 100 genes. For “run 2.0” the number of rounds is increased to n=10 and can thereby target 300 genes. For “run 2.1” an enhanced transcript detection probe is used which allows to target 500 transcripts with the same number of rounds (n=10).

Consecutive runs are run on the same tissue sections to detect transcripts beyond the diffraction limit.

The main steps of the method are 1) Hybridization of target probes of the two analytic sets happen simultaneously before the first run, 2) Tails of the target probes are unique for both runs. e.g., 100, 300 and 500 tails (for 100, 300, or 500 transcripts, respectively) are used for the first run with the first analytic set and 2, 25, 50 tails (for 2, 25, 50 transcripts, respectively) for the second run with the second analytic set. This requires also different decoder sets for both runs. Both runs generate independent datasets that can be combined in silico afterwards.

Thereby, 1) enhancing the multiplexing capability without increasing optical crowding and 2) detecting spatial overlapping transcripts which would be otherwise not possible due to the diffraction limit of the microscope. This allows for detection of fusion genes (cancer) or for detection of co-localization of different transcript types (e.g. transcriptional hubs in the nucleus). In addition, transcripts with higher expression levels can also be analyzed because the “signal spread” of the high number of abundant signals vs. the detection of other (especially lowly expressed) genes is improved.

In principle, both runs can be also interwoven, e.g.:
Detection round 1=1st round of detection of first analytical set.
Detection round 2=1st round of detection of second analytical set.
Detection round 3=2nd round of detection of first analytical set.
Detection round 4=2nd round of detection of second analytical set.
Etc.
   2. Detecting two transcripts derived from different genes that have a distance below the diffraction limit

Detecting two transcripts derived from different genes that have a distance below the diffraction limit requires two consecutive MC experiments on the same sample addressing different target probe sets/tails that have been hybridized together. To evaluate the feasibility of this concept, two consecutive experiments were run on one tissue sample. However, the same tails were addressed in both experiments to evaluate the sample stability by analyzing transcript detection, false-positive rates (FPRs) and signal intensities. For mouse brain tissue, the decrease in decoded transcripts was mild for multiple regions (˜20%) and the overall correlation of transcript counts was high (P=0.98). FPRs were consistently low in both experiments (˜0.02% detected blank barcodes). In addition, signal intensities across all genes were remarkably similar and showing only a minor decrease. Taken together, the results demonstrate the general feasibility in detecting transcripts in two consecutive experiments with high confidence.

FIG. 18A through FIG. 18E show the comparison of two consecutive MC runs targeting the same tails. FIG. 18A Overview of experimental setup. FIG. 18B Barplot showing transcript counts in different tissue regions of first and second set. FIG. 18C Scatterplot of transcript counts per genes of first and second set. FIG. 18D Barplot showing false positive rates (fractions of detected blank barcodes) in different tissue regions of first and second set. FIG. 18E Boxplot of signal intensities per imaging round. Top: Channel 1; Bottom: Channel 2. Left: first set; Right: second set.

### SEQUENCE LISTING

In the accompanying sequence listing SEQ ID Nos. 1-1247 refer to nucleotide sequences of exemplary target-specific oligonucleotides. The oligonucleotides listed consist of a target specific binding site (5′-end) a spacer/linker sequence (gtaac or tagac) and the unique identifier sequence, which is the same for all oligonucleotides of one probe set.

In the accompanying sequence listing SEQ ID Nos. 1248-1397 refer to nucleotide sequences of exemplary decoding oligonucleotides.

In the accompanying sequence listing SEQ ID Nos. 1398-1400 refer to the nucleotide sequences of exemplary signal oligonucleotides. For each signal oligonucleotide the corresponding fluorophore is present twice. One fluorophore is covalently linked to the 5′-end and one fluorophore is covalently linked to the 3′-end. SEQ ID No. 1398 comprises at its 5′ terminus “5Alex488N”, and at its 3′ terminus “3AlexF488N”. SEQ ID No. 1399 comprises at its 5′ terminus “5Alex546”, and at its 3′ terminus 3Alex546N. SEQ ID No. 1400 comprises at its 5′ terminus and at its 3′ terminus “Atto594”.

Example 2: Cancer tissue analysis (conventional method for comparison). A cancer tissue section is obtained and assayed for a transcript distribution pattern within the section using a conventional method rather than the methods disclosed herein. The section is obtained from an individual whose genome has not been sequenced, and whose genome differs from that of the sequenced consensus genome at a number of positions including SNPs and small IN/DELs. A uniform FISH probe population is used to determine distribution of the transcript in the cancer tissue section. The fish probe is designed in light of a consensus published human genome. Unknown to the designer of the FISH probe, the probe spans a transcribed region of the genome for which there is substantial variation between the consensus genome from which the FISH probe is designed, and the genome of the individual from which the cancer tissue section was obtained. The probe is used to determine a transcript accumulation pattern in the cancer tissue section. The probe fails to anneal to the transcript harboring the individual's variant relative to the consensus published genome sequence. Accordingly, the transcript pattern in the cancer tissue section is incorrectly inferred from the experimental data. As a consequence, the cancer is mischaracterized and an incorrect treatment regimen is proposed for the individual.

Example 3: Cancer tissue analysis (according to the method of the present application). A cancer tissue section is obtained and assayed for a transcript distribution pattern within the section according to the disclosure herein. The section is obtained from an individual whose genome has not been sequenced, and whose genome differs from that of the sequenced consensus genome at a number of positions including SNPs and small IN/DELs. A heterogeneous probe population comprising oligonucleotides that anneal to a plurality of nonoverlapping positions of the target of interest, and sharing a common tag sequence, is used to determine distribution of the transcript in the cancer tissue section. The heterogeneous probe population is designed in light of a consensus published human genome. Unknown to the designer of the heterogeneous probe population, the probe population comprises individual probes that span a transcribed region of the genome for which there is substantial variation between the consensus genome from which the probe population is designed, and the genome of the individual from which the cancer tissue section was obtained. The heterogeneous probe population is used to determine a transcript accumulation pattern in the cancer tissue section. Representatives of the heterogeneous probe population fail to anneal to the transcript at the regions of the transcript corresponding to the individual's variant relative to the consensus published genome sequence. Nonetheless, other representatives of the heterogeneous probe population anneal to the transcript at positions distal to the variant region, such that the transcript is correctly detected despite harboring the variant region. As all of the heterogeneous probe population members share a commonly identifiable barcode, the correctly annealing members of the heterogeneous probe population allow one to identify the transcript in its correct distribution pattern, facilitating a medical practitioner's efforts to identify a treatment for the cancer.

Accordingly, the transcript pattern in the cancer tissue section is correctly inferred from the experimental data. As a consequence, the cancer is correctly characterized and an appropriate treatment regimen is proposed for the individual despite the individual's genome not matching publicly available consensus genome sequence.

This pair of examples illustrates how the non-overlapping redundancy in the locus-targeting probes used to detect a given target allows one to identify transcripts or other nucleic acid targets even when their actual sequence differs from that of the genome of the target tissue.

Example 4: Cancer tissue analysis (conventional method for comparison) A cancer tissue section is obtained and assayed for a transcript distribution pattern within the section using a conventional approach. The target transcript is alternatively spliced in the tissue, such that a segment of the transcript expected to be present is instead spliced out of the mature transcript. A uniform FISH probe population is used to determine distribution of the transcript in the cancer tissue section. The fish probe is designed in light of a consensus published transcript sequence, which is generated without knowledge of the alternative splicing event. Unknown to the designer of the FISH probe, the probe targets the region of the transcript which is excised in the mature transcript in the cancer tissue. The probe is used to determine a transcript accumulation pattern in the cancer tissue section. The probe fails to anneal to the alternatively spliced transcript. Accordingly, the transcript pattern in the cancer tissue section is incorrectly inferred from the experimental data. As a consequence, the cancer is mischaracterized and an incorrect treatment regimen is proposed for the individual.

Example 5: Cancer tissue analysis (according to the method of the present application). A cancer tissue section is obtained as in Example 4 and assayed for a transcript distribution pattern within the section according to the disclosure herein. The target transcript is alternatively spliced in the tissue, such that a segment of the transcript expected to be present is instead spliced out of the mature transcript.

A heterogeneous probe population comprising oligonucleotides that anneal to a plurality of nonoverlapping positions of the target of interest, and sharing a common tag sequence, is used to determine distribution of the transcript in the cancer tissue section. The heterogeneous probe population is designed in light of the consensus published transcript sequence. Unknown to the designer of the heterogeneous probe population, the probe population comprises individual probes that target the segment of the transcript which is excised as a result of the alternative splicing event, in addition to comprising other probes that target segments of the transcript present in the mature transcript as it accumulates in the cancer tissue. The heterogeneous probe population is used to determine a transcript accumulation pattern in the cancer tissue section. Representatives of the heterogeneous probe population fail to anneal to the transcript at the regions of the transcript corresponding to excised or ‘spliced out’ regions relative to the consensus published genome sequence. Nonetheless, other representatives of the heterogeneous probe population anneal to the transcript at positions distal to the alternative splicing region, such that the transcript is correctly detected despite being alternatively spliced. As all of the heterogeneous probe population members share a commonly identifiable barcode, the correctly annealing members of the heterogeneous probe population allow one to identify the transcript in its correct distribution pattern, facilitating a medical practitioner's efforts to identify a treatment for the cancer.

Accordingly, the transcript pattern in the cancer tissue section is correctly inferred from the experimental data. As a consequence, the cancer is correctly characterized and an appropriate treatment regimen is proposed despite the unknown alternative splicing event being present in the cancer tissue.

This pair of examples illustrates how the non-overlapping redundancy in the locus-targeting probes used to detect a given target allows one to identify transcripts even when their mature processed sequence differs from a predicted sequence.

We claim:

1. A method for detecting a variant analyte in a sample, comprising: contacting the sample to a population of binding probes, wherein members of the population of binding probes comprise i) non-overlapping analyte binding regions and ii) a copy of a common identifier element, such that no individual member of the population of binding probes is identifiable by the identifier element, to form an identifier element bound analyte complex; contacting the identifier element bound analyte complex to a first decoder element population, the first decoder element population comprising probes having a region complementary to the identifier element and a region having a first signal-element binding domain; contacting the identifier element bound analyte complex to a first signal element probe comprising a first fluorophore; detecting a first fluorophore emission spectrum; removing the first decoder element population; contacting the identifier element bound analyte complex to a second decoder element population, the second decoder element population comprising probes having a region complementary to the identifier element and a region having a second signal-element binding domain; contacting the identifier element bound analyte complex to a second signal element probe comprising a second fluorophore; detecting a second fluorophore emission spectrum; and associating a pattern of first fluorophore emission spectra and second fluorophore emission spectra consistent with an order of addition of first decoder element populations and second decoder element populations with a pattern designated for an original analyte for which the variant analyte is a variant, wherein the variant analyte differs from the original analyte such that at least some of the members of the population of binding probes are capable of binding to the original analyte but not to the variant analyte.

2. The method of claim 1, wherein the variant analyte is an allelic variant of the original analyte, and the original analyte is a sequenced genomic locus.

3. The method of claim 1, wherein the variant analyte is an alternatively spliced transcript variant of the original analyte, and the original analyte is a transcript having an expected splice pattern.

4. The method of claim 1, wherein the variant analyte is a protein variant of the original analyte, and the original analyte is a protein having a known nucleic acid sequence.

5. The method of claim 4, wherein the protein variant comprises a post-translational modification.

6. The method of claim 4, wherein the population of binding probes comprises antibodies.

7. The method of claim 4, wherein the population of binding probes comprises aptamers.

8. The method of claim 1, wherein the identifier element is not specific to any individual probe of the population of binding probes.

9. The method of claim 1, wherein the identifier element does not identify any individual probe of the population of binding probes.

10. The method of claim 1, wherein the identifier element is common to at least two probes of the population of binding probes.

11. The method of claim 1, wherein the identifier element is not specific to any individual probe of the population of binding probes.

12. The method of claim 1, wherein failure of at least some members of the different members of the population of binding probes to bind the variant analyte does not preclude formation of the identifier element bound analyte complex.

13. The method of claim 1, wherein the order of addition of first decoder element populations and second decoder element populations is not specified by the common identifier.

14. The method of claim 1, wherein the order of observation of first fluorophore signal and second fluorophore signal is not specified by the common identifier.

15. A method for detecting an analyte in a sample, comprising: contacting the sample to a population of binding probes, wherein members of the population of binding probes comprise i) non-overlapping analyte binding regions and ii) a copy of a common identifier element, such that no individual member of the population of binding probes is identifiable by the identifier element, to form an identifier element bound analyte complex; contacting the identifier element bound analyte complex to a first decoder element population, the first decoder element population comprising probes having a region complementary to the identifier element and a region having a first signal-element binding domain; contacting the identifier element bound analyte complex to a first signal element probe comprising a first fluorophore; detecting a first fluorophore emission spectrum; removing the first decoder element population; contacting the identifier element bound analyte complex to a second decoder element population, the second decoder element population comprising probes having a region complementary to the identifier element and a region having a second signal-element binding domain; contacting the identifier element bound analyte complex to a second signal element probe comprising a second fluorophore; detecting a second fluorophore emission spectrum; and associating a pattern of first fluorophore emission spectra and second fluorophore emission spectra consistent with an order of addition of first decoder element populations and second decoder element populations with a pattern designated for the analyte.

16. The method of claim 15, wherein the identifier element is not specific to any individual probe, or does not identify any individual probe.

17. The method of claim 15, wherein at least two probes share a common identifier element.

18. The method of claim 15, wherein the pattern of first fluorophore emission spectra and second fluorophore emission spectra consistent with an order of addition of first decoder element populations and second decoder element populations is independent of sequence of the identifier element.

19. The method of claim 15, wherein the decoder probes bind the identifier element successively at a single binding site.

20. The method of claim 15, wherein the decoder probes and the signal element probes do not comprise analyte-specific sequence.


SEQUENCE LISTING (1400 sequences)

SEQ ID NO: 1 - DNA, 45 bp, synthetic construct
  misc_feature 1..45: note = Target Specific Oligonucleotide
  source 1..45: mol_type = other DNA
actactagagaccggtagaaatgagtaacgattaccgacttatcc

SEQ ID NO: 2 - DNA, 42 bp, synthetic construct
  misc_feature 1..42: note = Target Specific Oligonucleotide
  source 1..42: mol_type = other DNA
tggcggggaacgaagtatatagtaacgattaccgacttatcc

SEQ ID NO: 3 - DNA, 40 bp, synthetic construct
  misc_feature 1..40: note = Target Specific Oligonucleotide
  source 1..40: mol_type = other DNA
tggcgtcaatggttgcggtgtaacgattaccgacttatcc

SEQ ID NO: 4 - DNA, 41 bp, synthetic construct
  misc_feature 1..41: note = Target Specific Oligonucleotide
  source 1..41: mol_type = other DNA
tcggtcactcgaataacccggtaacgattaccgacttatcc

SEQ ID NO: 5 - DNA, 41 bp, synthetic construct
  misc_feature 1..41: note = Target Specific Oligonucleotide
  source 1..41: mol_type = other DNA
ctccaaatcgaggtgcaccagtaacgattaccgacttatcc

SEQ ID NO: 6 - DNA, 40 bp, synthetic construct
  misc_feature 1..40: note = Target Specific Oligonucleotide
  source 1..40: mol_type = other DNA
ggccagttcccgagttggtgtaacgattaccgacttatcc

SEQ ID NO: 7 - DNA, 42 bp, synthetic construct
  misc_feature 1..42: note = Target Specific Oligonucleotide
  source 1..42: mol_type = other DNA
agacttcaagcgacatgctctgtaacgattaccgacttatcc

SEQ ID NO: 8 - DNA, 42 bp, synthetic construct
  misc_feature 1..42: note = Target Specific Oligonucleotide
  source 1..42: mol_type = other DNA
ggagcaccaccgtagatacaagtaacgattaccgacttatcc

SEQ ID NO: 9 - DNA, 44 bp, synthetic construct
  misc_feature 1..44: note = Target Specific Oligonucleotide
  source 1..44: mol_type = other DNA
ctctccaaatcacgtatttgtgggtaacgattaccgacttatcc

SEQ ID NO: 10 - DNA, 45 bp, synthetic construct
  misc_feature 1..45: note = Target Specific Oligonucleotide
  source 1..45: mol_type = other DNA
agagtttgcctatcaggtcttattgtaacgattaccgacttatcc

SEQ ID NO: 11 - DNA, 40 bp, synthetic construct
  misc_feature 1..40: note = Target Specific Oligonucleotide
  source 1..40: mol_type = other DNA
ggccaagtcgcactccacagtaacgattaccgacttatcc

SEQ ID NO: 12 - DNA, 43 bp, synthetic construct
  misc_feature 1..43: note = Target Specific Oligonucleotide
  source 1..43: mol_type = other DNA
tccttttctacgtcatgacacagtaacgattaccgacttatcc

SEQ ID NO: 13 - DNA, 46 bp, synthetic construct
  misc_feature 1..46: note = Target Specific Oligonucleotide
  source 1..46: mol_type = other DNA
ttccattagacgaataagtttttcagtaacgattaccgacttatcc

SEQ ID NO: 14 - DNA, 44 bp, synthetic construct
  misc_feature 1..44: note = Target Specific Oligonucleotide
  source 1..44: mol_type = other DNA
tttctggtaagctcatcacatctgtaacgattaccgacttatcc

SEQ ID NO: 15 - DNA, 46 bp, synthetic construct
  misc_feature 1..46: note = Target Specific Oligonucleotide
  source 1..46: mol_type = other DNA
gagttagggtagtcataattgatgagtaacgattaccgacttatcc

SEQ ID NO: 16 - DNA, 47 bp, synthetic construct
  misc_feature 1..47: note = Target Specific Oligonucleotide
  source 1..47: mol_type = other DNA
ttcttccaattcgatgaatataatccgtaacgattaccgacttatcc

SEQ ID NO: 17 - DNA, 48 bp, synthetic construct
  misc_feature 1..48: note = Target Specific Oligonucleotide
  source 1..48: mol_type = other DNA
tgttattaggtgtaaagaaagtgtatggtaacgattaccgacttatcc

SEQ ID NO: 18 - DNA, 42 bp, synthetic construct
  misc_feature 1..42: note = Target Specific Oligonucleotide
  source 1..42: mol_type = other DNA
gagataaggtcgctcacttgcgtaacgattaccgacttatcc

SEQ ID NO: 19 - DNA, 42 bp, synthetic construct
  misc_feature 1..42: note = Target Specific Oligonucleotide
  source 1..42: mol_type = other DNA
acctctgtcttcgaccaactggtaacgattaccgacttatcc

SEQ ID NO: 20 - DNA, 41 bp, synthetic construct
  misc_feature 1..41: note = Target Specific Oligonucleotide
  source 1..41: mol_type = other DNA
acccctggaacgacctgaacgtaacgattaccgacttatcc

SEQ ID NO: 21 - DNA, 41 bp, synthetic construct
  misc_feature 1..41: note = Target Specific Oligonucleotide
  source 1..41: mol_type = other DNA
agtatctgtcccgacggtcagtaacgattaccgacttatcc

SEQ ID NO: 22 - DNA, 40 bp, synthetic construct
  misc_feature 1..40: note = Target Specific Oligonucleotide
  source 1..40: mol_type = other DNA
accccttttgcccgcagaggtaacgattaccgacttatcc

SEQ ID NO: 23 - DNA, 44 bp, synthetic construct
  misc_feature 1..44: note = Target Specific Oligonucleotide
  source 1..44: mol_type = other DNA
tttaagcaggctagagtaacctcgtaacgattaccgacttatcc

SEQ ID NO: 24 - DNA, 45 bp, synthetic construct
  misc_feature 1..45: note = Target Specific Oligonucleotide
  source 1..45: mol_type = other DNA
gcattggataaccaatcataggtggtaacgattaccgacttatcc

SEQ ID NO: 25 - DNA, 41 bp, synthetic construct
  misc_feature 1..41: note = Target Specific Oligonucleotide
  source 1..41: mol_type = other DNA
tcccctagtccgagttgctcgtaaccctaattatgggaatc

SEQ ID NO: 26 - DNA, 43 bp, synthetic construct
  misc_feature 1..43: note = Target Specific Oligonucleotide
  source 1..43: mol_type = other DNA
ccaatttgcacttggatgtgtagtaaccctaattatgggaatc

SEQ ID NO: 27 - DNA, 43 bp, synthetic construct
  misc_feature 1..43: note = Target Specific Oligonucleotide
  source 1..43: mol_type = other DNA
ttttgccagtagaacaagcatggtaaccctaattatgggaatc

SEQ ID NO: 28 - DNA, 40 bp, synthetic construct
  misc_feature 1..40: note = Target Specific Oligonucleotide
  source 1..40: mol_type = other DNA
ggaggcaaggttccgcaacgtaaccctaattatgggaatc

SEQ ID NO: 29 - DNA, 44 bp, synthetic construct
  misc_feature 1..44: note = Target Specific Oligonucleotide
  source 1..44: mol_type = other DNA
agaattccacgtaaatcacatgcgtaaccctaattatgggaatc

SEQ ID NO: 30 - DNA, 44 bp, synthetic construct
  misc_feature 1..44: note = Target Specific Oligonucleotide
  source 1..44: mol_type = other DNA
tatgcatgtacagggaagtatccgtaaccctaattatgggaatc

SEQ ID NO: 31 - DNA, 43 bp, synthetic construct
  misc_feature 1..43: note = Target Specific Oligonucleotide
  source 1..43: mol_type = other DNA
gtgtgcatgccttagaaaaagcgtaaccctaattatgggaatc

SEQ ID NO: 32 - DNA, 43 bp, synthetic construct
  misc_feature 1..43: note = Target Specific Oligonucleotide
  source 1..43: mol_type = other DNA
agcttttgtgcccttatcatgagtaaccctaattatgggaatc

SEQ ID NO: 33 - DNA, 46 bp, synthetic construct
  misc_feature 1..46: note = Target Specific Oligonucleotide
  source 1..46: mol_type = other DNA
cttcattagtatcttgtcacttggggtaaccctaattatgggaatc

SEQ ID NO: 34 - DNA, 45 bp, synthetic construct
  misc_feature 1..45: note = Target Specific Oligonucleotide
  source 1..45: mol_type = other DNA
agatgatgaccctaccaaatttgggtaaccctaattatgggaatc

SEQ ID NO: 35 - DNA, 44 bp, synthetic construct
  misc_feature 1..44: note = Target Specific Oligonucleotide
  source 1..44: mol_type = other DNA
aggcaagagatagatatgtgggcgtaaccctaattatgggaatc

SEQ ID NO: 36 - DNA, 44 bp, synthetic construct
  misc_feature 1..44: note = Target Specific Oligonucleotide
  source 1..44: mol_type = other DNA
cagctgccactataatcatgtttgtaaccctaattatgggaatc

SEQ ID NO: 37 - DNA, 45 bp, synthetic construct
  misc_feature 1..45: note = Target Specific Oligonucleotide
  source 1..45: mol_type = other DNA
ggaacatgctaatttaaggtgagtgtaaccctaattatgggaatc

SEQ ID NO: 38 - DNA, 46 bp, synthetic construct
  misc_feature 1..46: note = Target Specific Oligonucleotide
  source 1..46: mol_type = other DNA
aggactttgctatatcaagtagttcgtaaccctaattatgggaatc

SEQ ID NO: 39 - DNA, 46 bp, synthetic construct
  misc_feature 1..46: note = Target Specific Oligonucleotide
  source 1..46: mol_type = other DNA
aaaggagatagtaacaatggttttcgtaaccctaattatgggaatc

SEQ ID NO: 40 - DNA, 43 bp, synthetic construct
  misc_feature 1..43: note = Target Specific Oligonucleotide
  source 1..43: mol_type = other DNA
aaatcagtggttcaccctgttcgtaaccctaattatgggaatc

SEQ ID NO: 41 - DNA, 48 bp, synthetic construct
  misc_feature 1..48: note = Target Specific Oligonucleotide
  source 1..48: mol_type = other DNA
cttgtcataagataattaggcaaattagtaaccctaattatgggaatc

SEQ ID NO: 42 - DNA, 47 bp, synthetic construct
  misc_feature 1..47: note = Target Specific Oligonucleotide
  source 1..47: mol_type = other DNA
tcattaagttaatgctaaggatctttgtaaccctaattatgggaatc

SEQ ID NO: 43 - DNA, 43 bp, synthetic construct
  misc_feature 1..43: note = Target Specific Oligonucleotide
  source 1..43: mol_type = other DNA
tgtcactggtacaagtggacttgtaaccctaattatgggaatc

SEQ ID NO: 44 - DNA, 45 bp, synthetic construct
  misc_feature 1..45: note = Target Specific Oligonucleotide
  source 1..45: mol_type = other DNA
ttggtcactatacaagtgacttctgtaaccctaattatgggaatc

SEQ ID NO: 45 - DNA, 43 bp, synthetic construct
  misc_feature 1..43: note = Target Specific Oligonucleotide
  source 1..43: mol_type = other DNA
aggaaaatgggcgtaaaggagggtaaccctaattatgggaatc

SEQ ID NO: 46 - DNA, 46 bp, synthetic construct
  misc_feature 1..46: note = Target Specific Oligonucleotide
  source 1..46: mol_type = other DNA
ttcatctacaatagggacaacaaacgtaaccctaattatgggaatc

SEQ ID NO: 47 - DNA, 44 bp, synthetic construct
  misc_feature 1..44: note = Target Specific Oligonucleotide
  source 1..44: mol_type = other DNA
gataagtctacgtggaaaagcatgtaaccctaattatgggaatc

SEQ ID NO: 48 - DNA, 45 bp, synthetic construct
  misc_feature 1..45: note = Target Specific Oligonucleotide
  source 1..45: mol_type = other DNA
caggactacagttaagcatttactgtaaccctaattatgggaatc

SEQ ID NO: 49 - DNA, 43 bp, synthetic construct
  misc_feature 1..43: note = Target Specific Oligonucleotide
  source 1..43: mol_type = other DNA
gtctaggaaattgccgtggttggtaaccctaattatgggaatc

SEQ ID NO: 50 - DNA, 42 bp, synthetic construct
  misc_feature 1..42: note = Target Specific Oligonucleotide
  source 1..42: mol_type = other DNA
aatcatcgtctcgaaagcggtgtaaccctaattatgggaatc

SEQ ID NO: 51 - DNA, 43 bp, synthetic construct
  misc_feature 1..43: note = Target Specific Oligonucleotide
  source 1..43: mol_type = other DNA
cttgaccaaattcgaaggtccagtaaccctaattatgggaatc

SEQ ID NO: 52 - DNA, 43 bp, synthetic construct
  misc_feature 1..43: note = Target Specific Oligonucleotide
  source 1..43: mol_type = other DNA
gatgactctgttcgcatcctcggtaaccctaattatgggaatc

SEQ ID NO: 53 - DNA, 42 bp, synthetic construct
  misc_feature 1..42: note = Target Specific Oligonucleotide
  source 1..42: mol_type = other DNA
gatgctagtgtcaaacctgccgtaaccctaattatgggaatc

SEQ ID NO: 54 - DNA, 44 bp, synthetic construct
  misc_feature 1..44: note = Target Specific Oligonucleotide
  source 1..44: mol_type = other DNA
tgatacagggctcgtacttatccgtaaccctaattatgggaatc

SEQ ID NO: 55 - DNA, 43 bp, synthetic construct
  misc_feature 1..43: note = Target Specific Oligonucleotide
  source 1..43: mol_type = other DNA
aggagtcacacgagttgaaaaggtaaccctaattatgggaatc

SEQ ID NO: 56 - DNA, 44 bp, synthetic construct
  misc_feature 1..44: note = Target Specific Oligonucleotide
  source 1..44: mol_type = other DNA
ctcattacaccgaggtatgaagggtaaccctaattatgggaatc

SEQ ID NO: 57 - DNA, 40 bp, synthetic construct
  misc_feature 1..40: note = Target Specific Oligonucleotide
  source 1..40: mol_type = other DNA
ctctgtcacggctcgggtggtaaccctaattatgggaatc

SEQ ID NO: 58 - DNA, 43 bp, synthetic construct
  misc_feature 1..43: note = Target Specific Oligonucleotide
  source 1..43: mol_type = other DNA
ctaccatatgtacccgacctcagtaaccctaattatgggaatc

SEQ ID NO: 59 - DNA, 44 bp, synthetic construct
  misc_feature 1..44: note = Target Specific Oligonucleotide
  source 1..44: mol_type = other DNA
aaccttaggtcgggtatagagaggtaaccctaattatgggaatc

SEQ ID NO: 60 - DNA, 44 bp, synthetic construct
  misc_feature 1..44: note = Target Specific Oligonucleotide
  source 1..44: mol_type = other DNA
gaagcacttagacactgtaaggcgtaaccctaattatgggaatc

SEQ ID NO: 61 - DNA, 42 bp, synthetic construct
  misc_feature 1..42: note = Target Specific Oligonucleotide
  source 1..42: mol_type = other DNA
aataatcaccggcagtaacgggtaaccctaattatgggaatc

SEQ ID NO: 62 - DNA, 43 bp, synthetic construct
  misc_feature 1..43: note = Target Specific Oligonucleotide
  source 1..43: mol_type = other DNA
aggaagaaacctatggcagacagtaaccctaattatgggaatc

SEQ ID NO: 63 - DNA, 46 bp, synthetic construct
  misc_feature 1..46: note = Target Specific Oligonucleotide
  source 1..46: mol_type = other DNA
catgtgttattacgatgttctttgtgtaaccctaattatgggaatc

SEQ ID NO: 64 - DNA, 45 bp, synthetic construct
  misc_feature 1..45: note = Target Specific Oligonucleotide
  source 1..45: mol_type = other DNA
actactgtttggtacttgtatctggtaaccctaattatgggaatc

SEQ ID NO: 65 - DNA, 42 bp, synthetic construct
  misc_feature 1..42: note = Target Specific Oligonucleotide
  source 1..42: mol_type = other DNA
aaatcagtggcggacagtagcgtaaccctaattatgggaatc

SEQ ID NO: 66 - DNA, 41 bp, synthetic construct
  misc_feature 1..41: note = Target Specific Oligonucleotide
  source 1..41: mol_type = other DNA
caggcaatgccgactggattgtaaccctaattatgggaatc

SEQ ID NO: 67 - DNA, 48 bp, synthetic construct
  misc_feature 1..48: note = Target Specific Oligonucleotide
  source 1..48: mol_type = other DNA
tcttatctaagaccaactataggtatggtaaccctaattatgggaatc

SEQ ID NO: 68 - DNA, 43 bp, synthetic construct
  misc_feature 1..43: note = Target Specific Oligonucleotide
  source 1..43: mol_type = other DNA
gggaactgtctattgagcactcgtaaccctaattatgggaatc

SEQ ID NO: 69 - DNA, 47 bp, synthetic construct
  misc_feature 1..47: note = Target Specific Oligonucleotide
  source 1..47: mol_type = other DNA
attacaatctttagtactcatggaaagtaaccctaattatgggaatc

SEQ ID NO: 70 - DNA, 46 bp, synthetic construct
  misc_feature 1..46: note = Target Specific Oligonucleotide
  source 1..46: mol_type = other DNA
aaggtattctcatgcctagaatattgtaaccctaattatgggaatc

SEQ ID NO: 71 - DNA, 43 bp, synthetic construct
  misc_feature 1..43: note = Target Specific Oligonucleotide
  source 1..43: mol_type = other DNA
tggacagacacgttgtcatttggtaaccctaattatgggaatc

SEQ ID NO: 72 - DNA, 48 bp, synthetic construct
  misc_feature 1..48: note = Target Specific Oligonucleotide
  source 1..48: mol_type = other DNA
ctctcattacaatgctatacatttaacgtaaccctaattatgggaatc

SEQ ID NO: 73 - DNA, 41 bp, synthetic construct
  misc_feature 1..41: note = Target Specific Oligonucleotide
  source 1..41: mol_type = other DNA
tgcagctcgcaacggaacaagtaacggattttacaacttta

SEQ ID NO: 74 - DNA, 43 bp, synthetic construct
  misc_feature 1..43: note = Target Specific Oligonucleotide
  source 1..43: mol_type = other DNA
gagtccatccggacattgacctgtaacggattttacaacttta

SEQ ID NO: 75 - DNA, 41 bp, synthetic construct
  misc_feature 1..41: note = Target Specific Oligonucleotide
  source 1..41: mol_type = other DNA
cttgaacggcctcgacgagggtaacggattttacaacttta

SEQ ID NO: 76 - DNA, 43 bp, synthetic construct
  misc_feature 1..43: note = Target Specific Oligonucleotide
  source 1..43: mol_type = other DNA
cctcctcggttatattggccccgtaacggattttacaacttta

SEQ ID NO: 77 - DNA, 44 bp, synthetic construct
  misc_feature 1..44: note = Target Specific Oligonucleotide
  source 1..44: mol_type = other DNA
accgagacttcgaaaatgaacgagtaacggattttacaacttta

SEQ ID NO: 78 - DNA, 43 bp, synthetic construct
  misc_feature 1..43: note = Target Specific Oligonucleotide
  source 1..43: mol_type = other DNA
cctctggctcgaaactgaaagggtaacggattttacaacttta

SEQ ID NO: 79 - DNA, 44 bp, synthetic construct
  misc_feature 1..44: note = Target Specific Oligonucleotide
  source 1..44: mol_type = other DNA
tgctatggactagatctcggcaagtaacggattttacaacttta

SEQ ID NO: 80 - DNA, 42 bp, synthetic construct
  misc_feature 1..42: note = Target Specific Oligonucleotide
  source 1..42: mol_type = other DNA
gccttgaccggtcagaagacggtaacggattttacaacttta

SEQ ID NO: 81 - DNA, 42 bp, synthetic construct
  misc_feature 1..42: note = Target Specific Oligonucleotide
  source 1..42: mol_type = other DNA
taccacagagatcgcagctgcgtaacggattttacaacttta

SEQ ID NO: 82 - DNA, 40 bp, synthetic construct
  misc_feature 1..40: note = Target Specific Oligonucleotide
  source 1..40: mol_type = other DNA
accagcccgaacctcgcccgtaacggattttacaacttta

SEQ ID NO: 83 - DNA, 44 bp, synthetic construct
  misc_feature 1..44: note = Target Specific Oligonucleotide
  source 1..44: mol_type = other DNA
actgtatgcttgccggtaattctgtaacggattttacaacttta

SEQ ID NO: 84 - DNA, 41 bp, synthetic construct
  misc_feature 1..41: note = Target Specific Oligonucleotide
  source 1..41: mol_type = other DNA
gaacatgaaccggccccgacgtaacggattttacaacttta

SEQ ID NO: 85 - DNA, 42 bp, synthetic construct
  misc_feature 1..42: note = Target Specific Oligonucleotide
  source 1..42: mol_type = other DNA
caggaatcccgtcaagggtgggtaacggattttacaacttta

SEQ ID NO: 86 - DNA, 47 bp, synthetic construct
  misc_feature 1..47: note = Target Specific Oligonucleotide
  source 1..47: mol_type = other DNA
tctcgatttacctcgtttaagatctcgtaacggattttacaacttta

SEQ ID NO: 87 - DNA, 43 bp, synthetic construct
  misc_feature 1..43: note = Target Specific Oligonucleotide
  source 1..43: mol_type = other DNA
agaaccatggtcgaactgacctgtaacggattttacaacttta

SEQ ID NO: 88 - DNA, 41 bp, synthetic construct
  misc_feature 1..41: note = Target Specific Oligonucleotide
  source 1..41: mol_type = other DNA
tctgaggggctacgctgagagtaacggattttacaacttta

SEQ ID NO: 89 - DNA, 41 bp, synthetic construct
  misc_feature 1..41: note = Target Specific Oligonucleotide
  source 1..41: mol_type = other DNA
aagcctctgttacgcccacggtaacggattttacaacttta

SEQ ID NO: 90 - DNA, 46 bp, synthetic construct
  misc_feature 1..46: note = Target Specific Oligonucleotide
  source 1..46: mol_type = other DNA
aatgattcaactcgtactggatcccgtaacggattttacaacttta

SEQ ID NO: 91 - DNA, 44 bp, synthetic construct
  misc_feature 1..44: note = Target Specific Oligonucleotide
  source 1..44: mol_type = other DNA
cctcggaaatcccgattctgatagtaacggattttacaacttta

SEQ ID NO: 92 - DNA, 41 bp, synthetic construct
  misc_feature 1..41: note = Target Specific Oligonucleotide
  source 1..41: mol_type = other DNA
ggcccctactcggttgtggggtaacggattttacaacttta

SEQ ID NO: 93 - DNA, 45 bp, synthetic construct
  misc_feature 1..45: note = Target Specific Oligonucleotide
  source 1..45: mol_type = other DNA
ggaaaatggaacgaaactcctgttgtaacggattttacaacttta

SEQ ID NO: 94 - DNA, 40 bp, synthetic construct
  misc_feature 1..40: note = Target Specific Oligonucleotide
  source 1..40: mol_type = other DNA
ctgggtcgtgcgaggtcctgtaacggattttacaacttta

SEQ ID NO: 95 - DNA, 40 bp, synthetic construct
  misc_feature 1..40: note = Target Specific Oligonucleotide
  source 1..40: mol_type = other DNA
cacggggctaggacggggtgtaacggattttacaacttta

SEQ ID NO: 96 - DNA, 39 bp, synthetic construct
  misc_feature 1..39: note = Target Specific Oligonucleotide
  source 1..39: mol_type = other DNA
ccgatggcgtactcgtcgggtaacccttattcggtacta

SEQ ID NO: 97 - DNA, 42 bp, synthetic construct
  misc_feature 1..42: note = Target Specific Oligonucleotide
  source 1..42: mol_type = other DNA
gatcagatcagaatccgaggtggtaacccttattcggtacta

SEQ ID NO: 98 - DNA, 40 bp, synthetic construct
  misc_feature 1..40: note = Target Specific Oligonucleotide
  source 1..40: mol_type = other DNA
ggttgtactcgcgacagttggtaacccttattcggtacta

SEQ ID NO: 99 - DNA, 40 bp, synthetic construct
  misc_feature 1..40: note = Target Specific Oligonucleotide
  source 1..40: mol_type = other DNA
gacctgctcattcgaggtgagtaacccttattcggtacta

SEQ ID NO: 100 - DNA, 39 bp, synthetic construct
  misc_feature 1..39: note = Target Specific Oligonucleotide
  source 1..39: mol_type = other DNA
tcacgtagtagcggcttcggtaacccttattcggtacta

SEQ ID NO: 101 - DNA, 42 bp, synthetic construct
  misc_feature 1..42: note = Target Specific Oligonucleotide
  source 1..42: mol_type = other DNA
ccaaagatgggcctacttgtcagtaacccttattcggtacta

SEQ ID NO: 102 - DNA, 39 bp, synthetic construct
  misc_feature 1..39: note = Target Specific Oligonucleotide
  source 1..39: mol_type = other DNA
gacagagctgcacgcttgggtaacccttattcggtacta

SEQ ID NO: 103 - DNA, 39 bp, synthetic construct
  misc_feature 1..39: note = Target Specific Oligonucleotide
  source 1..39: mol_type = other DNA
ctgggcctgtttgcgctcagtaacccttattcggtacta

SEQ ID NO: 104 - DNA, 38 bp, synthetic construct
  misc_feature 1..38: note = Target Specific Oligonucleotide
  source 1..38: mol_type = other DNA
tccttaggcatgcgcggcgtaacccttattcggtacta

SEQ ID NO: 105 - DNA, 39 bp, synthetic construct
  misc_feature 1..39: note = Target Specific Oligonucleotide
  source 1..39: mol_type = other DNA
cagcggcagtactcgctgtgtaacccttattcggtacta

SEQ ID NO: 106 - DNA, 38 bp, synthetic construct
  misc_feature 1..38: note = Target Specific Oligonucleotide
  source 1..38: mol_type = other DNA
atgccaccgatgacccgcgtaacccttattcggtacta

SEQ ID NO: 107 - DNA, 38 bp, synthetic construct
  misc_feature 1..38: note = Target Specific Oligonucleotide
  source 1..38: mol_type = other DNA
atcagcgtcgctccctcggtaacccttattcggtacta

SEQ ID NO: 108 - DNA, 41 bp, synthetic construct
  misc_feature 1..41: note = Target Specific Oligonucleotide
  source 1..41: mol_type = other DNA
ctttgagagctcgaacatcgtgtaacccttattcggtacta

SEQ ID NO: 109 - DNA, 42 bp, synthetic construct
  misc_feature 1..42: note = Target Specific Oligonucleotide
  source 1..42: mol_type = other DNA
ttccagtagttaaggcagagcagtaacccttattcggtacta

SEQ ID NO: 110 - DNA, 39 bp, synthetic construct
  misc_feature 1..39: note = Target Specific Oligonucleotide
  source 1..39: mol_type = other DNA
gagctgcaggatcgggtccgtaacccttattcggtacta

SEQ ID NO: 111 - DNA, 41 bp, synthetic construct
  misc_feature 1..41: note = Target Specific Oligonucleotide
  source 1..41: mol_type = other DNA
acatgagtggtttcgtagcgggtaacccttattcggtacta

SEQ ID NO: 112 - DNA, 40 bp, synthetic construct
  misc_feature 1..40: note = Target Specific Oligonucleotide
  source 1..40: mol_type = other DNA
tgccgatgacatggaactcggtaacccttattcggtacta

SEQ ID NO: 113 - DNA, 43 bp, synthetic construct
  misc_feature 1..43: note = Target Specific Oligonucleotide
  source 1..43: mol_type = other DNA
tggtgtaattgaccttgtaggtagtaacccttattcggtacta

SEQ ID NO: 114 - DNA, 39 bp, synthetic construct
  misc_feature 1..39: note = Target Specific Oligonucleotide
  source 1..39: mol_type = other DNA
cattggccgaaagcaggctgtaacccttattcggtacta

SEQ ID NO: 115 - DNA, 40 bp, synthetic construct
  misc_feature 1..40: note = Target Specific Oligonucleotide
  source 1..40: mol_type = other DNA
gagacctctgccgaaactgggtaacccttattcggtacta

SEQ ID NO: 116 - DNA, 41 bp, synthetic construct
  misc_feature 1..41: note = Target Specific Oligonucleotide
  source 1..41: mol_type = other DNA
agtcttgtgcttcgggtcaaagtaacccttattcggtacta

SEQ ID NO: 117 - DNA, 41 bp, synthetic construct
  misc_feature 1..41: note = Target Specific Oligonucleotide
  source 1..41: mol_type = other DNA
cctcttctcgcctggcatagggtaacccttattcggtacta

SEQ ID NO: 118 - DNA, 41 bp, synthetic construct
  misc_feature 1..41: note = Target Specific Oligonucleotide
  source 1..41: mol_type = other DNA
gtaacggtgaaaatggaccgggtaacccttattcggtacta

SEQ ID NO: 119 - DNA, 42 bp, synthetic construct
  misc_feature 1..42: note = Target Specific Oligonucleotide
  source 1..42: mol_type = other DNA
tccctctaagggattaatgccagtaacccttattcggtacta

SEQ ID NO: 120 - DNA, 43 bp, synthetic construct
  misc_feature 1..43: note = Target Specific Oligonucleotide
  source 1..43: mol_type = other DNA
agttcaagtatcccgcgactagtaacaattagagtgaaatacc

SEQ ID NO: 121 - DNA, 43 bp, synthetic construct
  misc_feature 1..43: note = Target Specific Oligonucleotide
  source 1..43: mol_type = other DNA
gaacgcaggctgtttactgttgtaacaattagagtgaaatacc

SEQ ID NO: 122 - DNA, 44 bp, synthetic construct
  misc_feature 1..44: note = Target Specific Oligonucleotide
  source 1..44: mol_type = other DNA
ggtccaggtaaactaatggctggtaacaattagagtgaaatacc

SEQ ID NO: 123 - DNA, 44 bp, synthetic construct
  misc_feature 1..44: note = Target Specific Oligonucleotide
  source 1..44: mol_type = other DNA
ctctgttgagtttacctcgcaagtaacaattagagtgaaatacc

SEQ ID NO: 124 - DNA, 44 bp, synthetic construct
  misc_feature 1..44: note = Target Specific Oligonucleotide
  source 1..44: mol_type = other DNA
tacttcaactaaccagtccacggtaacaattagagtgaaatacc

SEQ ID NO: 125 - DNA, 44 bp, synthetic construct
  misc_feature 1..44: note = Target Specific Oligonucleotide
  source 1..44: mol_type = other DNA
ccatgagacaaggcttaagactgtaacaattagagtgaaatacc

SEQ ID NO: 126 - DNA, 43 bp, synthetic construct
  misc_feature 1..43: note = Target Specific Oligonucleotide
  source 1..43: mol_type = other DNA
cggccaaagaatagtcgtagcgtaacaattagagtgaaatacc

SEQ ID NO: 127 - DNA, 45 bp, synthetic construct
  misc_feature 1..45: note = Target Specific Oligonucleotide
  source 1..45: mol_type = other DNA
ccatttccctaaggtatgtgtgagtaacaattagagtgaaatacc

SEQ ID NO: 128 - DNA, 46 bp, synthetic construct
  misc_feature 1..46: note = Target Specific Oligonucleotide
  source 1..46: mol_type = other DNA
ctctcatactgttagtgatgtctggtaacaattagagtgaaatacc

SEQ ID NO: 129 - DNA, 42 bp, synthetic construct
  misc_feature 1..42: note = Target Specific Oligonucleotide
  source 1..42: mol_type = other DNA
cagctttgtcccgtgactgtgtaacaattagagtgaaatacc

SEQ ID NO: 130 - DNA, 41 bp, synthetic construct
  misc_feature 1..41: note = Target Specific Oligonucleotide
  source 1..41: mol_type = other DNA
tgctgcaatgctagcagcggtaacaattagagtgaaatacc

SEQ ID NO: 131 - DNA, 46 bp, synthetic construct
  misc_feature 1..46: note = Target Specific Oligonucleotide
  source 1..46: mol_type = other DNA
aagacaggaacctatcaatgtagtgtaacaattagagtgaaatacc

SEQ ID NO: 132 - DNA, 46 bp, synthetic construct
  misc_feature 1..46: note = Target Specific Oligonucleotide
  source 1..46: mol_type = other DNA
tagattagattatgcccaagtcaggtaacaattagagtgaaatacc

SEQ ID NO: 133 - DNA, 46 bp, synthetic construct
  misc_feature 1..46: note = Target Specific Oligonucleotide
  source 1..46: mol_type = other DNA
catgtaacccactttaggtttacagtaacaattagagtgaaatacc

SEQ ID NO: 134 - DNA, 47 bp, synthetic construct
  misc_feature 1..47: note = Target Specific Oligonucleotide
  source 1..47: mol_type = other DNA
ccagtctttcgtattaatgattcaggtaacaattagagtgaaatacc

SEQ ID NO: 135 - DNA, 42 bp, synthetic construct
  misc_feature 1..42: note = Target Specific Oligonucleotide
  source 1..42: mol_type = other DNA
tgcaacccgtctcgtcttcggtaacaattagagtgaaatacc

SEQ ID NO: 136 - DNA, 47 bp, synthetic construct
  misc_feature 1..47: note = Target Specific Oligonucleotide
  source 1..47: mol_type = other DNA
gtgtaggtatcatctgtaatgtacagtaacaattagagtgaaatacc

SEQ ID NO: 137 - DNA, 41 bp, synthetic construct
  misc_feature 1..41: note = Target Specific Oligonucleotide
  source 1..41: mol_type = other DNA
tcccggacttcagtaccgcgtaacaattagagtgaaatacc

SEQ ID NO: 138 - DNA, 41 bp, synthetic construct
  misc_feature 1..41: note = Target Specific Oligonucleotide
  source 1..41: mol_type = other DNA
cgaggaggttgcgaaaggcgtaacaattagagtgaaatacc

SEQ ID NO: 139 - DNA, 49 bp, synthetic construct
  misc_feature 1..49: note = Target Specific Oligonucleotide
  source 1..49: mol_type = other DNA
tatacatatactcaacacttatagagggtaacaattagagtgaaatacc

SEQ ID NO: 140 - DNA, 49 bp, synthetic construct
  misc_feature 1..49: note = Target Specific Oligonucleotide
  source 1..49: mol_type = other DNA
gtaatagataccataatttgtacttgggtaacaattagagtgaaatacc

SEQ ID NO: 141 - DNA, 40 bp, synthetic construct
  misc_feature 1..40: note = Target Specific Oligonucleotide
  source 1..40: mol_type = other DNA
tgctgctgcgctagacccgtaacaattagagtgaaatacc

SEQ ID NO: 142 - DNA, 40 bp, synthetic construct
  misc_feature 1..40: note = Target Specific Oligonucleotide
  source 1..40: mol_type = other DNA
gcgtggcgagccaaagacgtaacaattagagtgaaatacc

SEQ ID NO: 143 - DNA, 40 bp, synthetic construct
  misc_feature 1..40: note = Target Specific Oligonucleotide
  source 1..40: mol_type = other DNA
tccgcggttgttggacgggtaacaattagagtgaaatacc

SEQ ID NO: 144 - DNA, 41 bp, synthetic construct
  misc_feature 1..41: note = Target Specific Oligonucleotide
  source 1..41: mol_type = other DNA
ctgtcacatacgcaaactggtgtaacatcgttatagctaga

SEQ ID NO: 145 - DNA, 41 bp, synthetic construct
  misc_feature 1..41: note = Target Specific Oligonucleotide
  source 1..41: mol_type = other DNA
tcgccatataccggtcaaagagtaacatcgttatagctaga

SEQ ID NO: 146 - DNA, 42 bp, synthetic construct
  misc_feature 1..42: note = Target Specific Oligonucleotide
  source 1..42: mol_type = other DNA
tggatggtgcaataatccgagggtaacatcgttatagctaga

SEQ ID NO: 147 - DNA, 43 bp, synthetic construct
  misc_feature 1..43: note = Target Specific Oligonucleotide
  source 1..43: mol_type = other DNA
agtgctgatcccttaagtatgtcgtaacatcgttatagctaga

SEQ ID NO: 148 - DNA, 43 bp, synthetic construct
  misc_feature 1..43: note = Target Specific Oligonucleotide
  source 1..43: mol_type = other DNA
acctccattaaccaatccagaaggtaacatcgttatagctaga

SEQ ID NO: 149 - DNA, 43 bp, synthetic construct
  misc_feature 1..43: note = Target Specific Oligonucleotide
  source 1..43: mol_type = other DNA
tggtgtacttgacccacttatttgtaacatcgttatagctaga

SEQ ID NO: 150 - DNA, 41 bp, synthetic construct
  misc_feature 1..41: note = Target Specific Oligonucleotide
  source 1..41: mol_type = other DNA
cagaagagaacgtggagcagggtaacatcgttatagctaga

SEQ ID NO: 151 - DNA, 44 bp, synthetic construct
  misc_feature 1..44: note = Target Specific Oligonucleotide
  source 1..44: mol_type = other DNA
tccctgtgaagtttatagacttcagtaacatcgttatagctaga

SEQ ID NO: 152 - DNA, 40 bp, synthetic construct
  misc_feature 1..40: note = Target Specific Oligonucleotide
  source 1..40: mol_type = other DNA
tgtaaaagcaaacgcacgccgtaacatcgttatagctaga

SEQ ID NO: 153 - DNA, 44 bp, synthetic construct
  misc_feature 1..44: note = Target Specific Oligonucleotide
  source 1..44: mol_type = other DNA
attcagatgacgagaaatgatacagtaacatcgttatagctaga

SEQ ID NO: 154 - DNA, 39 bp, synthetic construct
  misc_feature 1..39: note = Target Specific Oligonucleotide
  source 1..39: mol_type = other DNA
tggtcacgccatttccggcgtaacatcgttatagctaga

SEQ ID NO: 155 - DNA, 45 bp, synthetic construct
  misc_feature 1..45: note = Target Specific Oligonucleotide
  source 1..45: mol_type = other DNA
agcaagtataccataaggaaattcagtaacatcgttatagctaga

SEQ ID NO: 156 - DNA, 40 bp, synthetic construct
  misc_feature 1..40: note = Target Specific Oligonucleotide
  source 1..40: mol_type = other DNA
ctcgccgtcctgtcgattttgtaacatcgttatagctaga

SEQ ID NO: 157 - DNA, 38 bp, synthetic construct
  misc_feature 1..38: note = Target Specific Oligonucleotide
  source 1..38: mol_type = other DNA
agcgagccgagaactccggtaacatcgttatagctaga

SEQ ID NO: 158 - DNA, 43 bp, synthetic construct
  misc_feature 1..43: note = Target Specific Oligonucleotide
  source 1..43: mol_type = other DNA
tattttgttcagacaacatggctgtaacatcgttatagctaga

SEQ ID NO: 159 - DNA, 40 bp, synthetic construct
  misc_feature 1..40: note = Target Specific Oligonucleotide
  source 1..40: mol_type = other DNA
ccacttggtacaacggagccgtaacatcgttatagctaga

SEQ ID NO: 160 - DNA, 40 bp, synthetic construct
  misc_feature 1..40: note = Target Specific Oligonucleotide
  source 1..40: mol_type = other DNA
cagaacacctgcgaggagaggtaacatcgttatagctaga

SEQ ID NO: 161 - DNA, 39 bp, synthetic construct
  misc_feature 1..39: note = Target Specific Oligonucleotide
  source 1..39: mol_type = other DNA
ttcatcagcgacgcccctggtaacatcgttatagctaga

SEQ ID NO: 162 - DNA, 41 bp, synthetic construct
  misc_feature 1..41: note = Target Specific Oligonucleotide
  source 1..41: mol_type = other DNA
cctgcaaaaaaacggtcacgtgtaacatcgttatagctaga

SEQ ID NO: 163 - DNA, 41 bp, synthetic construct
  misc_feature 1..41: note = Target Specific Oligonucleotide
  source 1..41: mol_type = other DNA
tgtgggagtcccttaggtcaagtaacatcgttatagctaga

SEQ ID NO: 164 - DNA, 38 bp, synthetic construct
  misc_feature 1..38: note = Target Specific Oligonucleotide
  source 1..38: mol_type = other DNA
gcatcgggagcacgcactgtaacatcgttatagctaga

SEQ ID NO: 165 - DNA, 39 bp, synthetic construct
  misc_feature 1..39: note = Target Specific Oligonucleotide
  source 1..39: mol_type = other DNA
ggctctgcacaacgcttgcgtaacatcgttatagctaga

SEQ ID NO: 166 - DNA, 41 bp, synthetic construct
  misc_feature 1..41: note = Target Specific Oligonucleotide
  source 1..41: mol_type = other DNA
agccaggacacaatagtcagggtaacatcgttatagctaga

SEQ ID NO: 167 - DNA, 41 bp, synthetic construct; identical to SEQ ID NO: 166
  misc_feature 1..41: note = Target Specific Oligonucleotide
  source 1..41: mol_type = other DNA

SEQ ID NO: 168 - DNA, 42 bp, synthetic construct
  misc_feature 1..42: note = Target Specific Oligonucleotide
  source 1..42: mol_type = other DNA
ttgcacatcctacggtcttctgtaacttactacggagttaac

SEQ ID NO: 169 - DNA, 43 bp, synthetic construct
  misc_feature 1..43: note = Target Specific Oligonucleotide
  source 1..43: mol_type = other DNA
gtagctcattcgcaaatcttgggtaacttactacggagttaac

SEQ ID NO: 170 - DNA, 41 bp, synthetic construct
  misc_feature 1..41: note = Target Specific Oligonucleotide
  source 1..41: mol_type = other DNA
cctttaagcgcgtcgtgtccgtaacttactacggagttaac

SEQ ID NO: 171 - DNA, 39 bp, synthetic construct
  misc_feature 1..39: note = Target Specific Oligonucleotide
  source 1..39: mol_type = other DNA
cctcgacgcatgatgccggtaacttactacggagttaac

SEQ ID NO: 172 - DNA, 40 bp, synthetic construct
  misc_feature 1..40: note = Target Specific Oligonucleotide
  source 1..40: mol_type = other DNA
gccccatggctcgtgtagggtaacttactacggagttaac

SEQ ID NO: 173 - DNA, 42 bp, synthetic construct
  misc_feature 1..42: note = Target Specific Oligonucleotide
  source 1..42: mol_type = other DNA
ccaattgtgttccggcaagttgtaacttactacggagttaac

SEQ ID NO: 174 - DNA, 43 bp, synthetic construct
  misc_feature 1..43: note = Target Specific Oligonucleotide
  source 1..43: mol_type = other DNA
gataggatttggtcggaaacctgtaacttactacggagttaac

SEQ ID NO: 175 - DNA, 41 bp, synthetic construct
  misc_feature 1..41: note = Target Specific Oligonucleotide
  source 1..41: mol_type = other DNA
tagttgccaacggtgttgtagtaacttactacggagttaac

SEQ ID NO: 176 - DNA, 42 bp, synthetic construct
  misc_feature 1..42: note = Target Specific Oligonucleotide
  source 1..42: mol_type = other DNA
cttggcctatgcggaagtaacgtaacttactacggagttaac

SEQ ID NO: 177 - DNA, 41 bp, synthetic construct
  misc_feature 1..41: note = Target Specific Oligonucleotide
  source 1..41: mol_type = other DNA
ctcgcagcgataggaaccatgtaacttactacggagttaac

SEQ ID NO: 178 - DNA, 44 bp, synthetic construct
  misc_feature 1..44: note = Target Specific Oligonucleotide
  source 1..44: mol_type = other DNA
tttgcattcgtccatatcaactggtaacttactacggagttaac

SEQ ID NO: 179 - DNA, 43 bp, synthetic construct
  misc_feature 1..43: note = Target Specific Oligonucleotide
  source 1..43: mol_type = other DNA
tcaatatcaacgggtaaaccgggtaacttactacggagttaac

SEQ ID NO: 180 - DNA, 45 bp, synthetic construct
  misc_feature 1..45: note = Target Specific Oligonucleotide
  source 1..45: mol_type = other DNA
ttgttactgacgtgggaaatattggtaacttactacggagttaac

SEQ ID NO: 181 - DNA, 40 bp, synthetic construct
  misc_feature 1..40: note = Target Specific Oligonucleotide
  source 1..40: mol_type = other DNA
tctccgctgatacccgggtgtaacttactacggagttaac

SEQ ID NO: 182 - DNA, 42 bp, synthetic construct
  misc_feature 1..42: note = Target Specific Oligonucleotide
  source 1..42: mol_type = other DNA
ggccagtcaccgaaatttcatgtaacttactacggagttaac

SEQ ID NO: 183 - DNA, 43 bp, synthetic construct
  misc_feature 1..43: note = Target Specific Oligonucleotide
  source 1..43: mol_type = other DNA
agttcgtagcctatctcacactgtaacttactacggagttaac

SEQ ID NO: 184 - DNA, 42 bp, synthetic construct
  misc_feature 1..42: note = Target Specific Oligonucleotide
  source 1..42: mol_type = other DNA
cacattcccgtacgtttgctggtaacttactacggagttaac

SEQ ID NO: 185 - DNA, 45 bp, synthetic construct
  misc_feature 1..45: note = Target Specific Oligonucleotide
  source 1..45: mol_type = other DNA
agtgagtccctatgtatcctttctgtaacttactacggagttaac

SEQ ID NO: 186 - DNA, 44 bp, synthetic construct
  misc_feature 1..44: note = Target Specific Oligonucleotide
  source 1..44: mol_type = other DNA
aagcattataacgtgatccacaggtaacttactacggagttaac

SEQ ID NO: 187 - DNA, 38 bp, synthetic construct
  misc_feature 1..38: note = Target Specific Oligonucleotide
  source 1..38: mol_type = other DNA
tgcgccctgaagcgcacgtaacttactacggagttaac

SEQ ID NO: 188 - DNA, 47 bp, synthetic construct
  misc_feature 1..47: note = Target Specific Oligonucleotide
  source 1..47: mol_type = other DNA
taaaaaatagcacgattacagtatacgtaacttactacggagttaac

SEQ ID NO: 189 - DNA, 44 bp, synthetic construct
  misc_feature 1..44: note = Target Specific Oligonucleotide
  source 1..44: mol_type = other DNA
catatcgacggattgagcctaacgtaacttactacggagttaac

SEQ ID NO: 190 - DNA, 42 bp, synthetic construct
  misc_feature 1..42: note = Target Specific Oligonucleotide
  source 1..42: mol_type = other DNA
cggtttctcgcgagagaaatagtaacttactacggagttaac

SEQ ID NO: 191 - DNA, 43 bp, synthetic construct
  misc_feature 1..43: note = Target Specific Oligonucleotide
  source 1..43: mol_type = other DNA
cttcccatctcgtgtaacatgagtaacttactacggagttaac

SEQ ID NO: 192 - DNA, 41 bp, synthetic construct
  misc_feature 1..41: note = Target Specific Oligonucleotide
  source 1..41: mol_type = other DNA
gcatttcacatcggactgtaccgtaacttcgaaacggaaac

SEQ ID NO: 193 - DNA, 38 bp, synthetic construct
  misc_feature 1..38: note = Target Specific Oligonucleotide
  source 1..38: mol_type = other DNA
tggaattctcggcggaccagtaacttcgaaacggaaac

SEQ ID NO: 194 - DNA, 41 bp, synthetic construct
  misc_feature 1..41: note = Target Specific Oligonucleotide
  source 1..41: mol_type = other DNA
gaagtggaaatacgacctttgcgtaacttcgaaacggaaac

SEQ ID NO: 195 - DNA, 39 bp, synthetic construct
  misc_feature 1..39: note = Target Specific Oligonucleotide
  source 1..39: mol_type = other DNA
gctggtcccttgcgtaacatgtaacttcgaaacggaaac

SEQ ID NO: 196 - DNA, 43 bp, synthetic construct
  misc_feature 1..43: note = Target Specific Oligonucleotide
  source 1..43: mol_type = other DNA
ttttcatctattcgagatgctcccgtaacttcgaaacggaaac

SEQ ID NO: 197 - DNA, 40 bp, synthetic construct
  misc_feature 1..40: note = Target Specific Oligonucleotide
  source 1..40: mol_type = other DNA
tattcatcccgtggggtagtagtaacttcgaaacggaaac

SEQ ID NO: 198 - DNA, 42 bp, synthetic construct
  misc_feature 1..42: note = Target Specific Oligonucleotide
  source 1..42: mol_type = other DNA
taaaaataggcccggatttgtctgtaacttcgaaacggaaac

SEQ ID NO: 199 - DNA, 42 bp, synthetic construct
  misc_feature 1..42: note = Target Specific Oligonucleotide
  source 1..42: mol_type = other DNA
tccaaccttgacgacaagatagggtaacttcgaaacggaaac

SEQ ID NO: 200 - DNA, 40 bp, synthetic construct
  misc_feature 1..40: note = Target Specific Oligonucleotide
  source 1..40: mol_type = other DNA
aggcaacagtcgaaccattctgtaacttcgaaacggaaac

SEQ ID NO: 201 - DNA, 40 bp, synthetic construct
  misc_feature 1..40: note = Target Specific Oligonucleotide
  source 1..40: mol_type = other DNA
gcttcatttgcgactgctcttgtaacttcgaaacggaaac

SEQ ID NO: 202 - DNA, 41 bp, synthetic construct
  misc_feature 1..41: note = Target Specific Oligonucleotide
  source 1..41: mol_type = other DNA
catcaggacgcttgtattggtggtaacttcgaaacggaaac

SEQ ID NO: 203 - DNA, 41 bp, synthetic construct
  misc_feature 1..41: note = Target Specific Oligonucleotide
  source 1..41: mol_type = other DNA
atcttcaatccgagaatccagcgtaacttcgaaacggaaac

SEQ ID NO: 204 - DNA, 42 bp, synthetic construct
  misc_feature 1..42: note = Target Specific Oligonucleotide
  source 1..42: mol_type = other DNA
ctgtagtgtggtagtaaggaagagtaacttcgaaacggaaac

SEQ ID NO: 205 - DNA, 42 bp, synthetic construct
  misc_feature 1..42: note = Target Specific Oligonucleotide
  source 1..42: mol_type = other DNA
gaacaccttacttacaacacctggtaacttcgaaacggaaac

SEQ ID NO: 206 - DNA, 43 bp, synthetic construct
  misc_feature 1..43: note = Target Specific Oligonucleotide
  source 1..43: mol_type = other DNA
gtacattgtacaccgttacaatgggtaacttcgaaacggaaac

SEQ ID NO: 207 - DNA, 42 bp, synthetic construct
  misc_feature 1..42: note = Target Specific Oligonucleotide
  source 1..42: mol_type = other DNA
tttcaagtcgtctatgttagctggtaacttcgaaacggaaac

SEQ ID NO: 208 - DNA, 44 bp, synthetic construct
  misc_feature 1..44: note = Target Specific Oligonucleotide
  source 1..44: mol_type = other DNA
caaagcaactatatgaagcttcattgtaacttcgaaacggaaac

SEQ ID NO: 209 - DNA, 42 bp, synthetic construct
  misc_feature 1..42: note = Target Specific Oligonucleotide
  source 1..42: mol_type = other DNA
catacttctatcgagagctcagggtaacttcgaaacggaaac

SEQ ID NO: 210 - DNA, 43 bp, synthetic construct
  misc_feature 1..43: note = Target Specific Oligonucleotide
  source 1..43: mol_type = other DNA
acgaagaaatcgagtaggtctagggtaacttcgaaacggaaac

SEQ ID NO: 211 - DNA, 41 bp, synthetic construct
  misc_feature 1..41: note = Target Specific Oligonucleotide
  source 1..41: mol_type = other DNA
gaatcattgagtcgacccttcagtaacttcgaaacggaaac

SEQ ID NO: 212 - DNA, 41 bp, synthetic construct
  misc_feature 1..41: note = Target Specific Oligonucleotide
  source 1..41: mol_type = other DNA
tgatgtgcttaagtagtgcagcgtaacttcgaaacggaaac

SEQ ID NO: 213 - DNA, 41 bp, synthetic construct
  misc_feature 1..41: note = Target Specific Oligonucleotide
  source 1..41: mol_type = other DNA
tccacggcatgatacatacaacgtaacttcgaaacggaaac

SEQ ID NO: 214 - DNA, 40 bp, synthetic construct
  misc_feature 1..40: note = Target Specific Oligonucleotide
  source 1..40: mol_type = other DNA
tctcagagcatcccgaatccagtaacttcgaaacggaaac

SEQ ID NO: 215 - DNA, 42 bp, synthetic construct
  misc_feature 1..42: note = Target Specific Oligonucleotide
  source 1..42: mol_type = other DNA
gtggaacgaagagtaggtagtttgtaacttcgaaacggaaac

SEQ ID NO: 216 - DNA, 39 bp, synthetic construct
  misc_feature 1..39: note = Target Specific Oligonucleotide
  source 1..39: mol_type = other DNA
gatcaatactggacggagtcaggtaacctcgtcggatca

SEQ ID NO: 217 - DNA, 38 bp, synthetic construct
  misc_feature 1..38: note = Target Specific Oligonucleotide
  source 1..38: mol_type = other DNA
gagcttgttactcgtgccttggtaacctcgtcggatca

SEQ ID NO: 218 - DNA, 37 bp, synthetic construct
  misc_feature 1..37: note = Target Specific Oligonucleotide
  source 1..37: mol_type = other DNA
cttcttacacttgcggacgcgtaacctcgtcggatca

SEQ ID NO: 219 - DNA, 41 bp, synthetic construct
  misc_feature 1..41: note = Target Specific Oligonucleotide
  source 1..41: mol_type = other DNA
caatacctattccgttacacacttgtaacctcgtcggatca

SEQ ID NO: 220 - DNA, 39 bp, synthetic construct
  misc_feature 1..39: note = Target Specific Oligonucleotide
  source 1..39: mol_type = other DNA
gctccttcagtccggttttattgtaacctcgtcggatca

SEQ ID NO: 221 - DNA, 36 bp, synthetic construct
  misc_feature 1..36: note = Target Specific Oligonucleotide
  source 1..36: mol_type = other DNA
gacgcacgagccgtgatctgtaacctcgtcggatca

SEQ ID NO: 222 - DNA, 41 bp, synthetic construct
  misc_feature 1..41: note = Target Specific Oligonucleotide
  source 1..41: mol_type = other DNA
gactgctaaggcataggaattttcgtaacctcgtcggatca

SEQ ID NO: 223 - DNA, 39 bp, synthetic construct
  misc_feature 1..39: note = Target Specific Oligonucleotide
  source 1..39: mol_type = other DNA
caccacataattacggggacacgtaacctcgtcggatca

SEQ ID NO: 224 - DNA, 34 bp, synthetic construct
  misc_feature 1..34: note = Target Specific Oligonucleotide
  source 1..34: mol_type = other DNA
cggcaaggcccttcgcagtaacctcgtcggatca

SEQ ID NO: 225 - DNA, 37 bp, synthetic construct
  misc_feature 1..37: note = Target Specific Oligonucleotide
  source 1..37: mol_type = other DNA
tccatctggtacgtggtggggtaacctcgtcggatca

SEQ ID NO: 226 - DNA, 38 bp, synthetic construct
  misc_feature 1..38: note = Target Specific Oligonucleotide
  source 1..38: mol_type = other DNA
gtcctgccgcgtatgatttctgtaacctcgtcggatca

SEQ ID NO: 227 - DNA, 37 bp, synthetic construct
  misc_feature 1..37: note = Target Specific Oligonucleotide
  source 1..37: mol_type = other DNA
tgttcaggctgacgactgcagtaacctcgtcggatca

SEQ ID NO: 228 - DNA, 36 bp, synthetic construct
  misc_feature 1..36: note = Target Specific Oligonucleotide
  source 1..36: mol_type = other DNA
gcatggaggtccgtcctgtgtaacctcgtcggatca

SEQ ID NO: 229 - DNA, 38 bp, synthetic construct
  misc_feature 1..38: note = Target Specific Oligonucleotide
  source 1..38: mol_type = other DNA
ttgagggagcgtaatcccaaggtaacctcgtcggatca

SEQ ID NO: 230 - DNA, 36 bp, synthetic construct
  misc_feature 1..36: note = Target Specific Oligonucleotide
  source 1..36: mol_type = other DNA
ccggcgtctgcgtacttccgtaacctcgtcggatca

SEQ ID NO: 231 - DNA, 39 bp, synthetic construct
  misc_feature 1..39: note = Target Specific Oligonucleotide
  source 1..39: mol_type = other DNA
cgactatctgcgtctatcatccgtaacctcgtcggatca

SEQ ID NO: 232 - DNA, 40 bp, synthetic construct
  misc_feature 1..40: note = Target Specific Oligonucleotide
  source 1..40: mol_type = other DNA
gagaattcgatgatcaactcacggtaacctcgtcggatca

SEQ ID NO: 233 - DNA, 35 bp, synthetic construct
  misc_feature 1..35: note = Target Specific Oligonucleotide
  source 1..35: mol_type = other DNA
tggcctgtcgtccggtctgtaacctcgtcggatca

SEQ ID NO: 234 - DNA, 38 bp, synthetic construct
  misc_feature 1..38: note = Target Specific Oligonucleotide
  source 1..38: mol_type = other DNA
cagggattccgtcatatggctgtaacctcgtcggatca

SEQ ID NO: 235 - DNA, 40 bp, synthetic construct
  misc_feature 1..40: note = Target Specific Oligonucleotide
  source 1..40: mol_type = other DNA
agacatcgatggtacatatgggtgtaacctcgtcggatca

SEQ ID NO: 236 - DNA, 37 bp, synthetic construct
  misc_feature 1..37: note = Target Specific Oligonucleotide
  source 1..37: mol_type = other DNA
tctgagctgtatcgctgcaagtaacctcgtcggatca

SEQ ID NO: 237 - DNA, 37 bp, synthetic construct
  misc_feature 1..37: note = Target Specific Oligonucleotide
  source 1..37: mol_type = other DNA
tcttaggcccattcgttggagtaacctcgtcggatca

SEQ ID NO: 238 - DNA, 37 bp, synthetic construct
  misc_feature 1..37: note = Target Specific Oligonucleotide
  source 1..37: mol_type = other DNA
ttatacaccgtgccgaacgcgtaacctcgtcggatca

SEQ ID NO: 239 - DNA, 37 bp, synthetic construct
  misc_feature 1..37: note = Target Specific Oligonucleotide
  source 1..37: mol_type = other DNA
atgccctttgcgatctgcacgtaacctcgtcggatca

SEQ ID NO: 240 - DNA, 42 bp, synthetic construct
  misc_feature 1..42: note = Target Specific Oligonucleotide
  source 1..42: mol_type = other DNA
ggcagaattccgaagttcagcgtaacataatcgtagtttcgg

SEQ ID NO: 241 - DNA, 42 bp, synthetic construct
  misc_feature 1..42: note = Target Specific Oligonucleotide
  source 1..42: mol_type = other DNA
gtttccttcacgacaggtgtggtaacataatcgtagtttcgg

SEQ ID NO: 242 - DNA, 44 bp, synthetic construct
  misc_feature 1..44: note = Target Specific Oligonucleotide
  source 1..44: mol_type = other DNA
ctggtagaaatgcgactaaagacgtaacataatcgtagtttcgg

SEQ ID NO: 243 - DNA, 44 bp, synthetic construct
  misc_feature 1..44: note = Target Specific Oligonucleotide
  source 1..44: mol_type = other DNA
tgaccagcatcgtttcatctaatgtaacataatcgtagtttcgg

SEQ ID NO: 244 - DNA, 42 bp, synthetic construct
  misc_feature 1..42: note = Target Specific Oligonucleotide
  source 1..42: mol_type = other DNA
ttcgaagttcaaccgagtgacgtaacataatcgtagtttcgg

SEQ ID NO: 245 - DNA, 42 bp, synthetic construct
  misc_feature 1..42: note = Target Specific Oligonucleotide
  source 1..42: mol_type = other DNA
ccaccaatccaatgcggaattgtaacataatcgtagtttcgg

SEQ ID NO: 246 - DNA, 42 bp, synthetic construct
  misc_feature 1..42: note = Target Specific Oligonucleotide
  source 1..42: mol_type = other DNA
agactcggtgccattcgtattgtaacataatcgtagtttcgg

SEQ ID NO: 247 - DNA, 42 bp, synthetic construct
  misc_feature 1..42: note = Target Specific Oligonucleotide
  source 1..42: mol_type = other DNA
ataactgctaactgcgcaaccgtaacataatcgtagtttcgg

SEQ ID NO: 248 - DNA, 43 bp, synthetic construct
  misc_feature 1..43: note = Target Specific Oligonucleotide
  source 1..43: mol_type = other DNA
gtagtgaggccgcttataaccagtaacataatcgtagtttcgg

SEQ ID NO: 249 - DNA, 43 bp, synthetic construct
  misc_feature 1..43: note = Target Specific Oligonucleotide
  source 1..43: mol_type = other DNA
ggtgatgattcgatggagtgaagtaacataatcgtagtttcgg

SEQ ID NO: 250 - DNA, 42 bp, synthetic construct
  misc_feature 1..42: note = Target Specific Oligonucleotide
  source 1..42: mol_type = other DNA
ctgctgctttacgtttggtgcgtaacataatcgtagtttcgg

SEQ ID NO: 251 - DNA, 43 bp, synthetic construct
  misc_feature 1..43: note = Target Specific Oligonucleotide
  source 1..43: mol_type = other DNA
tcatcaaccacgtctttggatagtaacataatcgtagtttcgg

SEQ ID NO: 252 - DNA, 44 bp, synthetic construct
  misc_feature 1..44: note = Target Specific Oligonucleotide
  source 1..44: mol_type = other DNA
aggcagatcttaaagtgttggttgtaacataatcgtagtttcgg

SEQ ID NO: 253 - DNA, 43 bp, synthetic construct
  misc_feature 1..43: note = Target Specific Oligonucleotide
  source 1..43: mol_type = other DNA
tgagggcttatacgaaagcaaggtaacataatcgtagtttcgg

SEQ ID NO: 254 - DNA, 44 bp, synthetic construct
  misc_feature 1..44: note = Target Specific Oligonucleotide
  source 1..44: mol_type = other DNA
gctcagtactgactttggtatgtgtaacataatcgtagtttcgg

SEQ ID NO: 255 - DNA, 45 bp, synthetic construct
  misc_feature 1..45: note = Target Specific Oligonucleotide
  source 1..45: mol_type = other DNA
cagtcaatcattagatccacatctgtaacataatcgtagtttcgg

SEQ ID NO: 256 - DNA, 40 bp, synthetic construct
  misc_feature 1..40: note = Target Specific Oligonucleotide
  source 1..40: mol_type = other DNA
ggaagctgctcgtcgaagcgtaacataatcgtagtttcgg

SEQ ID NO: 257 - DNA, 47 bp, synthetic construct
  misc_feature 1..47: note = Target Specific Oligonucleotide
  source 1..47: mol_type = other DNA
gagaagatacttatagcttcttgtctgtaacataatcgtagtttcgg

SEQ ID NO: 258 - DNA, 45 bp, synthetic construct
  misc_feature 1..45: note = Target Specific Oligonucleotide
  source 1..45: mol_type = other DNA
tcaatgctatctaactgatgaagagtaacataatcgtagtttcgg

SEQ ID NO: 259 - DNA, 46 bp, synthetic construct
  misc_feature 1..46: note = Target Specific Oligonucleotide
  source 1..46: mol_type = other DNA
atcttcccaattgatgtaagtacttgtaacataatcgtagtttcgg

SEQ ID NO: 260 - DNA, 40 bp, synthetic construct
  misc_feature 1..40: note = Target Specific Oligonucleotide
  source 1..40: mol_type = other DNA
catcacagtccgagacgccgtaacataatcgtagtttcgg

SEQ ID NO: 261 - DNA, 40 bp, synthetic construct
  misc_feature 1..40: note = Target Specific Oligonucleotide
  source 1..40: mol_type = other DNA
cattccaccggcctgtgcggtaacataatcgtagtttcgg

SEQ ID NO: 262 - DNA, 44 bp, synthetic construct
  misc_feature 1..44: note = Target Specific Oligonucleotide
  source 1..44: mol_type = other DNA
ggacagcactactctagagtaaggtaacataatcgtagtttcgg

SEQ ID NO: 263 - DNA, 43 bp, synthetic construct
  misc_feature 1..43: note = Target Specific Oligonucleotide
  source 1..43: mol_type = other DNA
tgccaagtaacttagcacacccgtaacataatcgtagtttcgg

SEQ ID NO: 264 - DNA, 38 bp, synthetic construct
  misc_feature 1..38: note = Target Specific Oligonucleotide
  source 1..38: mol_type = other DNA
gtgccacctttcaccgtgagtaaccggtagaattacgg

SEQ ID NO: 265 - DNA, 38 bp, synthetic construct
  misc_feature 1..38: note = Target Specific Oligonucleotide
  source 1..38: mol_type = other DNA
ccgcaggtacgacttgcctgtaaccggtagaattacgg

SEQ ID NO: 266 - DNA, 40 bp, synthetic construct
  misc_feature 1..40: note = Target Specific Oligonucleotide
  source 1..40: mol_type = other DNA
tgggcttcaatcagatggtcagtaaccggtagaattacgg

SEQ ID NO: 267 - DNA, 41 bp, synthetic construct
  misc_feature 1..41: note = Target Specific Oligonucleotide
  source 1..41: mol_type = other DNA
agaacaccagggtacgcatagtgtaaccggtagaattacgg

SEQ ID NO: 268 - DNA, 38 bp, synthetic construct
  misc_feature 1..38: note = Target Specific Oligonucleotide
  source 1..38: mol_type = other DNA
tctccgagagtgtcagcgggtaaccggtagaattacgg

SEQ ID NO: 269 - DNA, 38 bp, synthetic construct
  misc_feature 1..38: note = Target Specific Oligonucleotide
  source 1..38: mol_type = other DNA
gtcttcccggccggtcttggtaaccggtagaattacgg

SEQ ID NO: 270 - DNA, 39 bp, synthetic construct
  misc_feature 1..39: note = Target Specific Oligonucleotide
  source 1..39: mol_type = other DNA
catgttgcagattgtcgccagtaaccggtagaattacgg

SEQ ID NO: 271 - DNA, 39 bp, synthetic construct
  misc_feature 1..39: note = Target Specific Oligonucleotide
  source 1..39: mol_type = other DNA
ggttcagtcgtttgcgaacagtaaccggtagaattacgg

SEQ ID NO: 272 - DNA, 41 bp, synthetic construct
  misc_feature 1..41: note = Target Specific Oligonucleotide
  source 1..41: mol_type = other DNA
agctaccaattagacccactcggtaaccggtagaattacgg

SEQ ID NO: 273 - DNA, 40 bp, synthetic construct
  misc_feature 1..40: note = Target Specific Oligonucleotide
  source 1..40: mol_type = other DNA
cgtagttctcgtctccgatcagtaaccggtagaattacgg

SEQ ID NO: 274 - DNA, 39 bp, synthetic construct
  misc_feature 1..39: note = Target Specific Oligonucleotide
  source 1..39: mol_type = other DNA
ggagctgaacttgacgccaggtaaccggtagaattacgg

SEQ ID NO: 275 - DNA, 40 bp, synthetic construct
  misc_feature 1..40: note = Target Specific Oligonucleotide
  source 1..40: mol_type = other DNA
taatggatgtactcgttgggcgtaaccggtagaattacgg

SEQ ID NO: 276 - DNA, 38 bp, synthetic construct
  misc_feature 1..38: note = Target Specific Oligonucleotide
  source 1..38: mol_type = other DNA
tctgtgcatagccgtcccggtaaccggtagaattacgg

SEQ ID NO: 277 - DNA, 38 bp, synthetic construct
  misc_feature 1..38: note = Target Specific Oligonucleotide
  source 1..38: mol_type = other DNA
tggctggacggatcggatcgtaaccggtagaattacgg

SEQ ID NO: 278 - DNA, 37 bp, synthetic construct
  misc_feature 1..37: note = Target Specific Oligonucleotide
  source 1..37: mol_type = other DNA
ggtttgcgtcgttgcggcgtaaccggtagaattacgg

SEQ ID NO: 279 - DNA, 39 bp, synthetic construct
  misc_feature 1..39: note = Target Specific Oligonucleotide
  source 1..39: mol_type = other DNA
tctctggggacgtgacaaaggtaaccggtagaattacgg

SEQ ID NO: 280 - DNA, 42 bp, synthetic construct
  misc_feature 1..42: note = Target Specific Oligonucleotide
  source 1..42: mol_type = other DNA
gaattcatcagctagattggcaagtaaccggtagaattacgg

SEQ ID NO: 281 - DNA, 38 bp, synthetic construct
  misc_feature 1..38: note = Target Specific Oligonucleotide
  source 1..38: mol_type = other DNA
tcactccgtgggctaagcggtaaccggtagaattacgg

SEQ ID NO: 282 - DNA, 40 bp, synthetic construct
  misc_feature 1..40: note = Target Specific Oligonucleotide
  source 1..40: mol_type = other DNA
tgtaagggaacacggaagtgggtaaccggtagaattacgg

SEQ ID NO: 283 - DNA, 38 bp, synthetic construct
  misc_feature 1..38: note = Target Specific Oligonucleotide
  source 1..38: mol_type = other DNA
ctcaatggtggcgcggatcgtaaccggtagaattacgg

SEQ ID NO: 284 - DNA, 40 bp, synthetic construct
  misc_feature 1..40: note = Target Specific Oligonucleotide
  source 1..40: mol_type = other DNA
aaccactcaatctgcgtctcggtaaccggtagaattacgg

SEQ ID NO: 285 - DNA, 39 bp, synthetic construct
  misc_feature 1..39: note = Target Specific Oligonucleotide
  source 1..39: mol_type = other DNA
gctgtagggcgccattttgtgtaaccggtagaattacgg

SEQ ID NO: 286 - DNA, 38 bp, synthetic construct
  misc_feature 1..38: note = Target Specific Oligonucleotide
  source 1..38: mol_type = other DNA
tgagtgacggcattgcgcagtaaccggtagaattacgg

SEQ ID NO: 287 - DNA, 39 bp, synthetic construct
  misc_feature 1..39: note = Target Specific Oligonucleotide
  source 1..39: mol_type = other DNA
caaagtggctcatcgccaccgtaaccggtagaattacgg

SEQ ID NO: 288 - DNA, 45 bp, synthetic construct
  misc_feature 1..45: note = Target Specific Oligonucleotide
  source 1..45: mol_type = other DNA
tctatatgctaaatgtattgccatggtaacgcttctcataacact

SEQ ID NO: 289 - DNA, 43 bp, synthetic construct
  misc_feature 1..43: note = Target Specific Oligonucleotide
  source 1..43: mol_type = other DNA
ctatgaaacttcgtggtcactccgtaacgcttctcataacact

SEQ ID NO: 290 - DNA, 43 bp, synthetic construct
  misc_feature 1..43: note = Target Specific Oligonucleotide
  source 1..43: mol_type = other DNA
aactaactcatctgcagtaccatgtaacgcttctcataacact

SEQ ID NO: 291 - DNA, 43 bp, synthetic construct
  misc_feature 1..43: note = Target Specific Oligonucleotide
  source 1..43: mol_type = other DNA
aaaacctgcttgatccacattctgtaacgcttctcataacact

SEQ ID NO: 292 - DNA, 45 bp, synthetic construct
  misc_feature 1..45: note = Target Specific Oligonucleotide
  source 1..45: mol_type = other DNA
acattaacaaatcactcttgattcagtaacgcttctcataacact

SEQ ID NO: 293 - DNA, 43 bp, synthetic construct
  misc_feature 1..43: note = Target Specific Oligonucleotide
  source 1..43: mol_type = other DNA
ttttgcttagctcatggtaaacagtaacgcttctcataacact

SEQ ID NO: 294 - DNA, 45 bp, synthetic construct
  misc_feature 1..45: note = Target Specific Oligonucleotide
  source 1..45: mol_type = other DNA
gaggagctgttggataaataattttgtaacgcttctcataacact

SEQ ID NO: 295 - DNA, 43 bp, synthetic construct
  misc_feature 1..43: note = Target Specific Oligonucleotide
  source 1..43: mol_type = other DNA
aagcaggcaggtattgtatgattgtaacgcttctcataacact

SEQ ID NO: 296 - DNA, 45 bp, synthetic construct
  misc_feature 1..45: note = Target Specific Oligonucleotide
  source 1..45: mol_type = other DNA
gcaacttatatatcagtggtgaatggtaacgcttctcataacact

SEQ ID NO: 297 - DNA, 45 bp, synthetic construct
  misc_feature 1..45: note = Target Specific Oligonucleotide
  source 1..45: mol_type = other DNA
agattatgtatgcatgagaaccaacgtaacgcttctcataacact

SEQ ID NO: 298 - DNA, 45 bp, synthetic construct
  misc_feature 1..45: note = Target Specific Oligonucleotide
  source 1..45: mol_type = other DNA
gatcatgactctccttatggttaatgtaacgcttctcataacact

SEQ ID NO: 299 - DNA, 43 bp, synthetic construct
  misc_feature 1..43: note = Target Specific Oligonucleotide
  source 1..43: mol_type = other DNA
tgtgaaaccggtttataaacctagtaacgcttctcataacact

SEQ ID NO: 300 - DNA, 46 bp, synthetic construct
  misc_feature 1..46: note = Target Specific Oligonucleotide
  source 1..46: mol_type = other DNA
aataagattagctactgtctacagtggtaacgcttctcataacact

SEQ ID NO: 301 - DNA, 45 bp, synthetic construct
  misc_feature 1..45: note = Target Specific Oligonucleotide
  source 1..45: mol_type = other DNA
taaacaaatagtgatacatccacacgtaacgcttctcataacact

SEQ ID NO: 302 - DNA, 45 bp, synthetic construct
  misc_feature 1..45: note = Target Specific Oligonucleotide
  source 1..45: mol_type = other DNA
ttcagtcatctaacaatgtattcctgtaacgcttctcataacact

SEQ ID NO: 303 - DNA, 43 bp, synthetic construct
  misc_feature 1..43: note = Target Specific Oligonucleotide
  source 1..43: mol_type = other DNA
agctgtgggaaagtctttaactcgtaacgcttctcataacact

SEQ ID NO: 304 - DNA, 46 bp, synthetic construct
  misc_feature 1..46: note = Target Specific Oligonucleotide
  source 1..46: mol_type = other DNA
atagatgaactattgatgtacacaacgtaacgcttctcataacact

SEQ ID NO: 305 - DNA, 45 bp, synthetic construct
  misc_feature 1..45: note = Target Specific Oligonucleotide
  source 1..45: mol_type = other DNA
tacagatgaaattgagacctaaaacgtaacgcttctcataacact

SEQ ID NO: 306 - DNA, 45 bp, synthetic construct
  misc_feature 1..45: note = Target Specific Oligonucleotide
  source 1..45: mol_type = other DNA
agcaatttgggttaacagaaatagagtaacgcttctcataacact

SEQ ID NO: 307 - DNA, 46 bp, synthetic construct
  misc_feature 1..46: note = Target Specific Oligonucleotide
  source 1..46: mol_type = other DNA
aatccaatgggataagtactattagtgtaacgcttctcataacact

SEQ ID NO: 308 - DNA, 45 bp, synthetic construct
  misc_feature 1..45: note = Target Specific Oligonucleotide
  source 1..45: mol_type = other DNA
aatgttattccgtggaaaattacatgtaacgcttctcataacact

SEQ ID NO: 309 - DNA, 47 bp, synthetic construct
  misc_feature 1..47: note = Target Specific Oligonucleotide
  source 1..47: mol_type = other DNA
ttacagaattgtgtctgaaaaattatggtaacgcttctcataacact

SEQ ID NO: 310 - DNA, 40 bp, synthetic construct
  misc_feature 1..40: note = Target Specific Oligonucleotide
  source 1..40: mol_type = other DNA
atcagagaggcgctatgcctgtaacgcttctcataacact

SEQ ID NO: 311 - DNA, 38 bp, synthetic construct
  misc_feature 1..38: note = Target Specific Oligonucleotide
  source 1..38: mol_type = other DNA
cggcgacacgatacagcggtaacgcttctcataacact

SEQ ID NO: 312 - DNA, 40 bp, synthetic construct
  misc_feature 1..40: note = Target Specific Oligonucleotide
  source 1..40: mol_type = other DNA
cacttcaaatgcgcaacaagcgtaacgtcgctgaaaaatc

SEQ ID NO: 313 - DNA, 39 bp, synthetic construct
  misc_feature 1..39: note = Target Specific Oligonucleotide
  source 1..39: mol_type = other DNA
gtaagcactgcgcaagacaagtaacgtcgctgaaaaatc

SEQ ID NO: 314 - DNA, 41 bp, synthetic construct
  misc_feature 1..41: note = Target Specific Oligonucleotide
  source 1..41: mol_type = other DNA
gtgatgagctcgacaggatattgtaacgtcgctgaaaaatc

SEQ ID NO: 315 - DNA, 37 bp, synthetic construct
  misc_feature 1..37: note = Target Specific Oligonucleotide
  source 1..37: mol_type = other DNA
atggcgtcgtcggcacacgtaacgtcgctgaaaaatc

SEQ ID NO: 316 - DNA, 40 bp, synthetic construct
  misc_feature 1..40: note = Target Specific Oligonucleotide
  source 1..40: mol_type = other DNA
tttacaacagcgtggcaagtggtaacgtcgctgaaaaatc

SEQ ID NO: 317 - DNA, 40 bp, synthetic construct
  misc_feature 1..40: note = Target Specific Oligonucleotide
  source 1..40: mol_type = other DNA
aggcatgaagtgagacaatgcgtaacgtcgctgaaaaatc

SEQ ID NO: 318 - DNA, 41 bp, synthetic construct
  misc_feature 1..41: note = Target Specific Oligonucleotide
  source 1..41: mol_type = other DNA
cctggctagtggtatatgtcacgtaacgtcgctgaaaaatc

SEQ ID NO: 319 - DNA, 39 bp, synthetic construct
  misc_feature 1..39: note = Target Specific Oligonucleotide
  source 1..39: mol_type = other DNA
gctgatgatgttcaagcgcagtaacgtcgctgaaaaatc

SEQ ID NO: 320 - DNA, 41 bp, synthetic construct
  misc_feature 1..41: note = Target Specific Oligonucleotide
  source 1..41: mol_type = other DNA
ctgccagttagttaggcaagttgtaacgtcgctgaaaaatc

SEQ ID NO: 321 - DNA, 38 bp, synthetic construct
  misc_feature 1..38: note = Target Specific Oligonucleotide
  source 1..38: mol_type = other DNA
gcagctccgggctacaagtgtaacgtcgctgaaaaatc

SEQ ID NO: 322 - DNA, 40 bp, synthetic construct
  misc_feature 1..40: note = Target Specific Oligonucleotide
  source 1..40: mol_type = other DNA
tggcagctgtctaactggagcgtaacgtcgctgaaaaatc

SEQ ID NO: 323 - DNA, 38 bp, synthetic construct
  misc_feature 1..38: note = Target Specific Oligonucleotide
  source 1..38: mol_type = other DNA
acgcgtgtgcgagtagatggtaacgtcgctgaaaaatc

SEQ ID NO: 324 - DNA, 38 bp, synthetic construct
  misc_feature 1..38: note = Target Specific Oligonucleotide
  source 1..38: mol_type = other DNA
agctccacgaaggatgccagtaacgtcgctgaaaaatc

SEQ ID NO: 325 - DNA, 39 bp, synthetic construct
  misc_feature 1..39: note = Target Specific Oligonucleotide
  source 1..39: mol_type = other DNA
tgaggacggcatcgagatccgtaacgtcgctgaaaaatc

SEQ ID NO: 326 - DNA, 39 bp, synthetic construct
  misc_feature 1..39: note = Target Specific Oligonucleotide
  source 1..39: mol_type = other DNA
gtcttgagacccggtcttgggtaacgtcgctgaaaaatc

SEQ ID NO: 327 - DNA, 38 bp, synthetic construct
  misc_feature 1..38: note = Target Specific Oligonucleotide
  source 1..38: mol_type = other DNA
agtggtctggatcggtgcggtaacgtcgctgaaaaatc

SEQ ID NO: 328 - DNA, 39 bp, synthetic construct
  misc_feature 1..39: note = Target Specific Oligonucleotide
  source 1..39: mol_type = other DNA
tccattctgggtcgagtggagtaacgtcgctgaaaaatc

SEQ ID NO: 329 - DNA, 39 bp, synthetic construct
  misc_feature 1..39: note = Target Specific Oligonucleotide
  source 1..39: mol_type = other DNA
ggaccccaaggtgttccaaggtaacgtcgctgaaaaatc

SEQ ID NO: 330 - DNA, 38 bp, synthetic construct
  misc_feature 1..38: note = Target Specific Oligonucleotide
  source 1..38: mol_type = other DNA
agcagcttggctactccccgtaacgtcgctgaaaaatc

SEQ ID NO: 331 - DNA, 42 bp, synthetic construct
  misc_feature 1..42: note = Target Specific Oligonucleotide
  source 1..42: mol_type = other DNA
tttgggtatgggtactgtgtagagtaacgtcgctgaaaaatc

SEQ ID NO: 332 - DNA, 39 bp, synthetic construct
  misc_feature 1..39: note = Target Specific Oligonucleotide
  source 1..39: mol_type = other DNA
gccgcagggatagatccagggtaacgtcgctgaaaaatc

SEQ ID NO: 333 - DNA, 42 bp, synthetic construct
  misc_feature 1..42: note = Target Specific Oligonucleotide
  source 1..42: mol_type = other DNA
tgaagacagtcctatggacttccgtaacgtcgctgaaaaatc

SEQ ID NO: 334 - DNA, 37 bp, synthetic construct
  misc_feature 1..37: note = Target Specific Oligonucleotide
  source 1..37: mol_type = other DNA
aagctgaagcgcgggtcagtaacgtcgctgaaaaatc

SEQ ID NO: 335 - DNA, 37 bp, synthetic construct
  misc_feature 1..37: note = Target Specific Oligonucleotide
  source 1..37: mol_type = other DNA
ccacgcagcccttcgagagtaacgtcgctgaaaaatc

SEQ ID NO: 336 - DNA, 45 bp, synthetic construct
  misc_feature 1..45: note = Target Specific Oligonucleotide
  source 1..45: mol_type = other DNA
tagtaggtgtcgacaactagagcgtaactcttcaagattaatacc

SEQ ID NO: 337 - DNA, 44 bp, synthetic construct
  misc_feature 1..44: note = Target Specific Oligonucleotide
  source 1..44: mol_type = other DNA
gatggtcctgatcgagaaaccagtaactcttcaagattaatacc

SEQ ID NO: 338 - DNA, 44 bp, synthetic construct
  misc_feature 1..44: note = Target Specific Oligonucleotide
  source 1..44: mol_type = other DNA
aagaggacttcgctgaattgacgtaactcttcaagattaatacc

SEQ ID NO: 339 - DNA, 44 bp, synthetic construct
  misc_feature 1..44: note = Target Specific Oligonucleotide
  source 1..44: mol_type = other DNA
gaacctccgactgtatgtcagcgtaactcttcaagattaatacc

SEQ ID NO: 340 - DNA, 45 bp, synthetic construct
  misc_feature 1..45: note = Target Specific Oligonucleotide
  source 1..45: mol_type = other DNA
tctgaattagagcgatgttgacagtaactcttcaagattaatacc

SEQ ID NO: 341 - DNA, 41 bp, synthetic construct
  misc_feature 1..41: note = Target Specific Oligonucleotide
  source 1..41: mol_type = other DNA
gcattcctccgatcgcacagtaactcttcaagattaatacc

SEQ ID NO: 342 - DNA, 46 bp, synthetic construct
  misc_feature 1..46: note = Target Specific Oligonucleotide
  source 1..46: mol_type = other DNA
ctctatatttagctcgctgttcaagtaactcttcaagattaatacc

SEQ ID NO: 343 - DNA, 41 bp, synthetic construct
  misc_feature 1..41: note = Target Specific Oligonucleotide
  source 1..41: mol_type = other DNA
tattcatcacggcgcgcttgtaactcttcaagattaatacc

SEQ ID NO: 344 - DNA, 43 bp, synthetic construct
  misc_feature 1..43: note = Target Specific Oligonucleotide
  source 1..43: mol_type = other DNA
cattggggaccgtgcataaaagtaactcttcaagattaatacc

SEQ ID NO: 345 - DNA, 43 bp, synthetic construct
  misc_feature 1..43: note = Target Specific Oligonucleotide
  source 1..43: mol_type = other DNA
caaggggtgcactatttgggagtaactcttcaagattaatacc

SEQ ID NO: 346 - DNA, 43 bp, synthetic construct
  misc_feature 1..43: note = Target Specific Oligonucleotide
  source 1..43: mol_type = other DNA
ggaccgtatttcggcgaaatagtaactcttcaagattaatacc

SEQ ID NO: 347 - DNA, 43 bp, synthetic construct
  misc_feature 1..43: note = Target Specific Oligonucleotide
  source 1..43: mol_type = other DNA
atactgcacttgtcggcatgagtaactcttcaagattaatacc

SEQ ID NO: 348 - DNA, 43 bp, synthetic construct
  misc_feature 1..43: note = Target Specific Oligonucleotide
  source 1..43: mol_type = other DNA
atggcagatcgatccattggtgtaactcttcaagattaatacc

SEQ ID NO: 349 - DNA, 43 bp, synthetic construct
  misc_feature 1..43: note = Target Specific Oligonucleotide
  source 1..43: mol_type = other DNA
tccctttggaccgtcaagaaggtaactcttcaagattaatacc

SEQ ID NO: 350 - DNA, 42 bp, synthetic construct
  misc_feature 1..42: note = Target Specific Oligonucleotide
  source 1..42: mol_type = other DNA
aggcttgctgacatacgcaggtaactcttcaagattaatacc

SEQ ID NO: 351 - DNA, 42 bp, synthetic construct
  misc_feature 1..42: note = Target Specific Oligonucleotide
  source 1..42: mol_type = other DNA
cttgctttgtgcgaacacccgtaactcttcaagattaatacc

SEQ ID NO: 352 - DNA, 45 bp, synthetic construct
  misc_feature 1..45: note = Target Specific Oligonucleotide
  source 1..45: mol_type = other DNA
ctgatatcgaatgcaatggatgagtaactcttcaagattaatacc

SEQ ID NO: 353 - DNA, 43 bp, synthetic construct
  misc_feature 1..43: note = Target Specific Oligonucleotide
  source 1..43: mol_type = other DNA
taggactggtccgtcaaaaacgtaactcttcaagattaatacc

SEQ ID NO: 354 - DNA, 44 bp, synthetic construct
  misc_feature 1..44: note = Target Specific Oligonucleotide
  source 1..44: mol_type = other DNA
tgaccattctcgggacactaacgtaactcttcaagattaatacc

SEQ ID NO: 355 - DNA, 44 bp, synthetic construct
  misc_feature 1..44: note = Target Specific Oligonucleotide
  source 1..44: mol_type = other DNA
atgattgggtccgtaaaaatgcgtaactcttcaagattaatacc

SEQ ID NO: 356 - DNA, 45 bp, synthetic construct
  misc_feature 1..45: note = Target Specific Oligonucleotide
  source 1..45: mol_type = other DNA
gtttcatgtatggtaggaccaccgtaactcttcaagattaatacc

SEQ ID NO: 357 - DNA, 43 bp, synthetic construct
  misc_feature 1..43: note = Target Specific Oligonucleotide
  source 1..43: mol_type = other DNA
ctcttgcatcgtagcgaactagtaactcttcaagattaatacc

SEQ ID NO: 358 - DNA, 43 bp, synthetic construct
  misc_feature 1..43: note = Target Specific Oligonucleotide
  source 1..43: mol_type = other DNA
atgatgattccctcggtcagagtaactcttcaagattaatacc

SEQ ID NO: 359 - DNA, 46 bp, synthetic construct
  misc_feature 1..46: note = Target Specific Oligonucleotide
  source 1..46: mol_type = other DNA
agtagagaagatcgctgatatccggtaactcttcaagattaatacc

SEQ ID NO: 360 - DNA, 42 bp, synthetic construct
  misc_feature 1..42: note = Target Specific Oligonucleotide
  source 1..42: mol_type = other DNA
aacccttgacatcgccagtttgtaacgtaccgtttgtatatg

SEQ ID NO: 361 - DNA, 45 bp, synthetic construct
  misc_feature 1..45: note = Target Specific Oligonucleotide
  source 1..45: mol_type = other DNA
gaaggttaaacgagatttccaaaggtaacgtaccgtttgtatatg

SEQ ID NO: 362 - DNA, 41 bp, synthetic construct
  misc_feature 1..41: note = Target Specific Oligonucleotide
  source 1..41: mol_type = other DNA
gccactcgacatttctgccggtaacgtaccgtttgtatatg

SEQ ID NO: 363 - DNA, 42 bp, synthetic construct
  misc_feature 1..42: note = Target Specific Oligonucleotide
  source 1..42: mol_type = other DNA
tcatactgtccgcaacatccggtaacgtaccgtttgtatatg

SEQ ID NO: 364 - DNA, 41 bp, synthetic construct; identical to SEQ ID NO: 362
  misc_feature 1..41: note = Target Specific Oligonucleotide
  source 1..41: mol_type = other DNA

SEQ ID NO: 365 - DNA, 42 bp, synthetic construct; identical to SEQ ID NO: 363
  misc_feature 1..42: note = Target Specific Oligonucleotide
  source 1..42: mol_type = other DNA

SEQ ID NO: 366 - DNA, 43 bp, synthetic construct
  misc_feature 1..43: note = Target Specific Oligonucleotide
  source 1..43: mol_type = other DNA
tgaattttgcccgaacttcactgtaacgtaccgtttgtatatg

SEQ ID NO: 367 - DNA, 38 bp, synthetic construct
  misc_feature 1..38: note = Target Specific Oligonucleotide
  source 1..38: mol_type = other DNA
acacactcggagcgcgcgtaacgtaccgtttgtatatg

SEQ ID NO: 368 - DNA, 43 bp, synthetic construct
  misc_feature 1..43: note = Target Specific Oligonucleotide
  source 1..43: mol_type = other DNA
caaacctatatgcccgttgactgtaacgtaccgtttgtatatg

SEQ ID NO: 369 - DNA, 44 bp, synthetic construct
  misc_feature 1..44: note = Target Specific Oligonucleotide
  source 1..44: mol_type = other DNA
caggaacctttaccatgttcatggtaacgtaccgtttgtatatg

SEQ ID NO: 370 - DNA, 46 bp, synthetic construct
  misc_feature 1..46: note = Target Specific Oligonucleotide
  source 1..46: mol_type = other DNA
gaaaatctctacggatgaatttcttgtaacgtaccgtttgtatatg

SEQ ID NO: 371 - DNA, 43 bp, synthetic construct
  misc_feature 1..43: note = Target Specific Oligonucleotide
  source 1..43: mol_type = other DNA
gctgctaagatagccttgtgaggtaacgtaccgtttgtatatg

SEQ ID NO: 372 - DNA, 42 bp, synthetic construct
  misc_feature 1..42: note = Target Specific Oligonucleotide
  source 1..42: mol_type = other DNA
tggcactaaaaaccggagaacgtaacgtaccgtttgtatatg

SEQ ID NO: 373 - DNA, 39 bp, synthetic construct
  misc_feature 1..39: note = Target Specific Oligonucleotide
  source 1..39: mol_type = other DNA
tgcgggaccgccgatacagtaacgtaccgtttgtatatg

SEQ ID NO: 374 - DNA, 45 bp, synthetic construct
  misc_feature 1..45: note = Target Specific Oligonucleotide
  source 1..45: mol_type = other DNA
ggaaattaaacggagtcttacaacgtaacgtaccgtttgtatatg

SEQ ID NO: 375 - DNA, 40 bp, synthetic construct
  misc_feature 1..40: note = Target Specific Oligonucleotide
  source 1..40: mol_type = other DNA
agccagagagcggtatgccgtaacgtaccgtttgtatatg

SEQ ID NO: 376 - DNA, 41 bp, synthetic construct
  misc_feature 1..41: note = Target Specific Oligonucleotide
  source 1..41: mol_type = other DNA
ccaccaagtggggatgtgacgtaacgtaccgtttgtatatg

SEQ ID NO: 377 - DNA, 42 bp, synthetic construct
  misc_feature 1..42: note = Target Specific Oligonucleotide
  source 1..42: mol_type = other DNA
cacacatgacctttaagcgctgtaacgtaccgtttgtatatg

SEQ ID NO: 378 - DNA, 41 bp, synthetic construct
  misc_feature 1..41: note = Target Specific Oligonucleotide
  source 1..41: mol_type = other DNA
tccacagattgcgctgtctagtaacgtaccgtttgtatatg

SEQ ID NO: 379 - DNA, 41 bp, synthetic construct
  misc_feature 1..41: note = Target Specific Oligonucleotide
  source 1..41: mol_type = other DNA
ctccacggacaggttactgcgtaacgtaccgtttgtatatg

SEQ ID NO: 380 - DNA, 39 bp, synthetic construct
  misc_feature 1..39: note = Target Specific Oligonucleotide
  source 1..39: mol_type = other DNA
gcgtcatggcgtcagcacgtaacgtaccgtttgtatatg

SEQ ID NO: 381 - DNA, 39 bp, synthetic construct
  misc_feature 1..39: note = Target Specific Oligonucleotide
  source 1..39: mol_type = other DNA
tcctggccggcaacacacgtaacgtaccgtttgtatatg

SEQ ID NO: 382 - DNA, 42 bp, synthetic construct
  misc_feature 1..42: note = Target Specific Oligonucleotide
  source 1..42: mol_type = other DNA
agccattttgtcgaggtttgggtaacgtaccgtttgtatatg

SEQ ID NO: 383 - DNA, 44 bp, synthetic construct
  misc_feature 1..44: note = Target Specific Oligonucleotide
  source 1..44: mol_type = other DNA
catccgaagcatgatagttgatggtaacgtaccgtttgtatatg

SEQ ID NO: 384 - DNA, 43 bp, synthetic construct
  misc_feature 1..43: note = Target Specific Oligonucleotide
  source 1..43: mol_type = other DNA
tagtgtgttgcgtatcaagtatctagacgatgcgaattaacac

SEQ ID NO: 385 - DNA, 45 bp, synthetic construct
  misc_feature 1..45: note = Target Specific Oligonucleotide
  source 1..45: mol_type = other DNA
ttattgtgaactctagacatgagagtagacgatgcgaattaacac

SEQ ID NO: 386 - DNA, 46 bp, synthetic construct
  misc_feature 1..46: note = Target Specific Oligonucleotide
  source 1..46: mol_type = other DNA
acagtgaatactaagactgtaaaaactagacgatgcgaattaacac

SEQ ID NO: 387 - DNA, 41 bp, synthetic construct
  misc_feature 1..41: note = Target Specific Oligonucleotide
  source 1..41: mol_type = other DNA
acacaaggcacgtagaaacagtagacgatgcgaattaacac

SEQ ID NO: 388 - DNA, 41 bp, synthetic construct
  misc_feature 1..41: note = Target Specific Oligonucleotide
  source 1..41: mol_type = other DNA
caatggacagcggttgtgaaatagacgatgcgaattaacac

SEQ ID NO: 389 - DNA, 42 bp, synthetic construct
  misc_feature 1..42: note = Target Specific Oligonucleotide
  source 1..42: mol_type = other DNA
caccatcacctatcgacagagttagacgatgcgaattaacac

SEQ ID NO: 390 - DNA, 42 bp, synthetic construct
  misc_feature 1..42: note = Target Specific Oligonucleotide
  source 1..42: mol_type = other DNA
cattgcattacgttccacatggtagacgatgcgaattaacac

SEQ ID NO: 391 - DNA, 41 bp, synthetic construct
  misc_feature 1..41: note = Target Specific Oligonucleotide
  source 1..41: mol_type = other DNA
acacagagtgtccgatacccatagacgatgcgaattaacac

SEQ ID NO: 392 - DNA, 43 bp, synthetic construct
  misc_feature 1..43: note = Target Specific Oligonucleotide
  source 1..43: mol_type = other DNA
ggtgattacattgagtgctaggatagacgatgcgaattaacac

SEQ ID NO: 393 - DNA, 41 bp, synthetic construct
  misc_feature 1..41: note = Target Specific Oligonucleotide
  source 1..41: mol_type = other DNA
tccacataagcgttcaaggtatagacgatgcgaattaacac

SEQ ID NO: 394 - DNA, 44 bp, synthetic construct
  misc_feature 1..44: note = Target Specific Oligonucleotide
  source 1..44: mol_type = other DNA
cagaataatcgctattcctagctgtagacgatgcgaattaacac

SEQ ID NO: 395 - DNA, 40 bp, synthetic construct
  misc_feature 1..40: note = Target Specific Oligonucleotide
  source 1..40: mol_type = other DNA
ttacactcatacgtcgccggtagacgatgcgaattaacac

SEQ ID NO: 396 - DNA, 43 bp, synthetic construct
  misc_feature 1..43: note = Target Specific Oligonucleotide
  source 1..43: mol_type = other DNA
tggccataaaggctacttacaattagacgatgcgaattaacac

SEQ ID NO: 397 - DNA, 45 bp, synthetic construct
  misc_feature 1..45: note = Target Specific Oligonucleotide
  source 1..45: mol_type = other DNA
tttcaagttattcgatctgctaacctagacgatgcgaattaacac

SEQ ID NO: 398 - DNA, 45 bp, synthetic construct
  misc_feature 1..45: note = Target Specific Oligonucleotide
  source 1..45: mol_type = other DNA
agttagtcaaggactttactaaggttagacgatgcgaattaacac

SEQ ID NO: 399 - DNA, 42 bp, synthetic construct
  misc_feature 1..42: note = Target Specific Oligonucleotide
  source 1..42: mol_type = other DNA
ttacctcaaatacgggctaccatagacgatgcgaattaacac

SEQ ID NO: 400 - DNA, 47 bp, synthetic construct
  misc_feature 1..47: note = Target Specific Oligonucleotide
  source 1..47: mol_type = other DNA
gagttatattactctaactaaagccagtagacgatgcgaattaacac

SEQ ID NO: 401 - DNA, 43 bp, synthetic construct
  misc_feature 1..43: note = Target Specific Oligonucleotide
  source 1..43: mol_type = other DNA
ttgtcaacacgcataaaatctgctagacgatgcgaattaacac

SEQ ID NO: 402 - DNA, 43 bp, synthetic construct
  misc_feature 1..43: note = Target Specific Oligonucleotide
  source 1..43: mol_type = other DNA
tcatatacaatcggggatctgagtagacgatgcgaattaacac

SEQ ID NO: 403 - DNA, 43 bp, synthetic construct
  misc_feature 1..43: note = Target Specific Oligonucleotide
  source 1..43: mol_type = other DNA
cccacaaaaatacccgtaagttatagacgatgcgaattaacac

SEQ ID NO: 404 - DNA, 47 bp, synthetic construct
  misc_feature 1..47: note = Target Specific Oligonucleotide
  source 1..47: mol_type = other DNA
cactaaattattacgaattttgcaaagtagacgatgcgaattaacac

SEQ ID NO: 405 - DNA, 45 bp, synthetic construct
  misc_feature 1..45: note = Target Specific Oligonucleotide
  source 1..45: mol_type = other DNA
tcaaataaaaaccgtcaaaagtttatagacgatgcgaattaacac

SEQ ID NO: 406 - DNA, 42 bp, synthetic construct
  misc_feature 1..42: note = Target Specific Oligonucleotide
  source 1..42: mol_type = other DNA
agtgcaatggtatcacgtactgtagacgatgcgaattaacac

SEQ ID NO: 407 - DNA, 43 bp, synthetic construct
  misc_feature 1..43: note = Target Specific Oligonucleotide
  source 1..43: mol_type = other DNA
atgtgtcttgcaattggattccctagacgatgcgaattaacac

SEQ ID NO: 408 - DNA, 38 bp, synthetic construct
  misc_feature 1..38: note = Target Specific Oligonucleotide
  source 1..38: mol_type = other DNA
ggtgtgcgcgtcgtacactagacccgttataagtgttg

SEQ ID NO: 409 - DNA, 40 bp, synthetic construct
  misc_feature 1..40: note = Target Specific Oligonucleotide
  source 1..40: mol_type = other DNA
gtgtttagggtcgcggttgatagacccgttataagtgttg

SEQ ID NO: 410 - DNA, 42 bp, synthetic construct
  misc_feature 1..42: note = Target Specific Oligonucleotide
  source 1..42: mol_type = other DNA
gagaatggcgaagtaaatgccctagacccgttataagtgttg

SEQ ID NO: 411 - DNA, 42 bp, synthetic construct
  misc_feature 1..42: note = Target Specific Oligonucleotide
  source 1..42: mol_type = other DNA
gtagatggaatagacacggctgtagacccgttataagtgttg

SEQ ID NO: 412 - DNA, 43 bp, synthetic construct
  misc_feature 1..43: note = Target Specific Oligonucleotide
  source 1..43: mol_type = other DNA
gctcttaatgcatggtacaactgtagacccgttataagtgttg

SEQ ID NO: 413 - DNA, 43 bp, synthetic construct
  misc_feature 1..43: note = Target Specific Oligonucleotide
  source 1..43: mol_type = other DNA
aaaccagtatttcgtcacagtgatagacccgttataagtgttg

SEQ ID NO: 414 - DNA, 42 bp, synthetic construct
  misc_feature 1..42: note = Target Specific Oligonucleotide
  source 1..42: mol_type = other DNA
gatgggaacggtgtagagatgttagacccgttataagtgttg

SEQ ID NO: 415 - DNA, 42 bp, synthetic construct
  misc_feature 1..42: note = Target Specific Oligonucleotide
  source 1..42: mol_type = other DNA
ttccaaatgccgtcaaaactgttagacccgttataagtgttg

SEQ ID NO: 416 - DNA, 39 bp, synthetic construct
  misc_feature 1..39: note = Target Specific Oligonucleotide
  source 1..39: mol_type = other DNA
tgggtcacagacggtgtggtagacccgttataagtgttg

SEQ ID NO: 417 - DNA, 46 bp, synthetic construct
  misc_feature 1..46: note = Target Specific Oligonucleotide
  source 1..46: mol_type = other DNA
aatcaggtatacttctatccttgaaatagacccgttataagtgttg

SEQ ID NO: 418 - DNA, 43 bp, synthetic construct
  misc_feature 1..43: note = Target Specific Oligonucleotide
  source 1..43: mol_type = other DNA
tgctgtattagcaacttggaacttagacccgttataagtgttg

SEQ ID NO: 419 - DNA, 42 bp, synthetic construct
  misc_feature 1..42: note = Target Specific Oligonucleotide
  source 1..42: mol_type = other DNA
gccaataaagcgatggttgatctagacccgttataagtgttg

SEQ ID NO: 420 - DNA, 43 bp, synthetic construct
  misc_feature 1..43: note = Target Specific Oligonucleotide
  source 1..43: mol_type = other DNA
gtaaggatcactggatcctactgtagacccgttataagtgttg

SEQ ID NO: 421 - DNA, 43 bp, synthetic construct
  misc_feature 1..43: note = Target Specific Oligonucleotide
  source 1..43: mol_type = other DNA
gacctcattcagttgatgagaggtagacccgttataagtgttg

SEQ ID NO: 422 - DNA, 41 bp, synthetic construct
  misc_feature 1..41: note = Target Specific Oligonucleotide
  source 1..41: mol_type = other DNA
gcataacagacggttgcaagttagacccgttataagtgttg

SEQ ID NO: 423 - DNA, 41 bp, synthetic construct
  misc_feature 1..41: note = Target Specific Oligonucleotide
  source 1..41: mol_type = other DNA
acctaaagcgagttgctgagttagacccgttataagtgttg

SEQ ID NO: 424 - DNA, 41 bp, synthetic construct
  misc_feature 1..41: note = Target Specific Oligonucleotide
  source 1..41: mol_type = other DNA
gagtcagaaacacgcatggaatagacccgttataagtgttg

SEQ ID NO: 425 - DNA, 42 bp, synthetic construct
  misc_feature 1..42: note = Target Specific Oligonucleotide
  source 1..42: mol_type = other DNA
agcaactctgataggctcacactagacccgttataagtgttg

SEQ ID NO: 426 - DNA, 44 bp, synthetic construct
  misc_feature 1..44: note = Target Specific Oligonucleotide
  source 1..44: mol_type = other DNA
ccatacccactacggataaagatgtagacccgttataagtgttg

SEQ ID NO: 427 - DNA, 42 bp, synthetic construct
  misc_feature 1..42: note = Target Specific Oligonucleotide
  source 1..42: mol_type = other DNA
gctttctgtacgactcaggttttagacccgttataagtgttg

SEQ ID NO: 428 - DNA, 42 bp, synthetic construct
  misc_feature 1..42: note = Target Specific Oligonucleotide
  source 1..42: mol_type = other DNA
caatgttgagccactaaaccactagacccgttataagtgttg

SEQ ID NO: 429 - DNA, 42 bp, synthetic construct
  misc_feature 1..42: note = Target Specific Oligonucleotide
  source 1..42: mol_type = other DNA
atgcttggattaggtccaaagctagacccgttataagtgttg

SEQ ID NO: 430 - DNA, 41 bp, synthetic construct
  misc_feature 1..41: note = Target Specific Oligonucleotide
  source 1..41: mol_type = other DNA
cccggtgatggattagtttggtagacccgttataagtgttg

SEQ ID NO: 431 - DNA, 41 bp, synthetic construct
  misc_feature 1..41: note = Target Specific Oligonucleotide
  source 1..41: mol_type = other DNA
ccatggccataccctggaatttagacccgttataagtgttg

SEQ ID NO: 432 - DNA, 41 bp, synthetic construct
  misc_feature 1..41: note = Target Specific Oligonucleotide
  source 1..41: mol_type = other DNA
attatactgtccagcgtaggtggtagacaaccgtcgttaag

SEQ ID NO: 433 - DNA, 41 bp, synthetic construct
  misc_feature 1..41: note = Target Specific Oligonucleotide
  source 1..41: mol_type = other DNA
tcctggtaaggataggtaccatgtagacaaccgtcgttaag

SEQ ID NO: 434 - DNA, 41 bp, synthetic construct
  misc_feature 1..41: note = Target Specific Oligonucleotide
  source 1..41: mol_type = other DNA
cactgaaataggacggaatctgctagacaaccgtcgttaag

SEQ ID NO: 435 - DNA, 39 bp, synthetic construct
  misc_feature 1..39: note = Target Specific Oligonucleotide
  source 1..39: mol_type = other DNA
aaagtttattggcgcttgccgtagacaaccgtcgttaag

SEQ ID NO: 436 - DNA, 39 bp, synthetic construct
  misc_feature 1..39: note = Target Specific Oligonucleotide
  source 1..39: mol_type = other DNA
gcaatgccattagcgatacgatagacaaccgtcgttaag

SEQ ID NO: 437 - DNA, 38 bp, synthetic construct
  misc_feature 1..38: note = Target Specific Oligonucleotide
  source 1..38: mol_type = other DNA
tgattgaacaccacgcgacatagacaaccgtcgttaag

SEQ ID NO: 438 - DNA, 40 bp, synthetic construct
  misc_feature 1..40: note = Target Specific Oligonucleotide
  source 1..40: mol_type = other DNA
agtcttcgaagcactattgccatagacaaccgtcgttaag

SEQ ID NO: 439 - DNA, 39 bp, synthetic construct
  misc_feature 1..39: note = Target Specific Oligonucleotide
  source 1..39: mol_type = other DNA
tcaatggttgatcggcctctctagacaaccgtcgttaag

SEQ ID NO: 440 - DNA, 41 bp, synthetic construct
  misc_feature 1..41: note = Target Specific Oligonucleotide
  source 1..41: mol_type = other DNA
tggcatgatgtctaataggagtctagacaaccgtcgttaag

SEQ ID NO: 441 - DNA, 38 bp, synthetic construct
  misc_feature 1..38: note = Target Specific Oligonucleotide
  source 1..38: mol_type = other DNA
ctgcggtttagccttgacgttagacaaccgtcgttaag

SEQ ID NO: 442 - DNA, 38 bp, synthetic construct
  misc_feature 1..38: note = Target Specific Oligonucleotide
  source 1..38: mol_type = other DNA
cccagaggacgccatcattttagacaaccgtcgttaag

SEQ ID NO: 443 - DNA, 40 bp, synthetic construct
  misc_feature 1..40: note = Target Specific Oligonucleotide
  source 1..40: mol_type = other DNA
tttcttgaaagacgacagcagttagacaaccgtcgttaag

SEQ ID NO: 444 - DNA, 41 bp, synthetic construct
  misc_feature 1..41: note = Target Specific Oligonucleotide
  source 1..41: mol_type = other DNA
attaaatgttcggaaggatgacctagacaaccgtcgttaag

SEQ ID NO: 445 - DNA, 38 bp, synthetic construct
  misc_feature 1..38: note = Target Specific Oligonucleotide
  source 1..38: mol_type = other DNA
tcttttgtcagcacggttgctagacaaccgtcgttaag

SEQ ID NO: 446 - DNA, 38 bp, synthetic construct
  misc_feature 1..38: note = Target Specific Oligonucleotide
  source 1..38: mol_type = other DNA
ctggatcagctcgaccaggatagacaaccgtcgttaag

SEQ ID NO: 447 - DNA, 38 bp, synthetic construct
  misc_feature 1..38: note = Target Specific Oligonucleotide
  source 1..38: mol_type = other DNA
accatggtccgacttctgcctagacaaccgtcgttaag

SEQ ID NO: 448 - DNA, 40 bp, synthetic construct
  misc_feature 1..40: note = Target Specific Oligonucleotide
  source 1..40: mol_type = other DNA
ctggatattcggttatctgggctagacaaccgtcgttaag

SEQ ID NO: 449 - DNA, 41 bp, synthetic construct
  misc_feature 1..41: note = Target Specific Oligonucleotide
  source 1..41: mol_type = other DNA
atcccctttacgatactcttcagtagacaaccgtcgttaag

SEQ ID NO: 450 - DNA, 38 bp, synthetic construct
  misc_feature 1..38: note = Target Specific Oligonucleotide
  source 1..38: mol_type = other DNA
tccagagcgggaacagtatgtagacaaccgtcgttaag

SEQ ID NO: 451 - DNA, 38 bp, synthetic construct
  misc_feature 1..38: note = Target Specific Oligonucleotide
  source 1..38: mol_type = other DNA
gttgcctgcataagatgggctagacaaccgtcgttaag

SEQ ID NO: 452 - DNA, 38 bp, synthetic construct
  misc_feature 1..38: note = Target Specific Oligonucleotide
  source 1..38: mol_type = other DNA
ccttttgtgccgatgacccgtagacaaccgtcgttaag

SEQ ID NO: 453 - DNA, 37 bp, synthetic construct
  misc_feature 1..37: note = Target Specific Oligonucleotide
  source 1..37: mol_type = other DNA
gcttccgtggactgggacgtagacaaccgtcgttaag

SEQ ID NO: 454 - DNA, 41 bp, synthetic construct
  misc_feature 1..41: note = Target Specific Oligonucleotide
  source 1..41: mol_type = other DNA
ccatagaaggacgaggtatttcctagacaaccgtcgttaag

SEQ ID NO: 455 - DNA, 39 bp, synthetic construct
  misc_feature 1..39: note = Target Specific Oligonucleotide
  source 1..39: mol_type = other DNA
agaatgagtcgctgctcgatatagacaaccgtcgttaag

SEQ ID NO: 456 - DNA, 41 bp, synthetic construct
  misc_feature 1..41: note = Target Specific Oligonucleotide
  source 1..41: mol_type = other DNA
gcttcgacaaacgcaaagcgtagactcaatgatgataaaga

SEQ ID NO: 457 - DNA, 44 bp, synthetic construct
  misc_feature 1..44: note = Target Specific Oligonucleotide
  source 1..44: mol_type = other DNA
gctagttcacagaaagaccattttagactcaatgatgataaaga

SEQ ID NO: 458 - DNA, 41 bp, synthetic construct
  misc_feature 1..41: note = Target Specific Oligonucleotide
  source 1..41: mol_type = other DNA
cagctcatcgaaagcgaccctagactcaatgatgataaaga

SEQ ID NO: 459 - DNA, 40 bp, synthetic construct
  misc_feature 1..40: note = Target Specific Oligonucleotide
  source 1..40: mol_type = other DNA
cggctgctacaaacctcggtagactcaatgatgataaaga

SEQ ID NO: 460 - DNA, 42 bp, synthetic construct
  misc_feature 1..42: note = Target Specific Oligonucleotide
  source 1..42: mol_type = other DNA
aacaatttgaaggccccacaatagactcaatgatgataaaga

SEQ ID NO: 461 - DNA, 39 bp, synthetic construct
  misc_feature 1..39: note = Target Specific Oligonucleotide
  source 1..39: mol_type = other DNA
gcgtctgcaccggagacttagactcaatgatgataaaga

SEQ ID NO: 462 - DNA, 39 bp, synthetic construct
  misc_feature 1..39: note = Target Specific Oligonucleotide
  source 1..39: mol_type = other DNA
gcgctgctcaggcattggtagactcaatgatgataaaga

SEQ ID NO: 463 - DNA, 47 bp, synthetic construct
  misc_feature 1..47: note = Target Specific Oligonucleotide
  source 1..47: mol_type = other DNA
ttgttgtgtaacaacataatttcaagtagactcaatgatgataaaga

SEQ ID NO: 464 - DNA, 38 bp, synthetic construct
  misc_feature 1..38: note = Target Specific Oligonucleotide
  source 1..38: mol_type = other DNA
ctccacgtcggcgtgcatagactcaatgatgataaaga

SEQ ID NO: 465 - DNA, 40 bp, synthetic construct
  misc_feature 1..40: note = Target Specific Oligonucleotide
  source 1..40: mol_type = other DNA
ctgcggggattgtagccggtagactcaatgatgataaaga

SEQ ID NO: 466 - DNA, 42 bp, synthetic construct
  misc_feature 1..42: note = Target Specific Oligonucleotide
  source 1..42: mol_type = other DNA
atcttagctactgaccggctatagactcaatgatgataaaga

SEQ ID NO: 467 - DNA, 43 bp, synthetic construct
  misc_feature 1..43: note = Target Specific Oligonucleotide
  source 1..43: mol_type = other DNA
aagagattatacgcctcacggatagactcaatgatgataaaga

SEQ ID NO: 468 - DNA, 44 bp, synthetic construct
  misc_feature 1..44: note = Target Specific Oligonucleotide
  source 1..44: mol_type = other DNA
tttcgagtaacgaaattagctcctagactcaatgatgataaaga

SEQ ID NO: 469 - DNA, 39 bp, synthetic construct
  misc_feature 1..39: note = Target Specific Oligonucleotide
  source 1..39: mol_type = other DNA
gacccgcagattggcacgtagactcaatgatgataaaga

SEQ ID NO: 470 - DNA, 41 bp, synthetic construct
  misc_feature 1..41: note = Target Specific Oligonucleotide
  source 1..41: mol_type = other DNA
atggccccaatctcgtttggtagactcaatgatgataaaga

SEQ ID NO: 471 - DNA, 44 bp, synthetic construct
  misc_feature 1..44: note = Target Specific Oligonucleotide
  source 1..44: mol_type = other DNA
ccagtagctataacgaagtcctctagactcaatgatgataaaga

SEQ ID NO: 472 - DNA, 43 bp, synthetic construct
  misc_feature 1..43: note = Target Specific Oligonucleotide
  source 1..43: mol_type = other DNA
acaaattcccggacactatggatagactcaatgatgataaaga

SEQ ID NO: 473 - DNA, 43 bp, synthetic construct
  misc_feature 1..43: note = Target Specific Oligonucleotide
  source 1..43: mol_type = other DNA
ttcacagagttgataaggccactagactcaatgatgataaaga

SEQ ID NO: 474 - DNA, 43 bp, synthetic construct
  misc_feature 1..43: note = Target Specific Oligonucleotide
  source 1..43: mol_type = other DNA
aactttgccggtctctttacattagactcaatgatgataaaga

SEQ ID NO: 475 - DNA, 45 bp, synthetic construct
  misc_feature 1..45: note = Target Specific Oligonucleotide
  source 1..45: mol_type = other DNA
ttccaatgtgcaagaatgatttcttagactcaatgatgataaaga

SEQ ID NO: 476 - DNA, 43 bp, synthetic construct
  misc_feature 1..43: note = Target Specific Oligonucleotide
  source 1..43: mol_type = other DNA
ctccttctggggtatttcctgctagactcaatgatgataaaga

SEQ ID NO: 477 - DNA, 40 bp, synthetic construct
  misc_feature 1..40: note = Target Specific Oligonucleotide
  source 1..40: mol_type = other DNA
ccacttcagttggccggtatagactcaatgatgataaaga

SEQ ID NO: 478 - DNA, 39 bp, synthetic construct
  misc_feature 1..39: note = Target Specific Oligonucleotide
  source 1..39: mol_type = other DNA
tccgtcaacgtccgcagttagactcaatgatgataaaga

SEQ ID NO: 479 - DNA, 42 bp, synthetic construct
  misc_feature 1..42: note = Target Specific Oligonucleotide
  source 1..42: mol_type = other DNA
agtgagatcgccatagtgcaatagactcaatgatgataaaga

SEQ ID NO: 480 - DNA, 43 bp, synthetic construct
  misc_feature 1..43: note = Target Specific Oligonucleotide
  source 1..43: mol_type = other DNA
gaaggtgaccctataaggagtcatagactacaacaaaagatcg

SEQ ID NO: 481 - DNA, 41 bp, synthetic construct
  misc_feature 1..41: note = Target Specific Oligonucleotide
  source 1..41: mol_type = other DNA
aacaccgcttgcatagttgtgtagactacaacaaaagatcg

SEQ ID NO: 482 - DNA, 41 bp, synthetic construct
  misc_feature 1..41: note = Target Specific Oligonucleotide
  source 1..41: mol_type = other DNA
tacagacgcggaatcattctctagactacaacaaaagatcg

SEQ ID NO: 483 - DNA, 41 bp, synthetic construct
  misc_feature 1..41: note = Target Specific Oligonucleotide
  source 1..41: mol_type = other DNA
tggaggaaatgagcatgaccttagactacaacaaaagatcg

SEQ ID NO: 484 - DNA, 41 bp, synthetic construct
  misc_feature 1..41: note = Target Specific Oligonucleotide
  source 1..41: mol_type = other DNA
atggtgtacactcgaggctgatagactacaacaaaagatcg

SEQ ID NO: 485 - DNA, 41 bp, synthetic construct
  misc_feature 1..41: note = Target Specific Oligonucleotide
  source 1..41: mol_type = other DNA
ccttgtggtagacgttcagcttagactacaacaaaagatcg

SEQ ID NO: 486 - DNA, 44 bp, synthetic construct
  misc_feature 1..44: note = Target Specific Oligonucleotide
  source 1..44: mol_type = other DNA
ggatcaggtttaatggtcactatgtagactacaacaaaagatcg

SEQ ID NO: 487 - DNA, 40 bp, synthetic construct
  misc_feature 1..40: note = Target Specific Oligonucleotide
  source 1..40: mol_type = other DNA
cacacggtcaacgctgtacatagactacaacaaaagatcg

SEQ ID NO: 488 - DNA, 40 bp, synthetic construct
  misc_feature 1..40: note = Target Specific Oligonucleotide
  source 1..40: mol_type = other DNA
caggtgtttgcggaagttcctagactacaacaaaagatcg

SEQ ID NO: 489 - DNA, 40 bp, synthetic construct
  misc_feature 1..40: note = Target Specific Oligonucleotide
  source 1..40: mol_type = other DNA
gtgctcatggtcgtagaggatagactacaacaaaagatcg

SEQ ID NO: 490 - DNA, 43 bp, synthetic construct
  misc_feature 1..43: note = Target Specific Oligonucleotide
  source 1..43: mol_type = other DNA
aagtcaaagtacgtctcgatcattagactacaacaaaagatcg

SEQ ID NO: 491 - DNA, 39 bp, synthetic construct
  misc_feature 1..39: note = Target Specific Oligonucleotide
  source 1..39: mol_type = other DNA
tcgtagcagccgttggagatagactacaacaaaagatcg

SEQ ID NO: 492 - DNA, 39 bp, synthetic construct
  misc_feature 1..39: note = Target Specific Oligonucleotide
  source 1..39: mol_type = other DNA
tcgtggccagagtaggcattagactacaacaaaagatcg

SEQ ID NO: 493 - DNA, 40 bp, synthetic construct
  misc_feature 1..40: note = Target Specific Oligonucleotide
  source 1..40: mol_type = other DNA
ggtactctccaaacgctcggtagactacaacaaaagatcg

SEQ ID NO: 494 - DNA, 42 bp, synthetic construct
  misc_feature 1..42: note = Target Specific Oligonucleotide
  source 1..42: mol_type = other DNA
aggtacgaatagggatgtcgtctagactacaacaaaagatcg

SEQ ID NO: 495 - DNA, 42 bp, synthetic construct
  misc_feature 1..42: note = Target Specific Oligonucleotide
  source 1..42: mol_type = other DNA
agggtatcgtacatcgttccaatagactacaacaaaagatcg

SEQ ID NO: 496 - DNA, 39 bp, synthetic construct
  misc_feature 1..39: note = Target Specific Oligonucleotide
  source 1..39: mol_type = other DNA
caggtgatctgcgccgttgtagactacaacaaaagatcg

SEQ ID NO: 497 - DNA, 39 bp, synthetic construct
  misc_feature 1..39: note = Target Specific Oligonucleotide
  source 1..39: mol_type = other DNA
ccacaggcgatacaaccggtagactacaacaaaagatcg

SEQ ID NO: 498 - DNA, 39 bp, synthetic construct
  misc_feature 1..39: note = Target Specific Oligonucleotide
  source 1..39: mol_type = other DNA
cagcagcttcgagtgctggtagactacaacaaaagatcg

SEQ ID NO: 499 - DNA, 39 bp, synthetic construct
  misc_feature 1..39: note = Target Specific Oligonucleotide
  source 1..39: mol_type = other DNA
ccctcacaggacgtcgtcatagactacaacaaaagatcg

SEQ ID NO: 500 - DNA, 39 bp, synthetic construct
  misc_feature 1..39: note = Target Specific Oligonucleotide
  source 1..39: mol_type = other DNA
gcacgaagccttcggtgtctagactacaacaaaagatcg

SEQ ID NO: 501 - DNA, 41 bp, synthetic construct
  misc_feature 1..41: note = Target Specific Oligonucleotide
  source 1..41: mol_type = other DNA
tgggaagtgtcggctttcatgtagactacaacaaaagatcg

SEQ ID NO: 502 - DNA, 41 bp, synthetic construct; identical to SEQ ID NO: 501
  misc_feature 1..41: note = Target Specific Oligonucleotide
  source 1..41: mol_type = other DNA

SEQ ID NO: 503 - DNA, 38 bp, synthetic construct
  misc_feature 1..38: note = Target Specific Oligonucleotide
  source 1..38: mol_type = other DNA
tgctcgcactttggccgctagactacaacaaaagatcg

SEQ ID NO: 504 - DNA, 40 bp, synthetic construct
  misc_feature 1..40: note = Target Specific Oligonucleotide
  source 1..40: mol_type = other DNA
gtcttttggcacggtttctgttagactgtattcaacgtcc

SEQ ID NO: 505 - DNA, 40 bp, synthetic construct
  misc_feature 1..40: note = Target Specific Oligonucleotide
  source 1..40: mol_type = other DNA
tacttcttcaacgcgaagagctagactgtattcaacgtcc

SEQ ID NO: 506 - DNA, 40 bp, synthetic construct
  misc_feature 1..40: note = Target Specific Oligonucleotide
  source 1..40: mol_type = other DNA
tttgaggttgtatccgctgcttagactgtattcaacgtcc

SEQ ID NO: 507 - DNA, 44 bp, synthetic construct
  misc_feature 1..44: note = Target Specific Oligonucleotide
  source 1..44: mol_type = other DNA
caagttgactaaatctcgtactttctagactgtattcaacgtcc

SEQ ID NO: 508 - DNA, 43 bp, synthetic construct
  misc_feature 1..43: note = Target Specific Oligonucleotide
  source 1..43: mol_type = other DNA
gccttattaacggtatcttcagaatagactgtattcaacgtcc

SEQ ID NO: 509 - DNA, 40 bp, synthetic construct
  misc_feature 1..40: note = Target Specific Oligonucleotide
  source 1..40: mol_type = other DNA
tgttctggatttcgcaggtcctagactgtattcaacgtcc

SEQ ID NO: 510 - DNA, 42 bp, synthetic construct
  misc_feature 1..42: note = Target Specific Oligonucleotide
  source 1..42: mol_type = other DNA
gctttatcaggttatgttgcatgtagactgtattcaacgtcc

SEQ ID NO: 511 - DNA, 43 bp, synthetic construct
  misc_feature 1..43: note = Target Specific Oligonucleotide
  source 1..43: mol_type = other DNA
gattctggcttatagggtattcactagactgtattcaacgtcc

SEQ ID NO: 512 - DNA, 41 bp, synthetic construct
  misc_feature 1..41: note = Target Specific Oligonucleotide
  source 1..41: mol_type = other DNA
ttttcctcccgcaattcctagatagactgtattcaacgtcc

SEQ ID NO: 513 - DNA, 40 bp, synthetic construct
  misc_feature 1..40: note = Target Specific Oligonucleotide
  source 1..40: mol_type = other DNA
tgtttccgtcaaatcgtgtggtagactgtattcaacgtcc

SEQ ID NO: 514 - DNA, 41 bp, synthetic construct
  misc_feature 1..41: note = Target Specific Oligonucleotide
  source 1..41: mol_type = other DNA
ggttccctctagatcttgcctttagactgtattcaacgtcc

SEQ ID NO: 515 - DNA, 43 bp, synthetic construct
  misc_feature 1..43: note = Target Specific Oligonucleotide
  source 1..43: mol_type = other DNA
gcatgtaccacctatcatctaatgtagactgtattcaacgtcc

SEQ ID NO: 516 - DNA, 39 bp, synthetic construct
  misc_feature 1..39: note = Target Specific Oligonucleotide
  source 1..39: mol_type = other DNA
gttgccaacacgagctgacttagactgtattcaacgtcc

SEQ ID NO: 517 - DNA, 41 bp, synthetic construct
  misc_feature 1..41: note = Target Specific Oligonucleotide
  source 1..41: mol_type = other DNA
actgactactagttcaagcgcatagactgtattcaacgtcc

SEQ ID NO: 518 - DNA, 39 bp, synthetic construct
  misc_feature 1..39: note = Target Specific Oligonucleotide
  source 1..39: mol_type = other DNA
tctcttgctcgctttggacctagactgtattcaacgtcc

SEQ ID NO: 519 - DNA, 44 bp, synthetic construct
  misc_feature 1..44: note = Target Specific Oligonucleotide
  source 1..44: mol_type = other DNA
cactgctagaacaactatcaatttgtagactgtattcaacgtcc

SEQ ID NO: 520 - DNA, 41 bp, synthetic construct
  misc_feature 1..41: note = Target Specific Oligonucleotide
  source 1..41: mol_type = other DNA
ttacatggcttaagttggggagtagactgtattcaacgtcc

SEQ ID NO: 521 - DNA, 40 bp, synthetic construct
  misc_feature 1..40: note = Target Specific Oligonucleotide
  source 1..40: mol_type = other DNA
gtgaggggacgctcttgtatttagactgtattcaacgtcc

SEQ ID NO: 522 - DNA, 40 bp, synthetic construct
  misc_feature 1..40: note = Target Specific Oligonucleotide
  source 1..40: mol_type = other DNA
aacggtgctatgcctagtagatagactgtattcaacgtcc

SEQ ID NO: 523 - DNA, 40 bp, synthetic construct
  misc_feature 1..40: note = Target Specific Oligonucleotide
  source 1..40: mol_type = other DNA
tttgccttccctagagtgctatagactgtattcaacgtcc

SEQ ID NO: 524 - DNA, 43 bp, synthetic construct
  misc_feature 1..43: note = Target Specific Oligonucleotide
  source 1..43: mol_type = other DNA
atgtcccaatggatacttaaagcctagactgtattcaacgtcc

SEQ ID NO: 525 - DNA, 43 bp, synthetic construct
  misc_feature 1..43: note = Target Specific Oligonucleotide
  source 1..43: mol_type = other DNA
cttccagtaacgagatactttccttagactgtattcaacgtcc

SEQ ID NO: 526 - DNA, 42 bp, synthetic construct
  misc_feature 1..42: note = Target Specific Oligonucleotide
  source 1..42: mol_type = other DNA
caaccatgaattagtcccttgggtagactgtattcaacgtcc

SEQ ID NO: 527 - DNA, 44 bp, synthetic construct
  misc_feature 1..44: note = Target Specific Oligonucleotide
  source 1..44: mol_type = other DNA
cagcattattagacactttaactgttagactgtattcaacgtcc

SEQ ID NO: 528 - DNA, 43 bp, synthetic construct
  misc_feature 1..43: note = Target Specific Oligonucleotide
  source 1..43: mol_type = other DNA
tgcacatatgattgacgctcagtagactcttatattgagtggt

SEQ ID NO: 529 - DNA, 40 bp, synthetic construct
  misc_feature 1..40: note = Target Specific Oligonucleotide
  source 1..40: mol_type = other DNA
ttcaccgcctgcaacaagatagactcttatattgagtggt

SEQ ID NO: 530 - DNA, 44 bp, synthetic construct
  misc_feature 1..44: note = Target Specific Oligonucleotide
  source 1..44: mol_type = other DNA
ttcacttacaggtaacaccaagttagactcttatattgagtggt

SEQ ID NO: 531 - DNA, 45 bp, synthetic construct
  misc_feature 1..45: note = Target Specific Oligonucleotide
  source 1..45: mol_type = other DNA
aagcaatagatcgtccataagttatagactcttatattgagtggt

SEQ ID NO: 532 - DNA, 48 bp, synthetic construct
  misc_feature 1..48: note = Target Specific Oligonucleotide
  source 1..48: mol_type = other DNA
tacaagaaccctaattgtaataatagatagactcttatattgagtggt

SEQ ID NO: 533 - DNA, 44 bp, synthetic construct
  misc_feature 1..44: note = Target Specific Oligonucleotide
  source 1..44: mol_type = other DNA
gaataggaccaaagtgtcccttgtagactcttatattgagtggt

SEQ ID NO: 534 - DNA, 44 bp, synthetic construct
  misc_feature 1..44: note = Target Specific Oligonucleotide
  source 1..44: mol_type = other DNA
tccatcaggactaaatctcacactagactcttatattgagtggt

SEQ ID NO: 535 - DNA, 44 bp, synthetic construct
  misc_feature 1..44: note = Target Specific Oligonucleotide
  source 1..44: mol_type = other DNA
aaaggccatacgtttttcctacctagactcttatattgagtggt

SEQ ID NO: 536 - DNA, 44 bp, synthetic construct
  misc_feature 1..44: note = Target Specific Oligonucleotide
  source 1..44: mol_type = other DNA
gcagtgtacaatgaacgagaaattagactcttatattgagtggt

SEQ ID NO: 537 - DNA, 44 bp, synthetic construct
  misc_feature 1..44: note = Target Specific Oligonucleotide
  source 1..44: mol_type = other DNA
ggcattctgctttaagatcagaatagactcttatattgagtggt

SEQ ID NO: 538 - DNA, 40 bp, synthetic construct
  misc_feature 1..40: note = Target Specific Oligonucleotide
  source 1..40: mol_type = other DNA
ggccagccacgattcacagtagactcttatattgagtggt

SEQ ID NO: 539 - DNA, 39 bp, synthetic construct
  misc_feature 1..39: note = Target Specific Oligonucleotide
  source 1..39: mol_type = other DNA
gaagccagcgaccggacttagactcttatattgagtggt

SEQ ID NO: 540 - DNA, 41 bp, synthetic construct
  misc_feature 1..41: note = Target Specific Oligonucleotide
  source 1..41: mol_type = other DNA
ctgagggtcgtcgttgtctttagactcttatattgagtggt

SEQ ID NO: 541 - DNA, 43 bp, synthetic construct
  misc_feature 1..43: note = Target Specific Oligonucleotide
  source 1..43: mol_type = other DNA
ctgaaccactggcatagagttctagactcttatattgagtggt

SEQ ID NO: 542 - DNA, 40 bp, synthetic construct
  misc_feature 1..40: note = Target Specific Oligonucleotide
  source 1..40: mol_type = other DNA
aaatcaaccacgggtcgcgtagactcttatattgagtggt

SEQ ID NO: 543 - DNA, 40 bp, synthetic construct
  misc_feature 1..40: note = Target Specific Oligonucleotide
  source 1..40: mol_type = other DNA
agcaacagtcgacgagggatagactcttatattgagtggt

SEQ ID NO: 544 - DNA, 48 bp, synthetic construct
  misc_feature 1..48: note = Target Specific Oligonucleotide
  source 1..48: mol_type = other DNA
atatattccatactactaacagacatatagactcttatattgagtggt

SEQ ID NO: 545 - DNA, 38 bp, synthetic construct
  misc_feature 1..38: note = Target Specific Oligonucleotide
  source 1..38: mol_type = other DNA
tccacaccggcatcggctagactcttatattgagtggt

SEQ ID NO: 546 - DNA, 40 bp, synthetic construct
  misc_feature 1..40: note = Target Specific Oligonucleotide
  source 1..40: mol_type = other DNA
agaagggcaatccggtggctagactcttatattgagtggt

SEQ ID NO: 547 - DNA, 40 bp, synthetic construct
  misc_feature 1..40: note = Target Specific Oligonucleotide
  source 1..40: mol_type = other DNA
tcagggagtctgcgaccagtagactcttatattgagtggt

SEQ ID NO: 548 - DNA, 40 bp, synthetic construct
  misc_feature 1..40: note = Target Specific Oligonucleotide
  source 1..40: mol_type = other DNA
gctttgccagctcaccacttagactcttatattgagtggt

SEQ ID NO: 549 - DNA, 43 bp, synthetic construct
  misc_feature 1..43: note = Target Specific Oligonucleotide
  source 1..43: mol_type = other DNA
aaatacccataaggcgtgatgctagactcttatattgagtggt

SEQ ID NO: 550 - DNA, 44 bp, synthetic construct
  misc_feature 1..44: note = Target Specific Oligonucleotide
  source 1..44: mol_type = other DNA
attactatcctgcgtgaaatccatagactcttatattgagtggt

SEQ ID NO: 551 - DNA, 43 bp, synthetic construct
  misc_feature 1..43: note = Target Specific Oligonucleotide
  source 1..43: mol_type = other DNA
cccaaagtcgaacagttttgtctagactcttatattgagtggt

SEQ ID NO: 552 - DNA, 41 bp, synthetic construct
  misc_feature 1..41: note = Target Specific Oligonucleotide
  source 1..41: mol_type = other DNA
tcctcaaatgtccgaggatgatagacctattctatgcttcg

SEQ ID NO: 553 - DNA, 42 bp, synthetic construct
  misc_feature 1..42: note = Target Specific Oligonucleotide
  source 1..42: mol_type = other DNA
gtgaacacaacgtaagaacaggtagacctattctatgcttcg

SEQ ID NO: 554 - DNA, 42 bp, synthetic construct
  misc_feature 1..42: note = Target Specific Oligonucleotide
  source 1..42: mol_type = other DNA
actcctctggttacgcttcatttagacctattctatgcttcg

SEQ ID NO: 555 - DNA, 41 bp, synthetic construct
  misc_feature 1..41: note = Target Specific Oligonucleotide
  source 1..41: mol_type = other DNA
acctaacataaccgtgctgcctagacctattctatgcttcg

SEQ ID NO: 556 - DNA, 41 bp, synthetic construct
  misc_feature 1..41: note = Target Specific Oligonucleotide
  source 1..41: mol_type = other DNA
ttcagttgaccgtctggacactagacctattctatgcttcg

SEQ ID NO: 557 - DNA, 43 bp, synthetic construct
  misc_feature 1..43: note = Target Specific Oligonucleotide
  source 1..43: mol_type = other DNA
ctctgacacacgtaacaataacatagacctattctatgcttcg

SEQ ID NO: 558 - DNA, 43 bp, synthetic construct
  misc_feature 1..43: note = Target Specific Oligonucleotide
  source 1..43: mol_type = other DNA
agtaatgtgaggtacaactgcattagacctattctatgcttcg

SEQ ID NO: 559 - DNA, 41 bp, synthetic construct
  misc_feature 1..41: note = Target Specific Oligonucleotide
  source 1..41: mol_type = other DNA
gcttctttcccgcccaaaatatagacctattctatgcttcg

SEQ ID NO: 560 - DNA, 44 bp, synthetic construct
  misc_feature 1..44: note = Target Specific Oligonucleotide
  source 1..44: mol_type = other DNA
aaagaaaagcggtctactagattatagacctattctatgcttcg

SEQ ID NO: 561 - DNA, 41 bp, synthetic construct
  misc_feature 1..41: note = Target Specific Oligonucleotide
  source 1..41: mol_type = other DNA
tgacgtgcggcgaaatttatgtagacctattctatgcttcg

SEQ ID NO: 562 - DNA, 41 bp, synthetic construct
  misc_feature 1..41: note = Target Specific Oligonucleotide
  source 1..41: mol_type = other DNA
gcttattgcagcgatggatgatagacctattctatgcttcg

SEQ ID NO: 563 - DNA, 42 bp, synthetic construct
  misc_feature 1..42: note = Target Specific Oligonucleotide
  source 1..42: mol_type = other DNA
tgtcagtagacgatagaggaggtagacctattctatgcttcg

SEQ ID NO: 564 - DNA, 41 bp, synthetic construct
  misc_feature 1..41: note = Target Specific Oligonucleotide
  source 1..41: mol_type = other DNA
ttggttccgactacccaacagtagacctattctatgcttcg

SEQ ID NO: 565 - DNA, 45 bp, synthetic construct
  misc_feature 1..45: note = Target Specific Oligonucleotide
  source 1..45: mol_type = other DNA
ctcttaagtacgcaataattctctctagacctattctatgcttcg

SEQ ID NO: 566 - DNA, 43 bp, synthetic construct
  misc_feature 1..43: note = Target Specific Oligonucleotide
  source 1..43: mol_type = other DNA
gttccgtatatgtcggatctctctagacctattctatgcttcg

SEQ ID NO: 567 - DNA, 41 bp, synthetic construct
  misc_feature 1..41: note = Target Specific Oligonucleotide
  source 1..41: mol_type = other DNA
aaattcaccggacggagtgtttagacctattctatgcttcg

SEQ ID NO: 568 - DNA, 42 bp, synthetic construct
  misc_feature 1..42: note = Target Specific Oligonucleotide
  source 1..42: mol_type = other DNA
tttgtgaaaccgtagtggctcttagacctattctatgcttcg

SEQ ID NO: 569 - DNA, 42 bp, synthetic construct
  misc_feature 1..42: note = Target Specific Oligonucleotide
  source 1..42: mol_type = other DNA
acctctttaacgaaggtgtcagtagacctattctatgcttcg

SEQ ID NO: 570 - DNA, 41 bp, synthetic construct
  misc_feature 1..41: note = Target Specific Oligonucleotide
  source 1..41: mol_type = other DNA
ggactccggttctaacttggttagacctattctatgcttcg

SEQ ID NO: 571 - DNA, 43 bp, synthetic construct
  misc_feature 1..43: note = Target Specific Oligonucleotide
  source 1..43: mol_type = other DNA
atctctatatgacgtgctgttggtagacctattctatgcttcg

SEQ ID NO: 572 - DNA, 43 bp, synthetic construct
  misc_feature 1..43: note = Target Specific Oligonucleotide
  source 1..43: mol_type = other DNA
gttggaattattcggagactgagtagacctattctatgcttcg

SEQ ID NO: 573 - DNA, 40 bp, synthetic construct
  misc_feature 1..40: note = Target Specific Oligonucleotide
  source 1..40: mol_type = other DNA
gcacatttgacgacggcttctagacctattctatgcttcg

SEQ ID NO: 574 - DNA, 40 bp, synthetic construct
  misc_feature 1..40: note = Target Specific Oligonucleotide
  source 1..40: mol_type = other DNA
actccgtgtaaacgcagtggtagacctattctatgcttcg

SEQ ID NO: 575 - DNA, 43 bp, synthetic construct
  misc_feature 1..43: note = Target Specific Oligonucleotide
  source 1..43: mol_type = other DNA
acacataactcgttaacacgttctagacctattctatgcttcg

SEQ ID NO: 576 - DNA, 42 bp, synthetic construct
  misc_feature 1..42: note = Target Specific Oligonucleotide
  source 1..42: mol_type = other DNA
cagatctgttgagctaacagggtagacatgttataccatgcg

SEQ ID NO: 577 - DNA, 44 bp, synthetic construct
  misc_feature 1..44: note = Target Specific Oligonucleotide
  source 1..44: mol_type = other DNA
ttctagactatgcaaccctctaggtagacatgttataccatgcg

SEQ ID NO: 578 - DNA, 43 bp, synthetic construct
  misc_feature 1..43: note = Target Specific Oligonucleotide
  source 1..43: mol_type = other DNA
cacccatgtgagtaatacactgctagacatgttataccatgcg

SEQ ID NO: 579 - DNA, 42 bp, synthetic construct
  misc_feature 1..42: note = Target Specific Oligonucleotide
  source 1..42: mol_type = other DNA
agtcccatattgccgttcatgctagacatgttataccatgcg

SEQ ID NO: 580 - DNA, 42 bp, synthetic construct
  misc_feature 1..42: note = Target Specific Oligonucleotide
  source 1..42: mol_type = other DNA
atttcctcataacggtcatggctagacatgttataccatgcg

SEQ ID NO: 581 - DNA, 42 bp, synthetic construct
  misc_feature 1..42: note = Target Specific Oligonucleotide
  source 1..42: mol_type = other DNA
ttactgcctagctagcaggttatagacatgttataccatgcg

SEQ ID NO: 582 - DNA, 41 bp, synthetic construct
  misc_feature 1..41: note = Target Specific Oligonucleotide
  source 1..41: mol_type = other DNA
acgtgcccttggtactatgactagacatgttataccatgcg

SEQ ID NO: 583 - DNA, 41 bp, synthetic construct
  misc_feature 1..41: note = Target Specific Oligonucleotide
  source 1..41: mol_type = other DNA
cttgccaaaccctagcttggatagacatgttataccatgcg

SEQ ID NO: 584 - DNA, 45 bp, synthetic construct
  misc_feature 1..45: note = Target Specific Oligonucleotide
  source 1..45: mol_type = other DNA
attatcacttaacgaaggtcctttgtagacatgttataccatgcg

SEQ ID NO: 585 - DNA, 45 bp, synthetic construct
  misc_feature 1..45: note = Target Specific Oligonucleotide
  source 1..45: mol_type = other DNA
atatacagcctaagcaccaattatgtagacatgttataccatgcg

SEQ ID NO: 586 - DNA, 40 bp, synthetic construct
  misc_feature 1..40: note = Target Specific Oligonucleotide
  source 1..40: mol_type = other DNA
ggcaacaaaacgtcatggcatagacatgttataccatgcg

SEQ ID NO: 587 - DNA, 43 bp, synthetic construct
  misc_feature 1..43: note = Target Specific Oligonucleotide
  source 1..43: mol_type = other DNA
gtctgaggactaaccctaaagggtagacatgttataccatgcg

SEQ ID NO: 588 - DNA, 46 bp, synthetic construct
  misc_feature 1..46: note = Target Specific Oligonucleotide
  source 1..46: mol_type = other DNA
cttaaaatagatcgaaactctgtctctagacatgttataccatgcg

SEQ ID NO: 589 - DNA, 44 bp, synthetic construct
  misc_feature 1..44: note = Target Specific Oligonucleotide
  source 1..44: mol_type = other DNA
tgatctgtacaagctacgattttatagacatgttataccatgcg

SEQ ID NO: 590 - DNA, 43 bp, synthetic construct
  misc_feature 1..43: note = Target Specific Oligonucleotide
  source 1..43: mol_type = other DNA
aaatcagttataccaaggggagatagacatgttataccatgcg

SEQ ID NO: 591 - DNA, 45 bp, synthetic construct
  misc_feature 1..45: note = Target Specific Oligonucleotide
  source 1..45: mol_type = other DNA
taggaaaacatacgtatactgaatatagacatgttataccatgcg

SEQ ID NO: 592 - DNA, 40 bp, synthetic construct
  misc_feature 1..40: note = Target Specific Oligonucleotide
  source 1..40: mol_type = other DNA
agttggcctcacgttgcatttagacatgttataccatgcg

SEQ ID NO: 593 - DNA, 40 bp, synthetic construct
  misc_feature 1..40: note = Target Specific Oligonucleotide
  source 1..40: mol_type = other DNA
tttggtctcggcttgcgaattagacatgttataccatgcg

SEQ ID NO: 594 - DNA, 41 bp, synthetic construct
  misc_feature 1..41: note = Target Specific Oligonucleotide
  source 1..41: mol_type = other DNA
cagtagtcaacgcagcactcatagacatgttataccatgcg

SEQ ID NO: 595 - DNA, 44 bp, synthetic construct
  misc_feature 1..44: note = Target Specific Oligonucleotide
  source 1..44: mol_type = other DNA
caacaggatcacctaatattcccatagacatgttataccatgcg

SEQ ID NO: 596 - DNA, 43 bp, synthetic construct
  misc_feature 1..43: note = Target Specific Oligonucleotide
  source 1..43: mol_type = other DNA
tttggacagatatctcctcgaaatagacatgttataccatgcg

SEQ ID NO: 597 - DNA, 43 bp, synthetic construct
  misc_feature 1..43: note = Target Specific Oligonucleotide
  source 1..43: mol_type = other DNA
aaaggctatctacactttggcaatagacatgttataccatgcg

SEQ ID NO: 598 - DNA, 40 bp, synthetic construct
  misc_feature 1..40: note = Target Specific Oligonucleotide
  source 1..40: mol_type = other DNA
tgcctggatcctatcccgactagacatgttataccatgcg

SEQ ID NO: 599 - DNA, 40 bp, synthetic construct
  misc_feature 1..40: note = Target Specific Oligonucleotide
  source 1..40: mol_type = other DNA
tgtggtcacctacacgctgctagacatgttataccatgcg

SEQ ID NO: 600 - DNA, 43 bp, synthetic construct
  misc_feature 1..43: note = Target Specific Oligonucleotide
  source 1..43: mol_type = other DNA
ggtgagctggtaatctgaccttagacaatctatttaccctacg

SEQ ID NO: 601 - DNA, 41 bp, synthetic construct
  misc_feature 1..41: note = Target Specific Oligonucleotide
  source 1..41: mol_type = other DNA
gttgcaggcatgacgcaagtagacaatctatttaccctacg

SEQ ID NO: 602 - DNA, 43 bp, synthetic construct
  misc_feature 1..43: note = Target Specific Oligonucleotide
  source 1..43: mol_type = other DNA
aaaacgcctacgcatcatgtctagacaatctatttaccctacg

SEQ ID NO: 603 - DNA, 40 bp, synthetic construct
  misc_feature 1..40: note = Target Specific Oligonucleotide
  source 1..40: mol_type = other DNA
tggtcctgcgcggatgtctagacaatctatttaccctacg

SEQ ID NO: 604 - DNA, 41 bp, synthetic construct
  misc_feature 1..41: note = Target Specific Oligonucleotide
  source 1..41: mol_type = other DNA
ggtgagcttctccgtggcgtagacaatctatttaccctacg

SEQ ID NO: 605 - DNA, 41 bp, synthetic construct
  misc_feature 1..41: note = Target Specific Oligonucleotide
  source 1..41: mol_type = other DNA
agcgcagggccaattgacttagacaatctatttaccctacg

SEQ ID NO: 606 - DNA, 43 bp, synthetic construct
  misc_feature 1..43: note = Target Specific Oligonucleotide
  source 1..43: mol_type = other DNA
ccgggtacacggttttgatcttagacaatctatttaccctacg

SEQ ID NO: 607 - DNA, 40 bp, synthetic construct
  misc_feature 1..40: note = Target Specific Oligonucleotide
  source 1..40: mol_type = other DNA
ccggctcgatgtcgggtatagacaatctatttaccctacg

SEQ ID NO: 608 - DNA, 40 bp, synthetic construct
  misc_feature 1..40: note = Target Specific Oligonucleotide
  source 1..40: mol_type = other DNA
ggcgccttctcgccacattagacaatctatttaccctacg

SEQ ID NO: 609 - DNA, 40 bp, synthetic construct
  misc_feature 1..40: note = Target Specific Oligonucleotide
  source 1..40: mol_type = other DNA
cacgttgaagcgcgggtgtagacaatctatttaccctacg

SEQ ID NO: 610 - DNA, 44 bp, synthetic construct
  misc_feature 1..44: note = Target Specific Oligonucleotide
  source 1..44: mol_type = other DNA
gcccacagaaaccggtttaatgtagacaatctatttaccctacg

SEQ ID NO: 611 - DNA, 43 bp, synthetic construct
  misc_feature 1..43: note = Target Specific Oligonucleotide
  source 1..43: mol_type = other DNA
tcagaatgagcgcaatccaggtagacaatctatttaccctacg

SEQ ID NO: 612 - DNA, 42 bp, synthetic construct
  misc_feature 1..42: note = Target Specific Oligonucleotide
  source 1..42: mol_type = other DNA
ttcggacacaaagacgctcctagacaatctatttaccctacg

SEQ ID NO: 613 - DNA, 42 bp, synthetic construct
  misc_feature 1..42: note = Target Specific Oligonucleotide
  source 1..42: mol_type = other DNA
agcttgacgtaggtgtcggttagacaatctatttaccctacg

SEQ ID NO: 614 - DNA, 42 bp, synthetic construct
  misc_feature 1..42: note = Target Specific Oligonucleotide
  source 1..42: mol_type = other DNA
ggttcccgccaaatttcccgtagacaatctatttaccctacg

SEQ ID NO: 615 - DNA, 45 bp, synthetic construct
  misc_feature 1..45: note = Target Specific Oligonucleotide
  source 1..45: mol_type = other DNA
cagggctcatgatagtacaacagtagacaatctatttaccctacg

SEQ ID NO: 616 - DNA, 41 bp, synthetic construct
  misc_feature 1..41: note = Target Specific Oligonucleotide
  source 1..41: mol_type = other DNA
ggactttagcgcggtctcctagacaatctatttaccctacg

SEQ ID NO: 617 - DNA, 41 bp, synthetic construct
  misc_feature 1..41: note = Target Specific Oligonucleotide
  source 1..41: mol_type = other DNA
gcagccgttctaagcgctgtagacaatctatttaccctacg

SEQ ID NO: 618 - DNA, 41 bp, synthetic construct
  misc_feature 1..41: note = Target Specific Oligonucleotide
  source 1..41: mol_type = other DNA
gctggtaaaggcgcttccctagacaatctatttaccctacg

SEQ ID NO: 619 - DNA, 41 bp, synthetic construct
  misc_feature 1..41: note = Target Specific Oligonucleotide
  source 1..41: mol_type = other DNA
gaagaagtcggtgacgcggtagacaatctatttaccctacg

SEQ ID NO: 620 - DNA, 41 bp, synthetic construct
  misc_feature 1..41: note = Target Specific Oligonucleotide
  source 1..41: mol_type = other DNA
ggctggaaacctcgtccactagacaatctatttaccctacg

SEQ ID NO: 621 - DNA, 48 bp, synthetic construct
  misc_feature 1..48: note = Target Specific Oligonucleotide
  source 1..48: mol_type = other DNA
tttattagagtttaggaggtcagaaatagacaatctatttaccctacg

SEQ ID NO: 622 - DNA, 44 bp, synthetic construct
  misc_feature 1..44: note = Target Specific Oligonucleotide
  source 1..44: mol_type = other DNA
tgggaggtcccatattcagaggtagacaatctatttaccctacg

SEQ ID NO: 623 - DNA, 44 bp, synthetic construct
  misc_feature 1..44: note = Target Specific Oligonucleotide
  source 1..44: mol_type = other DNA
tccagggatgtcctacttgtcctagacaatctatttaccctacg

SEQ ID NO: 624 - DNA, 40 bp, synthetic construct
  misc_feature 1..40: note = Target Specific Oligonucleotide
  source 1..40: mol_type = other DNA
ccaggtttccggcacagtgggtaactgaattgtcacttta

SEQ ID NO: 625 - DNA, 46 bp, synthetic construct
  misc_feature 1..46: note = Target Specific Oligonucleotide
  source 1..46: mol_type = other DNA
tgttatactttgtcgctcttagtagagtaactgaattgtcacttta

SEQ ID NO: 626 - DNA, 44 bp, synthetic construct
  misc_feature 1..44: note = Target Specific Oligonucleotide
  source 1..44: mol_type = other DNA
agcttttaaccaggtttcgacttcgtaactgaattgtcacttta

SEQ ID NO: 627 - DNA, 45 bp, synthetic construct
  misc_feature 1..45: note = Target Specific Oligonucleotide
  source 1..45: mol_type = other DNA
gtatgagaatcccgataaaactggtgtaactgaattgtcacttta

SEQ ID NO: 628 - DNA, 43 bp, synthetic construct
  misc_feature 1..43: note = Target Specific Oligonucleotide
  source 1..43: mol_type = other DNA
aagaggacatcgaccaatcctcagtaactgaattgtcacttta

SEQ ID NO: 629 - DNA, 42 bp, synthetic construct
  misc_feature 1..42: note = Target Specific Oligonucleotide
  source 1..42: mol_type = other DNA
ggtttgtgcaagggtcgaaaacgtaactgaattgtcacttta

SEQ ID NO: 630 - DNA, 44 bp, synthetic construct
  misc_feature 1..44: note = Target Specific Oligonucleotide
  source 1..44: mol_type = other DNA
acacctctcgtagctatagatgtggtaactgaattgtcacttta

SEQ ID NO: 631 - DNA, 41 bp, synthetic construct
  misc_feature 1..41: note = Target Specific Oligonucleotide
  source 1..41: mol_type = other DNA
atgagagggcaaccgaggtgagtaactgaattgtcacttta

SEQ ID NO: 632 - DNA, 43 bp, synthetic construct
  misc_feature 1..43: note = Target Specific Oligonucleotide
  source 1..43: mol_type = other DNA
ccgctgtcagggtctatcattccgtaactgaattgtcacttta

SEQ ID NO: 633 - DNA, 41 bp, synthetic construct
  misc_feature 1..41: note = Target Specific Oligonucleotide
  source 1..41: mol_type = other DNA
gcaatgagcatatcctcgggcgtaactgaattgtcacttta

SEQ ID NO: 634 - DNA, 41 bp, synthetic construct
  misc_feature 1..41: note = Target Specific Oligonucleotide
  source 1..41: mol_type = other DNA
ggcttgagtgggttcacccaggtaactgaattgtcacttta

SEQ ID NO: 635 - DNA, 43 bp, synthetic construct
  misc_feature 1..43: note = Target Specific Oligonucleotide
  source 1..43: mol_type = other DNA
gcaaggtatggcaatagctgagtgtaactgaattgtcacttta

SEQ ID NO: 636 - DNA, 45 bp, synthetic construct
  misc_feature 1..45: note = Target Specific Oligonucleotide
  source 1..45: mol_type = other DNA
cagtaatagtagagacgccactacagtaactgaattgtcacttta

SEQ ID NO: 637 - DNA, 43 bp, synthetic construct
  misc_feature 1..43: note = Target Specific Oligonucleotide
  source 1..43: mol_type = other DNA
cttccaaagtgtcgcttcagagggtaactgaattgtcacttta

SEQ ID NO: 638 - DNA, 46 bp, synthetic construct
  misc_feature 1..46: note = Target Specific Oligonucleotide
  source 1..46: mol_type = other DNA
aggcaattttcccgatactttttattgtaactgaattgtcacttta

SEQ ID NO: 639 - DNA, 46 bp, synthetic construct
  misc_feature 1..46: note = Target Specific Oligonucleotide
  source 1..46: mol_type = other DNA
tgttagcttctatagtcactattcgagtaactgaattgtcacttta

SEQ ID NO: 640 - DNA, 45 bp, synthetic construct
  misc_feature 1..45: note = Target Specific Oligonucleotide
  source 1..45: mol_type = other DNA
tcaacaagtaatgtatcccgttcatgtaactgaattgtcacttta

SEQ ID NO: 641 - DNA, 44 bp, synthetic construct
  misc_feature 1..44: note = Target Specific Oligonucleotide
  source 1..44: mol_type = other DNA
tttccttggccgtaagttgttttcgtaactgaattgtcacttta

SEQ ID NO: 642 - DNA, 42 bp, synthetic construct
  misc_feature 1..42: note = Target Specific Oligonucleotide
  source 1..42: mol_type = other DNA
gggtcaacccagtctgttacctgtaactgaattgtcacttta

SEQ ID NO: 643 - DNA, 46 bp, synthetic construct
  misc_feature 1..46: note = Target Specific Oligonucleotide
  source 1..46: mol_type = other DNA
tctgagactgatagggaaacatatgggtaactgaattgtcacttta

SEQ ID NO: 644 - DNA, 42 bp, synthetic construct
  misc_feature 1..42: note = Target Specific Oligonucleotide
  source 1..42: mol_type = other DNA
tctttgccaccaatagcttggagtaactgaattgtcacttta

SEQ ID NO: 645 - DNA, 45 bp, synthetic construct
  misc_feature 1..45: note = Target Specific Oligonucleotide
  source 1..45: mol_type = other DNA
ctcattggaatggttagtagagcaagtaactgaattgtcacttta

SEQ ID NO: 646 - DNA, 44 bp, synthetic construct
  misc_feature 1..44: note = Target Specific Oligonucleotide
  source 1..44: mol_type = other DNA
ctgagtaaagagcgtgtattccaggtaactgaattgtcacttta

SEQ ID NO: 647 - DNA, 42 bp, synthetic construct
  misc_feature 1..42: note = Target Specific Oligonucleotide
  source 1..42: mol_type = other DNA
tggaggcataaagctggtaggcgtaactgaattgtcacttta

SEQ ID NO: 648 - DNA, 42 bp, synthetic construct
  misc_feature 1..42: note = Target Specific Oligonucleotide
  source 1..42: mol_type = other DNA
ggatctcaagcgttgcaggcgtaacgatagatttatacgacg

SEQ ID NO: 649 - DNA, 39 bp, synthetic construct
  misc_feature 1..39: note = Target Specific Oligonucleotide
  source 1..39: mol_type = other DNA
gcaccgcgagcggactagtaacgatagatttatacgacg

SEQ ID NO: 650 - DNA, 41 bp, synthetic construct
  misc_feature 1..41: note = Target Specific Oligonucleotide
  source 1..41: mol_type = other DNA
tgctgaggtcgctcacgaagtaacgatagatttatacgacg

SEQ ID NO: 651 - DNA, 44 bp, synthetic construct
  misc_feature 1..44: note = Target Specific Oligonucleotide
  source 1..44: mol_type = other DNA
ccggaccacgtagttaaatcttgtaacgatagatttatacgacg

SEQ ID NO: 652 - DNA, 43 bp, synthetic construct
  misc_feature 1..43: note = Target Specific Oligonucleotide
  source 1..43: mol_type = other DNA
aagttcttttgggcgatgccagtaacgatagatttatacgacg

SEQ ID NO: 653 - DNA, 48 bp, synthetic construct
  misc_feature 1..48: note = Target Specific Oligonucleotide
  source 1..48: mol_type = other DNA
gttcccttattagtctaatgttttgcgtaacgatagatttatacgacg

SEQ ID NO: 654 - DNA, 45 bp, synthetic construct
  misc_feature 1..45: note = Target Specific Oligonucleotide
  source 1..45: mol_type = other DNA
ttggaccagtgtacataaggatggtaacgatagatttatacgacg

SEQ ID NO: 655 - DNA, 41 bp, synthetic construct
  misc_feature 1..41: note = Target Specific Oligonucleotide
  source 1..41: mol_type = other DNA
tggcagtgcacggatggtcgtaacgatagatttatacgacg

SEQ ID NO: 656 - DNA, 47 bp, synthetic construct
  misc_feature 1..47: note = Target Specific Oligonucleotide
  source 1..47: mol_type = other DNA
ataccaattctagtgagaagttcacgtaacgatagatttatacgacg

SEQ ID NO: 657 - DNA, 44 bp, synthetic construct
  misc_feature 1..44: note = Target Specific Oligonucleotide
  source 1..44: mol_type = other DNA
tctctgtagggtaaggctctgtgtaacgatagatttatacgacg

SEQ ID NO: 658 - DNA, 45 bp, synthetic construct
  misc_feature 1..45: note = Target Specific Oligonucleotide
  source 1..45: mol_type = other DNA
tttagaggtagaccattgtgtgggtaacgatagatttatacgacg

SEQ ID NO: 659 - DNA, 42 bp, synthetic construct
  misc_feature 1..42: note = Target Specific Oligonucleotide
  source 1..42: mol_type = other DNA
agagagggcactctacgcttgtaacgatagatttatacgacg

SEQ ID NO: 660 - DNA, 44 bp, synthetic construct
  misc_feature 1..44: note = Target Specific Oligonucleotide
  source 1..44: mol_type = other DNA
caaaaagccctagctcagatgggtaacgatagatttatacgacg

SEQ ID NO: 661 - DNA, 43 bp, synthetic construct
  misc_feature 1..43: note = Target Specific Oligonucleotide
  source 1..43: mol_type = other DNA
cctctcctccctatttggctggtaacgatagatttatacgacg

SEQ ID NO: 662 - DNA, 46 bp, synthetic construct
  misc_feature 1..46: note = Target Specific Oligonucleotide
  source 1..46: mol_type = other DNA
ttttacaacgaactcattaggtccgtaacgatagatttatacgacg

SEQ ID NO: 663 - DNA, 45 bp, synthetic construct
  misc_feature 1..45: note = Target Specific Oligonucleotide
  source 1..45: mol_type = other DNA
ggagtagatcccccatctattccgtaacgatagatttatacgacg

SEQ ID NO: 664 - DNA, 44 bp, synthetic construct
  misc_feature 1..44: note = Target Specific Oligonucleotide
  source 1..44: mol_type = other DNA
gtctcttgcctcgtttctctccgtaacgatagatttatacgacg

SEQ ID NO: 665 - DNA, 43 bp, synthetic construct
  misc_feature 1..43: note = Target Specific Oligonucleotide
  source 1..43: mol_type = other DNA
aattctggcgtagcacaaacagtaacgatagatttatacgacg

SEQ ID NO: 666 - DNA, 45 bp, synthetic construct
  misc_feature 1..45: note = Target Specific Oligonucleotide
  source 1..45: mol_type = other DNA
ggccatgaacccctaaatctaaagtaacgatagatttatacgacg

SEQ ID NO: 667 - DNA, 42 bp, synthetic construct
  misc_feature 1..42: note = Target Specific Oligonucleotide
  source 1..42: mol_type = other DNA
gcccactggtaccatcttgggtaacgatagatttatacgacg

SEQ ID NO: 668 - DNA, 42 bp, synthetic construct
  misc_feature 1..42: note = Target Specific Oligonucleotide
  source 1..42: mol_type = other DNA
taccacgtggcactaacacagtaacgatagatttatacgacg

SEQ ID NO: 669 - DNA, 45 bp, synthetic construct
  misc_feature 1..45: note = Target Specific Oligonucleotide
  source 1..45: mol_type = other DNA
ttctagcttttagaccagcagttgtaacgatagatttatacgacg

SEQ ID NO: 670 - DNA, 44 bp, synthetic construct
  misc_feature 1..44: note = Target Specific Oligonucleotide
  source 1..44: mol_type = other DNA
cccaagtgctagtagaacctgcgtaacgatagatttatacgacg

SEQ ID NO: 671 - DNA, 47 bp, synthetic construct
  misc_feature 1..47: note = Target Specific Oligonucleotide
  source 1..47: mol_type = other DNA
aaacacatctatactcaaagacagagtaacgatagatttatacgacg

SEQ ID NO: 672 - DNA, 43 bp, synthetic construct
  misc_feature 1..43: note = Target Specific Oligonucleotide
  source 1..43: mol_type = other DNA
cacagacttgccgactctttggtaacgatagatttatacgacg

SEQ ID NO: 673 - DNA, 44 bp, synthetic construct
  misc_feature 1..44: note = Target Specific Oligonucleotide
  source 1..44: mol_type = other DNA
cacatgttaatgttcccgaccagtaacgatagatttatacgacg

SEQ ID NO: 674 - DNA, 44 bp, synthetic construct
  misc_feature 1..44: note = Target Specific Oligonucleotide
  source 1..44: mol_type = other DNA
attaatcgtttgcggatctgccgtaacgatagatttatacgacg

SEQ ID NO: 675 - DNA, 44 bp, synthetic construct
  misc_feature 1..44: note = Target Specific Oligonucleotide
  source 1..44: mol_type = other DNA
tgggtaacatcggacaagtttggtaacgatagatttatacgacg

SEQ ID NO: 676 - DNA, 44 bp, synthetic construct
  misc_feature 1..44: note = Target Specific Oligonucleotide
  source 1..44: mol_type = other DNA
tctgaagtttcgagccaatgttgtaacgatagatttatacgacg

SEQ ID NO: 677 - DNA, 44 bp, synthetic construct
  misc_feature 1..44: note = Target Specific Oligonucleotide
  source 1..44: mol_type = other DNA
ccattttcaccgctgaaatagagtaacgatagatttatacgacg

SEQ ID NO: 678 - DNA, 43 bp, synthetic construct
  misc_feature 1..43: note = Target Specific Oligonucleotide
  source 1..43: mol_type = other DNA
ctggatcacatgcgttgtcaggtaacgatagatttatacgacg

SEQ ID NO: 679 - DNA, 45 bp, synthetic construct
  misc_feature 1..45: note = Target Specific Oligonucleotide
  source 1..45: mol_type = other DNA
atatttgcctcgtaagatcccctgtaacgatagatttatacgacg

SEQ ID NO: 680 - DNA, 45 bp, synthetic construct
  misc_feature 1..45: note = Target Specific Oligonucleotide
  source 1..45: mol_type = other DNA
ctgaagcggtgttgtatcatagcgtaacgatagatttatacgacg

SEQ ID NO: 681 - DNA, 45 bp, synthetic construct
  misc_feature 1..45: note = Target Specific Oligonucleotide
  source 1..45: mol_type = other DNA
tagaataatggttcgtgaccttcgtaacgatagatttatacgacg

SEQ ID NO: 682 - DNA, 41 bp, synthetic construct
  misc_feature 1..41: note = Target Specific Oligonucleotide
  source 1..41: mol_type = other DNA
acctgaaggcgacgaggaggtaacgatagatttatacgacg

SEQ ID NO: 683 - DNA, 44 bp, synthetic construct
  misc_feature 1..44: note = Target Specific Oligonucleotide
  source 1..44: mol_type = other DNA
gcctggagtctatcgtacaatcgtaacgatagatttatacgacg

SEQ ID NO: 684 - DNA, 47 bp, synthetic construct
  misc_feature 1..47: note = Target Specific Oligonucleotide
  source 1..47: mol_type = other DNA
acatttaattagaccgtaacccttcgtaacgatagatttatacgacg

SEQ ID NO: 685 - DNA, 45 bp, synthetic construct
  misc_feature 1..45: note = Target Specific Oligonucleotide
  source 1..45: mol_type = other DNA
gctctggccatacttaacagatagtaacgatagatttatacgacg

SEQ ID NO: 686 - DNA, 46 bp, synthetic construct
  misc_feature 1..46: note = Target Specific Oligonucleotide
  source 1..46: mol_type = other DNA
cagcattgagtgataatgcaatctgtaacgatagatttatacgacg

SEQ ID NO: 687 - DNA, 44 bp, synthetic construct
  misc_feature 1..44: note = Target Specific Oligonucleotide
  source 1..44: mol_type = other DNA
atacacacccgctaccttactggtaacgatagatttatacgacg

SEQ ID NO: 688 - DNA, 44 bp, synthetic construct
  misc_feature 1..44: note = Target Specific Oligonucleotide
  source 1..44: mol_type = other DNA
acaaagacgagaactatggcaagtaacgatagatttatacgacg

SEQ ID NO: 689 - DNA, 44 bp, synthetic construct
  misc_feature 1..44: note = Target Specific Oligonucleotide
  source 1..44: mol_type = other DNA
aggcagacgctctaagtctaaagtaacgatagatttatacgacg

SEQ ID NO: 690 - DNA, 44 bp, synthetic construct
  misc_feature 1..44: note = Target Specific Oligonucleotide
  source 1..44: mol_type = other DNA
tctcttactcatcgtgaaaggcgtaacgatagatttatacgacg

SEQ ID NO: 691 - DNA, 42 bp, synthetic construct
  misc_feature 1..42: note = Target Specific Oligonucleotide
  source 1..42: mol_type = other DNA
gcctcaagttcgcattcagcgtaacgatagatttatacgacg

SEQ ID NO: 692 - DNA, 44 bp, synthetic construct
  misc_feature 1..44: note = Target Specific Oligonucleotide
  source 1..44: mol_type = other DNA
gaaatggaaacgttaacagccagtaacgatagatttatacgacg

SEQ ID NO: 693 - DNA, 43 bp, synthetic construct
  misc_feature 1..43: note = Target Specific Oligonucleotide
  source 1..43: mol_type = other DNA
cacctgcttcggaatctcagtgtaacgatagatttatacgacg

SEQ ID NO: 694 - DNA, 46 bp, synthetic construct
  misc_feature 1..46: note = Target Specific Oligonucleotide
  source 1..46: mol_type = other DNA
gacttgtgcttcgttaattaaacagtaacgatagatttatacgacg

SEQ ID NO: 695 - DNA, 46 bp, synthetic construct
  misc_feature 1..46: note = Target Specific Oligonucleotide
  source 1..46: mol_type = other DNA
gctgtaatatacgcccactttagcgtaacgatagatttatacgacg

SEQ ID NO: 696 - DNA, 44 bp, synthetic construct
  misc_feature 1..44: note = Target Specific Oligonucleotide
  source 1..44: mol_type = other DNA
ttccattgtgcggttgggattgtaacttataaaggatagacatc

SEQ ID NO: 697 - DNA, 45 bp, synthetic construct
  misc_feature 1..45: note = Target Specific Oligonucleotide
  source 1..45: mol_type = other DNA
ctccagtaatcgcttatggtccgtaacttataaaggatagacatc

SEQ ID NO: 698 - DNA, 45 bp, synthetic construct
  misc_feature 1..45: note = Target Specific Oligonucleotide
  source 1..45: mol_type = other DNA
cctcttctccgtcaagaagttcgtaacttataaaggatagacatc

SEQ ID NO: 699 - DNA, 45 bp, synthetic construct
  misc_feature 1..45: note = Target Specific Oligonucleotide
  source 1..45: mol_type = other DNA
tgattgagatggcggtatttgagtaacttataaaggatagacatc

SEQ ID NO: 700 - DNA, 45 bp, synthetic construct
  misc_feature 1..45: note = Target Specific Oligonucleotide
  source 1..45: mol_type = other DNA
gccaattataagtactcgggctgtaacttataaaggatagacatc

SEQ ID NO: 701 - DNA, 42 bp, synthetic construct
  misc_feature 1..42: note = Target Specific Oligonucleotide
  source 1..42: mol_type = other DNA
ccaggccagcgtatgttccgtaacttataaaggatagacatc

SEQ ID NO: 702 - DNA, 43 bp, synthetic construct
  misc_feature 1..43: note = Target Specific Oligonucleotide
  source 1..43: mol_type = other DNA
ctggggcaaccaacacttgtgtaacttataaaggatagacatc

SEQ ID NO: 703 - DNA, 43 bp, synthetic construct
  misc_feature 1..43: note = Target Specific Oligonucleotide
  source 1..43: mol_type = other DNA
ggctggacaataggctcccagtaacttataaaggatagacatc

SEQ ID NO: 704 - DNA, 44 bp, synthetic construct
  misc_feature 1..44: note = Target Specific Oligonucleotide
  source 1..44: mol_type = other DNA
aaagctatggcacgaaagccggtaacttataaaggatagacatc

SEQ ID NO: 705 - DNA, 44 bp, synthetic construct
  misc_feature 1..44: note = Target Specific Oligonucleotide
  source 1..44: mol_type = other DNA
gcagtccatctaaggtggactgtaacttataaaggatagacatc

SEQ ID NO: 706 - DNA, 46 bp, synthetic construct
  misc_feature 1..46: note = Target Specific Oligonucleotide
  source 1..46: mol_type = other DNA
tgtatatggagtaccctcttgctgtaacttataaaggatagacatc

SEQ ID NO: 707 - DNA, 44 bp, synthetic construct
  misc_feature 1..44: note = Target Specific Oligonucleotide
  source 1..44: mol_type = other DNA
ctggatcaccaaaacggcaatgtaacttataaaggatagacatc

SEQ ID NO: 708 - DNA, 45 bp, synthetic construct
  misc_feature 1..45: note = Target Specific Oligonucleotide
  source 1..45: mol_type = other DNA
ccaggataacctttacttgccagtaacttataaaggatagacatc

SEQ ID NO: 709 - DNA, 46 bp, synthetic construct
  misc_feature 1..46: note = Target Specific Oligonucleotide
  source 1..46: mol_type = other DNA
atttccagctgcgatatctactcgtaacttataaaggatagacatc

SEQ ID NO: 710 - DNA, 47 bp, synthetic construct
  misc_feature 1..47: note = Target Specific Oligonucleotide
  source 1..47: mol_type = other DNA
tttatagcagctagtcctttcttggtaacttataaaggatagacatc

SEQ ID NO: 711 - DNA, 46 bp, synthetic construct
  misc_feature 1..46: note = Target Specific Oligonucleotide
  source 1..46: mol_type = other DNA
tgatatgagattttcccggatgggtaacttataaaggatagacatc

SEQ ID NO: 712 - DNA, 43 bp, synthetic construct
  misc_feature 1..43: note = Target Specific Oligonucleotide
  source 1..43: mol_type = other DNA
tgacaacagttagggcggtggtaacttataaaggatagacatc

SEQ ID NO: 713 - DNA, 44 bp, synthetic construct
  misc_feature 1..44: note = Target Specific Oligonucleotide
  source 1..44: mol_type = other DNA
ttggttcccgagtactgtctagtaacttataaaggatagacatc

SEQ ID NO: 714 - DNA, 45 bp, synthetic construct
  misc_feature 1..45: note = Target Specific Oligonucleotide
  source 1..45: mol_type = other DNA
gtttagttccaacgccatctgtgtaacttataaaggatagacatc

SEQ ID NO: 715 - DNA, 45 bp, synthetic construct
  misc_feature 1..45: note = Target Specific Oligonucleotide
  source 1..45: mol_type = other DNA
aagagttgcgacagtatcacccgtaacttataaaggatagacatc

SEQ ID NO: 716 - DNA, 44 bp, synthetic construct
  misc_feature 1..44: note = Target Specific Oligonucleotide
  source 1..44: mol_type = other DNA
gctgctggatatccctcagaagtaacttataaaggatagacatc

SEQ ID NO: 717 - DNA, 46 bp, synthetic construct
  misc_feature 1..46: note = Target Specific Oligonucleotide
  source 1..46: mol_type = other DNA
tgcaagacagactatgtctatgggtaacttataaaggatagacatc

SEQ ID NO: 718 - DNA, 46 bp, synthetic construct
  misc_feature 1..46: note = Target Specific Oligonucleotide
  source 1..46: mol_type = other DNA
cttcaaggactaggtcaattgcagtaacttataaaggatagacatc

SEQ ID NO: 719 - DNA, 47 bp, synthetic construct
  misc_feature 1..47: note = Target Specific Oligonucleotide
  source 1..47: mol_type = other DNA
gatatgctgcactaactagtcttggtaacttataaaggatagacatc

SEQ ID NO: 720 - DNA, 40 bp, synthetic construct
  misc_feature 1..40: note = Target Specific Oligonucleotide
  source 1..40: mol_type = other DNA
atctcgagcaagacgttcagtgtaacggttatcgtggaaa

SEQ ID NO: 721 - DNA, 43 bp, synthetic construct
  misc_feature 1..43: note = Target Specific Oligonucleotide
  source 1..43: mol_type = other DNA
ctgtccataattagtccatgaggagtaacggttatcgtggaaa

SEQ ID NO: 722 - DNA, 43 bp, synthetic construct
  misc_feature 1..43: note = Target Specific Oligonucleotide
  source 1..43: mol_type = other DNA
atctttggattatactgcctgaccgtaacggttatcgtggaaa

SEQ ID NO: 723 - DNA, 39 bp, synthetic construct
  misc_feature 1..39: note = Target Specific Oligonucleotide
  source 1..39: mol_type = other DNA
cagcaagcttgcgaccttgagtaacggttatcgtggaaa

SEQ ID NO: 724 - DNA, 44 bp, synthetic construct
  misc_feature 1..44: note = Target Specific Oligonucleotide
  source 1..44: mol_type = other DNA
agttaaagttgagagatcatctccagtaacggttatcgtggaaa

SEQ ID NO: 725 - DNA, 44 bp, synthetic construct
  misc_feature 1..44: note = Target Specific Oligonucleotide
  source 1..44: mol_type = other DNA
aggaatggatctatcactatttctagtaacggttatcgtggaaa

SEQ ID NO: 726 - DNA, 44 bp, synthetic construct
  misc_feature 1..44: note = Target Specific Oligonucleotide
  source 1..44: mol_type = other DNA
cagcataatgattaggtatgcaaaagtaacggttatcgtggaaa

SEQ ID NO: 727 - DNA, 44 bp, synthetic construct
  misc_feature 1..44: note = Target Specific Oligonucleotide
  source 1..44: mol_type = other DNA
ttcagtctgataaaatctacagtcagtaacggttatcgtggaaa

SEQ ID NO: 728 - DNA, 39 bp, synthetic construct
  misc_feature 1..39: note = Target Specific Oligonucleotide
  source 1..39: mol_type = other DNA
tccaacacttcgtggggtccgtaacggttatcgtggaaa

SEQ ID NO: 729 - DNA, 39 bp, synthetic construct
  misc_feature 1..39: note = Target Specific Oligonucleotide
  source 1..39: mol_type = other DNA
agggctacaatgtgatggccgtaacggttatcgtggaaa

SEQ ID NO: 730 - DNA, 42 bp, synthetic construct
  misc_feature 1..42: note = Target Specific Oligonucleotide
  source 1..42: mol_type = other DNA
tcaaggtcataacctggttcatcgtaacggttatcgtggaaa

SEQ ID NO: 731 - DNA, 42 bp, synthetic construct
  misc_feature 1..42: note = Target Specific Oligonucleotide
  source 1..42: mol_type = other DNA
cctacaacaaacttgtctggaatgtaacggttatcgtggaaa

SEQ ID NO: 732 - DNA, 40 bp, synthetic construct
  misc_feature 1..40: note = Target Specific Oligonucleotide
  source 1..40: mol_type = other DNA
aaacaacaatccgcccaaagggtaacggttatcgtggaaa

SEQ ID NO: 733 - DNA, 41 bp, synthetic construct
  misc_feature 1..41: note = Target Specific Oligonucleotide
  source 1..41: mol_type = other DNA
aaagctctactaagcagatggcgtaacggttatcgtggaaa

SEQ ID NO: 734 - DNA, 41 bp, synthetic construct
  misc_feature 1..41: note = Target Specific Oligonucleotide
  source 1..41: mol_type = other DNA
actgctgacaaagattcactgggtaacggttatcgtggaaa

SEQ ID NO: 735 - DNA, 44 bp, synthetic construct
  misc_feature 1..44: note = Target Specific Oligonucleotide
  source 1..44: mol_type = other DNA
ctgatagtctataggctcatagtgcgtaacggttatcgtggaaa

SEQ ID NO: 736 - DNA, 40 bp, synthetic construct
  misc_feature 1..40: note = Target Specific Oligonucleotide
  source 1..40: mol_type = other DNA
atcactaatcacgacgccagggtaacggttatcgtggaaa

SEQ ID NO: 737 - DNA, 40 bp, synthetic construct
  misc_feature 1..40: note = Target Specific Oligonucleotide
  source 1..40: mol_type = other DNA
tggcgatgtcaataggactccgtaacggttatcgtggaaa

SEQ ID NO: 738 - DNA, 42 bp, synthetic construct
  misc_feature 1..42: note = Target Specific Oligonucleotide
  source 1..42: mol_type = other DNA
gaactctcatcttaggctttgtagtaacggttatcgtggaaa

SEQ ID NO: 739 - DNA, 44 bp, synthetic construct
  misc_feature 1..44: note = Target Specific Oligonucleotide
  source 1..44: mol_type = other DNA
tcaacattactgaaacactactaaagtaacggttatcgtggaaa

SEQ ID NO: 740 - DNA, 38 bp, synthetic construct
  misc_feature 1..38: note = Target Specific Oligonucleotide
  source 1..38: mol_type = other DNA
ggtcgccataacggagccggtaacggttatcgtggaaa

SEQ ID NO: 741 - DNA, 45 bp, synthetic construct
  misc_feature 1..45: note = Target Specific Oligonucleotide
  source 1..45: mol_type = other DNA
ttcttacagatatgagttcaatgtttgtaacggttatcgtggaaa

SEQ ID NO: 742 - DNA, 44 bp, synthetic construct
  misc_feature 1..44: note = Target Specific Oligonucleotide
  source 1..44: mol_type = other DNA
aggacagaacaaaacttcttagatggtaacggttatcgtggaaa

SEQ ID NO: 743 - DNA, 36 bp, synthetic construct
  misc_feature 1..36: note = Target Specific Oligonucleotide
  source 1..36: mol_type = other DNA
tcgcagccgcctgaagcgtaacggttatcgtggaaa

SEQ ID NO: 744 - DNA, 41 bp, synthetic construct
  misc_feature 1..41: note = Target Specific Oligonucleotide
  source 1..41: mol_type = other DNA
ggaatttggaaccggcttgcgtaacggtactttatctagct

SEQ ID NO: 745 - DNA, 46 bp, synthetic construct
  misc_feature 1..46: note = Target Specific Oligonucleotide
  source 1..46: mol_type = other DNA
agagtcttgagtataatcttggtaggtaacggtactttatctagct

SEQ ID NO: 746 - DNA, 46 bp, synthetic construct
  misc_feature 1..46: note = Target Specific Oligonucleotide
  source 1..46: mol_type = other DNA
attcaggaagcatacactaattcttgtaacggtactttatctagct

SEQ ID NO: 747 - DNA, 44 bp, synthetic construct
  misc_feature 1..44: note = Target Specific Oligonucleotide
  source 1..44: mol_type = other DNA
tccagaaataagcgaaaatagcagtaacggtactttatctagct

SEQ ID NO: 748 - DNA, 47 bp, synthetic construct
  misc_feature 1..47: note = Target Specific Oligonucleotide
  source 1..47: mol_type = other DNA
atcttttgaactactagctacaaaatgtaacggtactttatctagct

SEQ ID NO: 749 - DNA, 42 bp, synthetic construct
  misc_feature 1..42: note = Target Specific Oligonucleotide
  source 1..42: mol_type = other DNA
taacaagctcgtaatggcccagtaacggtactttatctagct

SEQ ID NO: 750 - DNA, 42 bp, synthetic construct
  misc_feature 1..42: note = Target Specific Oligonucleotide
  source 1..42: mol_type = other DNA
catccaaaccgtgtgaaagctgtaacggtactttatctagct

SEQ ID NO: 751 - DNA, 41 bp, synthetic construct
  misc_feature 1..41: note = Target Specific Oligonucleotide
  source 1..41: mol_type = other DNA
acaacgccatgtgcgatttggtaacggtactttatctagct

SEQ ID NO: 752 - DNA, 45 bp, synthetic construct
  misc_feature 1..45: note = Target Specific Oligonucleotide
  source 1..45: mol_type = other DNA
ctacctaccactaggtacctatcagtaacggtactttatctagct

SEQ ID NO: 753 - DNA, 43 bp, synthetic construct
  misc_feature 1..43: note = Target Specific Oligonucleotide
  source 1..43: mol_type = other DNA
aggacatttacgtgtacagcacgtaacggtactttatctagct

SEQ ID NO: 754 - DNA, 43 bp, synthetic construct
  misc_feature 1..43: note = Target Specific Oligonucleotide
  source 1..43: mol_type = other DNA
cgcctgaatacaacgagttaacgtaacggtactttatctagct

SEQ ID NO: 755 - DNA, 44 bp, synthetic construct
  misc_feature 1..44: note = Target Specific Oligonucleotide
  source 1..44: mol_type = other DNA
cttccagattgtcgccgttaattgtaacggtactttatctagct

SEQ ID NO: 756 - DNA, 44 bp, synthetic construct
  misc_feature 1..44: note = Target Specific Oligonucleotide
  source 1..44: mol_type = other DNA
cagatttacctaaaccaccaccagtaacggtactttatctagct

SEQ ID NO: 757 - DNA, 45 bp, synthetic construct
  misc_feature 1..45: note = Target Specific Oligonucleotide
  source 1..45: mol_type = other DNA
tttgagctgatctagtaggtcttcgtaacggtactttatctagct

SEQ ID NO: 758 - DNA, 41 bp, synthetic construct
  misc_feature 1..41: note = Target Specific Oligonucleotide
  source 1..41: mol_type = other DNA
tctgtagctcgatccctggagtaacggtactttatctagct

SEQ ID NO: 759 - DNA, 44 bp, synthetic construct
  misc_feature 1..44: note = Target Specific Oligonucleotide
  source 1..44: mol_type = other DNA
caactgtcattcggttaaactgagtaacggtactttatctagct

SEQ ID NO: 760 - DNA, 44 bp, synthetic construct
  misc_feature 1..44: note = Target Specific Oligonucleotide
  source 1..44: mol_type = other DNA
tttgcttccgttcatcattagtcgtaacggtactttatctagct

SEQ ID NO: 761 - DNA, 45 bp, synthetic construct
  misc_feature 1..45: note = Target Specific Oligonucleotide
  source 1..45: mol_type = other DNA
acttccaatagtctggaaatcacagtaacggtactttatctagct

SEQ ID NO: 762 - DNA, 45 bp, synthetic construct
  misc_feature 1..45: note = Target Specific Oligonucleotide
  source 1..45: mol_type = other DNA
tcatctgctggattaagctaatacgtaacggtactttatctagct

SEQ ID NO: 763 - DNA, 41 bp, synthetic construct
  misc_feature 1..41: note = Target Specific Oligonucleotide
  source 1..41: mol_type = other DNA
agataggctcccgtatgcccgtaacggtactttatctagct

SEQ ID NO: 764 - DNA, 41 bp, synthetic construct
  misc_feature 1..41: note = Target Specific Oligonucleotide
  source 1..41: mol_type = other DNA
gcttctcggctaccacagacgtaacggtactttatctagct

SEQ ID NO: 765 - DNA, 46 bp, synthetic construct
  misc_feature 1..46: note = Target Specific Oligonucleotide
  source 1..46: mol_type = other DNA
agacttactcttaagaccatacttcgtaacggtactttatctagct

SEQ ID NO: 766 - DNA, 42 bp, synthetic construct
  misc_feature 1..42: note = Target Specific Oligonucleotide
  source 1..42: mol_type = other DNA
gttgagaggtggcgtttttgagtaacggtactttatctagct

SEQ ID NO: 767 - DNA, 44 bp, synthetic construct
  misc_feature 1..44: note = Target Specific Oligonucleotide
  source 1..44: mol_type = other DNA
tcaatacgtttctgcaagagtttgtaacggtactttatctagct

SEQ ID NO: 768 - DNA, 41 bp, synthetic construct
  misc_feature 1..41: note = Target Specific Oligonucleotide
  source 1..41: mol_type = other DNA
ttatggcctctatagcatctcccgtaactccatctagaccg

SEQ ID NO: 769 - DNA, 40 bp, synthetic construct
  misc_feature 1..40: note = Target Specific Oligonucleotide
  source 1..40: mol_type = other DNA
cagtccgggcaactatcttattgtaactccatctagaccg

SEQ ID NO: 770 - DNA, 42 bp, synthetic construct
  misc_feature 1..42: note = Target Specific Oligonucleotide
  source 1..42: mol_type = other DNA
ttcaagatacgaataatcctccgagtaactccatctagaccg

SEQ ID NO: 771 - DNA, 40 bp, synthetic construct
  misc_feature 1..40: note = Target Specific Oligonucleotide
  source 1..40: mol_type = other DNA
gtaggcgcttagcttgattttcgtaactccatctagaccg

SEQ ID NO: 772 - DNA, 38 bp, synthetic construct
  misc_feature 1..38: note = Target Specific Oligonucleotide
  source 1..38: mol_type = other DNA
cttcaagtcgggctcttgcagtaactccatctagaccg

SEQ ID NO: 773 - DNA, 38 bp, synthetic construct
  misc_feature 1..38: note = Target Specific Oligonucleotide
  source 1..38: mol_type = other DNA
agaatgaggcacgcaaagctgtaactccatctagaccg

SEQ ID NO: 774 - DNA, 40 bp, synthetic construct
  misc_feature 1..40: note = Target Specific Oligonucleotide
  source 1..40: mol_type = other DNA
ctcagcatccgataggactttcgtaactccatctagaccg

SEQ ID NO: 775 - DNA, 40 bp, synthetic construct
  misc_feature 1..40: note = Target Specific Oligonucleotide
  source 1..40: mol_type = other DNA
aagtcactataacacccgtcctgtaactccatctagaccg

SEQ ID NO: 776 - DNA, 39 bp, synthetic construct
  misc_feature 1..39: note = Target Specific Oligonucleotide
  source 1..39: mol_type = other DNA
ttggcatggtagatcgtccaggtaactccatctagaccg

SEQ ID NO: 777 - DNA, 39 bp, synthetic construct
  misc_feature 1..39: note = Target Specific Oligonucleotide
  source 1..39: mol_type = other DNA
caccttccaacgactcaatccgtaactccatctagaccg

SEQ ID NO: 778 - DNA, 42 bp, synthetic construct
  misc_feature 1..42: note = Target Specific Oligonucleotide
  source 1..42: mol_type = other DNA
acaacctgtaagtgtagttctctggtaactccatctagaccg

SEQ ID NO: 779 - DNA, 43 bp, synthetic construct
  misc_feature 1..43: note = Target Specific Oligonucleotide
  source 1..43: mol_type = other DNA
aaatcagatactcgtctacagaatggtaactccatctagaccg

SEQ ID NO: 780 - DNA, 38 bp, synthetic construct
  misc_feature 1..38: note = Target Specific Oligonucleotide
  source 1..38: mol_type = other DNA
agcagcaagtcgcattttccgtaactccatctagaccg

SEQ ID NO: 781 - DNA, 39 bp, synthetic construct
  misc_feature 1..39: note = Target Specific Oligonucleotide
  source 1..39: mol_type = other DNA
actggcatggcggataaaaaggtaactccatctagaccg

SEQ ID NO: 782 - DNA, 38 bp, synthetic construct
  misc_feature 1..38: note = Target Specific Oligonucleotide
  source 1..38: mol_type = other DNA
cgggtgctctcgtgaacaaagtaactccatctagaccg

SEQ ID NO: 783 - DNA, 41 bp, synthetic construct
  misc_feature 1..41: note = Target Specific Oligonucleotide
  source 1..41: mol_type = other DNA
gatcgaagagtcgagataaggatgtaactccatctagaccg

SEQ ID NO: 784 - DNA, 41 bp, synthetic construct
  misc_feature 1..41: note = Target Specific Oligonucleotide
  source 1..41: mol_type = other DNA
ttgttgaagacgcttccataagtgtaactccatctagaccg

SEQ ID NO: 785 - DNA, 37 bp, synthetic construct
  misc_feature 1..37: note = Target Specific Oligonucleotide
  source 1..37: mol_type = other DNA
gtggtgacgcagaatcccggtaactccatctagaccg

SEQ ID NO: 786 - DNA, 45 bp, synthetic construct
  misc_feature 1..45: note = Target Specific Oligonucleotide
  source 1..45: mol_type = other DNA
cattataaagacgtaataatttagggtgtaactccatctagaccg

SEQ ID NO: 787 - DNA, 39 bp, synthetic construct
  misc_feature 1..39: note = Target Specific Oligonucleotide
  source 1..39: mol_type = other DNA
gctcccactcctagacatgttgtaactccatctagaccg

SEQ ID NO: 788 - DNA, 40 bp, synthetic construct
  misc_feature 1..40: note = Target Specific Oligonucleotide
  source 1..40: mol_type = other DNA
tttgttacacggttaaacagccgtaactccatctagaccg

SEQ ID NO: 789 - DNA, 42 bp, synthetic construct
  misc_feature 1..42: note = Target Specific Oligonucleotide
  source 1..42: mol_type = other DNA
gaggtaaataaacgtcgataggaagtaactccatctagaccg

SEQ ID NO: 790 - DNA, 40 bp, synthetic construct
  misc_feature 1..40: note = Target Specific Oligonucleotide
  source 1..40: mol_type = other DNA
ttaatgtggagtgcaagttggggtaactccatctagaccg

SEQ ID NO: 791 - DNA, 41 bp, synthetic construct
  misc_feature 1..41: note = Target Specific Oligonucleotide
  source 1..41: mol_type = other DNA
ccttgtatcttagcttgggatgtgtaactccatctagaccg

SEQ ID NO: 792 - DNA, 43 bp, synthetic construct
  misc_feature 1..43: note = Target Specific Oligonucleotide
  source 1..43: mol_type = other DNA
tcaatgatcagcggatacctattgtaactcagtagatatagcg

SEQ ID NO: 793 - DNA, 43 bp, synthetic construct
  misc_feature 1..43: note = Target Specific Oligonucleotide
  source 1..43: mol_type = other DNA
cgtgtgatcttacgatccttatagtaactcagtagatatagcg

SEQ ID NO: 794 - DNA, 43 bp, synthetic construct
  misc_feature 1..43: note = Target Specific Oligonucleotide
  source 1..43: mol_type = other DNA
tcatcctctacgagtctatcttggtaactcagtagatatagcg

SEQ ID NO: 795 - DNA, 40 bp, synthetic construct
  misc_feature 1..40: note = Target Specific Oligonucleotide
  source 1..40: mol_type = other DNA
tcaaagtaacccgcgtaggcgtaactcagtagatatagcg

SEQ ID NO: 796 - DNA, 41 bp, synthetic construct
  misc_feature 1..41: note = Target Specific Oligonucleotide
  source 1..41: mol_type = other DNA
ataggacgcatactgtcgcaagtaactcagtagatatagcg

SEQ ID NO: 797 - DNA, 41 bp, synthetic construct
  misc_feature 1..41: note = Target Specific Oligonucleotide
  source 1..41: mol_type = other DNA
ttatgtccgtcgaccttgcatgtaactcagtagatatagcg

SEQ ID NO: 798 - DNA, 42 bp, synthetic construct
  misc_feature 1..42: note = Target Specific Oligonucleotide
  source 1..42: mol_type = other DNA
ttccaaggaacggacttctagggtaactcagtagatatagcg

SEQ ID NO: 799 - DNA, 42 bp, synthetic construct
  misc_feature 1..42: note = Target Specific Oligonucleotide
  source 1..42: mol_type = other DNA
ataggtacgaacgctctcgatcgtaactcagtagatatagcg

SEQ ID NO: 800 - DNA, 42 bp, synthetic construct
  misc_feature 1..42: note = Target Specific Oligonucleotide
  source 1..42: mol_type = other DNA
tcttggatgatcgcatgaaaccgtaactcagtagatatagcg

SEQ ID NO: 801 - DNA, 42 bp, synthetic construct
  misc_feature 1..42: note = Target Specific Oligonucleotide
  source 1..42: mol_type = other DNA
acaacccaattaacgtgaagccgtaactcagtagatatagcg

SEQ ID NO: 802 - DNA, 41 bp, synthetic construct
  misc_feature 1..41: note = Target Specific Oligonucleotide
  source 1..41: mol_type = other DNA
atgcggtataacgcacgttccgtaactcagtagatatagcg

SEQ ID NO: 803 - DNA, 41 bp, synthetic construct
  misc_feature 1..41: note = Target Specific Oligonucleotide
  source 1..41: mol_type = other DNA
ctcaagtcacgacgcagttctgtaactcagtagatatagcg

SEQ ID NO: 804 - DNA, 40 bp, synthetic construct
  misc_feature 1..40: note = Target Specific Oligonucleotide
  source 1..40: mol_type = other DNA
gcaagtcacgaacgtgactcgtaactcagtagatatagcg

SEQ ID NO: 805 - DNA, 40 bp, synthetic construct
  misc_feature 1..40: note = Target Specific Oligonucleotide
  source 1..40: mol_type = other DNA
ttggcgttatactgcgccacgtaactcagtagatatagcg

SEQ ID NO: 806 - DNA, 40 bp, synthetic construct
  misc_feature 1..40: note = Target Specific Oligonucleotide
  source 1..40: mol_type = other DNA
agtggtgcgtatcgtaagcggtaactcagtagatatagcg

SEQ ID NO: 807 - DNA, 40 bp, synthetic construct
  misc_feature 1..40: note = Target Specific Oligonucleotide
  source 1..40: mol_type = other DNA
atccaggcgacgaaacgagagtaactcagtagatatagcg

SEQ ID NO: 808 - DNA, 40 bp, synthetic construct
  misc_feature 1..40: note = Target Specific Oligonucleotide
  source 1..40: mol_type = other DNA
ttggatgaatcggcaggctggtaactcagtagatatagcg

SEQ ID NO: 809 - DNA, 43 bp, synthetic construct
  misc_feature 1..43: note = Target Specific Oligonucleotide
  source 1..43: mol_type = other DNA
tcagtcaattcgtaatgtacaccgtaactcagtagatatagcg

SEQ ID NO: 810 - DNA, 41 bp, synthetic construct
  misc_feature 1..41: note = Target Specific Oligonucleotide
  source 1..41: mol_type = other DNA
catcgaagacgatccactggcgtaactcagtagatatagcg

SEQ ID NO: 811 - DNA, 39 bp, synthetic construct
  misc_feature 1..39: note = Target Specific Oligonucleotide
  source 1..39: mol_type = other DNA
cacggatcaagcggcacttgtaactcagtagatatagcg

SEQ ID NO: 812 - DNA, 39 bp, synthetic construct
  misc_feature 1..39: note = Target Specific Oligonucleotide
  source 1..39: mol_type = other DNA
tccaccgtatcgcaagctggtaactcagtagatatagcg

SEQ ID NO: 813 - DNA, 41 bp, synthetic construct
  misc_feature 1..41: note = Target Specific Oligonucleotide
  source 1..41: mol_type = other DNA
atgcaaagtttcgtagggcgagtaactcagtagatatagcg

SEQ ID NO: 814 - DNA, 38 bp, synthetic construct
  misc_feature 1..38: note = Target Specific Oligonucleotide
  source 1..38: mol_type = other DNA
gcgttggcacgacgacatgtaactcagtagatatagcg

SEQ ID NO: 815 - DNA, 38 bp, synthetic construct
  misc_feature 1..38: note = Target Specific Oligonucleotide
  source 1..38: mol_type = other DNA
ttgcagacggacgcccaagtaactcagtagatatagcg

SEQ ID NO: 816 - DNA, 40 bp, synthetic construct
  misc_feature 1..40: note = Target Specific Oligonucleotide
  source 1..40: mol_type = other DNA
agctgtatcgtcaaggcactcgtaaccttttgaaacgcta

SEQ ID NO: 817 - DNA, 39 bp, synthetic construct
  misc_feature 1..39: note = Target Specific Oligonucleotide
  source 1..39: mol_type = other DNA
ctcgctcccagtccgaaatggtaaccttttgaaacgcta

SEQ ID NO: 818 - DNA, 44 bp, synthetic construct
  misc_feature 1..44: note = Target Specific Oligonucleotide
  source 1..44: mol_type = other DNA
acttgtactagtatgccttaagaaagtaaccttttgaaacgcta

SEQ ID NO: 819 - DNA, 42 bp, synthetic construct
  misc_feature 1..42: note = Target Specific Oligonucleotide
  source 1..42: mol_type = other DNA
gtgaccatgactaatagcagtgggtaaccttttgaaacgcta

SEQ ID NO: 820 - DNA, 41 bp, synthetic construct
  misc_feature 1..41: note = Target Specific Oligonucleotide
  source 1..41: mol_type = other DNA
gctgtgtcgagaatatccaagagtaaccttttgaaacgcta

SEQ ID NO: 821 - DNA, 41 bp, synthetic construct
  misc_feature 1..41: note = Target Specific Oligonucleotide
  source 1..41: mol_type = other DNA
cctactgtcgctaatggattgggtaaccttttgaaacgcta

SEQ ID NO: 822 - DNA, 41 bp, synthetic construct
  misc_feature 1..41: note = Target Specific Oligonucleotide
  source 1..41: mol_type = other DNA
acactagcactacctaaggaccgtaaccttttgaaacgcta

SEQ ID NO: 823 - DNA, 41 bp, synthetic construct
  misc_feature 1..41: note = Target Specific Oligonucleotide
  source 1..41: mol_type = other DNA
gtctgtgaactagttcaggcacgtaaccttttgaaacgcta

SEQ ID NO: 824 - DNA, 40 bp, synthetic construct
  misc_feature 1..40: note = Target Specific Oligonucleotide
  source 1..40: mol_type = other DNA
ctaggtcagcgcaaccaaatggtaaccttttgaaacgcta

SEQ ID NO: 825 - DNA, 42 bp, synthetic construct
  misc_feature 1..42: note = Target Specific Oligonucleotide
  source 1..42: mol_type = other DNA
cctaataagctataactggcccagtaaccttttgaaacgcta

SEQ ID NO: 826 - DNA, 43 bp, synthetic construct
  misc_feature 1..43: note = Target Specific Oligonucleotide
  source 1..43: mol_type = other DNA
ctaaattcctatgcagtgtgactcgtaaccttttgaaacgcta

SEQ ID NO: 827 - DNA, 42 bp, synthetic construct
  misc_feature 1..42: note = Target Specific Oligonucleotide
  source 1..42: mol_type = other DNA
atcttgttgagctatccaaactggtaaccttttgaaacgcta

SEQ ID NO: 828 - DNA, 42 bp, synthetic construct
  misc_feature 1..42: note = Target Specific Oligonucleotide
  source 1..42: mol_type = other DNA
aaatacgcatcgtgttatctctggtaaccttttgaaacgcta

SEQ ID NO: 829 - DNA, 42 bp, synthetic construct
  misc_feature 1..42: note = Target Specific Oligonucleotide
  source 1..42: mol_type = other DNA
tgttaagagaactagccaaacctgtaaccttttgaaacgcta

SEQ ID NO: 830 - DNA, 43 bp, synthetic construct
  misc_feature 1..43: note = Target Specific Oligonucleotide
  source 1..43: mol_type = other DNA
cataaagtagcttgatcgaagagtgtaaccttttgaaacgcta

SEQ ID NO: 831 - DNA, 41 bp, synthetic construct
  misc_feature 1..41: note = Target Specific Oligonucleotide
  source 1..41: mol_type = other DNA
taagcaaacacgcctttacatagtaaccttttgaaacgcta

SEQ ID NO: 832 - DNA, 44 bp, synthetic construct
  misc_feature 1..44: note = Target Specific Oligonucleotide
  source 1..44: mol_type = other DNA
aaggccttagtaagatattacagacgtaaccttttgaaacgcta

SEQ ID NO: 833 - DNA, 45 bp, synthetic construct
  misc_feature 1..45: note = Target Specific Oligonucleotide
  source 1..45: mol_type = other DNA
acaatatacgtctgctatattcttccgtaaccttttgaaacgcta

SEQ ID NO: 834 - DNA, 41 bp, synthetic construct
  misc_feature 1..41: note = Target Specific Oligonucleotide
  source 1..41: mol_type = other DNA
tccactggatagggttctgtctgtaaccttttgaaacgcta

SEQ ID NO: 835 - DNA, 44 bp, synthetic construct
  misc_feature 1..44: note = Target Specific Oligonucleotide
  source 1..44: mol_type = other DNA
aagttatactatgaaagagcagtctgtaaccttttgaaacgcta

SEQ ID NO: 836 - DNA, 44 bp, synthetic construct
  misc_feature 1..44: note = Target Specific Oligonucleotide
  source 1..44: mol_type = other DNA
acatgttaatgcctaagtctatgtagtaaccttttgaaacgcta

SEQ ID NO: 837 - DNA, 40 bp, synthetic construct
  misc_feature 1..40: note = Target Specific Oligonucleotide
  source 1..40: mol_type = other DNA
gcctagaatgcctacttgggagtaaccttttgaaacgcta

SEQ ID NO: 838 - DNA, 41 bp, synthetic construct
  misc_feature 1..41: note = Target Specific Oligonucleotide
  source 1..41: mol_type = other DNA
tcagtggaatcgtagcaaaacagtaaccttttgaaacgcta

SEQ ID NO: 839 - DNA, 45 bp, synthetic construct
  misc_feature 1..45: note = Target Specific Oligonucleotide
  source 1..45: mol_type = other DNA
acaataattaggagtagtacagttcagtaaccttttgaaacgcta

SEQ ID NO: 840 - DNA, 42 bp, synthetic construct
  misc_feature 1..42: note = Target Specific Oligonucleotide
  source 1..42: mol_type = other DNA
ttccactcggataagatgctgagtaactcagacatgtagatt

SEQ ID NO: 841 - DNA, 42 bp, synthetic construct
  misc_feature 1..42: note = Target Specific Oligonucleotide
  source 1..42: mol_type = other DNA
aatactccacacgcaaatttccgtaactcagacatgtagatt

SEQ ID NO: 842 - DNA, 40 bp, synthetic construct
  misc_feature 1..40: note = Target Specific Oligonucleotide
  source 1..40: mol_type = other DNA
ggccagaccatcgctatctggtaactcagacatgtagatt

SEQ ID NO: 843 - DNA, 42 bp, synthetic construct
  misc_feature 1..42: note = Target Specific Oligonucleotide
  source 1..42: mol_type = other DNA
caccacactatgtcgaaaagtggtaactcagacatgtagatt

SEQ ID NO: 844 - DNA, 42 bp, synthetic construct
  misc_feature 1..42: note = Target Specific Oligonucleotide
  source 1..42: mol_type = other DNA
ccctttcttgcggagattctctgtaactcagacatgtagatt

SEQ ID NO: 845 - DNA, 40 bp, synthetic construct
  misc_feature 1..40: note = Target Specific Oligonucleotide
  source 1..40: mol_type = other DNA
acaaacacgcacctcaaagcgtaactcagacatgtagatt

SEQ ID NO: 846 - DNA, 42 bp, synthetic construct
  misc_feature 1..42: note = Target Specific Oligonucleotide
  source 1..42: mol_type = other DNA
cccagtagattaccactggagtgtaactcagacatgtagatt

SEQ ID NO: 847 - DNA, 39 bp, synthetic construct
  misc_feature 1..39: note = Target Specific Oligonucleotide
  source 1..39: mol_type = other DNA
gcagtgctcgcttagtgctgtaactcagacatgtagatt

SEQ ID NO: 848 - DNA, 40 bp, synthetic construct
  misc_feature 1..40: note = Target Specific Oligonucleotide
  source 1..40: mol_type = other DNA
ggctcgacgctaggatctgagtaactcagacatgtagatt

SEQ ID NO: 849 - DNA, 39 bp, synthetic construct
  misc_feature 1..39: note = Target Specific Oligonucleotide
  source 1..39: mol_type = other DNA
aggatgggcctccggttcagtaactcagacatgtagatt

SEQ ID NO: 850 - DNA, 40 bp, synthetic construct
  misc_feature 1..40: note = Target Specific Oligonucleotide
  source 1..40: mol_type = other DNA
ggaacatctcgaagcgctcagtaactcagacatgtagatt

SEQ ID NO: 851 - DNA, 40 bp, synthetic construct
  misc_feature 1..40: note = Target Specific Oligonucleotide
  source 1..40: mol_type = other DNA
agacggaaaccgtagctgccgtaactcagacatgtagatt

SEQ ID NO: 852 - DNA, 41 bp, synthetic construct
  misc_feature 1..41: note = Target Specific Oligonucleotide
  source 1..41: mol_type = other DNA
tgttcaatatcgtccggggacgtaactcagacatgtagatt

SEQ ID NO: 853 - DNA, 40 bp, synthetic construct
  misc_feature 1..40: note = Target Specific Oligonucleotide
  source 1..40: mol_type = other DNA
cattgcttgggacggcaagggtaactcagacatgtagatt

SEQ ID NO: 854 - DNA, 42 bp, synthetic construct
  misc_feature 1..42: note = Target Specific Oligonucleotide
  source 1..42: mol_type = other DNA
agtggagaatgtcagtctgagtgtaactcagacatgtagatt

SEQ ID NO: 855 - DNA, 43 bp, synthetic construct
  misc_feature 1..43: note = Target Specific Oligonucleotide
  source 1..43: mol_type = other DNA
agttttttatggcgggaggtagagtaactcagacatgtagatt

SEQ ID NO: 856 - DNA, 41 bp, synthetic construct
  misc_feature 1..41: note = Target Specific Oligonucleotide
  source 1..41: mol_type = other DNA
tagaaactaccaacccaccgagtaactcagacatgtagatt

SEQ ID NO: 857 - DNA, 42 bp, synthetic construct
  misc_feature 1..42: note = Target Specific Oligonucleotide
  source 1..42: mol_type = other DNA
ttccctggttagtacggtgaaggtaactcagacatgtagatt

SEQ ID NO: 858 - DNA, 42 bp, synthetic construct
  misc_feature 1..42: note = Target Specific Oligonucleotide
  source 1..42: mol_type = other DNA
taagctggtatgtcctactcccgtaactcagacatgtagatt

SEQ ID NO: 859 - DNA, 41 bp, synthetic construct
  misc_feature 1..41: note = Target Specific Oligonucleotide
  source 1..41: mol_type = other DNA
ctgacgcacacctattgcaaggtaactcagacatgtagatt

SEQ ID NO: 860 - DNA, 39 bp, synthetic construct
  misc_feature 1..39: note = Target Specific Oligonucleotide
  source 1..39: mol_type = other DNA
tcaccgtcgtggaaagcacgtaactcagacatgtagatt

SEQ ID NO: 861 - DNA, 44 bp, synthetic construct
  misc_feature 1..44: note = Target Specific Oligonucleotide
  source 1..44: mol_type = other DNA
gtaaactaacccttaactgcaagagtaactcagacatgtagatt

SEQ ID NO: 862 - DNA, 44 bp, synthetic construct
  misc_feature 1..44: note = Target Specific Oligonucleotide
  source 1..44: mol_type = other DNA
tttaggtactaaggttcaccaagagtaactcagacatgtagatt

SEQ ID NO: 863 - DNA, 46 bp, synthetic construct
  misc_feature 1..46: note = Target Specific Oligonucleotide
  source 1..46: mol_type = other DNA
tcatcatatacaagagatgaaatcctgtaactcagacatgtagatt

SEQ ID NO: 864 - DNA, 41 bp, synthetic construct
  misc_feature 1..41: note = Target Specific Oligonucleotide
  source 1..41: mol_type = other DNA
ccatggtgacgatcctcacggtaactaacataaattcgatg

SEQ ID NO: 865 - DNA, 42 bp, synthetic construct
  misc_feature 1..42: note = Target Specific Oligonucleotide
  source 1..42: mol_type = other DNA
gtgaacgtcttgggttgcttcgtaactaacataaattcgatg

SEQ ID NO: 866 - DNA, 39 bp, synthetic construct
  misc_feature 1..39: note = Target Specific Oligonucleotide
  source 1..39: mol_type = other DNA
ggcctggacggcgtaagagtaactaacataaattcgatg

SEQ ID NO: 867 - DNA, 40 bp, synthetic construct
  misc_feature 1..40: note = Target Specific Oligonucleotide
  source 1..40: mol_type = other DNA
gaagctcatgcgggaagcggtaactaacataaattcgatg

SEQ ID NO: 868 - DNA, 43 bp, synthetic construct
  misc_feature 1..43: note = Target Specific Oligonucleotide
  source 1..43: mol_type = other DNA
tgtcactgtacgagatgtttcggtaactaacataaattcgatg

SEQ ID NO: 869 - DNA, 43 bp, synthetic construct
  misc_feature 1..43: note = Target Specific Oligonucleotide
  source 1..43: mol_type = other DNA
ttgaagcttggtatgtcaggaggtaactaacataaattcgatg

SEQ ID NO: 870 - DNA, 42 bp, synthetic construct
  misc_feature 1..42: note = Target Specific Oligonucleotide
  source 1..42: mol_type = other DNA
ggttcccgatacatagctggagtaactaacataaattcgatg

SEQ ID NO: 871 - DNA, 44 bp, synthetic construct
  misc_feature 1..44: note = Target Specific Oligonucleotide
  source 1..44: mol_type = other DNA
atctttgaagtcgaaagacaggcgtaactaacataaattcgatg

SEQ ID NO: 872 - DNA, 44 bp, synthetic construct
  misc_feature 1..44: note = Target Specific Oligonucleotide
  source 1..44: mol_type = other DNA
gcttattaacacgaagctttgaggtaactaacataaattcgatg

SEQ ID NO: 873 - DNA, 42 bp, synthetic construct
  misc_feature 1..42: note = Target Specific Oligonucleotide
  source 1..42: mol_type = other DNA
tttcattccacgggaaggagagtaactaacataaattcgatg

SEQ ID NO: 874 - DNA, 41 bp, synthetic construct
  misc_feature 1..41: note = Target Specific Oligonucleotide
  source 1..41: mol_type = other DNA
tcagtgcttcgttggccattgtaactaacataaattcgatg

SEQ ID NO: 875 - DNA, 43 bp, synthetic construct
  misc_feature 1..43: note = Target Specific Oligonucleotide
  source 1..43: mol_type = other DNA
ctctgcgtaccaagcagtaattgtaactaacataaattcgatg

SEQ ID NO: 876 - DNA, 43 bp, synthetic construct
  misc_feature 1..43: note = Target Specific Oligonucleotide
  source 1..43: mol_type = other DNA
tttcaaggcctcgatttctgtcgtaactaacataaattcgatg

SEQ ID NO: 877 - DNA, 43 bp, synthetic construct
  misc_feature 1..43: note = Target Specific Oligonucleotide
  source 1..43: mol_type = other DNA
tagttcacaccgtacatctccagtaactaacataaattcgatg

SEQ ID NO: 878 - DNA, 42 bp, synthetic construct
  misc_feature 1..42: note = Target Specific Oligonucleotide
  source 1..42: mol_type = other DNA
cccacatttccggagtcatctgtaactaacataaattcgatg

SEQ ID NO: 879 - DNA, 45 bp, synthetic construct
  misc_feature 1..45: note = Target Specific Oligonucleotide
  source 1..45: mol_type = other DNA
ggtcctgagctatcttcagatattgtaactaacataaattcgatg

SEQ ID NO: 880 - DNA, 39 bp, synthetic construct
  misc_feature 1..39: note = Target Specific Oligonucleotide
  source 1..39: mol_type = other DNA
tcctccgtgcgaatcgctgtaactaacataaattcgatg

SEQ ID NO: 881 - DNA, 43 bp, synthetic construct
  misc_feature 1..43: note = Target Specific Oligonucleotide
  source 1..43: mol_type = other DNA
gccctttttattccggattgcagtaactaacataaattcgatg

SEQ ID NO: 882 - DNA, 40 bp, synthetic construct
  misc_feature 1..40: note = Target Specific Oligonucleotide
  source 1..40: mol_type = other DNA
gctgtctcctcagaccgcagtaactaacataaattcgatg

SEQ ID NO: 883 - DNA, 41 bp, synthetic construct
  misc_feature 1..41: note = Target Specific Oligonucleotide
  source 1..41: mol_type = other DNA
cacccctgtcggagttctcagtaactaacataaattcgatg

SEQ ID NO: 884 - DNA, 46 bp, synthetic construct
  misc_feature 1..46: note = Target Specific Oligonucleotide
  source 1..46: mol_type = other DNA
cagatttattaccctttttggaagcgtaactaacataaattcgatg

SEQ ID NO: 885 - DNA, 39 bp, synthetic construct
  misc_feature 1..39: note = Target Specific Oligonucleotide
  source 1..39: mol_type = other DNA
tcatgggcgggtacgtgggtaactaacataaattcgatg

SEQ ID NO: 886 - DNA, 42 bp, synthetic construct
  misc_feature 1..42: note = Target Specific Oligonucleotide
  source 1..42: mol_type = other DNA
caaaaatccccgcttgtgaacgtaactaacataaattcgatg

SEQ ID NO: 887 - DNA, 42 bp, synthetic construct
  misc_feature 1..42: note = Target Specific Oligonucleotide
  source 1..42: mol_type = other DNA
ctggggtcgtagtcaccatacgtaactaacataaattcgatg

SEQ ID NO: 888 - DNA, 39 bp, synthetic construct
  misc_feature 1..39: note = Target Specific Oligonucleotide
  source 1..39: mol_type = other DNA
cacacgataccggcaaagaaggtaacgtgacgattaccc

SEQ ID NO: 889 - DNA, 38 bp, synthetic construct
  misc_feature 1..38: note = Target Specific Oligonucleotide
  source 1..38: mol_type = other DNA
ggtgtcagtctccgacgtgagtaacgtgacgattaccc

SEQ ID NO: 890 - DNA, 39 bp, synthetic construct
  misc_feature 1..39: note = Target Specific Oligonucleotide
  source 1..39: mol_type = other DNA
gtgatggtgatcatctgggccgtaacgtgacgattaccc

SEQ ID NO: 891 - DNA, 36 bp, synthetic construct
  misc_feature 1..36: note = Target Specific Oligonucleotide
  source 1..36: mol_type = other DNA
ttggcctcgggaccaagcgtaacgtgacgattaccc

SEQ ID NO: 892 - DNA, 39 bp, synthetic construct
  misc_feature 1..39: note = Target Specific Oligonucleotide
  source 1..39: mol_type = other DNA
ctgtcatcttggtcaggtggtgtaacgtgacgattaccc

SEQ ID NO: 893 - DNA, 38 bp, synthetic construct
  misc_feature 1..38: note = Target Specific Oligonucleotide
  source 1..38: mol_type = other DNA
atcaccttgccgaaagtgccgtaacgtgacgattaccc

SEQ ID NO: 894 - DNA, 37 bp, synthetic construct
  misc_feature 1..37: note = Target Specific Oligonucleotide
  source 1..37: mol_type = other DNA
actgcacggccgtagtcatgtaacgtgacgattaccc

SEQ ID NO: 895 - DNA, 37 bp, synthetic construct
  misc_feature 1..37: note = Target Specific Oligonucleotide
  source 1..37: mol_type = other DNA
aggtactcaggtgtgccgcgtaacgtgacgattaccc

SEQ ID NO: 896 - DNA, 38 bp, synthetic construct
  misc_feature 1..38: note = Target Specific Oligonucleotide
  source 1..38: mol_type = other DNA
gaaggtgcgttcgatgacaggtaacgtgacgattaccc

SEQ ID NO: 897 - DNA, 39 bp, synthetic construct
  misc_feature 1..39: note = Target Specific Oligonucleotide
  source 1..39: mol_type = other DNA
ctcctcaggagtctccacatggtaacgtgacgattaccc

SEQ ID NO: 898 - DNA, 40 bp, synthetic construct
  misc_feature 1..40: note = Target Specific Oligonucleotide
  source 1..40: mol_type = other DNA
gtctggaaagagtacttcaggggtaacgtgacgattaccc

SEQ ID NO: 899 - DNA, 36 bp, synthetic construct
  misc_feature 1..36: note = Target Specific Oligonucleotide
  source 1..36: mol_type = other DNA
caggacgcggttctcggtgtaacgtgacgattaccc

SEQ ID NO: 900 - DNA, 38 bp, synthetic construct
  misc_feature 1..38: note = Target Specific Oligonucleotide
  source 1..38: mol_type = other DNA
atctcagcgccatagaagcggtaacgtgacgattaccc

SEQ ID NO: 901 - DNA, 37 bp, synthetic construct
  misc_feature 1..37: note = Target Specific Oligonucleotide
  source 1..37: mol_type = other DNA
tcgttcatggtcacgcggtgtaacgtgacgattaccc

SEQ ID NO: 902 - DNA, 39 bp, synthetic construct
  misc_feature 1..39: note = Target Specific Oligonucleotide
  source 1..39: mol_type = other DNA
cagcccgaagtctgtgatcttgtaacgtgacgattaccc

SEQ ID NO: 903 - DNA, 37 bp, synthetic construct
  misc_feature 1..37: note = Target Specific Oligonucleotide
  source 1..37: mol_type = other DNA
tcatggtggcaccgtccttgtaacgtgacgattaccc

SEQ ID NO: 904 - DNA, 38 bp, synthetic construct
  misc_feature 1..38: note = Target Specific Oligonucleotide
  source 1..38: mol_type = other DNA
ggtagtccagggctgacacagtaacgtgacgattaccc

SEQ ID NO: 905 - DNA, 41 bp, synthetic construct
  misc_feature 1..41: note = Target Specific Oligonucleotide
  source 1..41: mol_type = other DNA
ccatcattcttgaggaggaagtagtaacgtgacgattaccc

SEQ ID NO: 906 - DNA, 37 bp, synthetic construct
  misc_feature 1..37: note = Target Specific Oligonucleotide
  source 1..37: mol_type = other DNA
acaaagcagaggcggtcgtgtaacgtgacgattaccc

SEQ ID NO: 907 - DNA, 38 bp, synthetic construct
  misc_feature 1..38: note = Target Specific Oligonucleotide
  source 1..38: mol_type = other DNA
cggtcctcggagaacacacggtaacgtgacgattaccc

SEQ ID NO: 908 - DNA, 37 bp, synthetic construct
  misc_feature 1..37: note = Target Specific Oligonucleotide
  source 1..37: mol_type = other DNA
gagcctcacgttggtccacgtaacgtgacgattaccc

SEQ ID NO: 909 - DNA, 40 bp, synthetic construct
  misc_feature 1..40: note = Target Specific Oligonucleotide
  source 1..40: mol_type = other DNA
ctccttgtagccaatgaaggtggtaacgtgacgattaccc

SEQ ID NO: 910 - DNA, 38 bp, synthetic construct
  misc_feature 1..38: note = Target Specific Oligonucleotide
  source 1..38: mol_type = other DNA
ttcacaatagccacgtcgctgtaacgtgacgattaccc

SEQ ID NO: 911 - DNA, 37 bp, synthetic construct
  misc_feature 1..37: note = Target Specific Oligonucleotide
  source 1..37: mol_type = other DNA
ccctcgtttgtgcagccaagtaacgtgacgattaccc

SEQ ID NO: 912 - DNA, 43 bp, synthetic construct
  misc_feature 1..43: note = Target Specific Oligonucleotide
  source 1..43: mol_type = other DNA
agctatgtccgctatcttcatcggtaactctcttctattgcag

SEQ ID NO: 913 - DNA, 41 bp, synthetic construct
  misc_feature 1..41: note = Target Specific Oligonucleotide
  source 1..41: mol_type = other DNA
ttggcacatcgagacatggttgtaactctcttctattgcag

SEQ ID NO: 914 - DNA, 42 bp, synthetic construct
  misc_feature 1..42: note = Target Specific Oligonucleotide
  source 1..42: mol_type = other DNA
ccatcttggtcgtactttgtgagtaactctcttctattgcag

SEQ ID NO: 915 - DNA, 41 bp, synthetic construct
  misc_feature 1..41: note = Target Specific Oligonucleotide
  source 1..41: mol_type = other DNA
ccaactgagcatacgctaggagtaactctcttctattgcag

SEQ ID NO: 916 - DNA, 41 bp, synthetic construct
  misc_feature 1..41: note = Target Specific Oligonucleotide
  source 1..41: mol_type = other DNA
tcacgtactagccgttccatcgtaactctcttctattgcag

SEQ ID NO: 917 - DNA, 43 bp, synthetic construct
  misc_feature 1..43: note = Target Specific Oligonucleotide
  source 1..43: mol_type = other DNA
tttctccaagtcgtctctcatctgtaactctcttctattgcag

SEQ ID NO: 918 - DNA, 42 bp, synthetic construct
  misc_feature 1..42: note = Target Specific Oligonucleotide
  source 1..42: mol_type = other DNA
tcgtaagaaacgccactagaaagtaactctcttctattgcag

SEQ ID NO: 919 - DNA, 39 bp, synthetic construct
  misc_feature 1..39: note = Target Specific Oligonucleotide
  source 1..39: mol_type = other DNA
cactggcgtgcctaaaccagtaactctcttctattgcag

SEQ ID NO: 920 - DNA, 45 bp, synthetic construct
  misc_feature 1..45: note = Target Specific Oligonucleotide
  source 1..45: mol_type = other DNA
acatatcatacgtggacattagaacgtaactctcttctattgcag

SEQ ID NO: 921 - DNA, 40 bp, synthetic construct
  misc_feature 1..40: note = Target Specific Oligonucleotide
  source 1..40: mol_type = other DNA
gccgatggaatgctccatccgtaactctcttctattgcag

SEQ ID NO: 922 - DNA, 43 bp, synthetic construct
  misc_feature 1..43: note = Target Specific Oligonucleotide
  source 1..43: mol_type = other DNA
gattagcaatcggtcacaaagacgtaactctcttctattgcag

SEQ ID NO: 923 - DNA, 43 bp, synthetic construct
  misc_feature 1..43: note = Target Specific Oligonucleotide
  source 1..43: mol_type = other DNA
ggtgtagtagtacacattggagcgtaactctcttctattgcag

SEQ ID NO: 924 - DNA, 42 bp, synthetic construct
  misc_feature 1..42: note = Target Specific Oligonucleotide
  source 1..42: mol_type = other DNA
gatgaaacttcggttgtcagctgtaactctcttctattgcag

SEQ ID NO: 925 - DNA, 42 bp, synthetic construct
  misc_feature 1..42: note = Target Specific Oligonucleotide
  source 1..42: mol_type = other DNA
ttcttgaggctagcaacttgtggtaactctcttctattgcag

SEQ ID NO: 926 - DNA, 43 bp, synthetic construct
  misc_feature 1..43: note = Target Specific Oligonucleotide
  source 1..43: mol_type = other DNA
gtcccaaaactcgattagcttccgtaactctcttctattgcag

SEQ ID NO: 927 - DNA, 42 bp, synthetic construct
  misc_feature 1..42: note = Target Specific Oligonucleotide
  source 1..42: mol_type = other DNA
tattcgtggaacccctaacagcgtaactctcttctattgcag

SEQ ID NO: 928 - DNA, 43 bp, synthetic construct
  misc_feature 1..43: note = Target Specific Oligonucleotide
  source 1..43: mol_type = other DNA
caaagttgatatcgcccaaaatggtaactctcttctattgcag

SEQ ID NO: 929 - DNA, 42 bp, synthetic construct
  misc_feature 1..42: note = Target Specific Oligonucleotide
  source 1..42: mol_type = other DNA
tagatccaagcggttccatttcgtaactctcttctattgcag

SEQ ID NO: 930 - DNA, 42 bp, synthetic construct
  misc_feature 1..42: note = Target Specific Oligonucleotide
  source 1..42: mol_type = other DNA
tgaccagttcccgtaaaacactgtaactctcttctattgcag

SEQ ID NO: 931 - DNA, 41 bp, synthetic construct
  misc_feature 1..41: note = Target Specific Oligonucleotide
  source 1..41: mol_type = other DNA
tggcacatggtcgatacacatgtaactctcttctattgcag

SEQ ID NO: 932 - DNA, 41 bp, synthetic construct
  misc_feature 1..41: note = Target Specific Oligonucleotide
  source 1..41: mol_type = other DNA
tgacagcacaacgatcacaacgtaactctcttctattgcag

SEQ ID NO: 933 - DNA, 42 bp, synthetic construct
  misc_feature 1..42: note = Target Specific Oligonucleotide
  source 1..42: mol_type = other DNA
aagctcatgaactatgccagaggtaactctcttctattgcag

SEQ ID NO: 934 - DNA, 41 bp, synthetic construct
  misc_feature 1..41: note = Target Specific Oligonucleotide
  source 1..41: mol_type = other DNA
cgtcatacctgaccgagtactgtaactctcttctattgcag

SEQ ID NO: 935 - DNA, 43 bp, synthetic construct
  misc_feature 1..43: note = Target Specific Oligonucleotide
  source 1..43: mol_type = other DNA
tcttgattgtcgagaaagacttggtaactctcttctattgcag

SEQ ID NO: 936 - DNA, 42 bp, synthetic construct
  misc_feature 1..42: note = Target Specific Oligonucleotide
  source 1..42: mol_type = other DNA
tactgagccctatcggaaagtgtaactgatcttcatattacg

SEQ ID NO: 937 - DNA, 42 bp, synthetic construct
  misc_feature 1..42: note = Target Specific Oligonucleotide
  source 1..42: mol_type = other DNA
ccaaatcccttcgcagcaattgtaactgatcttcatattacg

SEQ ID NO: 938 - DNA, 42 bp, synthetic construct
  misc_feature 1..42: note = Target Specific Oligonucleotide
  source 1..42: mol_type = other DNA
gccatgatcaatcgcatcactgtaactgatcttcatattacg

SEQ ID NO: 939 - DNA, 43 bp, synthetic construct
  misc_feature 1..43: note = Target Specific Oligonucleotide
  source 1..43: mol_type = other DNA
atcatccagattcgaaccgtccgtaactgatcttcatattacg

SEQ ID NO: 940 - DNA, 43 bp, synthetic construct
  misc_feature 1..43: note = Target Specific Oligonucleotide
  source 1..43: mol_type = other DNA
ggactacattcgtgaagaagctgtaactgatcttcatattacg

SEQ ID NO: 941 - DNA, 42 bp, synthetic construct
  misc_feature 1..42: note = Target Specific Oligonucleotide
  source 1..42: mol_type = other DNA
ggagaggatccgtccaagaaagtaactgatcttcatattacg

SEQ ID NO: 942 - DNA, 42 bp, synthetic construct
  misc_feature 1..42: note = Target Specific Oligonucleotide
  source 1..42: mol_type = other DNA
gtcccagcattagaacctgtggtaactgatcttcatattacg

SEQ ID NO: 943 - DNA, 42 bp, synthetic construct
  misc_feature 1..42: note = Target Specific Oligonucleotide
  source 1..42: mol_type = other DNA
actctcagccagacctttagcgtaactgatcttcatattacg

SEQ ID NO: 944 - DNA, 44 bp, synthetic construct
  misc_feature 1..44: note = Target Specific Oligonucleotide
  source 1..44: mol_type = other DNA
catagtgtcatgtgcatgtagtcgtaactgatcttcatattacg

SEQ ID NO: 945 - DNA, 44 bp, synthetic construct
  misc_feature 1..44: note = Target Specific Oligonucleotide
  source 1..44: mol_type = other DNA
ccctgaccagcatattatctacagtaactgatcttcatattacg

SEQ ID NO: 946 - DNA, 41 bp, synthetic construct
  misc_feature 1..41: note = Target Specific Oligonucleotide
  source 1..41: mol_type = other DNA
cagcgcctgttacaagctctgtaactgatcttcatattacg

SEQ ID NO: 947 - DNA, 41 bp, synthetic construct
  misc_feature 1..41: note = Target Specific Oligonucleotide
  source 1..41: mol_type = other DNA
cgccagtcttaactgccatggtaactgatcttcatattacg

SEQ ID NO: 948 - DNA, 43 bp, synthetic construct
  misc_feature 1..43: note = Target Specific Oligonucleotide
  source 1..43: mol_type = other DNA
tcttcccaacaccgtaatctttgtaactgatcttcatattacg

SEQ ID NO: 949 - DNA, 43 bp, synthetic construct
  misc_feature 1..43: note = Target Specific Oligonucleotide
  source 1..43: mol_type = other DNA
gtgacttatgttcgggtctctcgtaactgatcttcatattacg

SEQ ID NO: 950 - DNA, 43 bp, synthetic construct
  misc_feature 1..43: note = Target Specific Oligonucleotide
  source 1..43: mol_type = other DNA
tgttaggttctagggccatagcgtaactgatcttcatattacg

SEQ ID NO: 951 - DNA, 42 bp, synthetic construct
  misc_feature 1..42: note = Target Specific Oligonucleotide
  source 1..42: mol_type = other DNA
ctggatagctttgggcaactcgtaactgatcttcatattacg

SEQ ID NO: 952 - DNA, 45 bp, synthetic construct
  misc_feature 1..45: note = Target Specific Oligonucleotide
  source 1..45: mol_type = other DNA
tgcagacagggtatgtatatttgggtaactgatcttcatattacg

SEQ ID NO: 953 - DNA, 44 bp, synthetic construct
  misc_feature 1..44: note = Target Specific Oligonucleotide
  source 1..44: mol_type = other DNA
aagaaaatatagctgagctccctgtaactgatcttcatattacg

SEQ ID NO: 954 - DNA, 40 bp, synthetic construct
  misc_feature 1..40: note = Target Specific Oligonucleotide
  source 1..40: mol_type = other DNA
gagaccggagaaacccggcgtaactgatcttcatattacg

SEQ ID NO: 955 - DNA, 40 bp, synthetic construct
  misc_feature 1..40: note = Target Specific Oligonucleotide
  source 1..40: mol_type = other DNA
ccgcaggagaccgaggtctgtaactgatcttcatattacg

SEQ ID NO: 956 - DNA, 42 bp, synthetic construct
  misc_feature 1..42: note = Target Specific Oligonucleotide
  source 1..42: mol_type = other DNA
aggctagggctcaagacaagggtaactgatcttcatattacg

SEQ ID NO: 957 - DNA, 38 bp, synthetic construct
  misc_feature 1..38: note = Target Specific Oligonucleotide
  source 1..38: mol_type = other DNA
gcgtccggtgtcgccatgtaactgatcttcatattacg

SEQ ID NO: 958 - DNA, 41 bp, synthetic construct
  misc_feature 1..41: note = Target Specific Oligonucleotide
  source 1..41: mol_type = other DNA
actctgggtcggcttgatccgtaactgatcttcatattacg

SEQ ID NO: 959 - DNA, 41 bp, synthetic construct
  misc_feature 1..41: note = Target Specific Oligonucleotide
  source 1..41: mol_type = other DNA
catgggaggatcgagcctgggtaactgatcttcatattacg

SEQ ID NO: 960 - DNA, 39 bp, synthetic construct
  misc_feature 1..39: note = Target Specific Oligonucleotide
  source 1..39: mol_type = other DNA
ggcactggaaacgattgacttgtaacggcaatacatcgt

SEQ ID NO: 961 - DNA, 38 bp, synthetic construct
  misc_feature 1..38: note = Target Specific Oligonucleotide
  source 1..38: mol_type = other DNA
tcaggcattcgttcacctgtgtaacggcaatacatcgt

SEQ ID NO: 962 - DNA, 40 bp, synthetic construct
  misc_feature 1..40: note = Target Specific Oligonucleotide
  source 1..40: mol_type = other DNA
actgtagcgattaatgccatccgtaacggcaatacatcgt

SEQ ID NO: 963 - DNA, 38 bp, synthetic construct
  misc_feature 1..38: note = Target Specific Oligonucleotide
  source 1..38: mol_type = other DNA
atagcctccattgcggttgggtaacggcaatacatcgt

SEQ ID NO: 964 - DNA, 40 bp, synthetic construct
  misc_feature 1..40: note = Target Specific Oligonucleotide
  source 1..40: mol_type = other DNA
cattcgggtgatcgatacacttgtaacggcaatacatcgt

SEQ ID NO: 965 - DNA, 39 bp, synthetic construct
  misc_feature 1..39: note = Target Specific Oligonucleotide
  source 1..39: mol_type = other DNA
gcacactcatcgagttgctccgtaacggcaatacatcgt

SEQ ID NO: 966 - DNA, 37 bp, synthetic construct
  misc_feature 1..37: note = Target Specific Oligonucleotide
  source 1..37: mol_type = other DNA
tcccgcttaatgcgcaggtgtaacggcaatacatcgt

SEQ ID NO: 967 - DNA, 37 bp, synthetic construct
  misc_feature 1..37: note = Target Specific Oligonucleotide
  source 1..37: mol_type = other DNA
ggtcccctgtatggcgtgagtaacggcaatacatcgt

SEQ ID NO: 968 - DNA, 38 bp, synthetic construct
  misc_feature 1..38: note = Target Specific Oligonucleotide
  source 1..38: mol_type = other DNA
aacacactggcggttgtcaagtaacggcaatacatcgt

SEQ ID NO: 969 - DNA, 39 bp, synthetic construct
  misc_feature 1..39: note = Target Specific Oligonucleotide
  source 1..39: mol_type = other DNA
cacacatcgacgaaggtttcagtaacggcaatacatcgt

SEQ ID NO: 970 - DNA, 39 bp, synthetic construct
  misc_feature 1..39: note = Target Specific Oligonucleotide
  source 1..39: mol_type = other DNA
ggctcttgcacgagttaacttgtaacggcaatacatcgt

SEQ ID NO: 971 - DNA, 39 bp, synthetic construct
  misc_feature 1..39: note = Target Specific Oligonucleotide
  source 1..39: mol_type = other DNA
atgtcaatggtacaccgctgagtaacggcaatacatcgt

SEQ ID NO: 972 - DNA, 38 bp, synthetic construct
  misc_feature 1..38: note = Target Specific Oligonucleotide
  source 1..38: mol_type = other DNA
gccatgcttacgctttcgttgtaacggcaatacatcgt

SEQ ID NO: 973 - DNA, 40 bp, synthetic construct
  misc_feature 1..40: note = Target Specific Oligonucleotide
  source 1..40: mol_type = other DNA
ggtataccagtaacacggaccagtaacggcaatacatcgt

SEQ ID NO: 974 - DNA, 40 bp, synthetic construct
  misc_feature 1..40: note = Target Specific Oligonucleotide
  source 1..40: mol_type = other DNA
gactctctcacgcataaacctggtaacggcaatacatcgt

SEQ ID NO: 975 - DNA, 40 bp, synthetic construct
  misc_feature 1..40: note = Target Specific Oligonucleotide
  source 1..40: mol_type = other DNA
aggagatgcgtaggtcaattcagtaacggcaatacatcgt

SEQ ID NO: 976 - DNA, 39 bp, synthetic construct
  misc_feature 1..39: note = Target Specific Oligonucleotide
  source 1..39: mol_type = other DNA
ccaacaggacgctagtgtagagtaacggcaatacatcgt

SEQ ID NO: 977 - DNA, 42 bp, synthetic construct
  misc_feature 1..42: note = Target Specific Oligonucleotide
  source 1..42: mol_type = other DNA
cagtaccaattaggttagcttctggtaacggcaatacatcgt

SEQ ID NO: 978 - DNA, 38 bp, synthetic construct
  misc_feature 1..38: note = Target Specific Oligonucleotide
  source 1..38: mol_type = other DNA
tccatggccgtcgatcaatggtaacggcaatacatcgt

SEQ ID NO: 979 - DNA, 40 bp, synthetic construct
  misc_feature 1..40: note = Target Specific Oligonucleotide
  source 1..40: mol_type = other DNA
tagtcaggaatatgcggtcattgtaacggcaatacatcgt

SEQ ID NO: 980 - DNA, 41 bp, synthetic construct
  misc_feature 1..41: note = Target Specific Oligonucleotide
  source 1..41: mol_type = other DNA
tatagcaactacttcgcatttccgtaacggcaatacatcgt

SEQ ID NO: 981 - DNA, 39 bp, synthetic construct
  misc_feature 1..39: note = Target Specific Oligonucleotide
  source 1..39: mol_type = other DNA
cgatggtgtcctacggatgtcgtaacggcaatacatcgt

SEQ ID NO: 982 - DNA, 44 bp, synthetic construct
  misc_feature 1..44: note = Target Specific Oligonucleotide
  source 1..44: mol_type = other DNA
gtaaccatagtaccacttattagtgggtaacggcaatacatcgt

SEQ ID NO: 983 - DNA, 37 bp, synthetic construct
  misc_feature 1..37: note = Target Specific Oligonucleotide
  source 1..37: mol_type = other DNA
cagcatcagcccgtgagtagtaacggcaatacatcgt

SEQ ID NO: 984 - DNA, 39 bp, synthetic construct
  misc_feature 1..39: note = Target Specific Oligonucleotide
  source 1..39: mol_type = other DNA
tcagatgctactggccgcttagacaagttaagatcggaa

SEQ ID NO: 985 - DNA, 41 bp, synthetic construct
  misc_feature 1..41: note = Target Specific Oligonucleotide
  source 1..41: mol_type = other DNA
cttgatcaaggcgctgaacaatagacaagttaagatcggaa

SEQ ID NO: 986 - DNA, 41 bp, synthetic construct
  misc_feature 1..41: note = Target Specific Oligonucleotide
  source 1..41: mol_type = other DNA
tcccttcgtatctcagcgagatagacaagttaagatcggaa

SEQ ID NO: 987 - DNA, 41 bp, synthetic construct
  misc_feature 1..41: note = Target Specific Oligonucleotide
  source 1..41: mol_type = other DNA
agactgttgactggcgtgatgtagacaagttaagatcggaa

SEQ ID NO: 988 - DNA, 41 bp, synthetic construct
  misc_feature 1..41: note = Target Specific Oligonucleotide
  source 1..41: mol_type = other DNA
ccaacatgctcgcatgagttctagacaagttaagatcggaa

SEQ ID NO: 989 - DNA, 41 bp, synthetic construct
  misc_feature 1..41: note = Target Specific Oligonucleotide
  source 1..41: mol_type = other DNA
ttccttcaccgaactgaggagtagacaagttaagatcggaa

SEQ ID NO: 990 - DNA, 40 bp, synthetic construct
  misc_feature 1..40: note = Target Specific Oligonucleotide
  source 1..40: mol_type = other DNA
agttccaacgagcggcttcatagacaagttaagatcggaa

SEQ ID NO: 991 - DNA, 40 bp, synthetic construct
  misc_feature 1..40: note = Target Specific Oligonucleotide
  source 1..40: mol_type = other DNA
ccatggctgacgagatctgatagacaagttaagatcggaa

SEQ ID NO: 992 - DNA, 40 bp, synthetic construct
  misc_feature 1..40: note = Target Specific Oligonucleotide
  source 1..40: mol_type = other DNA
tagcctaagacccggagctttagacaagttaagatcggaa

SEQ ID NO: 993 - DNA, 40 bp, synthetic construct
  misc_feature 1..40: note = Target Specific Oligonucleotide
  source 1..40: mol_type = other DNA
gcagatactcagcggcattgtagacaagttaagatcggaa

SEQ ID NO: 994 - DNA, 40 bp, synthetic construct
  misc_feature 1..40: note = Target Specific Oligonucleotide
  source 1..40: mol_type = other DNA
catagctcacgcagaacgtgtagacaagttaagatcggaa

SEQ ID NO: 995 - DNA, 39 bp, synthetic construct
  misc_feature 1..39: note = Target Specific Oligonucleotide
  source 1..39: mol_type = other DNA
gtcggccaccgttgaatgatagacaagttaagatcggaa

SEQ ID NO: 996 - DNA, 39 bp, synthetic construct
  misc_feature 1..39: note = Target Specific Oligonucleotide
  source 1..39: mol_type = other DNA
agaacggtcagcgagcgtatagacaagttaagatcggaa

SEQ ID NO: 997 - DNA, 44 bp, synthetic construct
  misc_feature 1..44: note = Target Specific Oligonucleotide
  source 1..44: mol_type = other DNA
gttatgcttagagtgttatctccatagacaagttaagatcggaa

SEQ ID NO: 998 - DNA, 40 bp, synthetic construct
  misc_feature 1..40: note = Target Specific Oligonucleotide
  source 1..40: mol_type = other DNA
tggtgatgtccgtgcgttcctagacaagttaagatcggaa

SEQ ID NO: 999 - DNA, 40 bp, synthetic construct
  misc_feature 1..40: note = Target Specific Oligonucleotide
  source 1..40: mol_type = other DNA
ggtccagaggatcgctctcttagacaagttaagatcggaa

SEQ ID NO: 1000 - DNA, 39 bp, synthetic construct
  misc_feature 1..39: note = Target Specific Oligonucleotide
  source 1..39: mol_type = other DNA
cgtggactgcaagtcccgatagacaagttaagatcggaa

SEQ ID NO: 1001 - DNA, 41 bp, synthetic construct
  misc_feature 1..41: note = Target Specific Oligonucleotide
  source 1..41: mol_type = other DNA
acaggtcaattcccgggtaagtagacaagttaagatcggaa

SEQ ID NO: 1002 - DNA, 41 bp, synthetic construct
  misc_feature 1..41: note = Target Specific Oligonucleotide
  source 1..41: mol_type = other DNA
ctgcaccaggttagggtgttttagacaagttaagatcggaa

SEQ ID NO: 1003 - DNA, 43 bp, synthetic construct
  misc_feature 1..43: note = Target Specific Oligonucleotide
  source 1..43: mol_type = other DNA
ggaaatggcatatacgtgggatttagacaagttaagatcggaa

SEQ ID NO: 1004 - DNA, 40 bp, synthetic construct
  misc_feature 1..40: note = Target Specific Oligonucleotide
  source 1..40: mol_type = other DNA
cttttgtctttgcgcccaggtagacaagttaagatcggaa

SEQ ID NO: 1005 - DNA, 38 bp, synthetic construct
  misc_feature 1..38: note = Target Specific Oligonucleotide
  source 1..38: mol_type = other DNA
tcctgccggttgcactcctagacaagttaagatcggaa

SEQ ID NO: 1006 - DNA, 43 bp, synthetic construct
  misc_feature 1..43: note = Target Specific Oligonucleotide
  source 1..43: mol_type = other DNA
atcatacagtgcaacgaaaaggttagacaagttaagatcggaa

SEQ ID NO: 1007 - DNA, 40 bp, synthetic construct
  misc_feature 1..40: note = Target Specific Oligonucleotide
  source 1..40: mol_type = other DNA
tcccacttgtcgtagttgggtagacaagttaagatcggaa

SEQ ID NO: 1008 - DNA, 39 bp, synthetic construct
  misc_feature 1..39: note = Target Specific Oligonucleotide
  source 1..39: mol_type = other DNA
aggtggtgttccgaacgagctagacagcccaattaaacg

SEQ ID NO: 1009 - DNA, 39 bp, synthetic construct
  misc_feature 1..39: note = Target Specific Oligonucleotide
  source 1..39: mol_type = other DNA
agatccgtgagcggaatgtatagacagcccaattaaacg

SEQ ID NO: 1010 - DNA, 41 bp, synthetic construct
  misc_feature 1..41: note = Target Specific Oligonucleotide
  source 1..41: mol_type = other DNA
ctcactcgctttagtggactcctagacagcccaattaaacg

SEQ ID NO: 1011 - DNA, 37 bp, synthetic construct
  misc_feature 1..37: note = Target Specific Oligonucleotide
  source 1..37: mol_type = other DNA
gccaaagaccgtggcgagtagacagcccaattaaacg

SEQ ID NO: 1012 - DNA, 41 bp, synthetic construct
  misc_feature 1..41: note = Target Specific Oligonucleotide
  source 1..41: mol_type = other DNA
agagttcttggtcgttggatcctagacagcccaattaaacg

SEQ ID NO: 1013 - DNA, 39 bp, synthetic construct
  misc_feature 1..39: note = Target Specific Oligonucleotide
  source 1..39: mol_type = other DNA
ctcctttgcaaccgggtctgtagacagcccaattaaacg

SEQ ID NO: 1014 - DNA, 41 bp, synthetic construct
  misc_feature 1..41: note = Target Specific Oligonucleotide
  source 1..41: mol_type = other DNA
cttgttattgacgtcgaaggcttagacagcccaattaaacg

SEQ ID NO: 1015 - DNA, 40 bp, synthetic construct
  misc_feature 1..40: note = Target Specific Oligonucleotide
  source 1..40: mol_type = other DNA
caggaacgtgtaactcttgcctagacagcccaattaaacg

SEQ ID NO: 1016 - DNA, 40 bp, synthetic construct
  misc_feature 1..40: note = Target Specific Oligonucleotide
  source 1..40: mol_type = other DNA
ggagtttcacacacgagttggtagacagcccaattaaacg

SEQ ID NO: 1017 - DNA, 39 bp, synthetic construct
  misc_feature 1..39: note = Target Specific Oligonucleotide
  source 1..39: mol_type = other DNA
aggttcccgctctacggatgtagacagcccaattaaacg

SEQ ID NO: 1018 - DNA, 38 bp, synthetic construct
  misc_feature 1..38: note = Target Specific Oligonucleotide
  source 1..38: mol_type = other DNA
accgtcatggactgccgtctagacagcccaattaaacg

SEQ ID NO: 1019 - DNA, 40 bp, synthetic construct
  misc_feature 1..40: note = Target Specific Oligonucleotide
  source 1..40: mol_type = other DNA
tgggacctttagacttcggtgtagacagcccaattaaacg

SEQ ID NO: 1020 - DNA, 37 bp, synthetic construct
  misc_feature 1..37: note = Target Specific Oligonucleotide
  source 1..37: mol_type = other DNA
gtccctgtagacgcgcgttagacagcccaattaaacg

SEQ ID NO: 1021 - DNA, 38 bp, synthetic construct
  misc_feature 1..38: note = Target Specific Oligonucleotide
  source 1..38: mol_type = other DNA
ccagagagggcgcatcttgtagacagcccaattaaacg

SEQ ID NO: 1022 - DNA, 40 bp, synthetic construct
  misc_feature 1..40: note = Target Specific Oligonucleotide
  source 1..40: mol_type = other DNA
tccactctgcacgctcatagttagacagcccaattaaacg

SEQ ID NO: 1023 - DNA, 40 bp, synthetic construct
  misc_feature 1..40: note = Target Specific Oligonucleotide
  source 1..40: mol_type = other DNA
gtgaaactcgacgttcacgtatagacagcccaattaaacg

SEQ ID NO: 1024 - DNA, 40 bp, synthetic construct
  misc_feature 1..40: note = Target Specific Oligonucleotide
  source 1..40: mol_type = other DNA
acacctctttgtcgttgaccttagacagcccaattaaacg

SEQ ID NO: 1025 - DNA, 39 bp, synthetic construct
  misc_feature 1..39: note = Target Specific Oligonucleotide
  source 1..39: mol_type = other DNA
actgttgcgagttctgcgagtagacagcccaattaaacg

SEQ ID NO: 1026 - DNA, 39 bp, synthetic construct
  misc_feature 1..39: note = Target Specific Oligonucleotide
  source 1..39: mol_type = other DNA
tggcaactccgtagttgtcctagacagcccaattaaacg

SEQ ID NO: 1027 - DNA, 38 bp, synthetic construct
  misc_feature 1..38: note = Target Specific Oligonucleotide
  source 1..38: mol_type = other DNA
tcctcggacgctaagctcatagacagcccaattaaacg

SEQ ID NO: 1028 - DNA, 38 bp, synthetic construct
  misc_feature 1..38: note = Target Specific Oligonucleotide
  source 1..38: mol_type = other DNA
ttacccggagaaccctcgctagacagcccaattaaacg

SEQ ID NO: 1029 - DNA, 38 bp, synthetic construct
  misc_feature 1..38: note = Target Specific Oligonucleotide
  source 1..38: mol_type = other DNA
tcccggaacatgcggtaggtagacagcccaattaaacg

SEQ ID NO: 1030 - DNA, 38 bp, synthetic construct
  misc_feature 1..38: note = Target Specific Oligonucleotide
  source 1..38: mol_type = other DNA
tgctcgatgtcgcccactgtagacagcccaattaaacg

SEQ ID NO: 1031 - DNA, 37 bp, synthetic construct
  misc_feature 1..37: note = Target Specific Oligonucleotide
  source 1..37: mol_type = other DNA
tgccggtgccgcttatggtagacagcccaattaaacg

SEQ ID NO: 1032 - DNA, 45 bp, synthetic construct
  misc_feature 1..45: note = Target Specific Oligonucleotide
  source 1..45: mol_type = other DNA
ccaaagtctgctagcttgatggtagacggtataatacttatttcc

SEQ ID NO: 1033 - DNA, 48 bp, synthetic construct
  misc_feature 1..48: note = Target Specific Oligonucleotide
  source 1..48: mol_type = other DNA
tattaggatggttaagctccttaagtagacggtataatacttatttcc

SEQ ID NO: 1034 - DNA, 45 bp, synthetic construct
  misc_feature 1..45: note = Target Specific Oligonucleotide
  source 1..45: mol_type = other DNA
catgggtgtaagtacgaacaggtagacggtataatacttatttcc

SEQ ID NO: 1035 - DNA, 43 bp, synthetic construct
  misc_feature 1..43: note = Target Specific Oligonucleotide
  source 1..43: mol_type = other DNA
ggcagaaagctaggccctggtagacggtataatacttatttcc

SEQ ID NO: 1036 - DNA, 43 bp, synthetic construct
  misc_feature 1..43: note = Target Specific Oligonucleotide
  source 1..43: mol_type = other DNA
gtacacaactccgtacgtgctagacggtataatacttatttcc

SEQ ID NO: 1037 - DNA, 45 bp, synthetic construct
  misc_feature 1..45: note = Target Specific Oligonucleotide
  source 1..45: mol_type = other DNA
atggaacgcagtatacctctcgtagacggtataatacttatttcc

SEQ ID NO: 1038 - DNA, 46 bp, synthetic construct
  misc_feature 1..46: note = Target Specific Oligonucleotide
  source 1..46: mol_type = other DNA
cttggcttgtaatcaggcatagatagacggtataatacttatttcc

SEQ ID NO: 1039 - DNA, 41 bp, synthetic construct
  misc_feature 1..41: note = Target Specific Oligonucleotide
  source 1..41: mol_type = other DNA
ctccatgaagcgccagcgtagacggtataatacttatttcc

SEQ ID NO: 1040 - DNA, 43 bp, synthetic construct
  misc_feature 1..43: note = Target Specific Oligonucleotide
  source 1..43: mol_type = other DNA
ccgcttgttagggtcgtagttagacggtataatacttatttcc

SEQ ID NO: 1041 - DNA, 42 bp, synthetic construct
  misc_feature 1..42: note = Target Specific Oligonucleotide
  source 1..42: mol_type = other DNA
ctcggtggaggacccgatgtagacggtataatacttatttcc

SEQ ID NO: 1042 - DNA, 44 bp, synthetic construct
  misc_feature 1..44: note = Target Specific Oligonucleotide
  source 1..44: mol_type = other DNA
ttactaaaatcttgccgggcctagacggtataatacttatttcc

SEQ ID NO: 1043 - DNA, 44 bp, synthetic construct
  misc_feature 1..44: note = Target Specific Oligonucleotide
  source 1..44: mol_type = other DNA
gcagcatttgcgataacaagctagacggtataatacttatttcc

SEQ ID NO: 1044 - DNA, 45 bp, synthetic construct
  misc_feature 1..45: note = Target Specific Oligonucleotide
  source 1..45: mol_type = other DNA
agaaggctatcagagtcgaagatagacggtataatacttatttcc

SEQ ID NO: 1045 - DNA, 43 bp, synthetic construct
  misc_feature 1..43: note = Target Specific Oligonucleotide
  source 1..43: mol_type = other DNA
ttcaggagctcggtaccacatagacggtataatacttatttcc

SEQ ID NO: 1046 - DNA, 44 bp, synthetic construct
  misc_feature 1..44: note = Target Specific Oligonucleotide
  source 1..44: mol_type = other DNA
ccccagagtccgaaagatccgtagacggtataatacttatttcc

SEQ ID NO: 1047 - DNA, 42 bp, synthetic construct
  misc_feature 1..42: note = Target Specific Oligonucleotide
  source 1..42: mol_type = other DNA
acagggcccagtaccgtggtagacggtataatacttatttcc

SEQ ID NO: 1048 - DNA, 46 bp, synthetic construct
  misc_feature 1..46: note = Target Specific Oligonucleotide
  source 1..46: mol_type = other DNA
tcagcaataactaagagaaggggtagacggtataatacttatttcc

SEQ ID NO: 1049 - DNA, 46 bp, synthetic construct
  misc_feature 1..46: note = Target Specific Oligonucleotide
  source 1..46: mol_type = other DNA
gtacggcaaatctaacgtgtaggtagacggtataatacttatttcc

SEQ ID NO: 1050 - DNA, 44 bp, synthetic construct
  misc_feature 1..44: note = Target Specific Oligonucleotide
  source 1..44: mol_type = other DNA
tgaaacaatgttgccgcctcctagacggtataatacttatttcc

SEQ ID NO: 1051 - DNA, 47 bp, synthetic construct
  misc_feature 1..47: note = Target Specific Oligonucleotide
  source 1..47: mol_type = other DNA
taataattatggggcattcagagatagacggtataatacttatttcc

SEQ ID NO: 1052 - DNA, 43 bp, synthetic construct
  misc_feature 1..43: note = Target Specific Oligonucleotide
  source 1..43: mol_type = other DNA
tctcacctgcctcataaggctagacggtataatacttatttcc

SEQ ID NO: 1053 - DNA, 47 bp, synthetic construct
  misc_feature 1..47: note = Target Specific Oligonucleotide
  source 1..47: mol_type = other DNA
atggtgaagcaatagattggaatgtagacggtataatacttatttcc

SEQ ID NO: 1054 - DNA, 49 bp, synthetic construct
  misc_feature 1..49: note = Target Specific Oligonucleotide
  source 1..49: mol_type = other DNA
cactaactagggtataaactgattggtagacggtataatacttatttcc

SEQ ID NO: 1055 - DNA, 44 bp, synthetic construct
  misc_feature 1..44: note = Target Specific Oligonucleotide
  source 1..44: mol_type = other DNA
agaagggacaaacaaggggactagacggtataatacttatttcc

SEQ ID NO: 1056 - DNA, 43 bp, synthetic construct
  misc_feature 1..43: note = Target Specific Oligonucleotide
  source 1..43: mol_type = other DNA
agtccgatactcaagcgcaaatagacaataaattctaacgagc

SEQ ID NO: 1057 - DNA, 42 bp, synthetic construct
  misc_feature 1..42: note = Target Specific Oligonucleotide
  source 1..42: mol_type = other DNA
cttgttgcgctcacggtatgtagacaataaattctaacgagc

SEQ ID NO: 1058 - DNA, 43 bp, synthetic construct
  misc_feature 1..43: note = Target Specific Oligonucleotide
  source 1..43: mol_type = other DNA
ccagagttgtagacgagcagctagacaataaattctaacgagc

SEQ ID NO: 1059 - DNA, 41 bp, synthetic construct
  misc_feature 1..41: note = Target Specific Oligonucleotide
  source 1..41: mol_type = other DNA
ttagcatggcaacacggcgtagacaataaattctaacgagc

SEQ ID NO: 1060 - DNA, 41 bp, synthetic construct
  misc_feature 1..41: note = Target Specific Oligonucleotide
  source 1..41: mol_type = other DNA
atgatgcgcacttcacgggtagacaataaattctaacgagc

SEQ ID NO: 1061 - DNA, 42 bp, synthetic construct
  misc_feature 1..42: note = Target Specific Oligonucleotide
  source 1..42: mol_type = other DNA
cccaccgctaggagggtagctagacaataaattctaacgagc

SEQ ID NO: 1062 - DNA, 42 bp, synthetic construct
  misc_feature 1..42: note = Target Specific Oligonucleotide
  source 1..42: mol_type = other DNA
gcagtgtggttaggccgatgtagacaataaattctaacgagc

SEQ ID NO: 1063 - DNA, 41 bp, synthetic construct
  misc_feature 1..41: note = Target Specific Oligonucleotide
  source 1..41: mol_type = other DNA
gacacggcggtgcactatctagacaataaattctaacgagc

SEQ ID NO: 1064 - DNA, 40 bp, synthetic construct
  misc_feature 1..40: note = Target Specific Oligonucleotide
  source 1..40: mol_type = other DNA
gcccaaagcggcttgcagtagacaataaattctaacgagc

SEQ ID NO: 1065 - DNA, 44 bp, synthetic construct
  misc_feature 1..44: note = Target Specific Oligonucleotide
  source 1..44: mol_type = other DNA
atacacacacggctaatactgctagacaataaattctaacgagc

SEQ ID NO: 1066 - DNA, 43 bp, synthetic construct
  misc_feature 1..43: note = Target Specific Oligonucleotide
  source 1..43: mol_type = other DNA
gtgaccgtgtacaccaacaactagacaataaattctaacgagc

SEQ ID NO: 1067 - DNA, 46 bp, synthetic construct
  misc_feature 1..46: note = Target Specific Oligonucleotide
  source 1..46: mol_type = other DNA
ctcttcatactagcatgactgtcatagacaataaattctaacgagc

SEQ ID NO: 1068 - DNA, 42 bp, synthetic construct
  misc_feature 1..42: note = Target Specific Oligonucleotide
  source 1..42: mol_type = other DNA
agaaggctttcgtcgttccctagacaataaattctaacgagc

SEQ ID NO: 1069 - DNA, 42 bp, synthetic construct
  misc_feature 1..42: note = Target Specific Oligonucleotide
  source 1..42: mol_type = other DNA
tttgctccgtgcgttcaaggtagacaataaattctaacgagc

SEQ ID NO: 1070 - DNA, 42 bp, synthetic construct
  misc_feature 1..42: note = Target Specific Oligonucleotide
  source 1..42: mol_type = other DNA
tccagtgcatccgtcacatctagacaataaattctaacgagc

SEQ ID NO: 1071 - DNA, 41 bp, synthetic construct
  misc_feature 1..41: note = Target Specific Oligonucleotide
  source 1..41: mol_type = other DNA
gatgaacagcggtgcgttgtagacaataaattctaacgagc

SEQ ID NO: 1072 - DNA, 42 bp, synthetic construct
  misc_feature 1..42: note = Target Specific Oligonucleotide
  source 1..42: mol_type = other DNA
cactgataccggctctccgttagacaataaattctaacgagc

SEQ ID NO: 1073 - DNA, 42 bp, synthetic construct
  misc_feature 1..42: note = Target Specific Oligonucleotide
  source 1..42: mol_type = other DNA
gaacgcttgcgttccctagctagacaataaattctaacgagc

SEQ ID NO: 1074 - DNA, 45 bp, synthetic construct
  misc_feature 1..45: note = Target Specific Oligonucleotide
  source 1..45: mol_type = other DNA
cagatagatttccgggatacagctagacaataaattctaacgagc

SEQ ID NO: 1075 - DNA, 40 bp, synthetic construct
  misc_feature 1..40: note = Target Specific Oligonucleotide
  source 1..40: mol_type = other DNA
tctgctgcggcgattgtgtagacaataaattctaacgagc

SEQ ID NO: 1076 - DNA, 41 bp, synthetic construct
  misc_feature 1..41: note = Target Specific Oligonucleotide
  source 1..41: mol_type = other DNA
ctcggtgatcatgcgtccctagacaataaattctaacgagc

SEQ ID NO: 1077 - DNA, 43 bp, synthetic construct
  misc_feature 1..43: note = Target Specific Oligonucleotide
  source 1..43: mol_type = other DNA
tccaatttcagggcgttctcgtagacaataaattctaacgagc

SEQ ID NO: 1078 - DNA, 40 bp, synthetic construct
  misc_feature 1..40: note = Target Specific Oligonucleotide
  source 1..40: mol_type = other DNA
tgcgcaggtcgacgtcagtagacaataaattctaacgagc

SEQ ID NO: 1079 - DNA, 43 bp, synthetic construct
  misc_feature 1..43: note = Target Specific Oligonucleotide
  source 1..43: mol_type = other DNA
gtccaggtagtatccggagggtagacaataaattctaacgagc

SEQ ID NO: 1080 - DNA, 40 bp, synthetic construct
  misc_feature 1..40: note = Target Specific Oligonucleotide
  source 1..40: mol_type = other DNA
attcccgacagactcatcgctagacgataaattgcgaacg

SEQ ID NO: 1081 - DNA, 40 bp, synthetic construct
  misc_feature 1..40: note = Target Specific Oligonucleotide
  source 1..40: mol_type = other DNA
cagttacatgttcgcgcacgtagacgataaattgcgaacg

SEQ ID NO: 1082 - DNA, 39 bp, synthetic construct
  misc_feature 1..39: note = Target Specific Oligonucleotide
  source 1..39: mol_type = other DNA
acgagcactagggtcgtcgtagacgataaattgcgaacg

SEQ ID NO: 1083 - DNA, 46 bp, synthetic construct
  misc_feature 1..46: note = Target Specific Oligonucleotide
  source 1..46: mol_type = other DNA
ttttgataatcgaatgaaaaagtcactagacgataaattgcgaacg

SEQ ID NO: 1084 - DNA, 43 bp, synthetic construct
  misc_feature 1..43: note = Target Specific Oligonucleotide
  source 1..43: mol_type = other DNA
ctgaatactgctagacgagaagctagacgataaattgcgaacg

SEQ ID NO: 1085 - DNA, 43 bp, synthetic construct
  misc_feature 1..43: note = Target Specific Oligonucleotide
  source 1..43: mol_type = other DNA
ctccttggaacgaactaatcctgtagacgataaattgcgaacg

SEQ ID NO: 1086 - DNA, 42 bp, synthetic construct
  misc_feature 1..42: note = Target Specific Oligonucleotide
  source 1..42: mol_type = other DNA
ttcaggcggttaattggtaccctagacgataaattgcgaacg

SEQ ID NO: 1087 - DNA, 42 bp, synthetic construct
  misc_feature 1..42: note = Target Specific Oligonucleotide
  source 1..42: mol_type = other DNA
gttaggaatggcgaacagacagtagacgataaattgcgaacg

SEQ ID NO: 1088 - DNA, 42 bp, synthetic construct
  misc_feature 1..42: note = Target Specific Oligonucleotide
  source 1..42: mol_type = other DNA
gatggtcttaaagcgtgaagcctagacgataaattgcgaacg

SEQ ID NO: 1089 - DNA, 42 bp, synthetic construct
  misc_feature 1..42: note = Target Specific Oligonucleotide
  source 1..42: mol_type = other DNA
ctgtgcctagccctaacttatatagacgataaattgcgaacg

SEQ ID NO: 1090 - DNA, 42 bp, synthetic construct
  misc_feature 1..42: note = Target Specific Oligonucleotide
  source 1..42: mol_type = other DNA
agagaccacccataacaagagttagacgataaattgcgaacg

SEQ ID NO: 1091 - DNA, 43 bp, synthetic construct
  misc_feature 1..43: note = Target Specific Oligonucleotide
  source 1..43: mol_type = other DNA
ttggagcaccgatatagatatggtagacgataaattgcgaacg

SEQ ID NO: 1092 - DNA, 41 bp, synthetic construct
  misc_feature 1..41: note = Target Specific Oligonucleotide
  source 1..41: mol_type = other DNA
tcaaagacgcaaacgtccaaatagacgataaattgcgaacg

SEQ ID NO: 1093 - DNA, 42 bp, synthetic construct
  misc_feature 1..42: note = Target Specific Oligonucleotide
  source 1..42: mol_type = other DNA
gagagctgattaggtgctcaggtagacgataaattgcgaacg

SEQ ID NO: 1094 - DNA, 40 bp, synthetic construct
  misc_feature 1..40: note = Target Specific Oligonucleotide
  source 1..40: mol_type = other DNA
tgcttgtacgcggagaaggttagacgataaattgcgaacg

SEQ ID NO: 1095 - DNA, 40 bp, synthetic construct
  misc_feature 1..40: note = Target Specific Oligonucleotide
  source 1..40: mol_type = other DNA
ctgcagacacgtgtatgtgctagacgataaattgcgaacg

SEQ ID NO: 1096 - DNA, 40 bp, synthetic construct
  misc_feature 1..40: note = Target Specific Oligonucleotide
  source 1..40: mol_type = other DNA
catccaatgcaagtccggtgtagacgataaattgcgaacg

SEQ ID NO: 1097 - DNA, 42 bp, synthetic construct
  misc_feature 1..42: note = Target Specific Oligonucleotide
  source 1..42: mol_type = other DNA
accaagtgataacgtgttcagatagacgataaattgcgaacg

SEQ ID NO: 1098 - DNA, 42 bp, synthetic construct
  misc_feature 1..42: note = Target Specific Oligonucleotide
  source 1..42: mol_type = other DNA
aatctgagctttgcgaaatggttagacgataaattgcgaacg

SEQ ID NO: 1099 - DNA, 44 bp, synthetic construct
  misc_feature 1..44: note = Target Specific Oligonucleotide
  source 1..44: mol_type = other DNA
caaagcccacataacactatcttttagacgataaattgcgaacg

SEQ ID NO: 1100 - DNA, 44 bp, synthetic construct
  misc_feature 1..44: note = Target Specific Oligonucleotide
  source 1..44: mol_type = other DNA
tgtactggactatagacaccaatttagacgataaattgcgaacg

SEQ ID NO: 1101 - DNA, 45 bp, synthetic construct
  misc_feature 1..45: note = Target Specific Oligonucleotide
  source 1..45: mol_type = other DNA
atcaatataccacgttagtagtcactagacgataaattgcgaacg

SEQ ID NO: 1102 - DNA, 42 bp, synthetic construct
  misc_feature 1..42: note = Target Specific Oligonucleotide
  source 1..42: mol_type = other DNA
tgtattttaacgccaagcaggctagacgataaattgcgaacg

SEQ ID NO: 1103 - DNA, 42 bp, synthetic construct
  misc_feature 1..42: note = Target Specific Oligonucleotide
  source 1..42: mol_type = other DNA
cactgaaaacacggttatgtcctagacgataaattgcgaacg

SEQ ID NO: 1104 - DNA, 40 bp, synthetic construct
  misc_feature 1..40: note = Target Specific Oligonucleotide
  source 1..40: mol_type = other DNA
cgagacagatcggagtagctgtagacgaccgaatttacgg

SEQ ID NO: 1105 - DNA, 41 bp, synthetic construct
  misc_feature 1..41: note = Target Specific Oligonucleotide
  source 1..41: mol_type = other DNA
tctctcaatgacgggacaaagatagacgaccgaatttacgg

SEQ ID NO: 1106 - DNA, 41 bp, synthetic construct
  misc_feature 1..41: note = Target Specific Oligonucleotide
  source 1..41: mol_type = other DNA
cagatgtggtgtatggtccacatagacgaccgaatttacgg

SEQ ID NO: 1107 - DNA, 40 bp, synthetic construct
  misc_feature 1..40: note = Target Specific Oligonucleotide
  source 1..40: mol_type = other DNA
ttctcctcggttacttcgtgatagacgaccgaatttacgg

SEQ ID NO: 1108 - DNA, 39 bp, synthetic construct
  misc_feature 1..39: note = Target Specific Oligonucleotide
  source 1..39: mol_type = other DNA
tgtgctaagccgaaggaggttagacgaccgaatttacgg

SEQ ID NO: 1109 - DNA, 44 bp, synthetic construct
  misc_feature 1..44: note = Target Specific Oligonucleotide
  source 1..44: mol_type = other DNA
gtaaacatagaacgactcatagtcatagacgaccgaatttacgg

SEQ ID NO: 1110 - DNA, 43 bp, synthetic construct
  misc_feature 1..43: note = Target Specific Oligonucleotide
  source 1..43: mol_type = other DNA
ttccacttcaattagctcttgaattagacgaccgaatttacgg

SEQ ID NO: 1111 - DNA, 40 bp, synthetic construct
  misc_feature 1..40: note = Target Specific Oligonucleotide
  source 1..40: mol_type = other DNA
ctggggtagagatagctcgcttagacgaccgaatttacgg

SEQ ID NO: 1112 - DNA, 41 bp, synthetic construct
  misc_feature 1..41: note = Target Specific Oligonucleotide
  source 1..41: mol_type = other DNA
gctataagctcgcctaccattctagacgaccgaatttacgg

SEQ ID NO: 1113 - DNA, 39 bp, synthetic construct
  misc_feature 1..39: note = Target Specific Oligonucleotide
  source 1..39: mol_type = other DNA
ttcttggggcgggtagagtgtagacgaccgaatttacgg

SEQ ID NO: 1114 - DNA, 40 bp, synthetic construct
  misc_feature 1..40: note = Target Specific Oligonucleotide
  source 1..40: mol_type = other DNA
aagacctcagaccgtcagaagtagacgaccgaatttacgg

SEQ ID NO: 1115 - DNA, 39 bp, synthetic construct
  misc_feature 1..39: note = Target Specific Oligonucleotide
  source 1..39: mol_type = other DNA
tcagatcctgacgacctgcatagacgaccgaatttacgg

SEQ ID NO: 1116 - DNA, 40 bp, synthetic construct
  misc_feature 1..40: note = Target Specific Oligonucleotide
  source 1..40: mol_type = other DNA
gcagaaatctcgagaggacgctagacgaccgaatttacgg

SEQ ID NO: 1117 - DNA, 39 bp, synthetic construct
  misc_feature 1..39: note = Target Specific Oligonucleotide
  source 1..39: mol_type = other DNA
ctcgcagtctaggccgaagatagacgaccgaatttacgg

SEQ ID NO: 1118 - DNA, 39 bp, synthetic construct
  misc_feature 1..39: note = Target Specific Oligonucleotide
  source 1..39: mol_type = other DNA
ccggtctccctcgagaatcatagacgaccgaatttacgg

SEQ ID NO: 1119 - DNA, 39 bp, synthetic construct
  misc_feature 1..39: note = Target Specific Oligonucleotide
  source 1..39: mol_type = other DNA
tcctggtgagaacgtcctgctagacgaccgaatttacgg

SEQ ID NO: 1120 - DNA, 38 bp, synthetic construct
  misc_feature 1..38: note = Target Specific Oligonucleotide
  source 1..38: mol_type = other DNA
tgccctttggtaaggcgcctagacgaccgaatttacgg

SEQ ID NO: 1121 - DNA, 38 bp, synthetic construct
  misc_feature 1..38: note = Target Specific Oligonucleotide
  source 1..38: mol_type = other DNA
cacagacacagccgaggactagacgaccgaatttacgg

SEQ ID NO: 1122 - DNA, 39 bp, synthetic construct
  misc_feature 1..39: note = Target Specific Oligonucleotide
  source 1..39: mol_type = other DNA
tcccaaaagctaccgctgagtagacgaccgaatttacgg

SEQ ID NO: 1123 - DNA, 39 bp, synthetic construct
  misc_feature 1..39: note = Target Specific Oligonucleotide
  source 1..39: mol_type = other DNA
taaggtagagtcgggcgaagtagacgaccgaatttacgg

SEQ ID NO: 1124 - DNA, 38 bp, synthetic construct
  misc_feature 1..38: note = Target Specific Oligonucleotide
  source 1..38: mol_type = other DNA
gagtggccggaaaaacccgtagacgaccgaatttacgg

SEQ ID NO: 1125 - DNA, 37 bp, synthetic construct
  misc_feature 1..37: note = Target Specific Oligonucleotide
  source 1..37: mol_type = other DNA
atgcggacatggtcgccctagacgaccgaatttacgg

SEQ ID NO: 1126 - DNA, 37 bp, synthetic construct
  misc_feature 1..37: note = Target Specific Oligonucleotide
  source 1..37: mol_type = other DNA
ggctcccaaacgccctgatagacgaccgaatttacgg

SEQ ID NO: 1127 - DNA, 40 bp, synthetic construct
  misc_feature 1..40: note = Target Specific Oligonucleotide
  source 1..40: mol_type = other DNA
tccatttcaccgtcagaaaggtagacgaccgaatttacgg

SEQ ID NO: 1128 - DNA, 43 bp, synthetic construct
  misc_feature 1..43: note = Target Specific Oligonucleotide
  source 1..43: mol_type = other DNA
ttgcctagacagcaccgtaatgtagacacgaatattccaattc

SEQ ID NO: 1129 - DNA, 40 bp, synthetic construct
  misc_feature 1..40: note = Target Specific Oligonucleotide
  source 1..40: mol_type = other DNA
ttcccagagcgtggttccatagacacgaatattccaattc

SEQ ID NO: 1130 - DNA, 42 bp, synthetic construct
  misc_feature 1..42: note = Target Specific Oligonucleotide
  source 1..42: mol_type = other DNA
taaggacatcgcttttccggttagacacgaatattccaattc

SEQ ID NO: 1131 - DNA, 40 bp, synthetic construct
  misc_feature 1..40: note = Target Specific Oligonucleotide
  source 1..40: mol_type = other DNA
ttctccacgtcgcggacactagacacgaatattccaattc

SEQ ID NO: 1132 - DNA, 46 bp, synthetic construct
  misc_feature 1..46: note = Target Specific Oligonucleotide
  source 1..46: mol_type = other DNA
gttctcatgatagaggttccttaagtagacacgaatattccaattc

SEQ ID NO: 1133 - DNA, 38 bp, synthetic construct
  misc_feature 1..38: note = Target Specific Oligonucleotide
  source 1..38: mol_type = other DNA
ggccacgtcggtgcagatagacacgaatattccaattc

SEQ ID NO: 1134 - DNA, 41 bp, synthetic construct
  misc_feature 1..41: note = Target Specific Oligonucleotide
  source 1..41: mol_type = other DNA
gttgtggacgatcaacggggtagacacgaatattccaattc

SEQ ID NO: 1135 - DNA, 42 bp, synthetic construct
  misc_feature 1..42: note = Target Specific Oligonucleotide
  source 1..42: mol_type = other DNA
tgtccccttcggggtcatacctagacacgaatattccaattc

SEQ ID NO: 1136 - DNA, 44 bp, synthetic construct
  misc_feature 1..44: note = Target Specific Oligonucleotide
  source 1..44: mol_type = other DNA
aatagaacctcatccggtagtggtagacacgaatattccaattc

SEQ ID NO: 1137 - DNA, 42 bp, synthetic construct
  misc_feature 1..42: note = Target Specific Oligonucleotide
  source 1..42: mol_type = other DNA
cagtgcagcgtatgtggtatttagacacgaatattccaattc

SEQ ID NO: 1138 - DNA, 42 bp, synthetic construct
  misc_feature 1..42: note = Target Specific Oligonucleotide
  source 1..42: mol_type = other DNA
aaagcttgtccgattggatggtagacacgaatattccaattc

SEQ ID NO: 1139 - DNA, 42 bp, synthetic construct
  misc_feature 1..42: note = Target Specific Oligonucleotide
  source 1..42: mol_type = other DNA
aacccttgtagatcggtggtatagacacgaatattccaattc

SEQ ID NO: 1140 - DNA, 42 bp, synthetic construct
  misc_feature 1..42: note = Target Specific Oligonucleotide
  source 1..42: mol_type = other DNA
agtagagaacgtttccaccgttagacacgaatattccaattc

SEQ ID NO: 1141 - DNA, 42 bp, synthetic construct
  misc_feature 1..42: note = Target Specific Oligonucleotide
  source 1..42: mol_type = other DNA
actgctcattgtcgttggttctagacacgaatattccaattc

SEQ ID NO: 1142 - DNA, 41 bp, synthetic construct
  misc_feature 1..41: note = Target Specific Oligonucleotide
  source 1..41: mol_type = other DNA
aacgtattgccgagaacccatagacacgaatattccaattc

SEQ ID NO: 1143 - DNA, 43 bp, synthetic construct
  misc_feature 1..43: note = Target Specific Oligonucleotide
  source 1..43: mol_type = other DNA
acaaagtgcatatagagtggcctagacacgaatattccaattc

SEQ ID NO: 1144 - DNA, 42 bp, synthetic construct
  misc_feature 1..42: note = Target Specific Oligonucleotide
  source 1..42: mol_type = other DNA
agccatccctagacactcgtttagacacgaatattccaattc

SEQ ID NO: 1145 - DNA, 42 bp, synthetic construct
  misc_feature 1..42: note = Target Specific Oligonucleotide
  source 1..42: mol_type = other DNA
gcgatttggagcataccagagtagacacgaatattccaattc

SEQ ID NO: 1146 - DNA, 42 bp, synthetic construct
  misc_feature 1..42: note = Target Specific Oligonucleotide
  source 1..42: mol_type = other DNA
aggatccgatcgaaactcagctagacacgaatattccaattc

SEQ ID NO: 1147 - DNA, 44 bp, synthetic construct
  misc_feature 1..44: note = Target Specific Oligonucleotide
  source 1..44: mol_type = other DNA
ctggaatatatcgcttgtagctgtagacacgaatattccaattc

SEQ ID NO: 1148 - DNA, 41 bp, synthetic construct
  misc_feature 1..41: note = Target Specific Oligonucleotide
  source 1..41: mol_type = other DNA
cttcaggtcatgcgtggacatagacacgaatattccaattc

SEQ ID NO: 1149 - DNA, 41 bp, synthetic construct
  misc_feature 1..41: note = Target Specific Oligonucleotide
  source 1..41: mol_type = other DNA
agagaagcgcacttccgtgttagacacgaatattccaattc

SEQ ID NO: 1150 - DNA, 41 bp, synthetic construct
  misc_feature 1..41: note = Target Specific Oligonucleotide
  source 1..41: mol_type = other DNA
tgatgttatccgtgcgcaggtagacacgaatattccaattc

SEQ ID NO: 1151 - DNA, 44 bp, synthetic construct
  misc_feature 1..44: note = Target Specific Oligonucleotide
  source 1..44: mol_type = other DNA
aaagaccgtaatcgttcacattgtagacacgaatattccaattc

SEQ ID NO: 1152 - DNA, 42 bp, synthetic construct
  misc_feature 1..42: note = Target Specific Oligonucleotide
  source 1..42: mol_type = other DNA
agagatttgtctacgtagagccgtagactcagaaatctcacg

SEQ ID NO: 1153 - DNA, 41 bp, synthetic construct
  misc_feature 1..41: note = Target Specific Oligonucleotide
  source 1..41: mol_type = other DNA
tcctttgcatacggtctcttcgtagactcagaaatctcacg

SEQ ID NO: 1154 - DNA, 43 bp, synthetic construct
  misc_feature 1..43: note = Target Specific Oligonucleotide
  source 1..43: mol_type = other DNA
aagtttgtttacatccgatgttactagactcagaaatctcacg

SEQ ID NO: 1155 - DNA, 39 bp, synthetic construct
  misc_feature 1..39: note = Target Specific Oligonucleotide
  source 1..39: mol_type = other DNA
ttcccatacagaacgtggcctagactcagaaatctcacg

SEQ ID NO: 1156 - DNA, 40 bp, synthetic construct
  misc_feature 1..40: note = Target Specific Oligonucleotide
  source 1..40: mol_type = other DNA
aacagatcaacggcaaaagcctagactcagaaatctcacg

SEQ ID NO: 1157 - DNA, 42 bp, synthetic construct
  misc_feature 1..42: note = Target Specific Oligonucleotide
  source 1..42: mol_type = other DNA
catccttgggtagtaggatgaactagactcagaaatctcacg

SEQ ID NO: 1158 - DNA, 40 bp, synthetic construct
  misc_feature 1..40: note = Target Specific Oligonucleotide
  source 1..40: mol_type = other DNA
gaatttgctagttgcagggcatagactcagaaatctcacg

SEQ ID NO: 1159 - DNA, 45 bp, synthetic construct
  misc_feature 1..45: note = Target Specific Oligonucleotide
  source 1..45: mol_type = other DNA
tgatcttacaattgatactgtcaatgtagactcagaaatctcacg

SEQ ID NO: 1160 - DNA, 38 bp, synthetic construct
  misc_feature 1..38: note = Target Specific Oligonucleotide
  source 1..38: mol_type = other DNA
tggatgtggaggcgaccgttagactcagaaatctcacg

SEQ ID NO: 1161 - DNA, 42 bp, synthetic construct
  misc_feature 1..42: note = Target Specific Oligonucleotide
  source 1..42: mol_type = other DNA
acttctggacctatagtagcctgtagactcagaaatctcacg

SEQ ID NO: 1162 - DNA, 42 bp, synthetic construct
  misc_feature 1..42: note = Target Specific Oligonucleotide
  source 1..42: mol_type = other DNA
caacacggatagcttatccaacctagactcagaaatctcacg

SEQ ID NO: 1163 - DNA, 40 bp, synthetic construct
  misc_feature 1..40: note = Target Specific Oligonucleotide
  source 1..40: mol_type = other DNA
tgtcaaggtaacgtgagcacttagactcagaaatctcacg

SEQ ID NO: 1164 - DNA, 41 bp, synthetic construct
  misc_feature 1..41: note = Target Specific Oligonucleotide
  source 1..41: mol_type = other DNA
tttacagaaatcggcatccacatagactcagaaatctcacg

SEQ ID NO: 1165 - DNA, 44 bp, synthetic construct
  misc_feature 1..44: note = Target Specific Oligonucleotide
  source 1..44: mol_type = other DNA
cagctactagaggtctataattccttagactcagaaatctcacg

SEQ ID NO: 1166 - DNA, 40 bp, synthetic construct
  misc_feature 1..40: note = Target Specific Oligonucleotide
  source 1..40: mol_type = other DNA
ctggcacctctatggaatccctagactcagaaatctcacg

SEQ ID NO: 1167 - DNA, 43 bp, synthetic construct
  misc_feature 1..43: note = Target Specific Oligonucleotide
  source 1..43: mol_type = other DNA
attctgatatcggctattgtgagatagactcagaaatctcacg

SEQ ID NO: 1168 - DNA, 39 bp, synthetic construct
  misc_feature 1..39: note = Target Specific Oligonucleotide
  source 1..39: mol_type = other DNA
gccaccagtctagtgccaactagactcagaaatctcacg

SEQ ID NO: 1169 - DNA, 40 bp, synthetic construct
  misc_feature 1..40: note = Target Specific Oligonucleotide
  source 1..40: mol_type = other DNA
caactaccccggacaatttgatagactcagaaatctcacg

SEQ ID NO: 1170 - DNA, 41 bp, synthetic construct
  misc_feature 1..41: note = Target Specific Oligonucleotide
  source 1..41: mol_type = other DNA
aggacttaacatgggctatgcctagactcagaaatctcacg

SEQ ID NO: 1171 - DNA, 41 bp, synthetic construct
  misc_feature 1..41: note = Target Specific Oligonucleotide
  source 1..41: mol_type = other DNA
ctctgcgaacgaagtgtttttgtagactcagaaatctcacg

SEQ ID NO: 1172 - DNA, 43 bp, synthetic construct
  misc_feature 1..43: note = Target Specific Oligonucleotide
  source 1..43: mol_type = other DNA
tgcagcatattaaggtgttgattttagactcagaaatctcacg

SEQ ID NO: 1173 - DNA, 44 bp, synthetic construct
  misc_feature 1..44: note = Target Specific Oligonucleotide
  source 1..44: mol_type = other DNA
aagtaactaaacccatagactgaaatagactcagaaatctcacg

SEQ ID NO: 1174 - DNA, 40 bp, synthetic construct
  misc_feature 1..40: note = Target Specific Oligonucleotide
  source 1..40: mol_type = other DNA
ctgccagggcttaacataaggtagactcagaaatctcacg

SEQ ID NO: 1175 - DNA, 42 bp, synthetic construct
  misc_feature 1..42: note = Target Specific Oligonucleotide
  source 1..42: mol_type = other DNA
ataacatttgatagggccactcctagactcagaaatctcacg

SEQ ID NO: 1176 - DNA, 41 bp, synthetic construct
  misc_feature 1..41: note = Target Specific Oligonucleotide
  source 1..41: mol_type = other DNA
ctgaagataatcgcagacaccctagacgtctcccgtatttt

SEQ ID NO: 1177 - DNA, 41 bp, synthetic construct
  misc_feature 1..41: note = Target Specific Oligonucleotide
  source 1..41: mol_type = other DNA
tgactgaacacgtgttctgttctagacgtctcccgtatttt

SEQ ID NO: 1178 - DNA, 39 bp, synthetic construct
  misc_feature 1..39: note = Target Specific Oligonucleotide
  source 1..39: mol_type = other DNA
tttcagccatacccggatggtagacgtctcccgtatttt

SEQ ID NO: 1179 - DNA, 41 bp, synthetic construct
  misc_feature 1..41: note = Target Specific Oligonucleotide
  source 1..41: mol_type = other DNA
tgagaagagtaaccgctgaatatagacgtctcccgtatttt

SEQ ID NO: 1180 - DNA, 38 bp, synthetic construct
  misc_feature 1..38: note = Target Specific Oligonucleotide
  source 1..38: mol_type = other DNA
acactgcaattccgccgtctagacgtctcccgtatttt

SEQ ID NO: 1181 - DNA, 38 bp, synthetic construct
  misc_feature 1..38: note = Target Specific Oligonucleotide
  source 1..38: mol_type = other DNA
ttgtcgtggccggtactgatagacgtctcccgtatttt

SEQ ID NO: 1182 - DNA, 39 bp, synthetic construct
  misc_feature 1..39: note = Target Specific Oligonucleotide
  source 1..39: mol_type = other DNA
ggcaggcgacactaccttcatagacgtctcccgtatttt

SEQ ID NO: 1183 - DNA, 40 bp, synthetic construct
  misc_feature 1..40: note = Target Specific Oligonucleotide
  source 1..40: mol_type = other DNA
ctgggccagtattgaccactgtagacgtctcccgtatttt

SEQ ID NO: 1184 - DNA, 40 bp, synthetic construct
  misc_feature 1..40: note = Target Specific Oligonucleotide
  source 1..40: mol_type = other DNA
tctcagatcagtgcggagaaatagacgtctcccgtatttt

SEQ ID NO: 1185 - DNA, 39 bp, synthetic construct
  misc_feature 1..39: note = Target Specific Oligonucleotide
  source 1..39: mol_type = other DNA
gcccacaaatatccgtctgctagacgtctcccgtatttt

SEQ ID NO: 1186 - DNA, 36 bp, synthetic construct
  misc_feature 1..36: note = Target Specific Oligonucleotide
  source 1..36: mol_type = other DNA
ccatgttcgcgcggcgatagacgtctcccgtatttt

SEQ ID NO: 1187 - DNA, 42 bp, synthetic construct
  misc_feature 1..42: note = Target Specific Oligonucleotide
  source 1..42: mol_type = other DNA
atgaaaggtcgctagagaagtcctagacgtctcccgtatttt

SEQ ID NO: 1188 - DNA, 40 bp, synthetic construct
  misc_feature 1..40: note = Target Specific Oligonucleotide
  source 1..40: mol_type = other DNA
aatcacaaacccggacttggatagacgtctcccgtatttt

SEQ ID NO: 1189 - DNA, 40 bp, synthetic construct
  misc_feature 1..40: note = Target Specific Oligonucleotide
  source 1..40: mol_type = other DNA
actagcctgtcacgtttgtcttagacgtctcccgtatttt

SEQ ID NO: 1190 - DNA, 41 bp, synthetic construct
  misc_feature 1..41: note = Target Specific Oligonucleotide
  source 1..41: mol_type = other DNA
atctttacaatgcggtcggtcctagacgtctcccgtatttt

SEQ ID NO: 1191 - DNA, 41 bp, synthetic construct
  misc_feature 1..41: note = Target Specific Oligonucleotide
  source 1..41: mol_type = other DNA
tgtcctggtatgatcccaacactagacgtctcccgtatttt

SEQ ID NO: 1192 - DNA, 41 bp, synthetic construct
  misc_feature 1..41: note = Target Specific Oligonucleotide
  source 1..41: mol_type = other DNA
ctgatcatttcgcatcacagtgtagacgtctcccgtatttt

SEQ ID NO: 1193 - DNA, 41 bp, synthetic construct
  misc_feature 1..41: note = Target Specific Oligonucleotide
  source 1..41: mol_type = other DNA
ctttccttacgtgtgtcacacatagacgtctcccgtatttt

SEQ ID NO: 1194 - DNA, 42 bp, synthetic construct
  misc_feature 1..42: note = Target Specific Oligonucleotide
  source 1..42: mol_type = other DNA
tagtttgatcgcctccttaaacctagacgtctcccgtatttt

SEQ ID NO: 1195 - DNA, 42 bp, synthetic construct
  misc_feature 1..42: note = Target Specific Oligonucleotide
  source 1..42: mol_type = other DNA
aaatccctaggatagcacctttgtagacgtctcccgtatttt

SEQ ID NO: 1196 - DNA, 42 bp, synthetic construct
  misc_feature 1..42: note = Target Specific Oligonucleotide
  source 1..42: mol_type = other DNA
cttacacttacgatgagcagatgtagacgtctcccgtatttt

SEQ ID NO: 1197 - DNA, 43 bp, synthetic construct
  misc_feature 1..43: note = Target Specific Oligonucleotide
  source 1..43: mol_type = other DNA
catttgctacctagcatatgagagtagacgtctcccgtatttt

SEQ ID NO: 1198 - DNA, 41 bp, synthetic construct
  misc_feature 1..41: note = Target Specific Oligonucleotide
  source 1..41: mol_type = other DNA
acagatgtcaaacggtcacatttagacgtctcccgtatttt

SEQ ID NO: 1199 - DNA, 43 bp, synthetic construct
  misc_feature 1..43: note = Target Specific Oligonucleotide
  source 1..43: mol_type = other DNA
ttctaagatacgtcactccaattctagacgtctcccgtatttt

SEQ ID NO: 1200 - DNA, 42 bp, synthetic construct
  misc_feature 1..42: note = Target Specific Oligonucleotide
  source 1..42: mol_type = other DNA
acagtccgtcgattctttacaatagactctataaatcagcgt

SEQ ID NO: 1201 - DNA, 41 bp, synthetic construct
  misc_feature 1..41: note = Target Specific Oligonucleotide
  source 1..41: mol_type = other DNA
tctccacgtaaatcccgatcttagactctataaatcagcgt

SEQ ID NO: 1202 - DNA, 40 bp, synthetic construct
  misc_feature 1..40: note = Target Specific Oligonucleotide
  source 1..40: mol_type = other DNA
cacgtgctgaaccaaccacttagactctataaatcagcgt

SEQ ID NO: 1203 - DNA, 39 bp, synthetic construct
  misc_feature 1..39: note = Target Specific Oligonucleotide
  source 1..39: mol_type = other DNA
tcttgccgaagttggccgttagactctataaatcagcgt

SEQ ID NO: 1204 - DNA, 41 bp, synthetic construct
  misc_feature 1..41: note = Target Specific Oligonucleotide
  source 1..41: mol_type = other DNA
agcagcagttggatctacagttagactctataaatcagcgt

SEQ ID NO: 1205 - DNA, 43 bp, synthetic construct
  misc_feature 1..43: note = Target Specific Oligonucleotide
  source 1..43: mol_type = other DNA
gtctagcagtaaacctgtaaacctagactctataaatcagcgt

SEQ ID NO: 1206 - DNA, 42 bp, synthetic construct
  misc_feature 1..42: note = Target Specific Oligonucleotide
  source 1..42: mol_type = other DNA
gtgcattattggacgaacatcatagactctataaatcagcgt

SEQ ID NO: 1207 - DNA, 45 bp, synthetic construct
  misc_feature 1..45: note = Target Specific Oligonucleotide
  source 1..45: mol_type = other DNA
gctgttgttaatggtctataatccatagactctataaatcagcgt

SEQ ID NO: 1208 - DNA, 45 bp, synthetic construct
  misc_feature 1..45: note = Target Specific Oligonucleotide
  source 1..45: mol_type = other DNA
cacacatatcctatgttcatcaatatagactctataaatcagcgt

SEQ ID NO: 1209 - DNA, 43 bp, synthetic construct
  misc_feature 1..43: note = Target Specific Oligonucleotide
  source 1..43: mol_type = other DNA
ttgagtggtcgttttcttacacatagactctataaatcagcgt

SEQ ID NO: 1210 - DNA, 45 bp, synthetic construct
  misc_feature 1..45: note = Target Specific Oligonucleotide
  source 1..45: mol_type = other DNA
cataatcaaaacgaaatgtttggtttagactctataaatcagcgt

SEQ ID NO: 1211 - DNA, 41 bp, synthetic construct
  misc_feature 1..41: note = Target Specific Oligonucleotide
  source 1..41: mol_type = other DNA
tttgtagcatcaacgtcctggtagactctataaatcagcgt

SEQ ID NO: 1212 - DNA, 41 bp, synthetic construct
  misc_feature 1..41: note = Target Specific Oligonucleotide
  source 1..41: mol_type = other DNA
caacacatttgacctcccgtttagactctataaatcagcgt

SEQ ID NO: 1213 - DNA, 43 bp, synthetic construct
  misc_feature 1..43: note = Target Specific Oligonucleotide
  source 1..43: mol_type = other DNA
ctaaccaccgaatagattcctggtagactctataaatcagcgt

SEQ ID NO: 1214 - DNA, 41 bp, synthetic construct
  misc_feature 1..41: note = Target Specific Oligonucleotide
  source 1..41: mol_type = other DNA
gcagatgtttgaccggatgtttagactctataaatcagcgt

SEQ ID NO: 1215 - DNA, 42 bp, synthetic construct
  misc_feature 1..42: note = Target Specific Oligonucleotide
  source 1..42: mol_type = other DNA
tgcttgatgtattcggccatcgtagactctataaatcagcgt

SEQ ID NO: 1216 - DNA, 42 bp, synthetic construct
  misc_feature 1..42: note = Target Specific Oligonucleotide
  source 1..42: mol_type = other DNA
tgagtttacttgcacggaaaggtagactctataaatcagcgt

SEQ ID NO: 1217 - DNA, 42 bp, synthetic construct
  misc_feature 1..42: note = Target Specific Oligonucleotide
  source 1..42: mol_type = other DNA
cactttatcccgcagttcagtttagactctataaatcagcgt

SEQ ID NO: 1218 - DNA, 41 bp, synthetic construct
  misc_feature 1..41: note = Target Specific Oligonucleotide
  source 1..41: mol_type = other DNA
gcaaagcatgtagccattccctagactctataaatcagcgt

SEQ ID NO: 1219 - DNA, 43 bp, synthetic construct
  misc_feature 1..43: note = Target Specific Oligonucleotide
  source 1..43: mol_type = other DNA
aaagacatctcgagctgctaatgtagactctataaatcagcgt

SEQ ID NO: 1220 - DNA, 43 bp, synthetic construct
  misc_feature 1..43: note = Target Specific Oligonucleotide
  source 1..43: mol_type = other DNA
gtcaccacccatagtatgagttttagactctataaatcagcgt

SEQ ID NO: 1221 - DNA, 43 bp, synthetic construct
  misc_feature 1..43: note = Target Specific Oligonucleotide
  source 1..43: mol_type = other DNA
tttctacctaaggctctgatgcatagactctataaatcagcgt

SEQ ID NO: 1222 - DNA, 41 bp, synthetic construct
  misc_feature 1..41: note = Target Specific Oligonucleotide
  source 1..41: mol_type = other DNA
tagagctgcacggaaagattttagactctataaatcagcgt

SEQ ID NO: 1223 - DNA, 43 bp, synthetic construct
  misc_feature 1..43: note = Target Specific Oligonucleotide
  source 1..43: mol_type = other DNA
gtgtagcatatgaatcgacatcatagactctataaatcagcgt

SEQ ID NO: 1224 - DNA, 43 bp, synthetic construct
  misc_feature 1..43: note = Target Specific Oligonucleotide
  source 1..43: mol_type = other DNA
tagccttgtatggcagaatcgtagacctaaattactgtgaatg

SEQ ID NO: 1225 - DNA, 45 bp, synthetic construct
  misc_feature 1..45: note = Target Specific Oligonucleotide
  source 1..45: mol_type = other DNA
tgaattgccctgggtataactcctagacctaaattactgtgaatg

SEQ ID NO: 1226 - DNA, 42 bp, synthetic construct
  misc_feature 1..42: note = Target Specific Oligonucleotide
  source 1..42: mol_type = other DNA
ttcctgttgcaacgtgagggtagacctaaattactgtgaatg

SEQ ID NO: 1227 - DNA, 46 bp, synthetic construct
  misc_feature 1..46: note = Target Specific Oligonucleotide
  source 1..46: mol_type = other DNA
atgaaagacacgatcaaaattgaatagacctaaattactgtgaatg

SEQ ID NO: 1228 - DNA, 43 bp, synthetic construct
  misc_feature 1..43: note = Target Specific Oligonucleotide
  source 1..43: mol_type = other DNA
gtcagcacgaagtgagttcaatagacctaaattactgtgaatg

SEQ ID NO: 1229 - DNA, 44 bp, synthetic construct
  misc_feature 1..44: note = Target Specific Oligonucleotide
  source 1..44: mol_type = other DNA
cagtttgcagcgtagaatctcttagacctaaattactgtgaatg

SEQ ID NO: 1230 - DNA, 45 bp, synthetic construct
  misc_feature 1..45: note = Target Specific Oligonucleotide
  source 1..45: mol_type = other DNA
gtgcagataatacgtgtctttgctagacctaaattactgtgaatg

SEQ ID NO: 1231 - DNA, 45 bp, synthetic construct
  misc_feature 1..45: note = Target Specific Oligonucleotide
  source 1..45: mol_type = other DNA
tccaatgagtatgacaagtccattagacctaaattactgtgaatg

SEQ ID NO: 1232 - DNA, 45 bp, synthetic construct
  misc_feature 1..45: note = Target Specific Oligonucleotide
  source 1..45: mol_type = other DNA
tgtaagaaagctcggtcttcaaatagacctaaattactgtgaatg

SEQ ID NO: 1233 - DNA, 46 bp, synthetic construct
  misc_feature 1..46: note = Target Specific Oligonucleotide
  source 1..46: mol_type = other DNA
ccatgtaagatacacgtaagagaatagacctaaattactgtgaatg

SEQ ID NO: 1234 - DNA, 46 bp, synthetic construct
  misc_feature 1..46: note = Target Specific Oligonucleotide
  source 1..46: mol_type = other DNA
gactcaagagcatttcgtaattgttagacctaaattactgtgaatg

SEQ ID NO: 1235 - DNA, 46 bp, synthetic construct
  misc_feature 1..46: note = Target Specific Oligonucleotide
  source 1..46: mol_type = other DNA
atgtcattagtcacctcaagttcttagacctaaattactgtgaatg

SEQ ID NO: 1236 - DNA, 39 bp, synthetic construct
  misc_feature 1..39: note = Target Specific Oligonucleotide
  source 1..39: mol_type = other DNA
ccgcactcgcacgcagatagacctaaattactgtgaatg

SEQ ID NO: 1237 - DNA, 47 bp, synthetic construct
  misc_feature 1..47: note = Target Specific Oligonucleotide
  source 1..47: mol_type = other DNA
tgttaacagtatcgtgaatatcacttagacctaaattactgtgaatg

SEQ ID NO: 1238 - DNA, 45 bp, synthetic construct
  misc_feature 1..45: note = Target Specific Oligonucleotide
  source 1..45: mol_type = other DNA
actttgccatctataagggaggttagacctaaattactgtgaatg

SEQ ID NO: 1239 - DNA, 48 bp, synthetic construct
  misc_feature 1..48: note = Target Specific Oligonucleotide
  source 1..48: mol_type = other DNA
cttgctatctcgataatttatgaaactagacctaaattactgtgaatg

SEQ ID NO: 1240 - DNA, 48 bp, synthetic construct
  misc_feature 1..48: note = Target Specific Oligonucleotide
  source 1..48: mol_type = other DNA
ttaatgtatcaagagtgttactgaaatagacctaaattactgtgaatg

SEQ ID NO: 1241 - DNA, 46 bp, synthetic construct
  misc_feature 1..46: note = Target Specific Oligonucleotide
  source 1..46: mol_type = other DNA
tcaaatgggataccttaacagatctagacctaaattactgtgaatg

SEQ ID NO: 1242 - DNA, 50 bp, synthetic construct
  misc_feature 1..50: note = Target Specific Oligonucleotide
  source 1..50: mol_type = other DNA
ataaataagcttcgatttatattacagctagacctaaattactgtgaatg

SEQ ID NO: 1243 - DNA, 45 bp, synthetic construct
  misc_feature 1..45: note = Target Specific Oligonucleotide
  source 1..45: mol_type = other DNA
cttgtgtggtaggattaacctggtagacctaaattactgtgaatg

SEQ ID NO: 1244 - DNA, 43 bp, synthetic construct
  misc_feature 1..43: note = Target Specific Oligonucleotide
  source 1..43: mol_type = other DNA
gttggtcaactcgagcatctttagacctaaattactgtgaatg

SEQ ID NO: 1245 - DNA, 44 bp, synthetic construct
  misc_feature 1..44: note = Target Specific Oligonucleotide
  source 1..44: mol_type = other DNA
tttggtttgtaggcctgtagcttagacctaaattactgtgaatg

SEQ ID NO: 1246 - DNA, 45 bp, synthetic construct
  misc_feature 1..45: note = Target Specific Oligonucleotide
  source 1..45: mol_type = other DNA
cattaggcagtaactcttgttcctagacctaaattactgtgaatg

SEQ ID NO: 1247 - DNA, 45 bp, synthetic construct
  misc_feature 1..45: note = Target Specific Oligonucleotide
  source 1..45: mol_type = other DNA
cttctcagatacgcttcttcttatagacctaaattactgtgaatg

SEQ ID NO: 1248 - DNA, 31 bp, synthetic construct
  misc_feature 1..31: note = Decoding Oligonucleotide
  source 1..31: mol_type = other DNA
ggctaatttattaccttctagctataacgat

SEQ ID NO: 1249 - DNA, 31 bp, synthetic construct
  misc_feature 1..31: note = Decoding Oligonucleotide
  source 1..31: mol_type = other DNA
accttaagaatggaattctagctataacgat

SEQ ID NO: 1250 - DNA, 30 bp, synthetic construct
  misc_feature 1..30: note = Decoding Oligonucleotide
  source 1..30: mol_type = other DNA
acttaacttgagtggtctagctataacgat

SEQ ID NO: 1251 - DNA, 29 bp, synthetic construct
  misc_feature 1..29: note = Decoding Oligonucleotide
  source 1..29: mol_type = other DNA
ggctaatttattacctcggtctagatgga

SEQ ID NO: 1252 - DNA, 29 bp, synthetic construct
  misc_feature 1..29: note = Decoding Oligonucleotide
  source 1..29: mol_type = other DNA
accttaagaatggaatcggtctagatgga

SEQ ID NO: 1253 - DNA, 28 bp, synthetic construct
  misc_feature 1..28: note = Decoding Oligonucleotide
  source 1..28: mol_type = other DNA
acttaacttgagtggcggtctagatgga

SEQ ID NO: 1254 - DNA, 32 bp, synthetic construct
  misc_feature 1..32: note = Decoding Oligonucleotide
  source 1..32: mol_type = other DNA
ggctaatttattacctgttaactccgtagtaa

SEQ ID NO: 1255 - DNA, 32 bp, synthetic construct
  misc_feature 1..32: note = Decoding Oligonucleotide
  source 1..32: mol_type = other DNA
accttaagaatggaatgttaactccgtagtaa

SEQ ID NO: 1256 - DNA, 31 bp, synthetic construct
  misc_feature 1..31: note = Decoding Oligonucleotide
  source 1..31: mol_type = other DNA
acttaacttgagtgggttaactccgtagtaa

SEQ ID NO: 1257 - DNA, 31 bp, synthetic construct
  misc_feature 1..31: note = Decoding Oligonucleotide
  source 1..31: mol_type = other DNA
ggctaatttattacctcgctatatctactga

SEQ ID NO: 1258 - DNA, 31 bp, synthetic construct
  misc_feature 1..31: note = Decoding Oligonucleotide
  source 1..31: mol_type = other DNA
accttaagaatggaatcgctatatctactga

SEQ ID NO: 1259 - DNA, 30 bp, synthetic construct
  misc_feature 1..30: note = Decoding Oligonucleotide
  source 1..30: mol_type = other DNA
acttaacttgagtggcgctatatctactga

SEQ ID NO: 1260 - DNA, 30 bp, synthetic construct
  misc_feature 1..30: note = Decoding Oligonucleotide
  source 1..30: mol_type = other DNA
ggctaatttattacctgtttccgtttcgaa

SEQ ID NO: 1261 - DNA, 30 bp, synthetic construct
  misc_feature 1..30: note = Decoding Oligonucleotide
  source 1..30: mol_type = other DNA
accttaagaatggaatgtttccgtttcgaa

SEQ ID NO: 1262 - DNA, 29 bp, synthetic construct
  misc_feature 1..29: note = Decoding Oligonucleotide
  source 1..29: mol_type = other DNA
acttaacttgagtgggtttccgtttcgaa

SEQ ID NO: 1263 - DNA, 30 bp, synthetic construct
  misc_feature 1..30: note = Decoding Oligonucleotide
  source 1..30: mol_type = other DNA
ggctaatttattaccttagcgtttcaaaag

SEQ ID NO: 1264 - DNA, 30 bp, synthetic construct
  misc_feature 1..30: note = Decoding Oligonucleotide
  source 1..30: mol_type = other DNA
accttaagaatggaattagcgtttcaaaag

SEQ ID NO: 1265 - DNA, 29 bp, synthetic construct
  misc_feature 1..29: note = Decoding Oligonucleotide
  source 1..29: mol_type = other DNA
acttaacttgagtggtagcgtttcaaaag

SEQ ID NO: 1266 - DNA, 28 bp, synthetic construct
  misc_feature 1..28: note = Decoding Oligonucleotide
  source 1..28: mol_type = other DNA
ggctaatttattaccttgatccgacgag

SEQ ID NO: 1267 - DNA, 28 bp, synthetic construct
  misc_feature 1..28: note = Decoding Oligonucleotide
  source 1..28: mol_type = other DNA
accttaagaatggaattgatccgacgag

SEQ ID NO: 1268 - DNA, 27 bp, synthetic construct
  misc_feature 1..27: note = Decoding Oligonucleotide
  source 1..27: mol_type = other DNA
acttaacttgagtggtgatccgacgag

SEQ ID NO: 1269 - DNA, 31 bp, synthetic construct
  misc_feature 1..31: note = Decoding Oligonucleotide
  source 1..31: mol_type = other DNA
ggctaatttattacctaatctacatgtctga

SEQ ID NO: 1270 - DNA, 31 bp, synthetic construct
  misc_feature 1..31: note = Decoding Oligonucleotide
  source 1..31: mol_type = other DNA
accttaagaatggaataatctacatgtctga

SEQ ID NO: 1271 - DNA, 30 bp, synthetic construct
  misc_feature 1..30: note = Decoding Oligonucleotide
  source 1..30: mol_type = other DNA
acttaacttgagtggaatctacatgtctga

SEQ ID NO: 1272 - DNA, 30 bp, synthetic construct
  misc_feature 1..30: note = Decoding Oligonucleotide
  source 1..30: mol_type = other DNA
ggctaatttattacctggacgttgaataca

SEQ ID NO: 1273 - DNA, 30 bp, synthetic construct
  misc_feature 1..30: note = Decoding Oligonucleotide
  source 1..30: mol_type = other DNA
accttaagaatggaatggacgttgaataca

SEQ ID NO: 1274 - DNA, 29 bp, synthetic construct
  misc_feature 1..29: note = Decoding Oligonucleotide
  source 1..29: mol_type = other DNA
acttaacttgagtggggacgttgaataca

SEQ ID NO: 1275 - DNA, 31 bp, synthetic construct
  misc_feature 1..31: note = Decoding Oligonucleotide
  source 1..31: mol_type = other DNA
ggctaatttattaccttaaagtgacaattca

SEQ ID NO: 1276 - DNA, 31 bp, synthetic construct
  misc_feature 1..31: note = Decoding Oligonucleotide
  source 1..31: mol_type = other DNA
accttaagaatggaattaaagtgacaattca

SEQ ID NO: 1277 - DNA, 30 bp, synthetic construct
  misc_feature 1..30: note = Decoding Oligonucleotide
  source 1..30: mol_type = other DNA
acttaacttgagtggtaaagtgacaattca

SEQ ID NO: 1278 - DNA, 32 bp, synthetic construct
  misc_feature 1..32: note = Decoding Oligonucleotide
  source 1..32: mol_type = other DNA
ggctaatttattacctgattcccataattagg

SEQ ID NO: 1279 - DNA, 32 bp, synthetic construct
  misc_feature 1..32: note = Decoding Oligonucleotide
  source 1..32: mol_type = other DNA
accttaagaatggaatgattcccataattagg

SEQ ID NO: 1280 - DNA, 31 bp, synthetic construct
  misc_feature 1..31: note = Decoding Oligonucleotide
  source 1..31: mol_type = other DNA
acttaacttgagtgggattcccataattagg

SEQ ID NO: 1281 - DNA, 33 bp, synthetic construct
  misc_feature 1..33: note = Decoding Oligonucleotide
  source 1..33: mol_type = other DNA
ggctaatttattacctcgtcgtataaatctatc

SEQ ID NO: 1282 - DNA, 33 bp, synthetic construct
  misc_feature 1..33: note = Decoding Oligonucleotide
  source 1..33: mol_type = other DNA
accttaagaatggaatcgtcgtataaatctatc

SEQ ID NO: 1283 - DNA, 32 bp, synthetic construct
  misc_feature 1..32: note = Decoding Oligonucleotide
  source 1..32: mol_type = other DNA
acttaacttgagtggcgtcgtataaatctatc

SEQ ID NO: 1284 - DNA, 34 bp, synthetic construct
  misc_feature 1..34: note = Decoding Oligonucleotide
  source 1..34: mol_type = other DNA
ggctaatttattacctgatgtctatcctttataa

SEQ ID NO: 1285 - DNA, 34 bp, synthetic construct
  misc_feature 1..34: note = Decoding Oligonucleotide
  source 1..34: mol_type = other DNA
accttaagaatggaatgatgtctatcctttataa

SEQ ID NO: 1286 - DNA, 33 bp, synthetic construct
  misc_feature 1..33: note = Decoding Oligonucleotide
  source 1..33: mol_type = other DNA
acttaacttgagtgggatgtctatcctttataa

SEQ ID NO: 1287 - DNA, 30 bp, synthetic construct
  misc_feature 1..30: note = Decoding Oligonucleotide
  source 1..30: mol_type = other DNA
ggctaatttattaccttttccacgataacc

SEQ ID NO: 1288 - DNA, 30 bp, synthetic construct
  misc_feature 1..30: note = Decoding Oligonucleotide
  source 1..30: mol_type = other DNA
accttaagaatggaattttccacgataacc

SEQ ID NO: 1289 - DNA, 29 bp, synthetic construct
  misc_feature 1..29: note = Decoding Oligonucleotide
  source 1..29: mol_type = other DNA
acttaacttgagtggtttccacgataacc

SEQ ID NO: 1290 - DNA, 31 bp, synthetic construct
  misc_feature 1..31: note = Decoding Oligonucleotide
  source 1..31: mol_type = other DNA
ggctaatttattaccttagtaccgaataagg

SEQ ID NO: 1291 - DNA, 31 bp, synthetic construct
  misc_feature 1..31: note = Decoding Oligonucleotide
  source 1..31: mol_type = other DNA
accttaagaatggaattagtaccgaataagg

SEQ ID NO: 1292 - DNA, 30 bp, synthetic construct
  misc_feature 1..30: note = Decoding Oligonucleotide
  source 1..30: mol_type = other DNA
acttaacttgagtggtagtaccgaataagg

SEQ ID NO: 1293 - DNA, 32 bp, synthetic construct
  misc_feature 1..32: note = Decoding Oligonucleotide
  source 1..32: mol_type = other DNA
ggctaatttattaccttaaagttgtaaaatcc

SEQ ID NO: 1294 - DNA, 32 bp, synthetic construct
  misc_feature 1..32: note = Decoding Oligonucleotide
  source 1..32: mol_type = other DNA
accttaagaatggaattaaagttgtaaaatcc

SEQ ID NO: 1295 - DNA, 31 bp, synthetic construct
  misc_feature 1..31: note = Decoding Oligonucleotide
  source 1..31: mol_type = other DNA
acttaacttgagtggtaaagttgtaaaatcc

SEQ ID NO: 1296 - DNA, 32 bp, synthetic construct
  misc_feature 1..32: note = Decoding Oligonucleotide
  source 1..32: mol_type = other DNA
ggctaatttattacctggataagtcggtaatc

SEQ ID NO: 1297 - DNA, 32 bp, synthetic construct
  misc_feature 1..32: note = Decoding Oligonucleotide
  source 1..32: mol_type = other DNA
accttaagaatggaatggataagtcggtaatc

SEQ ID NO: 1298 - DNA, 31 bp, synthetic construct
  misc_feature 1..31: note = Decoding Oligonucleotide
  source 1..31: mol_type = other DNA
acttaacttgagtggggataagtcggtaatc

SEQ ID NO: 1299 - DNA, 33 bp, synthetic construct
  misc_feature 1..33: note = Decoding Oligonucleotide
  source 1..33: mol_type = other DNA
ggctaatttattacctggtatttcactctaatt

SEQ ID NO: 1300 - DNA, 33 bp, synthetic construct
  misc_feature 1..33: note = Decoding Oligonucleotide
  source 1..33: mol_type = other DNA
accttaagaatggaatggtatttcactctaatt

SEQ ID NO: 1301 - DNA, 32 bp, synthetic construct
  misc_feature 1..32: note = Decoding Oligonucleotide
  source 1..32: mol_type = other DNA
acttaacttgagtggggtatttcactctaatt

SEQ ID NO: 1302 - DNA, 32 bp, synthetic construct
  misc_feature 1..32: note = Decoding Oligonucleotide
  source 1..32: mol_type = other DNA
ggctaatttattacctagctagataaagtacc

SEQ ID NO: 1303 - DNA, 32 bp, synthetic construct
  misc_feature 1..32: note = Decoding Oligonucleotide
  source 1..32: mol_type = other DNA
accttaagaatggaatagctagataaagtacc

SEQ ID NO: 1304 - DNA, 31 bp, synthetic construct
  misc_feature 1..31: note = Decoding Oligonucleotide
  source 1..31: mol_type = other DNA
acttaacttgagtggagctagataaagtacc

SEQ ID NO: 1305 - DNA, 32 bp, synthetic construct
  misc_feature 1..32: note = Decoding Oligonucleotide
  source 1..32: mol_type = other DNA
ggctaatttattacctccgaaactacgattat

SEQ ID NO: 1306 - DNA, 32 bp, synthetic construct
  misc_feature 1..32: note = Decoding Oligonucleotide
  source 1..32: mol_type = other DNA
accttaagaatggaatccgaaactacgattat

SEQ ID NO: 1307 - DNA, 31 bp, synthetic construct
  misc_feature 1..31: note = Decoding Oligonucleotide
  source 1..31: mol_type = other DNA
acttaacttgagtggccgaaactacgattat

SEQ ID NO: 1308 - DNA, 32 bp, synthetic construct
  misc_feature 1..32: note = Decoding Oligonucleotide
  source 1..32: mol_type = other DNA
ggctaatttattacctcatcgaatttatgtta

SEQ ID NO: 1309 - DNA, 32 bp, synthetic construct
  misc_feature 1..32: note = Decoding Oligonucleotide
  source 1..32: mol_type = other DNA
accttaagaatggaatcatcgaatttatgtta

SEQ ID NO: 1310 - DNA, 31 bp, synthetic construct
  misc_feature 1..31: note = Decoding Oligonucleotide
  source 1..31: mol_type = other DNA
acttaacttgagtggcatcgaatttatgtta

SEQ ID NO: 1311 - DNA, 30 bp, synthetic construct
  misc_feature 1..30: note = Decoding Oligonucleotide
  source 1..30: mol_type = other DNA
ggctaatttattacctccgtaattctaccg

SEQ ID NO: 1312 - DNA, 30 bp, synthetic construct
  misc_feature 1..30: note = Decoding Oligonucleotide
  source 1..30: mol_type = other DNA
accttaagaatggaatccgtaattctaccg

SEQ ID NO: 1313 - DNA, 29 bp, synthetic construct
  misc_feature 1..29: note = Decoding Oligonucleotide
  source 1..29: mol_type = other DNA
acttaacttgagtggccgtaattctaccg

SEQ ID NO: 1314 - DNA, 29 bp, synthetic construct
  misc_feature 1..29: note = Decoding Oligonucleotide
  source 1..29: mol_type = other DNA
ggctaatttattacctgggtaatcgtcac

SEQ ID NO: 1315 - DNA, 29 bp, synthetic construct
  misc_feature 1..29: note = Decoding Oligonucleotide
  source 1..29: mol_type = other DNA
accttaagaatggaatgggtaatcgtcac

SEQ ID NO: 1316 - DNA, 28 bp, synthetic construct
  misc_feature 1..28: note = Decoding Oligonucleotide
  source 1..28: mol_type = other DNA
acttaacttgagtgggggtaatcgtcac

SEQ ID NO: 1317 - DNA, 31 bp, synthetic construct
  misc_feature 1..31: note = Decoding Oligonucleotide
  source 1..31: mol_type = other DNA
ggctaatttattacctagtgttatgagaagc

SEQ ID NO: 1318 - DNA, 31 bp, synthetic construct
  misc_feature 1..31: note = Decoding Oligonucleotide
  source 1..31: mol_type = other DNA
accttaagaatggaatagtgttatgagaagc

SEQ ID NO: 1319 - DNA, 30 bp, synthetic construct
  misc_feature 1..30: note = Decoding Oligonucleotide
  source 1..30: mol_type = other DNA
acttaacttgagtggagtgttatgagaagc

SEQ ID NO: 1320 - DNA, 31 bp, synthetic construct
  misc_feature 1..31: note = Decoding Oligonucleotide
  source 1..31: mol_type = other DNA
ggctaatttattacctctgcaatagaagaga

SEQ ID NO: 1321 - DNA, 31 bp, synthetic construct
  misc_feature 1..31: note = Decoding Oligonucleotide
  source 1..31: mol_type = other DNA
accttaagaatggaatctgcaatagaagaga

SEQ ID NO: 1322 - DNA, 30 bp, synthetic construct
  misc_feature 1..30: note = Decoding Oligonucleotide
  source 1..30: mol_type = other DNA
acttaacttgagtggctgcaatagaagaga

SEQ ID NO: 1323 - DNA, 30 bp, synthetic construct
  misc_feature 1..30: note = Decoding Oligonucleotide
  source 1..30: mol_type = other DNA
ggctaatttattacctgatttttcagcgac

SEQ ID NO: 1324 - DNA, 30 bp, synthetic construct
  misc_feature 1..30: note = Decoding Oligonucleotide
  source 1..30: mol_type = other DNA
accttaagaatggaatgatttttcagcgac

SEQ ID NO: 1325 - DNA, 29 bp, synthetic construct
  misc_feature 1..29: note = Decoding Oligonucleotide
  source 1..29: mol_type = other DNA
acttaacttgagtgggatttttcagcgac

SEQ ID NO: 1326 - DNA, 32 bp, synthetic construct
  misc_feature 1..32: note = Decoding Oligonucleotide
  source 1..32: mol_type = other DNA
ggctaatttattacctcgtaatatgaagatca

SEQ ID NO: 1327 - DNA, 32 bp, synthetic construct
  misc_feature 1..32: note = Decoding Oligonucleotide
  source 1..32: mol_type = other DNA
accttaagaatggaatcgtaatatgaagatca

SEQ ID NO: 1328 - DNA, 31 bp, synthetic construct
  misc_feature 1..31: note = Decoding Oligonucleotide
  source 1..31: mol_type = other DNA
acttaacttgagtggcgtaatatgaagatca

SEQ ID NO: 1329 - DNA, 32 bp, synthetic construct
  misc_feature 1..32: note = Decoding Oligonucleotide
  source 1..32: mol_type = other DNA
ggctaatttattacctgaattggaatattcgt

SEQ ID NO: 1330 - DNA, 32 bp, synthetic construct
  misc_feature 1..32: note = Decoding Oligonucleotide
  source 1..32: mol_type = other DNA
accttaagaatggaatgaattggaatattcgt

SEQ ID NO: 1331 - DNA, 31 bp, synthetic construct
  misc_feature 1..31: note = Decoding Oligonucleotide
  source 1..31: mol_type = other DNA
acttaacttgagtgggaattggaatattcgt

SEQ ID NO: 1332 - DNA, 33 bp, synthetic construct
  misc_feature 1..33: note = Decoding Oligonucleotide
  source 1..33: mol_type = other DNA
ggctaatttattacctggtattaatcttgaaga

SEQ ID NO: 1333 - DNA, 33 bp, synthetic construct
  misc_feature 1..33: note = Decoding Oligonucleotide
  source 1..33: mol_type = other DNA
accttaagaatggaatggtattaatcttgaaga

SEQ ID NO: 1334 - DNA, 32 bp, synthetic construct
  misc_feature 1..32: note = Decoding Oligonucleotide
  source 1..32: mol_type = other DNA
acttaacttgagtggggtattaatcttgaaga

SEQ ID NO: 1335 - DNA, 29 bp, synthetic construct
  misc_feature 1..29: note = Decoding Oligonucleotide
  source 1..29: mol_type = other DNA
ggctaatttattacctacgatgtattgcc

SEQ ID NO: 1336 - DNA, 29 bp, synthetic construct
  misc_feature 1..29: note = Decoding Oligonucleotide
  source 1..29: mol_type = other DNA
accttaagaatggaatacgatgtattgcc

SEQ ID NO: 1337 - DNA, 28 bp, synthetic construct
  misc_feature 1..28: note = Decoding Oligonucleotide
  source 1..28: mol_type = other DNA
acttaacttgagtggacgatgtattgcc

SEQ ID NO: 1338 - DNA, 32 bp, synthetic construct
  misc_feature 1..32: note = Decoding Oligonucleotide
  source 1..32: mol_type = other DNA
ggctaatttattacctcatatacaaacggtac

SEQ ID NO: 1339 - DNA, 32 bp, synthetic construct
  misc_feature 1..32: note = Decoding Oligonucleotide
  source 1..32: mol_type = other DNA
accttaagaatggaatcatatacaaacggtac

SEQ ID NO: 1340 - DNA, 31 bp, synthetic construct
  misc_feature 1..31: note = Decoding Oligonucleotide
  source 1..31: mol_type = other DNA
acttaacttgagtggcatatacaaacggtac

SEQ ID NO: 1341 - DNA, 31 bp, synthetic construct
  misc_feature 1..31: note = Decoding Oligonucleotide
  source 1..31: mol_type = other DNA
ggctaatttattacctttccgatcttaactt

SEQ ID NO: 1342 - DNA, 31 bp, synthetic construct
  misc_feature 1..31: note = Decoding Oligonucleotide
  source 1..31: mol_type = other DNA
accttaagaatggaatttccgatcttaactt

SEQ ID NO: 1343 - DNA, 30 bp, synthetic construct
  misc_feature 1..30: note = Decoding Oligonucleotide
  source 1..30: mol_type = other DNA
acttaacttgagtggttccgatcttaactt

SEQ ID NO: 1344 - DNA, 31 bp, synthetic construct
  misc_feature 1..31: note = Decoding Oligonucleotide
  source 1..31: mol_type = other DNA
ggctaatttattacctgtgttaattcgcatc

SEQ ID NO: 1345 - DNA, 31 bp, synthetic construct
  misc_feature 1..31: note = Decoding Oligonucleotide
  source 1..31: mol_type = other DNA
accttaagaatggaatgtgttaattcgcatc

SEQ ID NO: 1346 - DNA, 30 bp, synthetic construct
  misc_feature 1..30: note = Decoding Oligonucleotide
  source 1..30: mol_type = other DNA
acttaacttgagtgggtgttaattcgcatc

SEQ ID NO: 1347 - DNA, 30 bp, synthetic construct
  misc_feature 1..30: note = Decoding Oligonucleotide
  source 1..30: mol_type = other DNA
ggctaatttattacctcgtttaattgggct

SEQ ID NO: 1348 - DNA, 30 bp, synthetic construct
  misc_feature 1..30: note = Decoding Oligonucleotide
  source 1..30: mol_type = other DNA
accttaagaatggaatcgtttaattgggct

SEQ ID NO: 1349 - DNA, 29 bp, synthetic construct
  misc_feature 1..29: note = Decoding Oligonucleotide
  source 1..29: mol_type = other DNA
acttaacttgagtggcgtttaattgggct

SEQ ID NO: 1350 - DNA, 31 bp, synthetic construct
  misc_feature 1..31: note = Decoding Oligonucleotide
  source 1..31: mol_type = other DNA
ggctaatttattacctcaacacttataacgg

SEQ ID NO: 1351 - DNA, 31 bp, synthetic construct
  misc_feature 1..31: note = Decoding Oligonucleotide
  source 1..31: mol_type = other DNA
accttaagaatggaatcaacacttataacgg

SEQ ID NO: 1352 - DNA, 30 bp, synthetic construct
  misc_feature 1..30: note = Decoding Oligonucleotide
  source 1..30: mol_type = other DNA
acttaacttgagtggcaacacttataacgg

SEQ ID NO: 1353 - DNA, 34 bp, synthetic construct
  misc_feature 1..34: note = Decoding Oligonucleotide
  source 1..34: mol_type = other DNA
ggctaatttattacctggaaataagtattatacc

SEQ ID NO: 1354 - DNA, 34 bp, synthetic construct
  misc_feature 1..34: note = Decoding Oligonucleotide
  source 1..34: mol_type = other DNA
accttaagaatggaatggaaataagtattatacc

SEQ ID NO: 1355 - DNA, 33 bp, synthetic construct
  misc_feature 1..33: note = Decoding Oligonucleotide
  source 1..33: mol_type = other DNA
acttaacttgagtggggaaataagtattatacc

SEQ ID NO: 1356 - DNA, 29 bp, synthetic construct
  misc_feature 1..29: note = Decoding Oligonucleotide
  source 1..29: mol_type = other DNA
ggctaatttattacctcttaacgacggtt

SEQ ID NO: 1357 - DNA, 29 bp, synthetic construct
  misc_feature 1..29: note = Decoding Oligonucleotide
  source 1..29: mol_type = other DNA
accttaagaatggaatcttaacgacggtt

SEQ ID NO: 1358 - DNA, 28 bp, synthetic construct
  misc_feature 1..28: note = Decoding Oligonucleotide
  source 1..28: mol_type = other DNA
acttaacttgagtggcttaacgacggtt

SEQ ID NO: 1359 - DNA, 33 bp, synthetic construct
  misc_feature 1..33: note = Decoding Oligonucleotide
  source 1..33: mol_type = other DNA
ggctaatttattacctgctcgttagaatttatt

SEQ ID NO: 1360 - DNA, 33 bp, synthetic construct
  misc_feature 1..33: note = Decoding Oligonucleotide
  source 1..33: mol_type = other DNA
accttaagaatggaatgctcgttagaatttatt

SEQ ID NO: 1361 - DNA, 32 bp, synthetic construct
  misc_feature 1..32: note = Decoding Oligonucleotide
  source 1..32: mol_type = other DNA
acttaacttgagtgggctcgttagaatttatt

SEQ ID NO: 1362 - DNA, 32 bp, synthetic construct
  misc_feature 1..32: note = Decoding Oligonucleotide
  source 1..32: mol_type = other DNA
ggctaatttattaccttctttatcatcattga

SEQ ID NO: 1363 - DNA, 32 bp, synthetic construct
  misc_feature 1..32: note = Decoding Oligonucleotide
  source 1..32: mol_type = other DNA
accttaagaatggaattctttatcatcattga

SEQ ID NO: 1364 - DNA, 31 bp, synthetic construct
  misc_feature 1..31: note = Decoding Oligonucleotide
  source 1..31: mol_type = other DNA
acttaacttgagtggtctttatcatcattga

SEQ ID NO: 1365 - DNA, 31 bp, synthetic construct
  misc_feature 1..31: note = Decoding Oligonucleotide
  source 1..31: mol_type = other DNA
ggctaatttattacctcgttcgcaatttatc

SEQ ID NO: 1366 - DNA, 31 bp, synthetic construct
  misc_feature 1..31: note = Decoding Oligonucleotide
  source 1..31: mol_type = other DNA
accttaagaatggaatcgttcgcaatttatc

SEQ ID NO: 1367 - DNA, 30 bp, synthetic construct
  misc_feature 1..30: note = Decoding Oligonucleotide
  source 1..30: mol_type = other DNA
acttaacttgagtggcgttcgcaatttatc

SEQ ID NO: 1368 - DNA, 31 bp, synthetic construct
  misc_feature 1..31: note = Decoding Oligonucleotide
  source 1..31: mol_type = other DNA
ggctaatttattacctcgatcttttgttgta

SEQ ID NO: 1369 - DNA, 31 bp, synthetic construct
  misc_feature 1..31: note = Decoding Oligonucleotide
  source 1..31: mol_type = other DNA
accttaagaatggaatcgatcttttgttgta

SEQ ID NO: 1370 - DNA, 30 bp, synthetic construct
  misc_feature 1..30: note = Decoding Oligonucleotide
  source 1..30: mol_type = other DNA
acttaacttgagtggcgatcttttgttgta

SEQ ID NO: 1371 - DNA, 32 bp, synthetic construct
  misc_feature 1..32: note = Decoding Oligonucleotide
  source 1..32: mol_type = other DNA
ggctaatttattacctaccactcaatataaga

SEQ ID NO: 1372 - DNA, 32 bp, synthetic construct
  misc_feature 1..32: note = Decoding Oligonucleotide
  source 1..32: mol_type = other DNA
accttaagaatggaataccactcaatataaga

SEQ ID NO: 1373 - DNA, 31 bp, synthetic construct
  misc_feature 1..31: note = Decoding Oligonucleotide
  source 1..31: mol_type = other DNA
acttaacttgagtggaccactcaatataaga

SEQ ID NO: 1374 - DNA, 30 bp, synthetic construct
  misc_feature 1..30: note = Decoding Oligonucleotide
  source 1..30: mol_type = other DNA
ggctaatttattacctccgtaaattcggtc

SEQ ID NO: 1375 - DNA, 30 bp, synthetic construct
  misc_feature 1..30: note = Decoding Oligonucleotide
  source 1..30: mol_type = other DNA
accttaagaatggaatccgtaaattcggtc

SEQ ID NO: 1376 - DNA, 29 bp, synthetic construct
  misc_feature 1..29: note = Decoding Oligonucleotide
  source 1..29: mol_type = other DNA
acttaacttgagtggccgtaaattcggtc

SEQ ID NO: 1377 - DNA, 30 bp, synthetic construct
  misc_feature 1..30: note = Decoding Oligonucleotide
  source 1..30: mol_type = other DNA
ggctaatttattacctcgtgagatttctga

SEQ ID NO: 1378 - DNA, 30 bp, synthetic construct
  misc_feature 1..30: note = Decoding Oligonucleotide
  source 1..30: mol_type = other DNA
accttaagaatggaatcgtgagatttctga

SEQ ID NO: 1379 - DNA, 29 bp, synthetic construct
  misc_feature 1..29: note = Decoding Oligonucleotide
  source 1..29: mol_type = other DNA
acttaacttgagtggcgtgagatttctga

SEQ ID NO: 1380 - DNA, 31 bp, synthetic construct
  misc_feature 1..31: note = Decoding Oligonucleotide
  source 1..31: mol_type = other DNA
ggctaatttattacctcgaagcatagaatag

SEQ ID NO: 1381 - DNA, 31 bp, synthetic construct
  misc_feature 1..31: note = Decoding Oligonucleotide
  source 1..31: mol_type = other DNA
accttaagaatggaatcgaagcatagaatag

SEQ ID NO: 1382 - DNA, 30 bp, synthetic construct
  misc_feature 1..30: note = Decoding Oligonucleotide
  source 1..30: mol_type = other DNA
acttaacttgagtggcgaagcatagaatag

SEQ ID NO: 1383 - DNA, 30 bp, synthetic construct
  misc_feature 1..30: note = Decoding Oligonucleotide
  source 1..30: mol_type = other DNA
ggctaatttattacctaaaatacgggagac

SEQ ID NO: 1384 - DNA, 30 bp, synthetic construct
  misc_feature 1..30: note = Decoding Oligonucleotide
  source 1..30: mol_type = other DNA
accttaagaatggaataaaatacgggagac

SEQ ID NO: 1385 - DNA, 29 bp, synthetic construct
  misc_feature 1..29: note = Decoding Oligonucleotide
  source 1..29: mol_type = other DNA
acttaacttgagtggaaaatacgggagac

SEQ ID NO: 1386 - DNA, 31 bp, synthetic construct
  misc_feature 1..31: note = Decoding Oligonucleotide
  source 1..31: mol_type = other DNA
ggctaatttattacctcgcatggtataacat

SEQ ID NO: 1387 - DNA, 31 bp, synthetic construct
  misc_feature 1..31: note = Decoding Oligonucleotide
  source 1..31: mol_type = other DNA
accttaagaatggaatcgcatggtataacat

SEQ ID NO: 1388 - DNA, 30 bp, synthetic construct
  misc_feature 1..30: note = Decoding Oligonucleotide
  source 1..30: mol_type = other DNA
acttaacttgagtggcgcatggtataacat

SEQ ID NO: 1389 - DNA, 31 bp, synthetic construct
  misc_feature 1..31: note = Decoding Oligonucleotide
  source 1..31: mol_type = other DNA
ggctaatttattacctacgctgatttataga

SEQ ID NO: 1390 - DNA, 31 bp, synthetic construct
  misc_feature 1..31: note = Decoding Oligonucleotide
  source 1..31: mol_type = other DNA
accttaagaatggaatacgctgatttataga

SEQ ID NO: 1391 - DNA, 30 bp, synthetic construct
  misc_feature 1..30: note = Decoding Oligonucleotide
  source 1..30: mol_type = other DNA
acttaacttgagtggacgctgatttataga

SEQ ID NO: 1392 - DNA, 33 bp, synthetic construct
  misc_feature 1..33: note = Decoding Oligonucleotide
  source 1..33: mol_type = other DNA
ggctaatttattacctcgtagggtaaatagatt

SEQ ID NO: 1393 - DNA, 33 bp, synthetic construct
  misc_feature 1..33: note = Decoding Oligonucleotide
  source 1..33: mol_type = other DNA
accttaagaatggaatcgtagggtaaatagatt

SEQ ID NO: 1394 - DNA, 32 bp, synthetic construct
  misc_feature 1..32: note = Decoding Oligonucleotide
  source 1..32: mol_type = other DNA
acttaacttgagtggcgtagggtaaatagatt

SEQ ID NO: 1395 - DNA, 33 bp, synthetic construct
  misc_feature 1..33: note = Decoding Oligonucleotide
  source 1..33: mol_type = other DNA
ggctaatttattacctcattcacagtaatttag

SEQ ID NO: 1396 - DNA, 33 bp, synthetic construct
  misc_feature 1..33: note = Decoding Oligonucleotide
  source 1..33: mol_type = other DNA
accttaagaatggaatcattcacagtaatttag

SEQ ID NO: 1397 - DNA, 32 bp, synthetic construct
  misc_feature 1..32: note = Decoding Oligonucleotide
  source 1..32: mol_type = other DNA
acttaacttgagtggcattcacagtaatttag

SEQ ID NO: 1398 - DNA, 15 bp, synthetic construct
  misc_feature 1..15: note = Signal Oligonucleotide
  source 1..15: mol_type = other DNA
ccactcaagttaagt

SEQ ID NO: 1399 - DNA, 16 bp, synthetic construct
  misc_feature 1..16: note = Signal Oligonucleotide
  source 1..16: mol_type = other DNA
attccattcttaaggt

SEQ ID NO: 1400 - DNA, 16 bp, synthetic construct
  misc_feature 1..16: note = Signal Oligonucleotide
  source 1..16: mol_type = other DNA
aggtaataaattagcc